(19) United States
(12) Patent Application Publication
(10) Pub. No.: US 2025/0048922 A1
(43) Pub. Date: Feb. 6, 2025

(54) NITROGEN-CONTAINING HETEROCYCLIC COMPOUNDS FOR FORMING A PATTERNING COATING AND DEVICES INCORPORATING SAME

(71) Applicant: OTI Lumionics Inc., Mississauga (CA)

(72) Inventors: Michael HELANDER, Mississauga (CA); Yudai NAKAGAWA, Mississauga (CA)

(73) Assignee: OTI Lumionics Inc., Mississauga, ON (CA)

(21) Appl. No.: 18/900,522

(22) Filed: Sep. 27, 2024

Related U.S. Application Data
(63) Continuation of application No. PCT/IB2023/053152, filed on Mar. 30, 2023.
(60) Provisional application No. 63/325,455, filed on Mar. 30, 2022.

(51) Int. Cl. H10K 85/60; C07D 251/34; H10K 50/814

(52) U.S. Cl. CPC H10K 85/654; C07D 251/34; H10K 50/814

(57) ABSTRACT

A layered semiconductor device comprises a compound. The compound comprises a heterocyclic moiety and at least one fluorine (F)-containing moiety each bonded thereto. The heterocyclic moiety comprises nitrogen (N). The device may comprise first and second electrodes, and an active region comprising at least one semiconducting layer, where the active region is bounded, in a longitudinal aspect of the device by the electrodes and confined, in a lateral aspect of the device, to an emissive region defined by the electrodes. The active region may be substantially devoid of the compound. The device may comprise a patterning coating comprising the compound and disposed on a first layer surface of an underlying layer, in a first portion of the lateral aspect and a deposited layer comprising a deposited material and disposed on a second layer surface, where the first portion is substantially devoid of a closed coating of the deposited material.

### RELATED APPLICATIONS

The present application is a continuation application of International Application No. PCT/IB2023/053152, filed Mar. 29, 2023, which claims the benefit of priority to U.S. Provisional Application No. 63/325,455 filed Mar. 30, 2022, the contents of which are incorporated herein by reference in their entirety.

### TECHNICAL FIELD

The present disclosure relates to layered semiconductor devices, and in some non-limiting examples to a patterning coating that may at least one of act, and be, a nucleation inhibiting coating for patterning at least one conductive deposited material, such as may be deposited during a device fabrication process, and in some non-limiting examples, in a fabrication process for an opto-electronic device having a plurality of (sub-) pixel emissive regions, each comprising first and second electrodes separated by a semiconducting layer.

### BACKGROUND

In an opto-electronic device such as an organic light emitting diode (OLED), at least one semiconducting layer comprising an emissive layer may be disposed between a pair of electrodes, such as an anode and a cathode. The anode and cathode may be electrically coupled with a power source and respectively generate holes and electrons that migrate toward each other through the at least one semiconducting layer. When a pair of holes and electrons combine, EM radiation, in the form of a photon, may be emitted by the emissive layer.

OLED display panels, such as an active-matrix OLED (AMOLED) panel, may comprise a plurality of pixels, each pixel further comprising a plurality of (including without limitation, one of: three, and four) sub-pixels. In some non-limiting examples, the various sub-pixels of a pixel may be characterized by one of: three, and four, different colors, including without limitation, R(ed), G(reen), and B(lue). Each (sub-) pixel may have an associated emissive region, comprising a stack of an associated pair of electrodes and at least one semiconducting layer between them. In some non-limiting examples, each sub-pixel of a pixel may emit EM radiation, including without limitation, photons, that have an associated wavelength spectrum characterized by a given color, including without limitation, one of, R(ed), G(reen), B(lue), and W(hite). In some non-limiting examples, the (sub-) pixels may be selectively driven by a driving circuit comprising at least one thin-film transistor (TFT) structure electrically coupled with conductive metal lines, in some non-limiting examples, within a substrate upon which the electrodes and the at least one semiconducting layer are deposited. Various coatings (layers) of such panels may, in some non-limiting examples, be formed by vacuum-based deposition processes.

In AMOLED panels, EM radiation may be emitted by a sub-pixel when a voltage is applied across an anode and a cathode of the sub-pixel. By controlling the voltage applied across the anode and the cathode, it may be possible to control the emission of EM radiation from each sub-pixel of such panel. In cases where a common cathode is provided across multiple sub-pixels, the voltage across the anode and the cathode in each sub-pixel may be controlled by modulating the voltage of the anode. In some non-limiting examples, the adjacent anodes may be spaced apart in a lateral aspect, and at least one non-emissive region may be provided therebetween.

In some applications, there may be an aim to provide a conductive deposited layer in a pattern for each (sub-) pixel of the panel across at least one of: cross-sectional, and lateral, aspect thereof, by selective deposition of a closed coating of the conductive deposited material to form a device feature, such as, without limitation, at least one of: an electrode, and a conductive element electrically coupled therewith, during the OLED manufacturing process.

One method for doing so, in some non-limiting applications, involves the interposition of a fine metal mask (FMM) during deposition of at least one of: an electrode, and a conductive element electrically coupled therewith. However, in some non-limiting examples, material used as electrodes may have substantially high evaporation temperatures, which may impact at least one of: the ability to re-use the FMM, and the accuracy of the pattern that may be achieved, with attendant increases in cost, effort, and complexity.

One method for doing so, in some non-limiting examples, involves depositing the electrode material and thereafter removing, including by a laser drilling process, unwanted regions thereof to form the pattern. However, the removal process often involves the creation and/or presence of debris, which may affect the yield of the manufacturing process.

In some non-limiting examples, such methods may have reduced applicability in certain applications. In some non-limiting examples, such methods may have reduced applicability with devices having certain topographical features.

In some non-limiting applications, there may be an aim to provide an improved mechanism for providing selective deposition of a conductive deposited material.

### BRIEF DESCRIPTION OF THE DRAWINGS

Examples of the present disclosure will now be described by reference to the following figures, in which identical reference numerals in different figures indicate at least one of: identical, and in some non-limiting examples, at least one of: analogous, and corresponding elements, and in which:

FIG. 1 is a simplified block diagram from a longitudinal aspect, of an example device having a plurality of layers in a lateral aspect, formed by selective deposition of a patterning coating in a first portion of the lateral aspect, followed by deposition of a closed coating of deposited material in a second portion thereof, according to an example in the present disclosure;

FIG. 2 is a schematic diagram showing an example process for depositing a patterning coating in a pattern on an exposed layer surface of an underlying layer in an example version of the device of FIG. 1, according to an example in the present disclosure;

FIG. 3 is a schematic diagram showing an example process for depositing a deposited material in the second portion on an exposed layer surface that comprises the deposited pattern of the patterning coating of FIG. 3, where the patterning coating is a nucleation-inhibiting coating (NIC);

FIG. 4A is a schematic diagram illustrating an example version of the device of FIG. 1 in a cross-sectional view;

FIG. 4B is a schematic diagram illustrating the device of FIG. 4A in a complementary plan view;

FIGS. 5A-5B are schematic diagrams that show various potential behaviours of a patterning coating at a deposition interface with a deposited layer in an example version of the device of FIG. 1 according to various examples in the present disclosure;

FIG. 6 is a simplified diagram, from a longitudinal aspect, of an example version of the device of FIG. 1, in which the closed coating of deposited material in the second portion forms a second electrode of an opto-electronic device, according to an example in the present disclosure;

FIG. 7 is a schematic diagram illustrating an example cross-sectional view of an example display panel having a plurality of layers, comprising at least one aperture therewithin, through which at least one electromagnetic signal may be exchanged according to an example in the present disclosure;

FIGS. 8A-8H are simplified block diagrams from a cross-sectional aspect, of example versions of the device of FIG. 1, showing various examples of possible interactions between the particle structure patterning coating and the particle structures according to examples in the present disclosure;

FIG. 9 is a schematic diagram illustrating an example cross-sectional view of an example version of the device of FIG. 6 with additional example deposition steps according to an example in the present disclosure;

FIG. 10 is a schematic diagram that may show example stages of an example process for manufacturing an example version of an OLED device having sub-pixel regions having a second electrode of different thickness according to an example in the present disclosure;

FIG. 11 is a schematic diagram illustrating an example cross-sectional view of an example version of an OLED device in which a second electrode is coupled with an auxiliary electrode according to an example in the present disclosure;

FIG. 12 is a schematic diagram illustrating an example cross-sectional view of an example version of an OLED device having a partition and a sheltered region, such as a recess, in a non-emissive region thereof according to an example in the present disclosure;

FIGS. 13A-13B are schematic diagrams that show example cross-sectional views of an example OLED device having a partition and a sheltered region, such as an aperture, in a non-emissive region, according to various examples in the present disclosure;

FIG. 14 is an example energy profile illustrating energy states of an adatom absorbed onto a surface according to an example in the present disclosure;

FIG. 15 is a schematic diagram illustrating the formation of a film nucleus according to an example in the present disclosure; and

FIG. 16 is a block diagram of an example computer device within a computing and communications environment that may be used for implementing devices and methods in accordance with representative examples of the present disclosure.

In the present disclosure, a reference numeral having at least one of: at least one numeric value (including without limitation, in at least one of: superscript, and subscript), and at least one alphabetic character (including without limitation, in lower-case) appended thereto, may be considered to refer to at least one of: a particular instance, and subset thereof, of the feature (element) described by the reference numeral. Reference to the reference numeral without reference to the at least one of: the appended value(s), and the character(s), may, as the context dictates, refer generally to the feature(s) described by at least one of: the reference numeral, and the set of all instances described thereby. Similarly, a reference numeral may have the letter “x’ in the place of a numeric digit. Reference to such reference numeral may, as the context dictates, refer generally to feature(s) described by the reference numeral, where the character “x” is replaced by at least one of: a numeric digit, and the set of all instances described thereby.

In the present disclosure, for purposes of explanation and not limitation, specific details are set forth to provide a thorough understanding of the present disclosure, including without limitation, particular architectures, interfaces and techniques. In some instances, detailed descriptions of well-known systems, technologies, components, devices, circuits, methods, and applications are omitted to not obscure the description of the present disclosure with unnecessary detail.

Further, it will be appreciated that block diagrams reproduced herein can represent conceptual views of illustrative components embodying the principles of the technology.

Accordingly, the system and method components have been represented where appropriate by conventional symbols in the drawings, showing only those specific details that are pertinent to understanding the examples of the present disclosure, to not obscure the disclosure with details that will be readily apparent to those of ordinary skill in the art having the benefit of the description herein.

Any drawings provided herein may not be drawn to scale and may not be considered to limit the present disclosure in any way.

Any feature shown in dashed outline may in some examples be considered as optional.

### SUMMARY

It is an object of the present disclosure to obviate or mitigate at least one disadvantage of the prior art.

The present disclosure discloses a layered semiconductor device comprises a compound. The compound comprises a heterocyclic moiety and at least one fluorine (F)-containing moiety each bonded thereto. The heterocyclic moiety comprises nitrogen (N). The device may comprise first and second electrodes, and an active region comprising at least one semiconducting layer, where the active region is bounded, in a longitudinal aspect of the device by the electrodes and confined, in a lateral aspect of the device, to an emissive region defined by the electrodes. The active region may be substantially devoid of the compound. The device may comprise a patterning coating comprising the compound and disposed on a first layer surface of an underlying layer, in a first portion of the lateral aspect and a deposited layer comprising a deposited material and disposed on a second layer surface, where the first portion is substantially devoid of a closed coating of the deposited material.

According to a broad aspect, there is disclosed a layered semiconductor device comprising a compound, the compound comprising a heterocyclic moiety and at least one fluorine (F)-containing moiety each bonded thereto, wherein the heterocyclic moiety comprises nitrogen (N).

In some non-limiting examples, the compound may comprise a plurality of F-containing moieties.

In some non-limiting examples, the heterocyclic moiety may be a heterocyclic moiety of between about 5-24 members.

In some non-limiting examples, the F-containing moiety may be bonded to the heterocyclic moiety by a linker moiety.

In some non-limiting examples, a molecular structure of the compound may be represented by one of: Formula (HC-1), and Formula (HC-2):

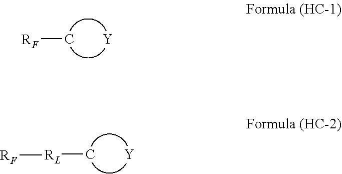

wherein:
   Y represents a member comprising at least one N atom of the heterocyclic moiety;
   R_{F} represents the F-containing moiety; and
   R_{L} represents a linker moiety.

In some non-limiting examples, Y may represent a member of between about 4-17 atoms.

In some non-limiting examples, Y may represent a member of between about 4-17 atoms.

In some non-limiting examples, the F-containing moiety may comprise at least one of: a substituted fluoroalkyl, an unsubstituted fluoroalkyl, a substituted fluoroalkoxy, an unsubstituted fluoroalkoxy, a substituted fluoroalkylsiloxy, an unsubstituted fluoroalkylsiloxy, a substituted fluorocycloalkyl, an unsubstituted fluorocycloalkyl, a substituted fluoroaryl, and an unsubstituted fluoroaryl.

In some non-limiting examples, the F-containing moiety may comprise a terminal unit comprising one of: CF₂CF₂H, CF₂CF₃, CH₂CF₂H, and CH₂CF₃.

In some non-limiting examples, the heterocyclic moiety may comprise at least one of: a monocyclic structure, a polycyclic structure, and a fused polycyclic structure.

In some non-limiting examples, the heterocyclic moiety may be a heteroaryl moiety.

In some non-limiting examples, the heterocyclic moiety may be a six-membered heterocyclic moiety.

In some non-limiting examples, the heterocyclic moiety may be a triazine moiety.

In some non-limiting examples, the F-containing moiety may comprise at least one of: a fluoroalkyl moiety, and a fluoroalkoxy moiety.

In some non-limiting examples, the linker moiety may comprise at least one of: an ether, a secondary amine, a tertiary amine, a substituted alkylene, an unsubstituted alkylene, a substituted fluoroalkylene, an unsubstituted fluoroalkylene, a substituted aryl, an unsubstituted aryl, a substituted fluoroaryl, an unsubstituted fluoroaryl, a substituted heteroaryl, and an unsubstituted heteroaryl, group.

In some non-limiting examples, the F-containing moiety may comprise at least one of: a C₃-C₁₅ fluoroalkyl, and a C₃-C₁₅ fluoroalkoxy.

In some non-limiting examples, the device may further comprise first and second electrodes, and an active region comprising at least one semiconducting layer, the active region bounded, in a longitudinal aspect of the device by the electrodes and confined, in a lateral aspect of the device, to an emissive region defined by the electrodes.

In some non-limiting examples, the active region may be substantially devoid of the compound.

In some non-limiting examples, the device may further comprise: a patterning coating comprising the compound and disposed on a first layer surface of an underlying layer, in a first portion of the lateral aspect; and a deposited layer comprising a deposited material and disposed on a second layer surface; wherein the first portion is substantially devoid of a closed coating of the deposited material.

In some non-limiting examples, the first portion may exclude at least a part of the emissive region.

In some non-limiting examples, the second electrode may comprise at least a part of the deposited layer as a layer thereof.

In some non-limiting examples, the first portion may include at least a part of the emissive region.

In some non-limiting examples, the device may further comprise an auxiliary electrode comprising the deposited layer as a layer thereof.

In some non-limiting examples, the device may further comprise a conductor electrically coupled with the second electrode.

### DESCRIPTION

### Layered Device

The present disclosure relates generally to layered semiconductor devices 100, and more specifically, to opto-electronic devices 600. An opto-electronic device 600 may generally encompass any device that converts electrical signals into EM radiation in the form of photons and vice versa. Non-limiting examples of opto-electronic devices 600 include organic light-emitting diodes (OLEDs).

Those having ordinary skill in the relevant art will appreciate that, while the present disclosure is directed to opto-electronic devices 600, the principles thereof may be applicable to any panel having a plurality of layers, including without limitation, at least one layer of conductive deposited material 331, including as a thin film, and in some non-limiting examples, through which electromagnetic (EM) signals may pass, including without limitation, one of partially, and entirely, at a non-zero angle relative to a plane of at least one of the layers.

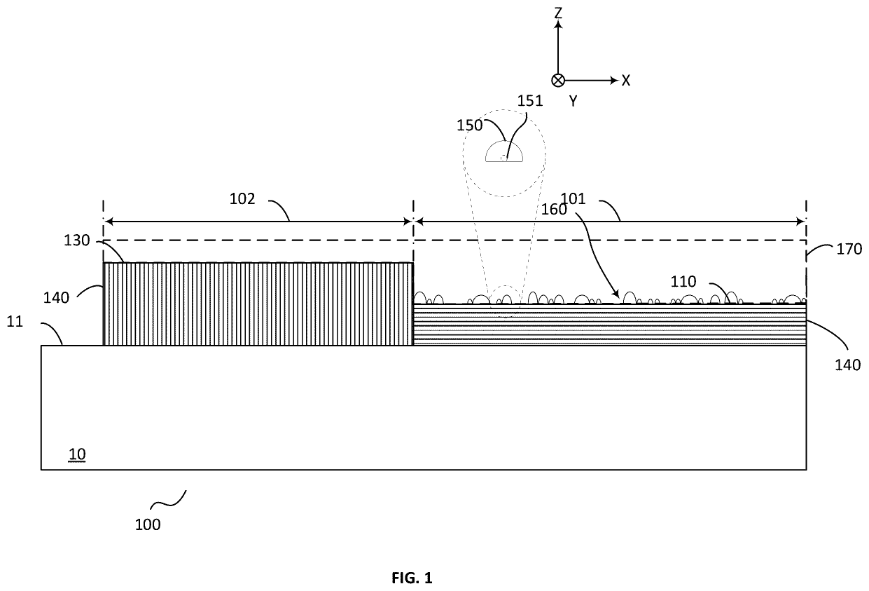

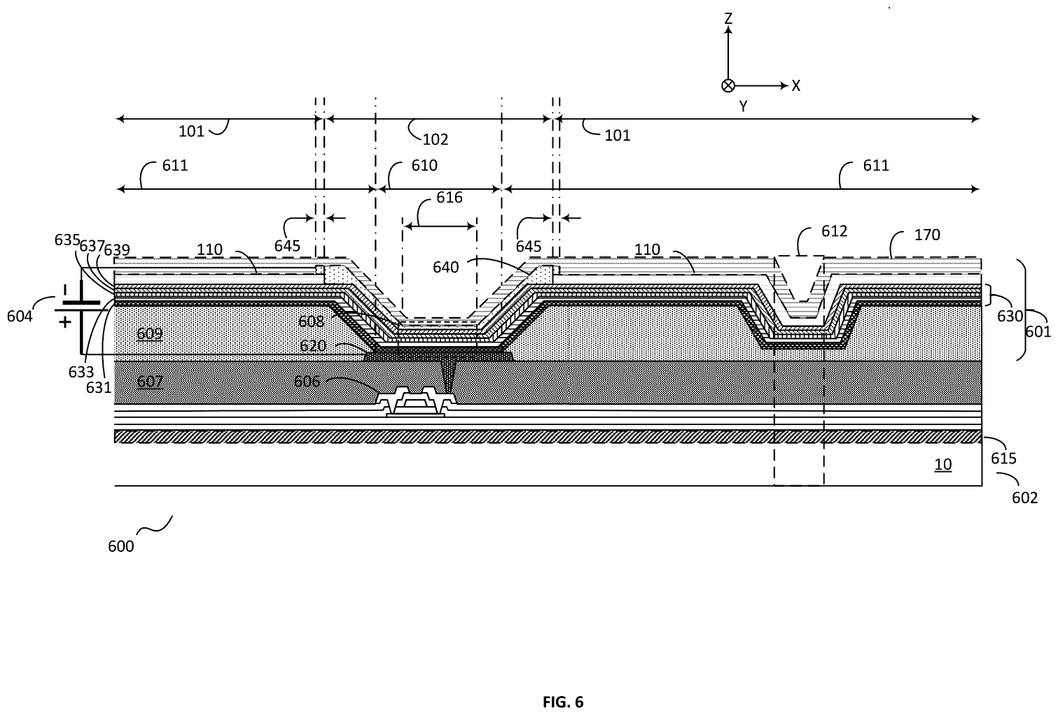

Turning now to FIG. 1, there may be shown a cross-sectional view of an example layered semiconductor device 100. In some non-limiting examples, as shown in greater detail in FIG. 6, the device 100 may comprise a plurality of layers deposited upon a substrate 10.

A lateral axis, identified as the X-axis, may be shown, together with a longitudinal axis, identified as the Z-axis. A second lateral axis, identified as the Y-axis, may be shown as being substantially transverse to both the X-axis and the Z-axis. At least one of the lateral axes may define a lateral aspect of the device 100. The longitudinal axis may define a longitudinal aspect of the device 100.

The layers of the device 100 may extend, in the lateral aspect, substantially parallel to a plane defined by the lateral axes. Those having ordinary skill in the relevant art will appreciate that the substantially planar representation shown in FIG. 1 may be, in some non-limiting examples, an abstraction for purposes of illustration. In some non-limiting examples, there may be, across a lateral extent of the device 100, localized substantially planar strata of different thicknesses and dimension, including, in some non-limiting examples, the substantially complete absence of at least one layer separated by non-planar transition areas (including lateral gaps and even discontinuities).

Thus, while for illustrative purposes, the device 100 may be shown in its longitudinal aspect as a substantially stratified structure of substantially parallel planar layers, such device may illustrate locally, a diverse topography to define features, each of which may substantially exhibit the stratified profile discussed in the longitudinal aspect.

In some non-limiting examples, a lateral aspect of an exposed layer surface 11 of the device 100 may comprise a first portion 101 and a second portion 102. In some non-limiting examples, the second portion 102 may comprise that part of the exposed layer surface 11 of the device 100 that lies beyond the first portion 101.

### Patterning

In some non-limiting examples, in the first portion 101, a patterning coating 110, which may, in some non-limiting examples, be a nucleation inhibiting compound, comprising a patterning material 211, which in some non-limiting examples, may be an NIC material, may be selectively deposited as a closed coating 140 on the exposed layer surface 11 of an underlying layer 210, including without limitation, a substrate 10, of the device 100, only in the first portion 101. However, in the second portion 102, the exposed layer surface 11 of the underlying layer 210 may be substantially devoid of a closed coating 140 of the patterning material 211.

### Patterning Coating

The patterning coating 110 may comprise a patterning material 211. In some non-limiting examples, the patterning coating 110 may comprise a closed coating 140 of the patterning material 211.

The patterning coating 110 may provide an exposed layer surface 11 with a substantially low propensity (including without limitation, a substantially low initial sticking probability) (in some non-limiting examples, under the conditions identified in the dual QCM technique described by Walker et al.) against the deposition of a deposited material 331 to be deposited thereon upon exposing such surface to a vapor flux 332 of the deposited material 331, which, in some non-limiting examples, may be substantially less than the propensity against the deposition of the deposited material 331 to be deposited on the exposed layer surface 11 of the underlying layer 210 of the device 100, upon which the patterning coating 110 has been deposited.

Because of the attributes, including without limitation, a low initial sticking probability, of at least one of: the patterning coating 110, and the patterning material 211, in some non-limiting examples, when deposited as at least one of: a film, and a coating, in a form, and under similar circumstances to the deposition of the patterning coating 110 within the device 100, against the deposition of the deposited material 331, the exposed layer surface 11 of the first portion 101 comprising the patterning coating 110 may be substantially devoid of a closed coating 140 of the deposited material 331.

However, exposure of the device 100 to a vapor flux 332 of the deposited material 331 may, in some non-limiting examples, result in the formation of a closed coating 140 of a deposited layer 130 of the deposited material 331 in the second portion 102, where the exposed layer surface 11 of the underlying layer 210 may be substantially devoid of the patterning coating 110.

Thus, in some non-limiting examples, the patterning coating 110 may be a nucleation inhibiting coating (NIC) that provides high deposition (patterning) contrast against subsequent deposition of the deposited material 331, such that the deposited material 331 tends not to be deposited, in some non-limiting examples, as a closed coating 140, where the patterning coating 110 has been deposited.

In some non-limiting examples, the patterning coating 110 may comprise a patterning material 211. In some non-limiting examples, the patterning material 211 may comprise an NIC material. In some non-limiting examples, the patterning coating 110 may comprise a closed coating 140 of the patterning material 211.

In some non-limiting examples, there may be scenarios calling for providing a patterning coating 110 for causing formation of a discontinuous layer 160 of at least one particle structure 150, upon the patterning coating 110 in the first portion 101 being subjected to a vapor flux 332 of a deposited material 331. In at least some applications, the attributes of the patterning coating 110 may be such that a closed coating 140 of the deposited material 331 may be formed in the second portion 102, which may be substantially devoid of the patterning coating 110, while only a discontinuous layer 160 of at least one particle structure 150 having at least one characteristic may be formed in the first portion 101 on the patterning coating 110.

For purposes of simplicity of discussion, in the present disclosure, to the extent that a patterning coating 110 is deposited to act as a base for the deposition of at least one particle structure 150 thereon, such patterning coating 110 may be designated as a particle structure patterning coating 110ₚ. By contrast, to the extent that a patterning coating 110 is deposited in a first portion 101 to substantially preclude formation in such first portion 101 of a closed coating 140 of the deposited layer 130, thus restricting the deposition of a closed coating 140 of the deposited layer 130 to a second portion 102, such patterning coating 110 may be designated as a non-particle structure patterning coating 110ₙ. Those having ordinary skill in the relevant art will appreciate that in some non-limiting examples, a patterning coating 110 may act as both a particle structure patterning coating 110ₚ and a non-particle structure patterning coating 110ₙ.

In some non-limiting examples, there may be scenarios calling for formation of a discontinuous layer 160 of at least one particle structure 150 of a deposited material 331, which may be, in some non-limiting examples, of one of: a metal, and a metal alloy (metal/alloy), including without limitation, at least one of: Yb, Ag, Mg, and Ag-containing materials, including without limitation, MgAg, in the second portion 102, while depositing a closed coating 140 of the deposited material 331 having a thickness of, without limitation, one of no more than about: 100 nm, 50 nm, 25 nm, and 15 nm. In some non-limiting examples, an amount of the deposited material 331 deposited as a discontinuous layer 160 of at least one particle structure 150 in the first portion 101 may correspond to one of between about: 1-50%, 2-25%, 5-20%, and 7-10% of the amount of the deposited material 331 deposited as a closed coating 140 in the second portion 102, which, by way of non-limiting example may correspond to a thickness of one of no more than about: 100 nm, 75 nm, 50 nm, 25 nm, and 15 nm.

In some non-limiting examples, the patterning coating 110 may be disposed in a pattern that may be defined by at least one region therein that may be substantially devoid of a closed coating 140 of the patterning coating 110.

In some non-limiting examples, the at least one region may separate the patterning coating 110 into a plurality of discrete fragments thereof. In some non-limiting examples, the plurality of discrete fragments of the patterning coating 110 may be physically spaced apart from one another in the lateral aspect thereof. In some non-limiting examples, the plurality of the discrete fragments of the patterning coating 110 may be arranged in a regular structure, including without limitation, an array (matrix), such that in some non-limiting examples, the discrete fragments of the patterning coating 110 may be configured in a repeating pattern.

In some non-limiting examples, at least one of the plurality of the discrete fragments of the patterning coating 110 may each correspond to an emissive region 610. In some non-limiting examples, an aperture ratio of the emissive regions 610 may be one of no more than about: 50%, 40%, 30%, and 20%.

In some non-limiting examples, the patterning coating 110 may be formed as a single monolithic coating.

### Attributes of Patterning Coating/Material

### Composition

In some non-limiting examples, the layered semiconductor device 100 may comprise a compound comprising a heterocyclic moiety and at least one fluorine (F)-containing moiety. In some non-limiting examples, at least one of: the patterning coating 110, and the patterning material 211 may comprise such a compound.

In some non-limiting examples, the heterocyclic moiety may comprise at least one nitrogen (N) atom.

In some non-limiting examples, the heterocyclic moiety and the F-containing moiety may be bonded together, including without limitation, one of: directly, and via other moieties, including without limitation, a linker moiety.

In some non-limiting examples, the heterocyclic moiety may comprise at least one of: a monocyclic structure, a polycyclic structure, and a fused polycyclic structure. In some non-limiting examples, the heterocyclic moiety may comprise one of: an aromatic structure, and a non-aromatic structure.

As used herein, the term “heterocyclic moiety” may generally refer to a cyclic moiety comprising at least one element that is not carbon (C), including without limitation, a cyclic moiety derived by substituting at least one C atom constituting at least one ring member of one of: a monocyclic, and polycyclic, hydrocarbon moiety, with a corresponding number of heteroatom.

In some non-limiting examples, the heterocyclic moiety may be a structure containing between one of about: 5-24, and 5-18 members. In some non-limiting examples, the heterocyclic moiety may comprise a plurality of C atoms and at least one N atom. In some non-limiting examples, the heterocyclic moiety may comprise additional heteroatoms, including without limitation, oxygen (O), sulfur (S), and boron (B).

In some non-limiting examples, the molecular structure of the compound may be represented by at least one of:

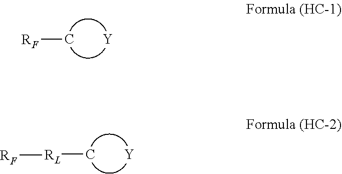

where:
Y represents the presence of at least one member comprising at least one N atom, which, together with the C atom shown in each formula, form the heterocyclic moiety;
R_{L} represents a linker moiety; and
R_{F} represents the F-containing moiety.

In the present disclosure, a reference to at least one of: the linker moiety R_{L}, and the F-containing moiety R_{F}, may be understood to comprise a moiety selected from any suitable moiety referenced herein.

In some non-limiting examples, the member Y may represent a member of between about 4-17 atoms, which, together with the C atom shown in each formula, may form the heterocyclic moiety. In some non-limiting examples, the member Y may represent one of between about: 4-13 atoms, 5-17 atoms, 5-13 atoms, 5-11 atoms, and 5-9 atoms. In some non-limiting examples, the member Y may comprise at least one N atom. In some non-limiting examples, the member Y may comprise at least one of: a C atom, and a heteroatom, including without limitation, N, O, B, and S, in addition to the at least one N atom. In some non-limiting examples, the member Y may be one of: unsubstituted, and substituted. In some non-limiting examples, at least one substituent of the member Y may comprise at least one of: substituted alkyl, unsubstituted alkyl, substituted alkoxy, unsubstituted alkoxy, substituted fluoroalkyl, unsubstituted fluoroalkyl, substituted fluoroalkoxy, unsubstituted fluoroalkoxy, substituted fluoroalkylsiloxy, unsubstituted fluoroalkylsiloxy, substituted fluorocycloalkyl, unsubstituted fluorocycloalkyl, substituted fluoroaryl, and unsubstituted fluoroaryl. In some non-limiting examples, at least one additional F-containing moiety may be bonded to at least one member of the member Y, including without limitation, one of: directly, and via other moieties, including without limitation, an additional linker moiety. In some non-limiting examples, such at least one additional F-containing moiety may be bonded to at least one C atom of the member Y.

In some non-limiting examples, the molecular structure of the compound may comprise a heterocyclic moiety comprising at least one N atom directly bonded to at least one C atom to form a ring, wherein at least one C atom directly bonded to the at least one N atom is bonded to the F-containing moiety. In some non-limiting examples, the at least one C atom may be bonded, including without limitation, one of: directly, and via a linker moiety, to the F-containing moiety. In some non-limiting examples, the molecular structure of such compound may be represented by at least one of:

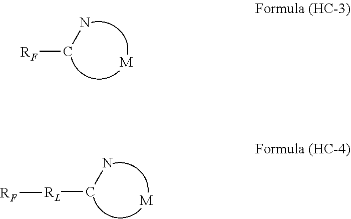

where:
M represents the presence of members which, together with the C and N atoms shown in each formula, form the heterocyclic moiety;
R_{L} represents a linker moiety; and
R_{F} represents the F-containing moiety.

In some non-limiting examples, the molecular structure of the compound may comprise a heterocyclic moiety comprising at least one N atom directly bonded to two adjacent C atoms to form a ring, wherein each C atom directly bonded to the at least one N atom is bonded to the F-containing moiety, including without limitation, one of: directly, and via a linker moiety. In some non-limiting examples, the molecular structure of such compound may be represented by at least one of:

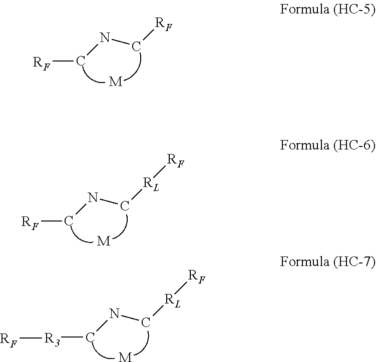

where:
M represents the presence of members which, together with the C and N atoms shown in each formula, form the heterocyclic moiety;
R_{L} each independently represents a linker moiety; and
R_{F} each independently represents the F-containing moiety.

Without wishing to be bound by any particular theory, there may be some scenarios calling for a compound, in which the molecular structure is configured such that a C atom bonded to the F-containing moiety is arranged adjacent, and bonded, to the at least one N atom of the heterocyclic moiety, in some applications for forming a patterning coating, since the presence of the F-containing moiety, which generally exhibits low surface tension, in proximity to the N atom of the heterocyclic moiety, which generally exhibits high surface tension, may lower the surface energy of at least one of: a thin film, and coating, formed by such compound, compared to other arrangements of the molecular structure. In some non-limiting examples, this may be achieved as a result of an increased likelihood that the molecular structure may be configured such that the F-containing moiety is oriented proximate to an exposed layer surface 11 of such thin film/coating, and the heterocyclic moiety being arranged distal to such exposed surface.

In some non-limiting examples, the heterocyclic moiety may comprise a monocyclic structure wherein the member Ytogether with the C atom shown in each of Formula (HC-1) and (HC-2), may form a single cyclic structure. In some non-limiting examples, such cyclic structure may comprise between about 3-6 atoms. In some non-limiting examples, the heterocyclic moiety may be a six-membered heterocyclic moiety. In some non-limiting examples, the heterocyclic moiety may be a heteroaryl moiety. In some non-limiting examples, the heterocyclic moiety may be a triazine moiety. In some non-limiting examples, the triazine moiety may be a 1,3,5-triazine moiety.

In some non-limiting examples, a monocyclic heterocyclic moiety may be represented by at least one of:

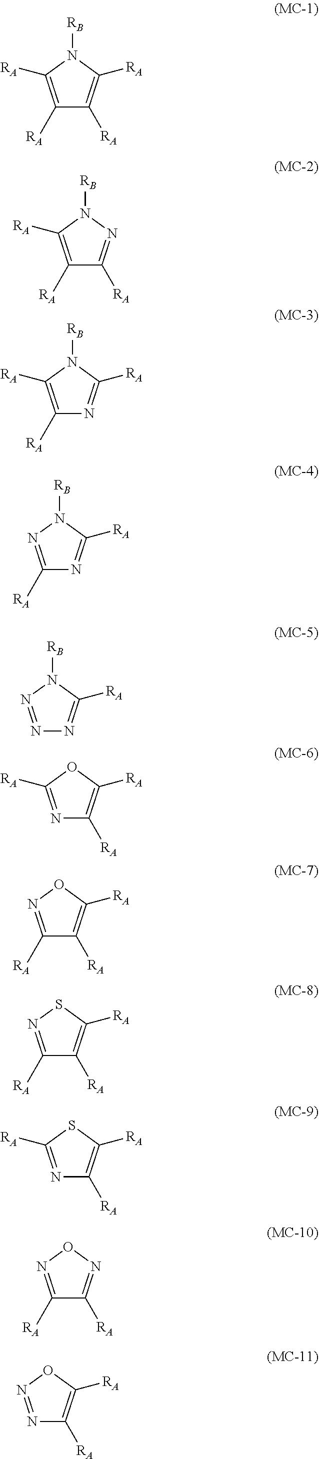

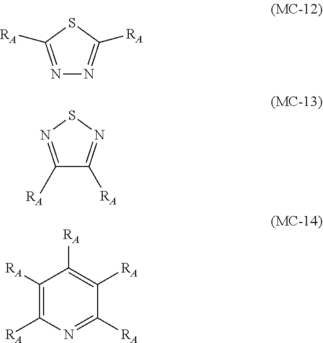

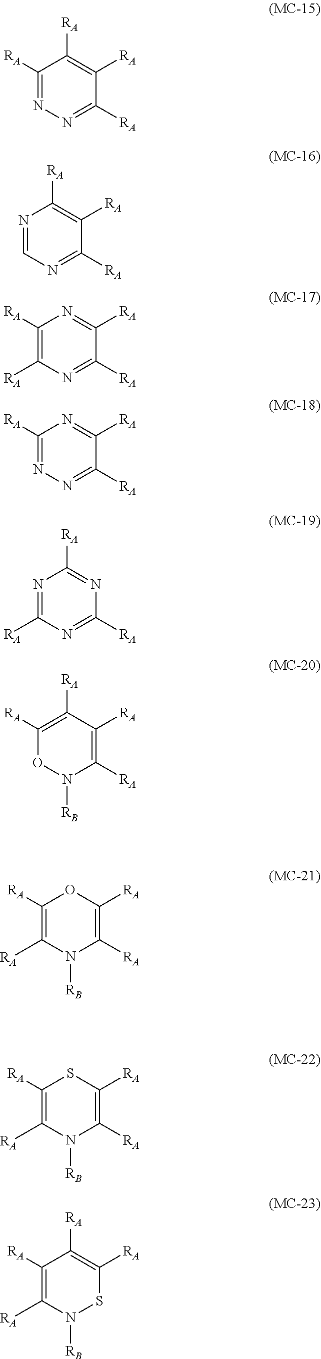

In each of the foregoing formulae (MC-1) to (MC-23), each of: R_{A} and R_{B}, may independently represent, upon each occurrence, one of: hydrogen (H), deutero (D,) R_{F}, R_{L}-R_{F}, substituted alkyl, unsubstituted alkyl, substituted fluoroalkyl, unsubstituted fluoroalkyl, substituted alkoxy, unsubstituted alkoxy, substituted fluoroalkoxy, unsubstituted fluoroalkoxy, substituted siloxy, unsubstituted siloxy, substituted fluoroalkylsiloxy, unsubstituted fluoroalkylsiloxy, substituted cycloalkyl, unsubstituted cycloalkyl, substituted fluorocycloalkyl, unsubstituted fluorocycloalkyl, substituted aryl, unsubstituted aryl, substituted fluoroaryl, unsubstituted fluoroaryl, substituted heteroaryl, and unsubstituted heteroaryl.

In some non-limiting examples, at least one R group of the molecular structure, which may correspond to at least one of: R_{A} and R_{B}, may comprise an F-containing moiety.

In some non-limiting examples, the heterocyclic moiety may comprise a fused polycyclic structure wherein the member Y, together with the C atom shown in each of Formula (HC-1) and (HC-2), may form a plurality of cyclic structures that are fused together such that adjacent cyclic structures may share a plurality of adjacent atoms.

In some non-limiting examples, such polycyclic heterocyclic moiety may be represented by at least one of:

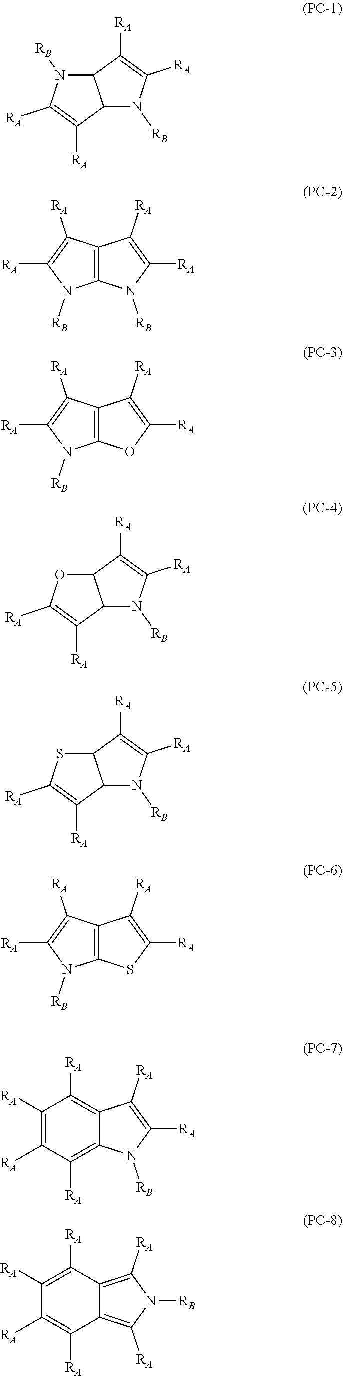

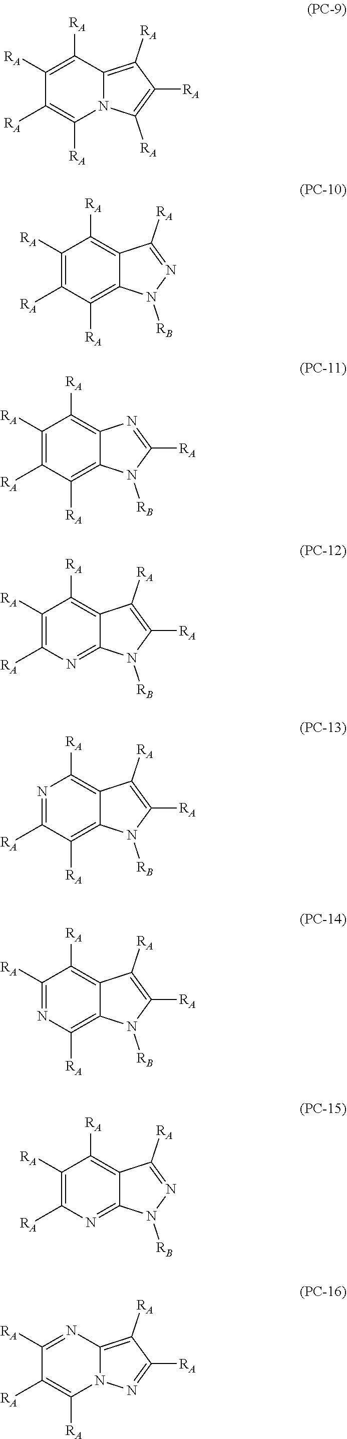

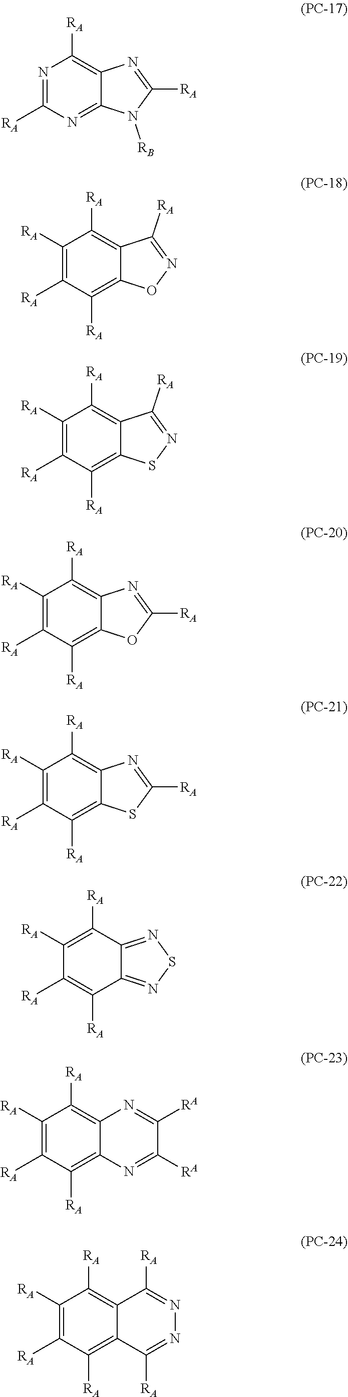

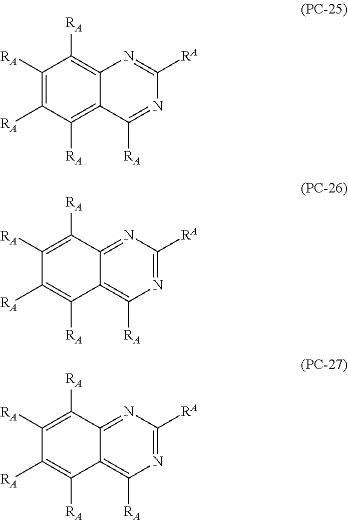

In each of the foregoing formulae, each of: R_{A} and R_{B}, may independently represent, upon each occurrence, one of: H, D, R_{F}, R_{L}-R_{F}, substituted alkyl, unsubstituted alkyl, substituted fluoroalkyl, unsubstituted fluoroalkyl, substituted alkoxy, unsubstituted alkoxy, substituted fluoroalkoxy, unsubstituted fluoroalkoxy, substituted siloxy, unsubstituted siloxy, substituted fluoroalkylsiloxy, unsubstituted fluoroalkylsiloxy, substituted cycloalkyl, unsubstituted cycloalkyl, substituted fluorocycloalkyl, unsubstituted fluorocycloalkyl, substituted aryl, unsubstituted aryl, substituted fluoroaryl, unsubstituted fluoroaryl, substituted heteroaryl, and unsubstituted heteroaryl.

In some non-limiting examples, at least one R group of the molecular structure, which may correspond to at least one of: R_{A}, and R_{B}, may comprise the F-containing moiety.

In some non-limiting examples, the heterocyclic moiety may comprise a polycyclic structure wherein the member Y, together with the C atom shown in each of Formula (HC-1) and (HC-2), may form an unfused polycyclic structure wherein a plurality of cyclic structures may be bonded together, in some non-limiting examples, by tethering, wherein covalent bond(s) are formed between members of the cyclic structures, by linking via at least one of: a single member of the cyclic structure(s), and a linker group that is bonded to a plurality of cyclic structures, to form the polycyclic structure.

In some non-limiting examples, such polycyclic heterocyclic moiety may comprise those formed by bonding a plurality of structures, selected from among Formulae: (MC-1)-(MC-23), and (PC-1)-(PC-27), with one another. In some non-limiting examples, the bond(s) formed between such plurality of structures may be formed in place of at least one of: any R_{A}, and R_{B}, of each such formula. The plurality of structures selected from at least one of Formulae: (MC-1)-(MC-23), and (PC-1)-(PC-27), which are bonded to one another to form the polycyclic heterocyclic moiety, may be selected independently of one another. In some non-limiting examples, the plurality of structures may be at least one of: substantially identical to, and different from, one another.

In some non-limiting examples, such polycyclic heterocyclic moiety may be represented by at least one of:

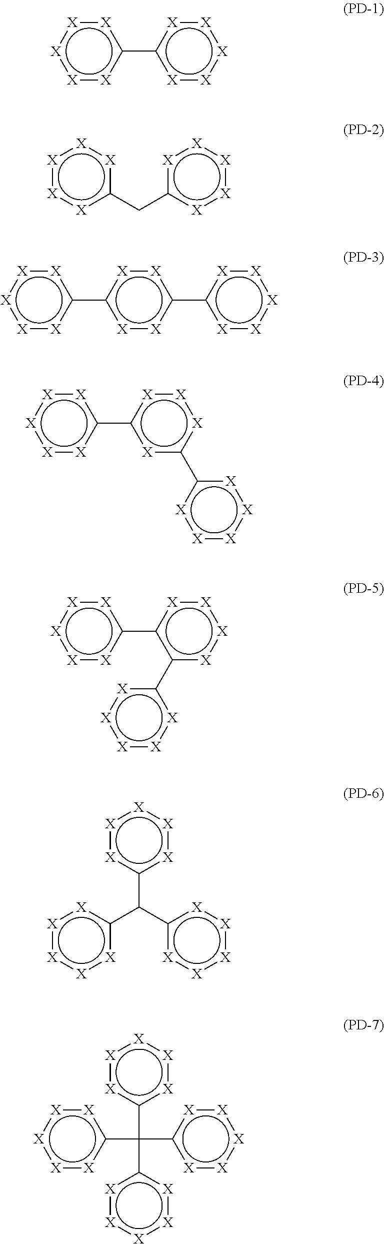

In each of the foregoing formulae (PD-1)-(PD-7), X independently represents, upon each occurrence, one of: C—R_{A}, and N—R_{B}, provided that any X that is immediately adjacent, and is bonded, to an X representing N—R_{B}, shall represent C—R_{A}.

In some non-limiting examples, R_{A} independently represents, upon each occurrence, one of: H, D, R_{F}, R_{L}-R_{F}, substituted alkyl, unsubstituted alkyl, substituted fluoroalkyl, unsubstituted fluoroalkyl, substituted alkoxy, unsubstituted alkoxy, substituted fluoroalkoxy, unsubstituted fluoroalkoxy, substituted siloxy, unsubstituted siloxy, substituted fluoroalkylsiloxy, unsubstituted fluoroalkylsiloxy, substituted cycloalkyl, unsubstituted cycloalkyl, substituted fluorocycloalkyl, unsubstituted fluorocycloalkyl, substituted aryl, unsubstituted aryl, substituted fluoroaryl, unsubstituted fluoroaryl, substituted heteroaryl, and unsubstituted heteroaryl.

In some non-limiting examples, at least one R group of the molecular structure, which may correspond to any of: R_{A}, or R_{B}, may comprise the F-containing moiety. In some non-limiting examples, the F-containing moiety may be bonded, including without limitation, one of: directly, and via a linker moiety, to a C atom.

In some non-limiting examples, the F-containing moiety may comprise at least one of: substituted fluoroalkyl, unsubstituted fluoroalkyl, substituted fluoroalkoxy, unsubstituted fluoroalkoxy, substituted fluoroalkylsiloxy, unsubstituted fluoroalkylsiloxy, substituted fluorocycloalkyl, unsubstituted fluorocycloalkyl, substituted fluoroaryl, and unsubstituted fluoroaryl. In some non-limiting examples, the F-containing moiety may comprise a fluorocarbon unit, including without limitation, at least one of: CF, CF₂, CF₃, and a CF₂H unit. In some non-limiting examples, the F-containing moiety may comprise a terminal unit comprising one of: CF₂CF₂H, CF₂CF₃, CH₂CF₂H, and CH₂CF₃. In some non-limiting examples, the terminal unit may correspond to a terminal part of the F-containing moiety that is distal with respect to the heterocyclic moiety, to which the F-containing moiety is attached.

In some non-limiting examples, the F-containing moiety may comprise a fluoroalkyl moiety, including without limitation, a C₃-C₁₅ fluoroalkyl.

In some non-limiting examples, the F-containing moiety may comprise a fluoroalkyl moiety of:

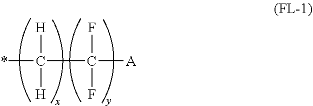

wherein:
x is an integer of between about: 0-6,
y is an integer of between about: 1-12, and
A is one of: H, and F.

In some non-limiting examples, x may be an integer of between about: 1-4, y may be an integer of between about: 3-10, and A may be one of: H, and F. In some non-limiting examples, x may be one of: 1, and 2, y may be one of: 3, 4, 6, and 8, and A may be one of: H, and F. In some non-limiting examples, x may be 2, y may be 1, and A may be one of: H, and F. In some non-limiting examples x and y may sum to one of no more than: 15, 12, 10, and 8.

In some non-limiting examples, the F-containing moiety may be a fluoroalkyl of:

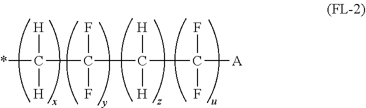

wherein:
x is an integer of between about: 1-6,
y is an integer of between about: 1-6,
z is an integer of between about: 1-6,
u is an integer of between about: 1-6, and
A is one of: H, and F.

In some non-limiting examples, x may be an integer of between about: 1-3, y may be an integer of between about: 1-6, z may be an integer of between about: 1-3, and u may be an integer of between about: 1-6. In some non-limiting examples, at least one of: y, and u, may be one of no more than: 5, 4, and 3. In some non-limiting examples, x, y, z, and u may sum to one of no more than: 15, 12, 10, and 8.

In some non-limiting examples, the F-containing moiety may comprise a fluoroalkoxy moiety, including without limitation, a C₃-C₁₅ fluoroalkoxy.

In some non-limiting examples, one of: a substituted fluoroalkoxy, and an unsubstituted fluoroalkoxy, may comprise one derived by substituting at least one H atom of an alkoxy group comprising, without limitation, between about: 1-15 C atoms, with a corresponding number of F atoms. In some non-limiting examples, a fluoroalkoxy may comprise one derived by attaching an ether bridging group to at least one of such one of: a substituted fluoroalkyl, and an unsubstituted fluoroalkyl.

In some non-limiting examples, one of: a substituted fluoroalkylsiloxy, and an unsubstituted fluoroalkylsiloxy, may comprise one derived by substituting at least one H atom of an alkylsiloxy group comprising, without limitation, between about: 1-15 C atoms, with a corresponding number of F atoms. In some non-limiting examples, a fluoroalkylsiloxy may comprise one derived by attaching a siloxane bridge to at least one of such one of: a substituted fluoroalkyl, and an unsubstituted fluoroalkyl.

In some non-limiting examples, the F-containing moiety may comprise a continuous fluorinated chain of C species with no more than 6 fluorinated C atoms. In some non-limiting examples, such moiety may comprise at least one of: a substituted fluoroalkyl, an unsubstituted fluoroalkyl, a substituted fluoroalkoxy, an unsubstituted fluoroalkoxy, a substituted fluoroalkylsiloxy, and an unsubstituted fluoroalkylsiloxy, in which no more than 6 fluorinated C atoms form a continuous fluorinated chain. In some non-limiting examples, the F-containing moiety may comprise a continuous fluorinated chain of C species with one of no more than: 5, 4, and 3 fluorinated C atoms.

As used herein, the term “non-fluorinated moiety” may generally refer to a moiety that is substantially devoid of F. In some non-limiting examples, the compound may comprise a non-fluorinated moiety. In some non-limiting examples, the compound may comprise an F-containing moiety and a non-fluorinated moiety. In some non-limiting examples, such a non-fluorinated moiety may comprise at least one of: substituted alkyl, unsubstituted alkyl, substituted alkoxy, unsubstituted alkoxy, substituted siloxy, unsubstituted siloxy, substituted cycloalkyl, unsubstituted cycloalkyl, substituted aryl, unsubstituted aryl, substituted heteroaryl, and unsubstituted heteroaryl, that are substantially devoid of F. In some non-limiting examples, alkyl may comprise between about 1-15 C atoms, alkoxy may comprise between about 1-15 C atoms, siloxy may comprise between about 1-15 Si atoms, cycloalkyl may comprise between about 1-15 C atoms, aryl may comprise between about 1-15 C atoms, and heteroaryl may comprise between about 1-15 C atoms. It will be appreciated that, in some non-limiting examples, at least one of the foregoing groups may comprise at least one substituent, which may comprise additional atoms.

In some non-limiting examples, the linker moiety, R_{L}, may comprise at least one of: an ether, a secondary amine, a tertiary amine, a substituted alkylene, an unsubstituted alkylene, a substituted fluoroalkylene, an unsubstituted fluoroalkylene, a substituted aryl, an unsubstituted aryl, a substituted fluoroaryl, an unsubstituted fluoroaryl, a substituted heteroaryl, and an unsubstituted heteroaryl, group.

In some non-limiting examples, the linker moiety may be an amine. In some non-limiting examples, the linker moiety may be an alkylene.

In some non-limiting examples, the compound may comprise an F-containing moiety selected from:

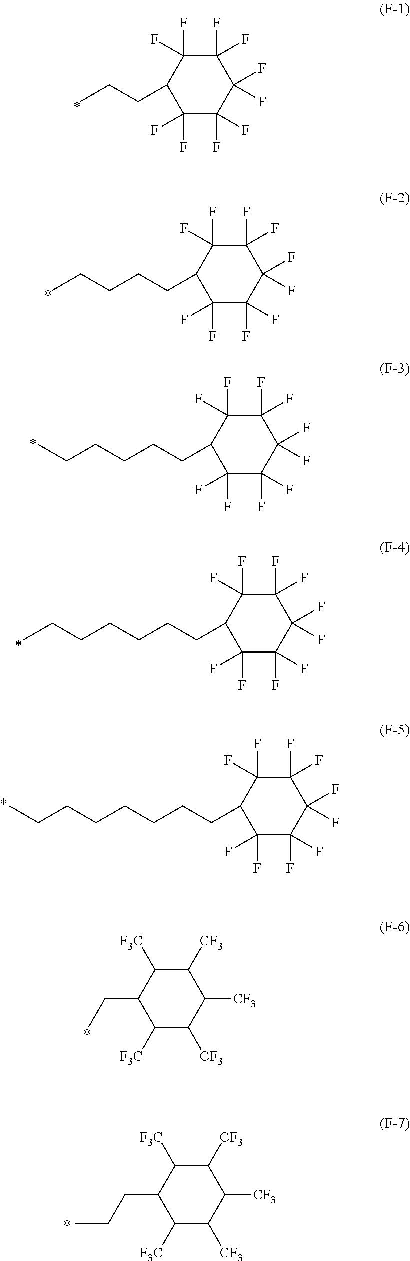

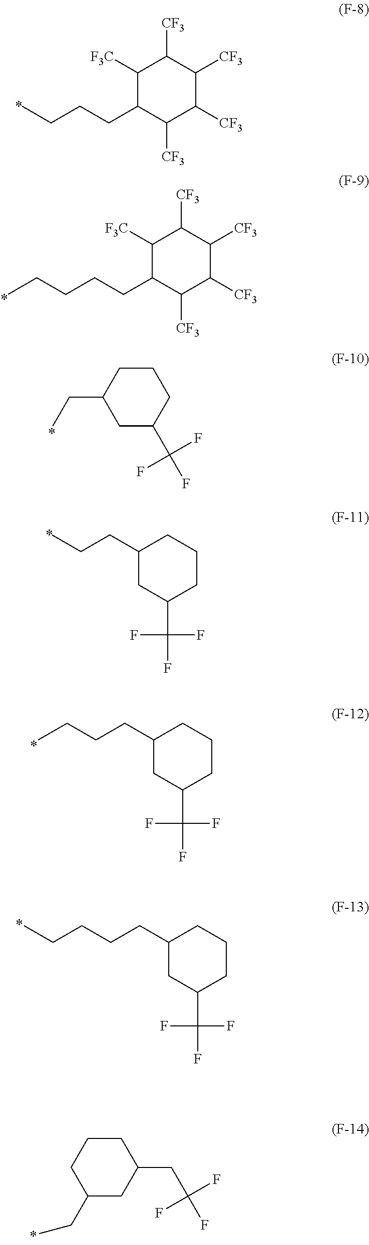

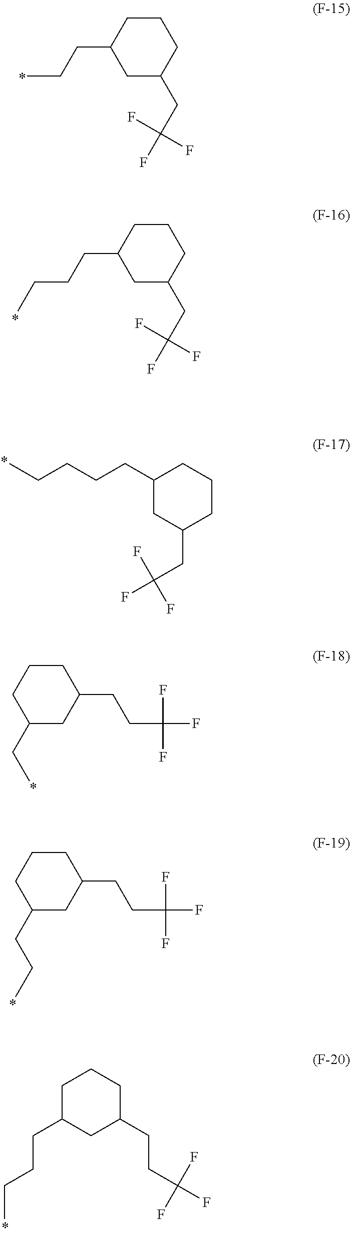 [SMILES: CCCC1CCCC(CC(F)(F)F)C1 ; CCCCC1CCCC(CC(F)(F)F)C1 ; CCCCCC1CCCC(CC(F)(F)F)C1 ; CCC1CCCC(CCC(F)(F)F)C1 ; CCCC1CCCC(CCC(F)(F)F)C1 ; CCCCC1CCCC(CCC(F)(F)F)C1]

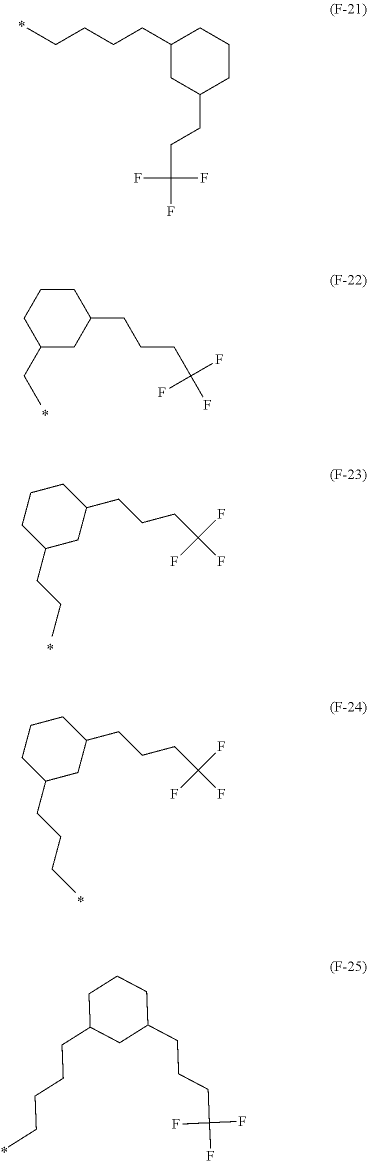 [SMILES: CCCCCC1CCCC(CCC(F)(F)F)C1 ; CCC1CCCC(CCCC(F)(F)F)C1 ; CCCC1CCCC(CCCC(F)(F)F)C1 ; CCCCC1CCCC(CCCC(F)(F)F)C1 ; CCCCCC1CCCC(CCCC(F)(F)F)C1]

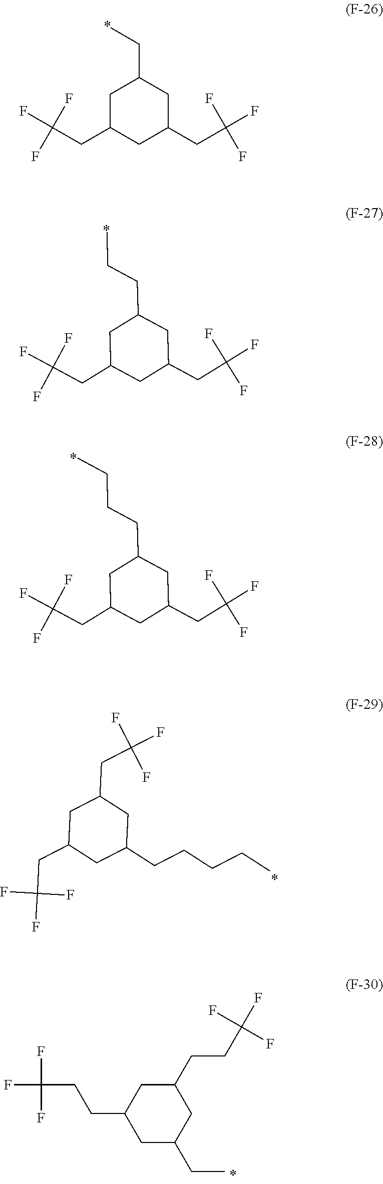 [SMILES: CCC1CC(CC(F)(F)F)CC(CC(F)(F)F)C1 ; CCCC1CC(CC(F)(F)F)CC(CC(F)(F)F)C1 ; CCCCC1CC(CC(F)(F)F)CC(CC(F)(F)F)C1 ; CCCCCC1CC(CC(F)(F)F)CC(CC(F)(F)F)C1 ; CCC1CC(CCC(F)(F)F)CC(CCC(F)(F)F)C1]

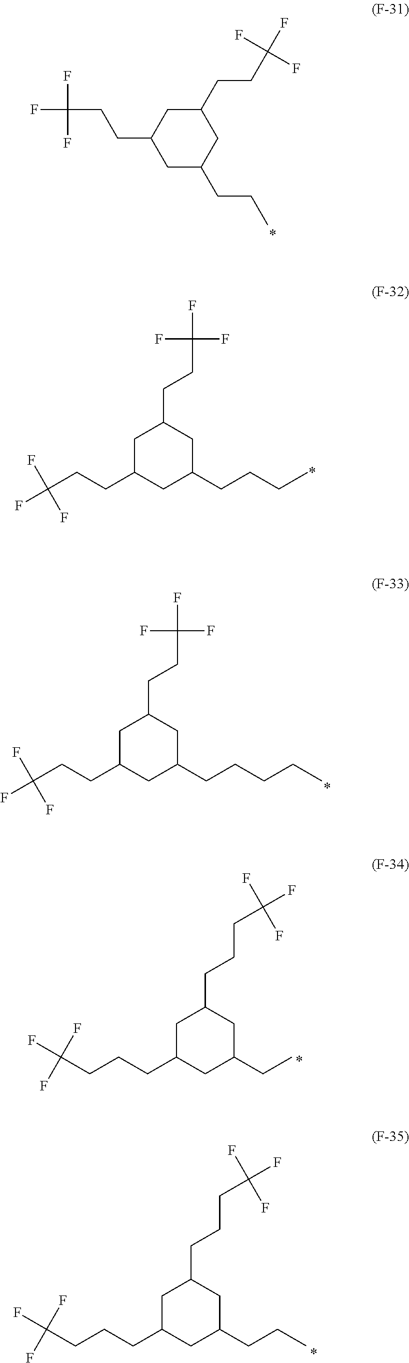 [SMILES: CCCC1CC(CCC(F)(F)F)CC(CCC(F)(F)F)C1 ; CCCCC1CC(CCC(F)(F)F)CC(CCC(F)(F)F)C1 ; CCCCCC1CC(CCC(F)(F)F)CC(CCC(F)(F)F)C1 ; CCC1CC(CCCC(F)(F)F)CC(CCCC(F)(F)F)C1 ; CCCC1CC(CCCC(F)(F)F)CC(CCCC(F)(F)F)C1]

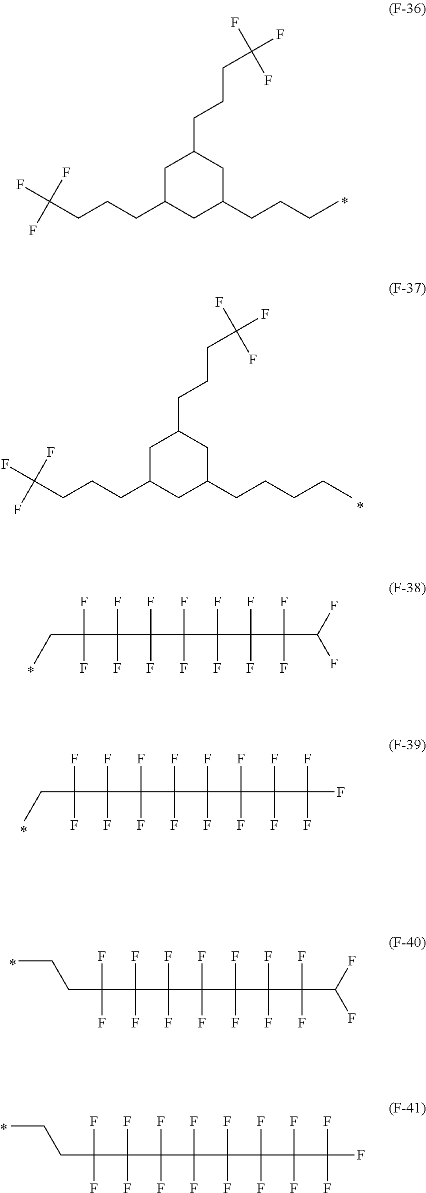

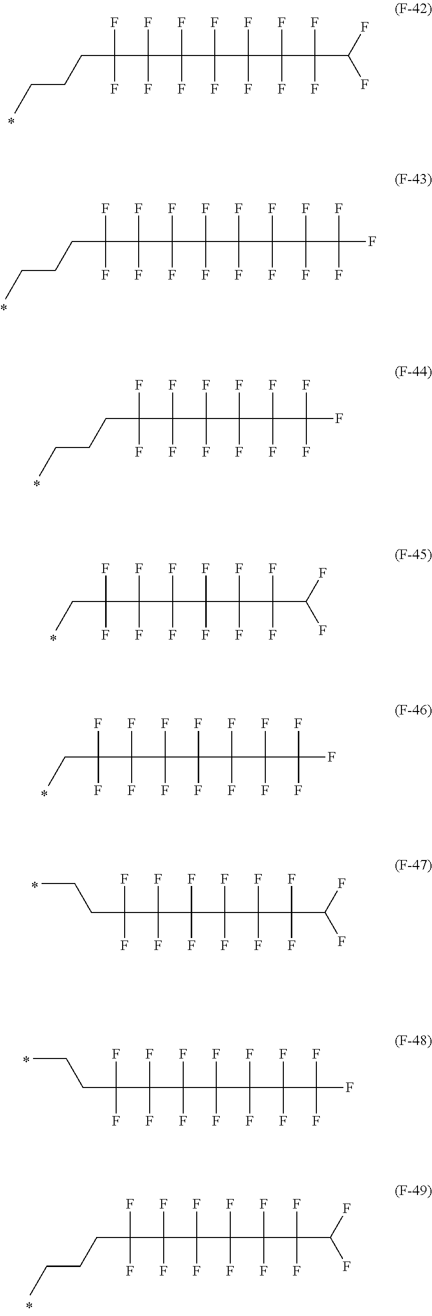

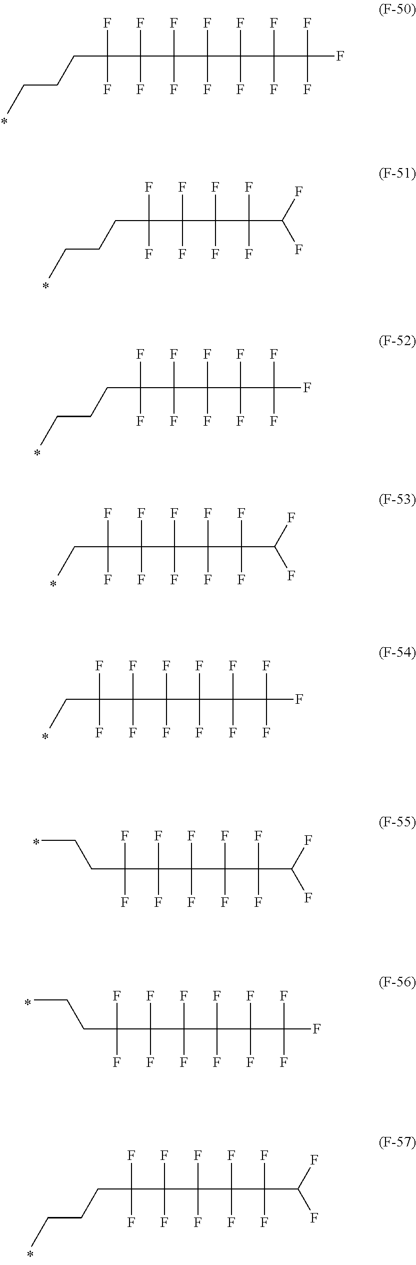

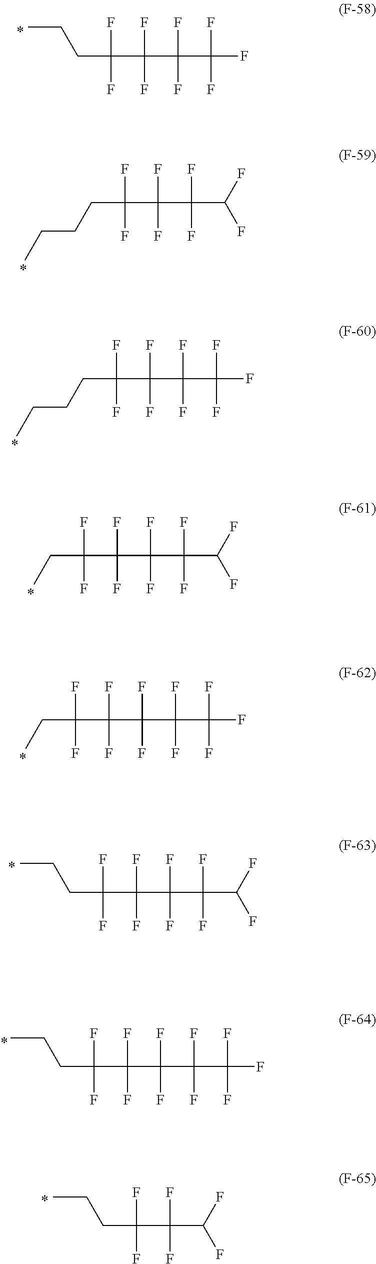

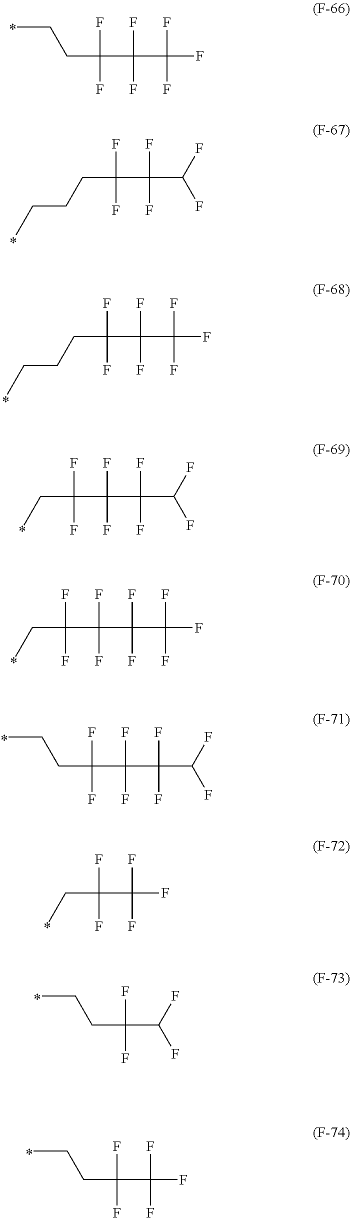

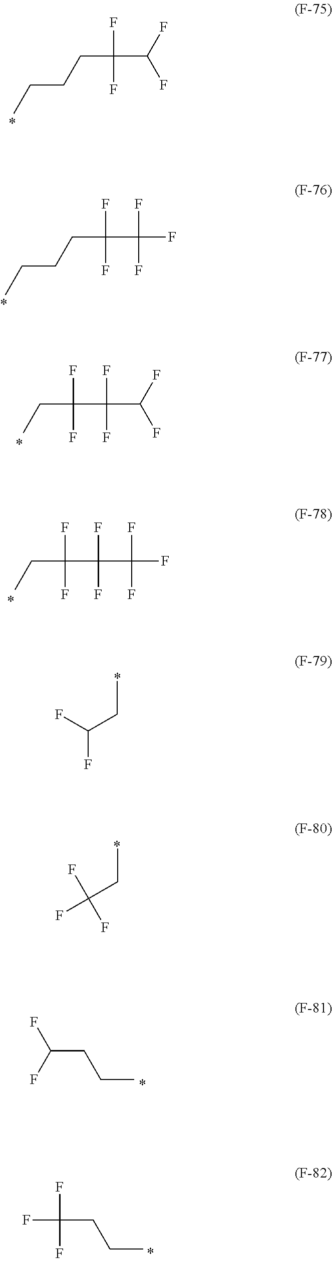

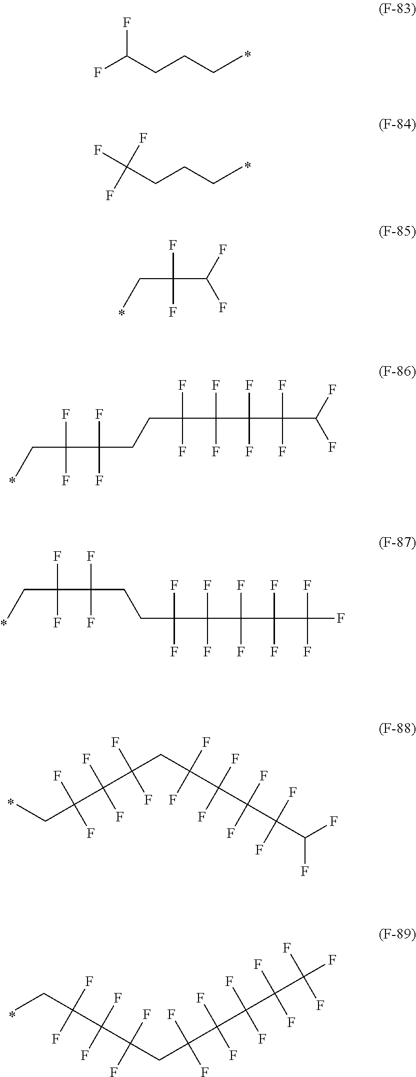

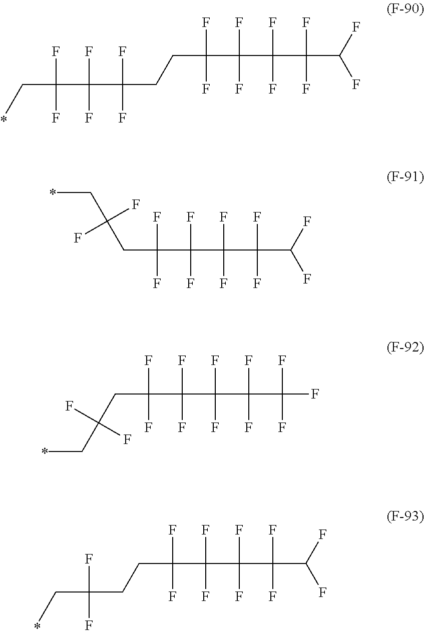 [SMILES: CCC(F)(F)C(F)(F)C(F)(F)CCC(F)(F)C(F)(F)C(F)(F)C(F)(F)C(F)F ; CCC(F)(F)CC(F)(F)C(F)(F)C(F)(F)C(F)(F)C(F)F ; CCC(F)(F)CC(F)(F)C(F)(F)C(F)(F)C(F)(F)C(F)(F)F ; CCC(F)(F)CCC(F)(F)C(F)(F)C(F)(F)C(F)(F)C(F)F]

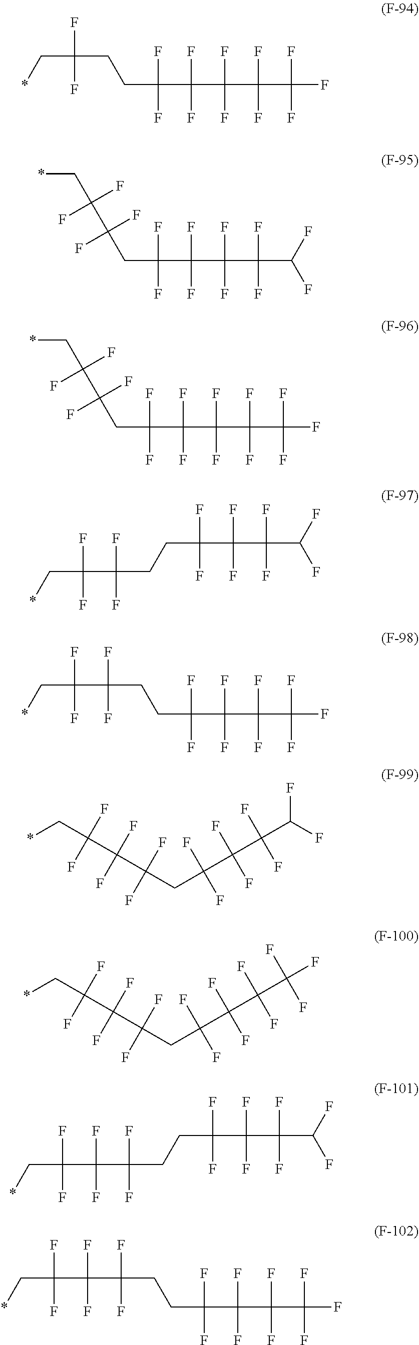

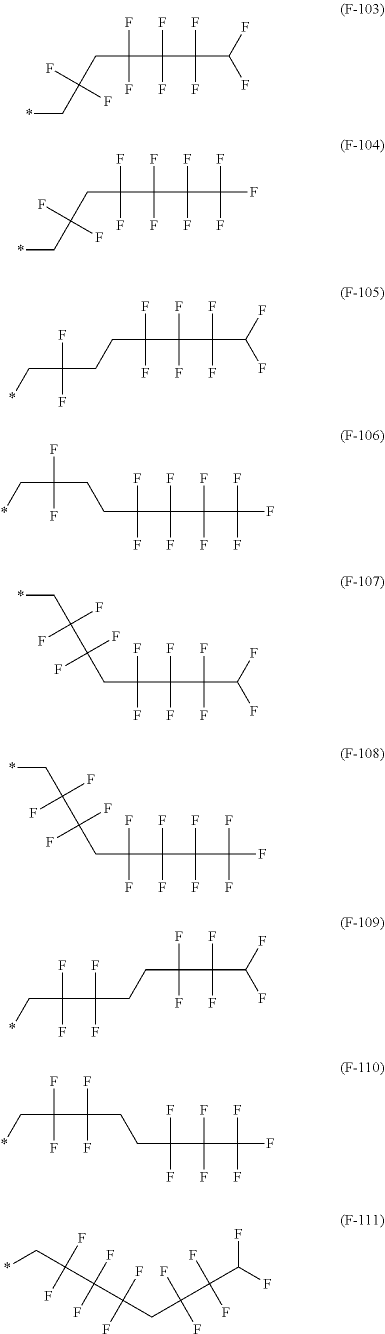

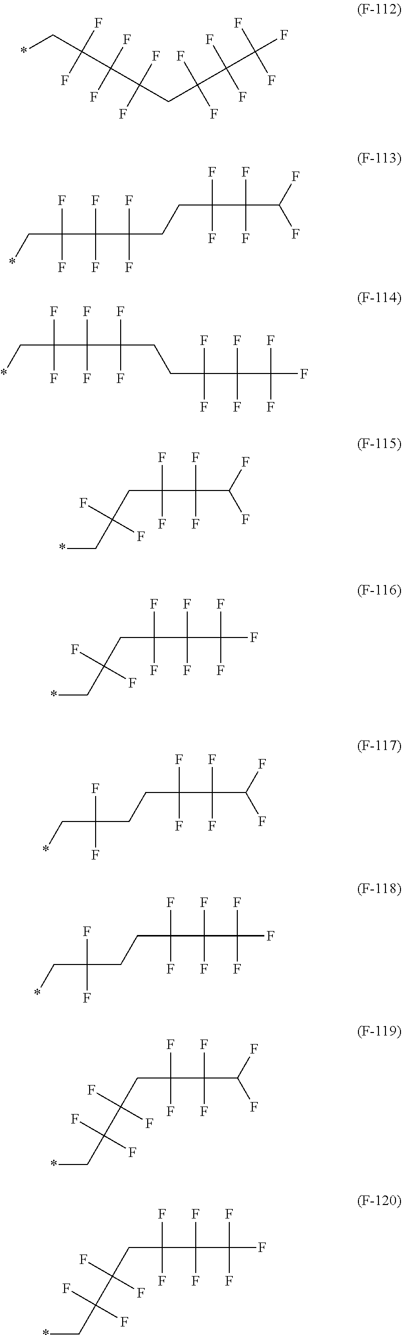

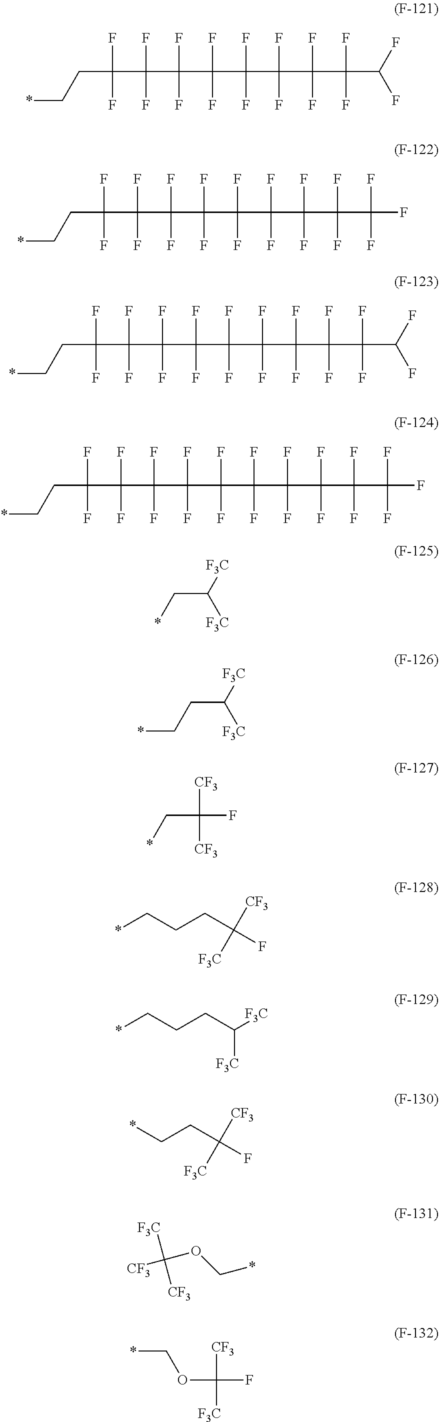

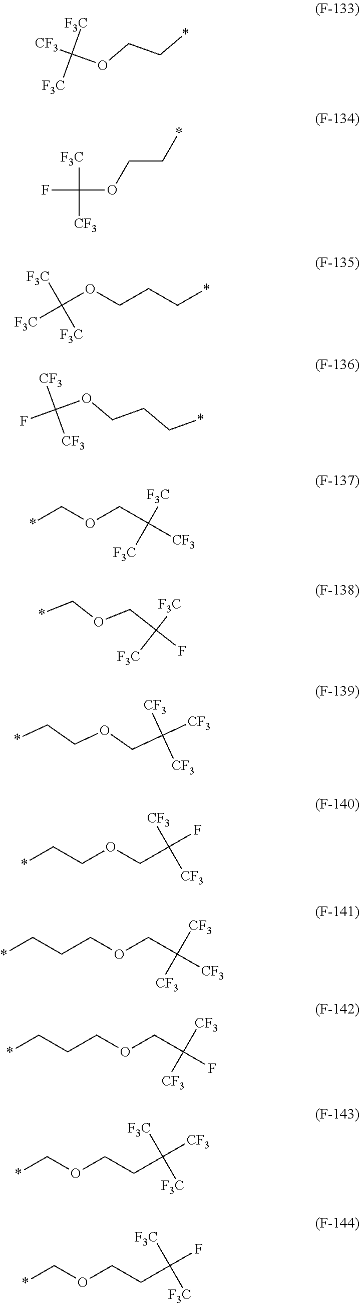

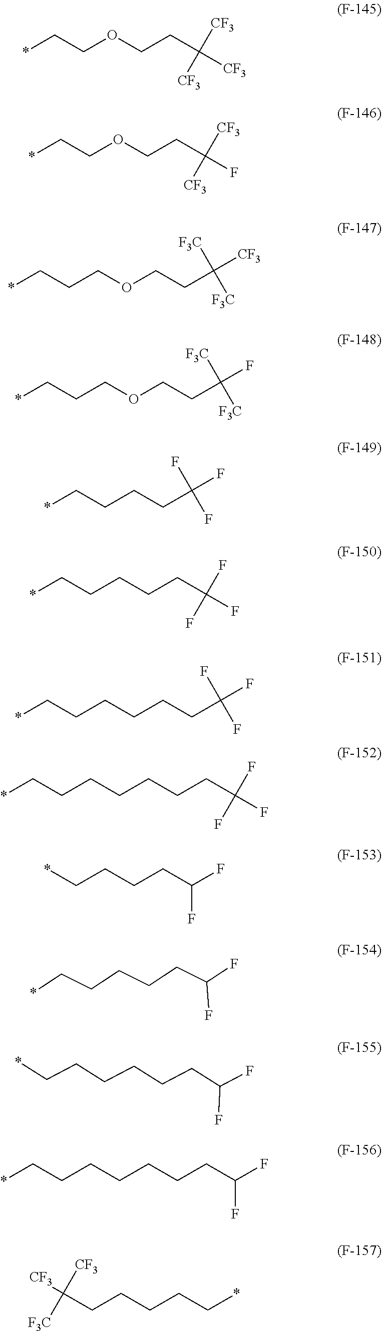

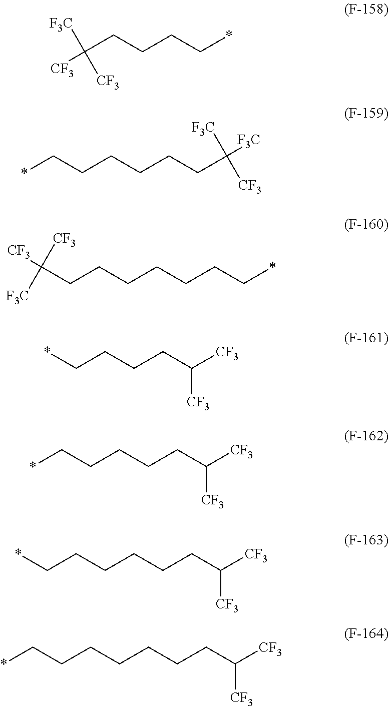

The synthesis of non-limiting examples of compounds containing a heterocylic moiety and at least one F-containing moiety will now be provided.

### Synthesis of 1,3,5-triazine-2,4,6-perfluorononanoate

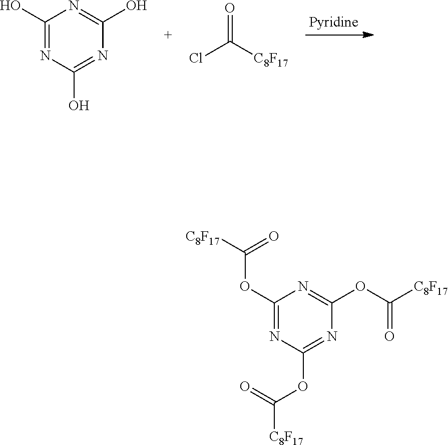

A simmering water bath (80-85° C.) was used to heat a 25 ml round flask. Cyanuric acid (0.33 g, 2.56 mmol) was added into the flask and dissolved in a minimal volume of pyridine (˜5 ml). The mixture was stirred until the cyanuric acid was fully dissolved. Following this, perfluorononanoyl chloride (3.95 g, 8.19 mmol) was added dropwise into the flask. The reaction was left to run for 30 minutes. Once the reaction was complete, the flask was cooled to room temperature and then transferred to an ice-water bath for 20 minutes to induce precipitation of the product. The resulting mixture was dried using rotary evaporation to obtain the crude product (1.41 g). This solid was then sublimed under vacuum to obtain a white solid (1.05 g, yield: 74%).

### Synthesis of 2,4,6-1H,1H-pentafluoropropanoxy-1,3,5-triazine

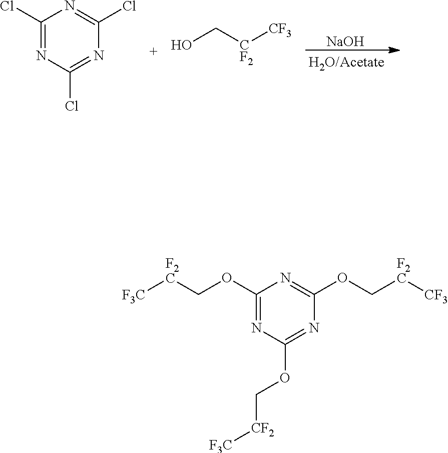

In a 250 mL round-bottom flask, 0.35 g of sodium hydroxide (8.75 mmol) was dissolved in 90 mL of water. Once the dissolution was complete, 1.30 g of 1H,1H-pentafluoropropanol (8.68 mmol) was added to the aqueous solution and stirred. In a 250 mL beaker, 500 mg of cyanuric chloride (2.72 mmol) was dissolved in 90 mL of acetone. The cyanuric chloride/acetone solution was added slowly to the aqueous solution and the resulting mixture was stirred overnight at room temperature. The reaction mixture was dried via rotary evaporation to produce a solid. This product was collected and rinsed with water before drying under vacuum. The final product was a white solid (1.35 g, yield: 94%).

### Synthesis of 2,4,6-1H,1H,2H,2H,2H-perfluorodecanoxy-1,3,5-triazine

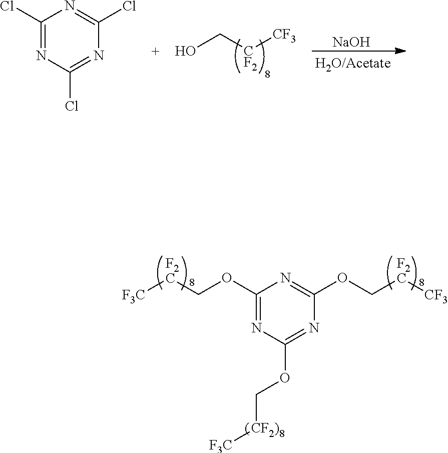

In a 100 mL round-bottom flask, 0.23 g of sodium hydroxide (5.57 mmol) was dissolved in 20 mL of water. Once the dissolution was complete was complete, 0.87 g of 1H,1H-perfluoro-1-decanol (1.74 mmol) was added to the aqueous solution and stirred. In a 250 mL beaker, 0.10 g of cyanuric chloride (0.54 mmol) was dissolved in 20 mL of acetone. The cyanuric chloride/acetone solution was added slowly to the aqueous solution and the resulting mixture was stirred overnight at room temperature. The reaction mixture was dried via rotary evaporation to produce a solid. This product was collected, rinsed with water, and then dried under vacuum. The final product was a white solid (0.97 g; yield: 9.1%).

### Synthesis of 2,4,6-bis(2,2,2-trifluoroethyl)amino-1,3,5-triazine

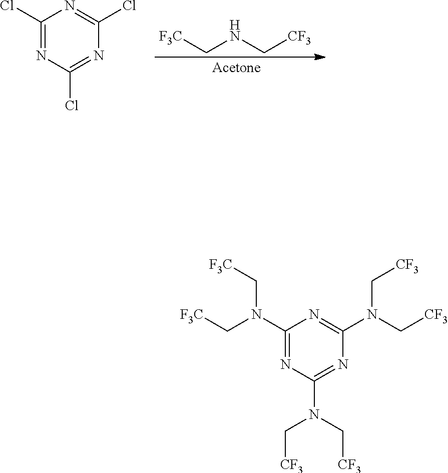 [SMILES: Clc1nc(Cl)nc(Cl)n1 ; FC(F)(F)CNCC(F)(F)F ; FC(F)(F)CN(CC(F)(F)F)c1nc(N(CC(F)(F)F)CC(F)(F)F)nc(N(CC(F)(F)F)CC(F)(F)F)n1]

In a 25 mL round bottom flask, 0.19 g of cyanuric chloride (1.04 mmol) was dissolved in 5 mL of acetone. Once the dissolution was complete, 0.60 g of bis(2,2,2-trifluoroethyl)amine was added slowly and the solution was stirred at room temperature. After 4 hours, the reaction was dried under rotary evaporation and the remaining solid was rinsed with water. The off-white solid crude product was dried under vacuum and then sublimed under reduced pressure at 40° C. The final product was a crystalline solid (0.22 g; yield: 42.3%).

### Synthesis of 2,4,6-2,3,4,5,6-pentafluorophenoxy-1,3,5-triazine

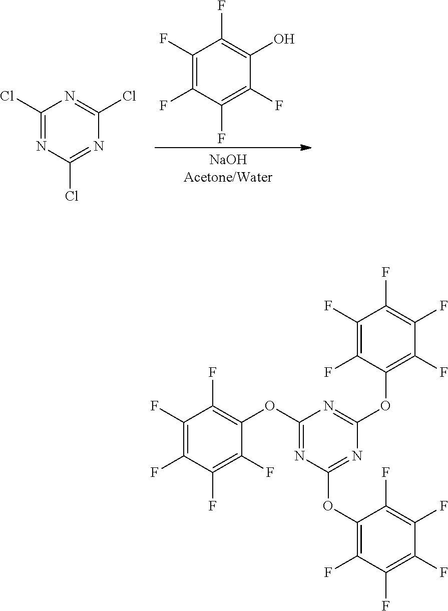

In a 500 mL round-bottom flask, 0.52 g of sodium hydroxide (13.1 mmol) was dissolved in 180 mL of water. Once the dissolution was complete, 2.4 g of 2,3,4,5,6-pentafluorophenol (13.1 mmol) was added to the aqueous solution and stirred. In a 500 mL beaker, 0.74 g of cyanuric chloride (4.02 mmol) was dissolved in 180 mL of acetone. The cyanuric chloride/acetone solution was added slowly to the aqueous solution and the resulting mixture was stirred overnight for approximately 16 hours at room temperature. The reaction mixture was dried using rotary evaporation to produce a white solid. This crude product was recrystallized in ethanol (2.51 g) and sublimed under vacuum to yield the final product (2.19 g, yield: 87%).

### Initial Sticking Probability

In some non-limiting examples, the initial sticking probability of the patterning material 211 may be determined by depositing such material as at least one of: a film, and coating, in a form, and under similar circumstances to the deposition of the patterning coating 110 within the device 100, having sufficient thickness so as to mitigate/reduce any effects on the degree of inter-molecular interaction with the underlying layer upon deposition on a surface thereof. In some non-limiting examples, the initial sticking probability may be measured on a film/coating having a thickness of one of at least about: 20 nm, 25 nm, 30 nm, 50 nm, 60 nm, and 100 nm.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may have an initial sticking probability against the deposition of the deposited material 331, that is one of no more than about: 0.3, 0.2, 0.15, 0.1, 0.08, 0.05, 0.03, 0.02, 0.01, 0.008, 0.005, 0.003, 0.001, 0.0008, 0.0005, 0.0003, and 0.0001.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may have an initial sticking probability against the deposition of at least one of: Ag, and Mg that is one of no more than about: 0.3, 0.2, 0.15, 0.1, 0.08, 0.05, 0.03, 0.02, 0.01, 0.008, 0.005, 0.003, 0.001, 0.0008, 0.0005, 0.0003, and 0.0001.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may have an initial sticking probability against the deposition of a deposited material 331 of one of between about: 0.15-0.0001, 0.1-0.0003, 0.08-0.0005, 0.08-0.0008, 0.05-0.001, 0.03-0.0001, 0.03-0.0003, 0.03-0.0005, 0.03-0.0008, 0.03-0.001, 0.03-0.005, 0.03-0.008, 0.03-0.01, 0.02-0.0001, 0.02-0.0003, 0.02-0.0005, 0.02-0.0008, 0.02-0.001, 0.02-0.005, 0.02-0.008, 0.02-0.01, 0.01-0.0001, 0.01-0.0003, 0.01-0.0005, 0.01-0.0008, 0.01-0.001, 0.01-0.005, 0.01-0.008, 0.008-0.0001, 0.008-0.0003, 0.008-0.0005, 0.008-0.0008, 0.008-0.001, 0.008-0.005, 0.005-0.0001, 0.005-0.0003, 0.005-0.0005, 0.005-0.0008, and 0.005-0.001.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may have an initial sticking probability against the deposition of a plurality of deposited materials 331 that is no more than a threshold value. In some non-limiting examples, such threshold value may be one of about: 0.3, 0.2, 0.18, 0.15, 0.13, 0.1, 0.08, 0.05, 0.03, 0.02, 0.01, 0.008, 0.005, 0.003, and 0.001.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may have an initial sticking probability that is no more than such threshold value against the deposition of a plurality of deposited materials 331 selected from at least one of: silver (Ag), magnesium (Mg), ytterbium (Yb), cadmium (Cd), and zinc (Zn). In some non-limiting examples, the patterning coating 110 may exhibit an initial sticking probability of no more than such threshold value against the deposition of a plurality of deposited materials 331 selected from at least one of: Ag, Mg, and Yb.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may exhibit an initial sticking probability against the deposition of a first deposited material 331 of, including without limitation, below, a first threshold value, and an initial sticking probability against the deposition of a second deposited material 331 of, including without limitation, below, a second threshold value. In some non-limiting examples, the first deposited material 331 may be Ag, and the second deposited material 331 may be Mg. In some non-limiting examples, the first deposited material 331 may be Ag, and the second deposited material may be Yb. In some non-limiting examples, the first deposited material 331 may be Yb, and the second deposited material 331 may be Mg. In some non-limiting examples, the first threshold value may exceed the second threshold value.

In some non-limiting examples, there may be scenarios calling for providing a patterning coating 110 for causing formation of a discontinuous layer 160 of at least one particle structure 150, upon the patterning coating 110 being subjected to a vapor flux 332 of a deposited material 331. In some non-limiting examples, the patterning coating 110 may exhibit a substantially low initial sticking probability such that a closed coating 140 of the deposited material 331 may be formed in the second portion 102, which may be substantially devoid of the patterning coating 110, while the discontinuous layer 160 of at least one particle structure 150 having at least one characteristic may be formed in the first portion 101 on the patterning coating 110. In some non-limiting examples, there may be scenarios calling for formation of a discontinuous layer 160 of at least one particle structure 150 of a deposited material 331, which may be, in some non-limiting examples, of one of: a metal, and a metal alloy, in the second portion 102, while depositing a closed coating 140 of the deposited material 331 having a thickness of, for example, one of no more than about: 100 nm, 50 nm, 25 nm, and 15 nm. In some non-limiting examples, an amount of the deposited material 331 deposited as a discontinuous layer 160 of at least one particle structure 150 in the first portion 101 may correspond to one of between about: 1-50%, 2-25%, 5-20%, and 7-10% of the amount of the deposited material 331 deposited as a closed coating 140 in the second portion 102, which in some non-limiting examples may correspond to a thickness of one of no more than about: 100 nm, 75 nm, 50 nm, 25 nm, and 15 nm.

In some non-limiting examples, there may be a positive correlation between the initial sticking probability of at least one of: the patterning coating 110, and the patterning material 211, in some non-limiting examples, when deposited as at least one of: a film, and a coating, in a form, and under circumstances similar to the deposition of the patterning coating 110 within the device 100, against the deposition of the deposited material 331 and an average layer thickness of the deposited material 331 thereon.

### Transmittance

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may have a transmittance for EM radiation of at least a threshold transmittance value, after being subjected to a vapor flux 332 of the deposited material 331, including without limitation, Ag.

In some non-limiting examples, such transmittance may be measured after exposing the exposed layer surface 11 of at least one of: the patterning coating 110 and the patterning material 211, formed as a thin film, to a vapor flux 332 of the deposited material 331, including without limitation, at least one of: a metal, and an alloy, including without limitation, at least one of: Yb, Ag, Mg, and Ag-containing materials, including without limitation, MgAg, under typical conditions that may be used for depositing an electrode of an opto-electronic device, which in some non-limiting examples, may be a cathode of an organic light-emitting diode (OLED) device.

In some non-limiting examples, the conditions for subjecting the exposed layer surface 11 to the vapor flux 332 of the deposited material 331, including without limitation, at least one of: a metal, and an alloy, including without limitation, at least one of: Yb, Ag, Mg, and Ag-containing materials, including without limitation, MgAg, may comprise: maintaining a vacuum pressure at a reference pressure, including without limitation, of one of about: 10⁻⁴ Torr and 10⁻⁵ Torr; the vapor flux 332 of the deposited material 331, including without limitation, at least one of: a metal, and an alloy, including without limitation, at least one of: Yb, Ag, Mg, and Ag-containing materials, including without limitation, MgAg, being substantially consistent with a reference deposition rate, including without limitation, of about 1 angstrom (Å)/sec, which in some non-limiting examples, may be monitored using a QCM; the vapor flux 332 of the deposited material 331 being directed toward the exposed layer surface 11 at an angle that is substantially close to normal to a plane of the exposed layer surface 11; the exposed layer surface 11 being subjected to the vapor flux 332 of the deposited material 331, including without limitation, at least one of: a metal, and an alloy, including without limitation, at least one of: Yb, Ag, Mg, and Ag-containing materials, including without limitation, MgAg, until a reference average layer thickness, including without limitation, of about 15 nm, is reached, and upon such reference average layer thickness being attained, the exposed layer surface 11 not being further subjected to the vapor flux of the deposited material 331, including without limitation, at least one of: a metal, and an alloy, including without limitation, at least one of: Yb, Ag, Mg, and Ag-containing materials, including without limitation, MgAg.

In some non-limiting examples, the exposed layer surface 11 being subjected to the vapor flux 332 of the deposited material 331, including without limitation, at least one of: Yb, Ag, Mg, and Ag-containing materials, including without limitation, MgAg, may be substantially at room temperature (e.g. about 25° C.). In some non-limiting examples, the exposed layer surface 11 being subjected to the vapor flux 332 of the deposited material 331, including without limitation, at least one of: a metal, and an alloy, including without limitation, at least one of: Yb, Ag, Mg, and Ag-containing materials, including without limitation, MgAg, may be positioned about 65 cm away from an evaporation source by which the deposited material 331, including without limitation, at least one of: a metal, and an alloy, including without limitation, at least one of: Yb, Ag, Mg, and Ag-containing materials, including without limitation, MgAg, is evaporated.

In some non-limiting examples, the threshold transmittance value may be measured at a wavelength in the visible spectrum, which may be one of at least about: 460 nm, 500 nm, 550 nm, and 600 nm. In some non-limiting examples, the threshold transmittance value may be measured at a wavelength in at least one of: the IR, and NIR, spectrum. In some non-limiting examples, the threshold transmittance value may be measured at a wavelength of one of about: 700 nm, 900 nm, and 1,000 nm. In some non-limiting examples, the threshold transmittance value may be expressed as a percentage of incident EM power that may be transmitted through a sample. In some non-limiting examples, the threshold transmittance value may be one of at least about: 60%, 65%, 70%, 75%, 80%, 85%, and 90%.

It would be appreciated by a person having ordinary skill in the relevant art that high transmittance may generally indicate an absence of a closed coating 140 of the deposited material 331, including without limitation, at least one of: Yb, Ag, Mg, and Ag-containing materials, including without limitation, MgAg. On the other hand, low transmittance may generally indicate presence of a closed coating 140 of the deposited material 331, including without limitation, at least one of: Yb, Ag, Mg, and Ag-containing materials, including without limitation, MgAg, since metallic thin films, particularly when formed as a closed coating 140, may exhibit a high degree of absorption of EM radiation.

A series of samples was fabricated to measure the transmittance of an example material, as well as to visually observe whether a closed coating 140 of Ag was formed on the exposed layer surface 11 of such example material. Each sample was prepared by depositing, on a glass substrate 10, an approximately 50 nm thick coating of an example material, then subjecting the exposed layer surface 11 of the coating to a vapor flux 332 of Ag at a rate of about 1 Å/see until a reference layer thickness of about 15 nm was reached. Each sample was then visually analyzed and the transmittance through each sample was measured.

The molecular structures of the example materials used in the samples herein are set out in Table 1 below:

TABLE 1

| Material | Molecular Structure/Name |
|---|---|
| HT211 | 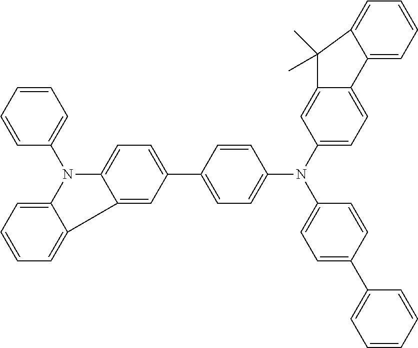 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| HT01 | 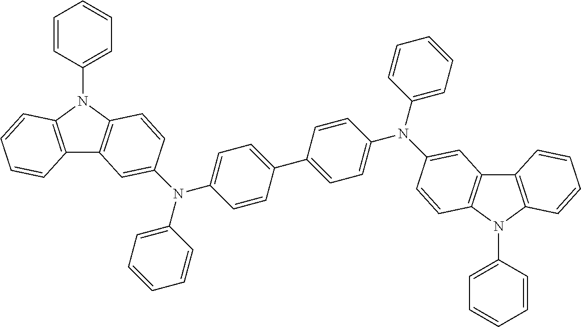 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1] |
| TAZ | 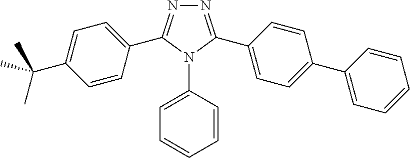 |
| Balq | 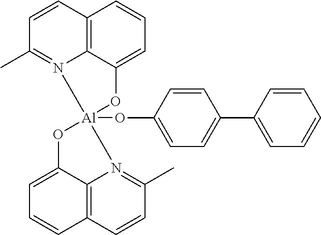 |
| Liq | 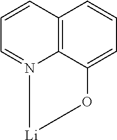 |
| Example Material 1 | 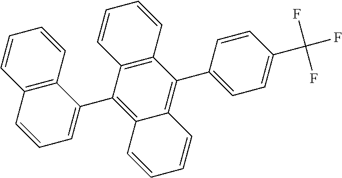 [SMILES: FC(F)(F)c1ccc(-c2c3ccccc3c(-c3cccc4ccccc34)c3ccccc23)cc1] |
| Example Material 2 | 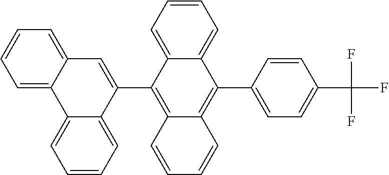 [SMILES: FC(F)(F)c1ccc(-c2c3ccccc3c(-c3cc4ccccc4c4ccccc34)c3ccccc23)cc1] |
| Example Material 3 | 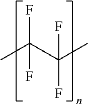 |
| Example Material 4 | 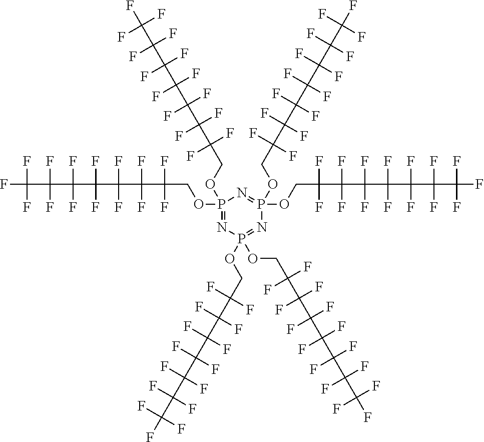 [SMILES: FC(F)(F)C(F)(F)C(F)(F)C(F)(F)C(F)(F)C(F)(F)C(F)(F)COP1(OCC(F)(F)C(F)(F)C(F)(F)C(F)(F)C(F)(F)C(F)(F)C(F)(F)F)=NP(OCC(F)(F)C(F)(F)C(F)(F)C(F)(F)C(F)(F)C(F)(F)C(F)(F)F)(OCC(F)(F)C(F)(F)C(F)(F)C(F)(F)C(F)(F)C(F)(F)C(F)(F)F)=NP(OCC(F)(F)C(F)(F)C(F)(F)C(F)(F)C(F)(F)C(F)(F)C(F)(F)F)(OCC(F)(F)C(F)(F)C(F)(F)C(F)(F)C(F)(F)C(F)(F)C(F)(F)F)=N1] |
| Example Material 5 | 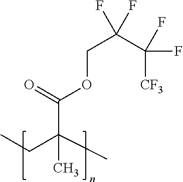 |
| Example Material 6 | 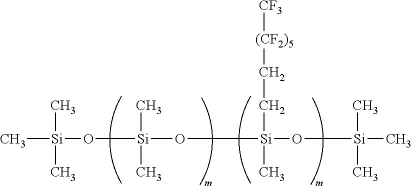 |
| Example Material 7 | 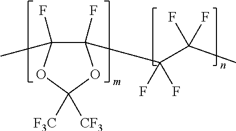 |
| Example Material 8 | 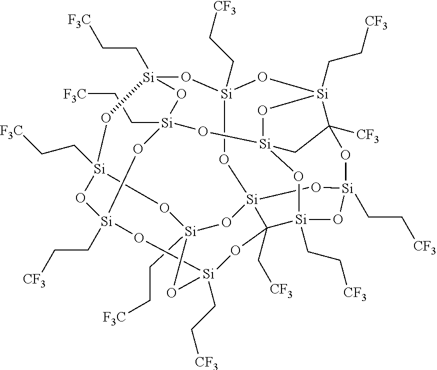 [SMILES: FC(F)(F)CC[Si]12OC3(CC(F)(F)F)[Si]4(CCC(F)(F)F)O[Si]56CC7(C(F)(F)F)O[Si](CCC(F)(F)F)(O4)O[Si]34O[Si](CCC(F)(F)F)(O1)O[Si]1(CCC(F)(F)F)O[Si](CCC(F)(F)F)(O2)O[Si](CCC(F)(F)F)(O5)O[Si@](CCC(F)(F)F)(O1)O[Si](CCC(F)(F)F)(O[Si]7(CCC(F)(F)F)O6)O4] |
| Example Material 9 | 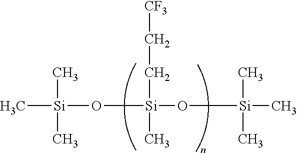 |

Those having ordinary skill in the relevant art will appreciate that samples having little to no deposited material 331, including without limitation, at least one of: a metal, and an alloy, including without limitation, at least one of: Yb, Ag, Mg, and Ag-containing materials, including without limitation, MgAg, present thereon may be substantially transparent, while samples with substantial amounts of at least one of: a metal, and an alloy, deposited thereon, including without limitation, as a closed coating 140, may in some non-limiting examples, exhibit a substantially reduced transmittance. Accordingly, the performance of various example coatings as a patterning coating 110 may be assessed by measuring transmission through the samples, which may be inversely correlated to at least one of: an amount, and an average layer thickness, of the deposited material 331, including without limitation, at least one of: a metal, and an alloy, including without limitation, in the form of at least one of Yb, Ag, Mg, and Ag-containing materials, including without limitation, MgAg, being deposited thereon, since metallic thin films, including without limitation, when formed as a closed coating 140, may exhibit a high degree of absorption of EM radiation.

The samples in which a substantially closed coating 140 of a deposited material 331, in the form of Ag, had formed were visually identified, and the presence of such closed coating 140 in these samples was further confirmed by measurement of transmittance therethrough, which showed transmittance of no more than about 50% at a wavelength of about 460 nm.

In addition, for samples in which the absence of formation of a closed coating 140 of a deposited material 331, in the form of Ag, was identified, the absence of such closed coating 140 in these samples was further confirmed by measurement of EM transmittance therethrough, which showed transmittance (of EM radiation at a wavelength of about 460 nm) of at least about 70%.

The results are summarized in Table 2 below:

TABLE 2

| Material | Closed Coating of Ag? |
|---|---|
| HT211 | Present |
| HT01 | Present |
| TAZ | Present |
| Balq | Present |
| Liq | Present |
| Example Material 1 | Present |
| Example Material 2 | Present |
| Example Material 3 | Not Present |
| Example Material 4 | Not Present |
| Example Material 5 | Not Present |
| Example Material 6 | Not Present |
| Example Material 7 | Not Present |
| Example Material 8 | Not Present |
| Example Material 9 | Present |

Based on the foregoing, it was found that the materials used in the first 7 samples (HT211 to Example Material 2) and Example Material 9 in Tables 1 and 2 may have reduced applicability in some scenarios for inhibiting the deposition of the deposited material 331 thereon, including without limitation, at least one of: a metal, and an alloy, including without limitation, at least one of: Yb, Ag, Mg, and Ag-containing materials, including without limitation, MgAg.

On the other hand, it was found that Example Material 3 to Example Material 8 may have applicability in some scenarios, to act as a patterning coating 110 for inhibiting the deposition of the deposited material 331 including without limitation, at least one of: a metal, and an alloy, including without limitation, at least one of: Yb, Ag, Mg, and Ag-containing materials, including without limitation, MgAg, thereon.

### Deposition Contrast

In some non-limiting examples, a material, including without limitation, a patterning material 211, that may function as an NIC for a given at least one of: a metal, and an alloy, including without limitation, at least one of: Mg, Ag, and MgAg, may have a substantially high deposition contrast when deposited on a substrate 10.

In some non-limiting examples, if a substrate 10 tends to act as a nucleation-promoting coating (NPC) 520, and a portion thereof is coated with a material, including without limitation, a patterning material 211, that may tend to function as an NIC against deposition of a deposited material 331, including without limitation, at least one of: a metal, and an alloy, including without limitation, at least one of: Yb, Ag, Mg, and Ag-containing materials, including without limitation, MgAg, a coated portion (first portion 101) and an uncoated portion (second portion 102) may tend to have different at least one of: initial sticking probabilities, and nucleation rates, such that the deposited material 331 deposited thereon may tend to have different average film thicknesses.

As used herein, a quotient of an average film thickness of the deposited material 331 deposited in the second portion 102 divided by the average film thickness of the deposited material in the first portion 101 in such scenario may be generally referred to as a deposition contrast. Thus, if the deposition contrast is substantially high, the average film thickness of the deposited material 331 in the second portion 102 may be substantially greater than the average film thickness of the deposited material 331 in the first portion 101.

In some non-limiting examples, a material, including without limitation, a patterning material 211, that may function as an NIC for a given deposited material 331, may have a substantially high deposition contrast when deposited on a substrate 10.

In some non-limiting examples, there may be a negative correlation between the initial sticking probability of at least one of: the patterning coating 110, and the patterning material 211, in some non-limiting examples, when deposited as at least one of: a film, and a coating, in a form, and under circumstances similar to the deposition of the patterning coating 110 within the device 100, against the deposition of the deposited material 331 and a deposition contrast thereof, that is, a low initial sticking probability may be highly correlated with a high deposition contrast.

In some non-limiting examples, if the deposition contrast is substantially high, there may be little to no deposited material 331 deposited in the first portion 101, when there is sufficient deposition of the deposited material 331 to form a closed coating 140 thereof in the second portion 102.

In some non-limiting examples, if the deposition contrast is substantially low, there may be a discontinuous layer 160 of at least one particle structure 150 of the deposited material 331 deposited in the first portion 101, when there is sufficient deposition of the deposited material 331 to form a closed coating 140 in the second portion 102.

In some non-limiting examples, a material, including without limitation, a patterning material 211, having a substantially high deposition contrast against deposition of a given deposited material 331, including without limitation, at least one of: a metal, and an alloy, including without limitation, at least one of: Yb, Ag, Mg, and Ag-containing materials, including without limitation, MgAg, may have reduced applicability in some scenarios calling for a reduced deposition contrast, in some non-limiting examples, where the average layer thickness of the deposited material 331 in the first portion 101 is substantially low, including without limitation, one of no more than about: 100 nm, 50 nm, 25 nm, and 15 nm, including without limitation, in some scenarios that call for a deposition of a discontinuous layer 160 of at least one particle structure 150 in the second portion 102.

In some non-limiting examples, there may be scenarios calling for the formation of a discontinuous layer 160 of at least one particle structure 150 of the deposited material 331, in the first portion 101, when an average layer thickness of a closed coating 140 of the deposited material 331 in the second portion 102 is substantially small, including without limitation, one of no more than about: 100 nm, 50 nm, 25 nm, and 15 nm, including without limitation, the formation of nanoparticles (NPs) in the first portion 101, where absorption of EM radiation by such NPs is called for, including without limitation, to protect an underlying layer 210 from EM radiation having a wavelength of no more than about 460 nm.

In some non-limiting examples, in such scenarios, there may be applicability for a deposition contrast of one of between about: 2-100, 4-50, 5-20, and 10-15.

In some non-limiting examples, a material, including without limitation, a patterning material 211, having a substantially low deposition contrast against deposition of a deposited material 331, may have reduced applicability in some scenarios calling for substantially high deposition contrast, including without limitation, where the average layer thickness of the deposited material 331 in the first portion 101 is large, including without limitation, one of at least about: 95 nm, 45 nm, 20 nm, 10 nm, and 8 nm.

In some non-limiting examples, a material, including without limitation, a patterning material 211, having a substantially low deposition contrast against deposition of a deposited material 331, may have reduced applicability in some scenarios calling for substantially high deposition contrast, including without limitation, scenarios calling for at least one of: the substantial absence of a closed coating 140, and a high density of, particle structures 150 in the first portion 101, including without limitation, when an average layer thickness of the deposited material 331 in the second portion 102 is large, including without limitation, one of at least about: 95 nm, 45 nm, 20 nm, 10 nm, and 8 nm, including without limitation, in some scenarios calling for the substantial absence of absorption of EM radiation in at least one of the visible spectrum and the NIR spectrum, including without limitation, scenarios calling for an increased transparency to EM radiation having a wavelength that is at least about 460 nm.

In some non-limiting examples, a material, including without limitation, a patterning material 211, having a substantially low deposition contrast against the deposition of a deposited material 331, may have applicability in some scenarios calling for at least one of: a discontinuous layer 160 of, and a low density of, particle structures 150 of the deposited material 331 in the first portion 101, when an average layer thickness of a closed coating 140 of the deposited material 331 in the second portion 102 is substantially high, including without limitation, one of at least about: 95 nm, 45 nm, 20 nm, 10 nm, and 8 nm. In some non-limiting examples, a deposition contrast of one of between about: 2-100, 4-50, 5-20, and 10-15 may have applicability in some scenarios when an average layer thickness of the deposited material 331 in the second portion 102 is substantially high, including without limitation, one of at least about: 95 nm, 45 nm, 20 nm, 10 nm, and 8 nm.

In some non-limiting examples, a material, including without limitation, a patterning material 211, may tend to have a substantially low deposition contrast if the initial sticking probability of such material against deposition of at least one of: a metal, and an alloy, including without limitation, at least one of: Mg, Ag, and MgAg, is substantially high.

### Surface Energy

A characteristic surface energy, as used herein, in some non-limiting examples, with respect to a material, may generally refer to a surface energy determined from such material.

In some non-limiting examples, a characteristic surface energy may be measured from a surface formed by the material deposited (coated) in a thin film form.

Various methods and theories for determining the surface energy of a solid are known.

In some non-limiting examples, a surface energy may be calculated (derived) based on a series of contact angle measurements, in which various liquids may be brought into contact with a surface of a solid to measure the contact angle between the liquid-vapor interface and the surface. In some non-limiting examples, a surface energy of a solid surface may be equal to the surface tension of a liquid with the highest surface tension that completely wets the surface.

In some non-limiting examples, the critical surface tension of a surface may be determined according to the Zisman method, as further detailed in W. A. Zisman, Advances in Chemistry 43 (1964), pp. 1-51.

In some non-limiting examples, a characteristic surface energy of a material, including without limitation, a patterning material 211, in a coating, including without limitation, a patterning coating 110, may be determined by depositing the material as a substantially pure coating (e.g. a coating formed by a substantially pure material) on a substrate 10 and measuring a contact angle thereof with an applicable series of probe liquids.

In some non-limiting examples, a Zisman plot may be used to determine a maximum value of surface tension that would result in complete wetting (i.e. a contact angle θ_{c} of 0°) of the surface.

In some non-limiting examples, a material, including without limitation, a patterning material 211 that may tend to function as an NIC for a given deposited material 331, including without limitation, at least one of: a metal, and an alloy, including without limitation, at least one of: Yb, Ag, Mg, and Ag-containing materials, including without limitation, MgAg, may tend to exhibit a substantially low surface energy when deposited as a thin film/coating on an exposed layer surface 11.

Without wishing to be bound by any particular theory, it has now been found that a patterning coating 110 comprising a material which, when deposited as a thin film, exhibits a substantially high surface energy, may, in some non-limiting examples, form a discontinuous layer 160 of at least one particle structure 150 of a deposited material 331 in the first portion 101, and a closed coating 140 of the deposited material 331 in the second portion 102, including without limitation, in cases where the thickness of the closed coating is, by way of non-limiting example, one of no more than about: 100 nm, 75 nm, 50 nm, 25 nm, and 15 nm.

In some non-limiting examples, a series of samples was fabricated to measure the critical surface tension of the surfaces formed by the various materials. The results of the measurement are summarized in Table 3:

TABLE 3

| | Critical Surface |
|---|---|
| Material | Tension (dynes/cm) |
| HT211 | 25.6 |
| HT01 | >24 |
| TAZ | 22.4 |
| Balq | 25.9 |
| Liq | 24 |
| Example Material 1 | 26.3 |
| Example Material 2 | 24.8 |
| Example Material 3 | 20.0 |
| Example Material 4 | 12.4 |
| Example Material 5 | 15.9 |
| Example Material 6 | 21.1 |
| Example Material 7 | 13.1 |
| Example Material 8 | 21 |
| Example Material 9 | 18.9 |

Based on the foregoing measurement of the critical surface tension in Table 3 and the previous observation regarding one of: the presence, and absence, of a substantially closed coating 140 of a deposited material 331, in the form of Ag, it was found that materials that form substantially low surface energy surfaces when deposited as a coating, including without limitation, a patterning coating 110, which in some non-limiting examples, may be those having a critical surface tension of one of between about: 13-20 dynes/cm, and 13-19 dynes/cm, may have applicability for forming the patterning coating 110 to inhibit deposition of a deposited material 331 thereon, including without limitation, at least one of Yb, Ag, Mg, and Ag-containing materials, including without limitation, MgAg.

In some non-limiting examples, a material, including without limitation, a patterning material 211 that may tend to function as an NIC for a deposited material 331, including without limitation, at least one of: a metal, and an alloy, including without limitation, at least one of: Mg, Ag, and Ag-containing materials, including without limitation, MgAg, may tend to exhibit a substantially low surface energy when deposited as a thin film (coating) on an exposed layer surface 11.

In some non-limiting examples, a material, including without limitation, a patterning material 211, with a substantially low surface energy may tend to exhibit substantially low inter-molecular forces.

In some non-limiting examples, a material, including without limitation, a patterning material 211 that may tend to function as an NIC for a deposited material 331, including without limitation, at least one of: a metal, and an alloy, including without limitation, at least one of: Mg, Ag, and Ag-containing materials, including without limitation, MgAg, may tend to have a substantially high initial sticking probability against deposition of the deposited material 331, if the material has a substantially high surface energy.

In some non-limiting examples, there may be scenarios calling for a patterning material 211 that has a substantially low surface energy that is not unduly low.

In some non-limiting examples, a material, including without limitation, a patterning material 211, with a substantially high surface energy may have applicability for some scenarios to detect a film of such material using optical techniques.

Without wishing to be bound by any particular theory, it may be postulated that, in some non-limiting examples, a material, including without limitation, a patterning material 211, having a substantially high surface energy may have applicability for some scenarios that call for substantially high temperature reliability.

In some non-limiting examples, a patterning material 211 that has a substantially low surface tension that is not unduly low, may have applicability in some scenarios calling for a substantially high melting point.

In some non-limiting examples, a material, including without limitation, a patterning material 211, having a substantially low surface energy may have applicability in some scenarios calling for weak, including without limitation, substantially no, photoluminescence/absorption in a wavelength range that is one of at least about: 365 nm and 460 nm.

In some non-limiting examples, a material, including without limitation, a patterning material 211, that may function as an NIC for at least one of: a metal, and an alloy, including without limitation, at least one of Mg, Ag, and Ag-containing materials, including without limitation, MgAg, having a substantially high surface energy may have applicability in some scenarios calling for a discontinuous layer 160 of particle structures 150 of at least one of: the metal, and the alloy, in the first portion 101, when an average layer thickness of a continuous coating 140 of at least one of: the metal, and the alloy, in the second portion 102 is substantially low, including without limitation, one of no more than about: 100 nm, 50 nm, 25 nm, and 15 nm.

In some non-limiting examples, a material, including without limitation, a patterning material 211, that may function as an NIC for a deposited material 331, including without limitation, at least one of: a metal, and an alloy, including without limitation, at least one of: Yb, Ag, Mg, and Ag-containing materials, including without limitation, MgAg, having a substantially low surface energy may have applicability in some scenarios calling for one of: a discontinuous layer 160 of, and a low density of, particle structures 150 of the deposited material 331 in the first portion 101, when an average layer thickness of a closed coating 140 of the deposited material 331 in the second portion 102 is substantially high, including without limitation, one of at least about: 95 nm, 45 nm, 20 nm, 10 nm, and 8 nm.

In some non-limiting examples, the surface of at least one of: the patterning coating 110, and the patterning material 211, in some non-limiting examples, when deposited as at least one of: a film, and a coating, in a form, and under circumstances similar to the deposition of the patterning coating 110 within the device 100, comprising the compounds described herein, may exhibit a surface energy of one of no more than about: 24 dynes/cm, 22 dynes/cm, 20 dynes/cm, 18 dynes/cm, 16 dynes/cm, 15 dynes/cm, 13 dynes/cm, 12 dynes/cm, and 11 dynes/cm.

In some non-limiting examples, the surface values in various non-limiting examples herein may correspond to such values measured at around normal temperature and pressure (NTP), which may correspond to a temperature of 20° C., and an absolute pressure of 1 atm.

In some non-limiting examples, the surface energy may be one of at least about: 6 dynes/cm, 7 dynes/cm, and 8 dynes/cm.

In some non-limiting examples, the surface energy may be one of between about: 10-20 dynes/cm, and 13-19 dynes/cm.

Without wishing to be bound by any particular theory, it may be postulated that materials that form a surface having a surface energy of no more than, in some non-limiting examples, about 13 dynes/cm, may reduced applicability as a patterning material 211 in some scenarios, as such materials may exhibit at least one of: substantially low adhesion to layer(s) surrounding such materials, a substantially low melting point, and a substantially low sublimation temperature.

It has also now been found, that a patterning coating 110 formed by a compound exhibiting a substantially low surface energy may also exhibit a substantially low refractive index.

### Temperature

### Glass Transition Temperature

In some non-limiting examples, at least one of: the patterning coating 110, and the patterning material 211, in some non-limiting examples, when deposited as at least one of: a film, and coating, in a form, and under circumstances similar to the deposition of the patterning coating 110 within the device 100, may have a glass transition temperature that is one of: one of at least about: 300° C., 150° C., and 130° C., and one of no more than about: 30° C., 0° C., −30° C., and −50° C.

### Sublimation Temperature

In some non-limiting examples, a material, including without limitation, a patterning material 211, having substantially low inter-molecular forces may tend to exhibit a substantially low sublimation temperature.

In some non-limiting examples, a material, including without limitation, a patterning material 211, having a substantially low sublimation temperature, may have reduced applicability for manufacturing processes that may call for substantially precise control of an average layer thickness in a deposited film of the material.

In some non-limiting examples, a material, including without limitation, a patterning material 211, having a sublimation temperature that is one of no more than about: 140° C., 120° C., 110° C., 100° C. and 90° C., may tend to encounter constraints on at least one of: the deposition rate and the average layer thickness, of a film comprising such material that may be deposited using known deposition methods, including without limitation, vacuum thermal evaporation.

In some non-limiting examples, a material, including without limitation, a patterning material 211, having a substantially high sublimation temperature may have applicability in some scenarios calling for substantially high precision in the control of the average layer thickness of a film comprising such material.

In some non-limiting examples, the patterning material 211 may have a sublimation temperature of one of between about: 100-320° C., 120-300° C., 140-280° C., and 150-250° C. In some non-limiting examples, such sublimation temperature may allow the patterning material 211 to be substantially readily deposited as a coating using PVD.

In some non-limiting examples, a material with substantially low intermolecular forces may exhibit a substantially low sublimation temperature.

In some non-limiting examples, a material, including without limitation, a patterning material 211, having a substantially low sublimation temperature, may have reduced applicability for manufacturing processes that may call for substantially precise control of an average layer thickness of a closed coating 140 of the material.

In some non-limiting examples, a material, including without limitation, a patterning material 211, having a sublimation temperature that is one of no more than about: 140° C., 120° C., 110° C., 100° C. and 90° C., may tend to encounter constraints on at least one of: the deposition rate and the average layer thickness, of a film comprising such material that may be deposited using known deposition methods, including without limitation, vacuum thermal evaporation.

In some non-limiting examples, a material, including without limitation, a patterning material 211, having a substantially high sublimation temperature may have applicability in some scenarios calling for substantially high precision in the control of the average layer thickness of a film comprising such material.

The sublimation temperature of a material, including without limitation, a patterning material 211, may be determined using various methods apparent to those having ordinary skill in the relevant art, including without limitation, by heating the material in an evaporation source under a substantially high vacuum environment, in some non-limiting examples, about 10⁻⁴ Torr, and including without limitation, in a crucible and by determining a temperature that may be attained, to at least one of:
observe commencement of the deposition of the material onto an exposed layer surface 11 on a QCM mounted a fixed distance from the crucible;
observe a specific deposition rate, in some non-limiting examples, 0.1 Å/sec, onto an exposed layer surface 11 on a QCM mounted a fixed distance from the crucible; and
reach a threshold vapor pressure of the material, in some non-limiting examples, one of about 10⁻⁴ and 10⁻⁵ Torr.

In some non-limiting examples, the QCM may be mounted about 65 cm away from the crucible for the purpose of determining the sublimation temperature.

In some non-limiting examples, the patterning material 211 may have a sublimation temperature of one of between about: 100-320° C., 100-300° C., 120-300° C., 100-250° C., 140-280° C., 120-230° C., 130-220′C, 140-210° C., 140-200° C., 150-250° C., and 140-190° C.

### Melting Point

In some non-limiting examples, a material, including without limitation, a patterning material 211, with substantially low inter-molecular forces may tend to exhibit a substantially low melting point.

In some non-limiting examples, a material, including without limitation, a patterning material 211, having a substantially low melting point may have reduced applicability in some scenarios calling for substantial temperature reliability for temperatures of one of no more than about: 60° C., 80° C., and 100° C., in some non-limiting examples, because of changes in physical properties of such material at operating temperatures that approach the melting point.

In some non-limiting examples, a material with a melting point of about 120° C. may have reduced applicability in some scenarios calling for substantially high temperature reliability, including without limitation, of at least about: 100° C.

In some non-limiting examples, a material, including without limitation, a patterning material 211, having a substantially high melting point may have applicability in some scenarios calling for substantially high temperature reliability.

In some non-limiting examples, at least one of: the patterning coating 110 and the compound thereof may have a melting temperature that is one of at least about: 90° C., 100° C., 110° C., 120° C., 140° C., 150° C., and 180° C.,

### Cohesion Energy

According to Young's equation (Equation 13) the cohesion energy (fracture toughness/cohesion strength) of a material may tend to be proportional to its surface energy (cf. Young, Thomas (1805) “An essay on the cohesion of fluids”, Philosophical Transactions of the Royal Society of London, 95: 65-87).

According to Lindemann's criterion, the cohesion energy of a material may tend to be proportional to its melting temperature (cf Nanda, K. K., Sahu, S. N, and Behera, S. N (2002), “Liquid-drop model for the size-dependent melting of low-dimensional systems” Phys. Rev. A. 66 (1): 013208).

In some non-limiting examples, a material, including without limitation, a patterning material 211, having substantially low inter-molecular forces may tend to exhibit a substantially low cohesion energy.

In some non-limiting examples, a material, including without limitation, a patterning material 211, having a substantially low cohesion energy may have reduced applicability in some scenarios that call for substantial fracture toughness, including without limitation, in a device that may tend to undergo at least one of: sheer, and bending, stress during at least one of: manufacture, and use, as such material may tend to crack (fracture) in such scenarios. In some non-limiting examples, a material, including without limitation, a patterning material 211, having a cohesion energy of no more than about 30 dynes/cm may have reduced applicability in some scenarios in a device manufactured on a flexible substrate 10.

In some non-limiting examples, a material, including without limitation, a patterning material 211, that has a substantially high cohesion energy, may have applicability in some scenarios calling for substantially high reliability under at least one of: sheer, and bending, stress, including without limitation, a device manufactured on a flexible substrate 10.

In some non-limiting examples, a material, including without limitation, a patterning material 211, having a surface energy that is substantially low but is not unduly low may have applicability in some scenarios that call for substantial reliability under at least one of: sheer, and bending, stress, including without limitation, a device manufactured on a flexible substrate 10.

### Optical/Band Gap

In the present disclosure, a semiconductor material may be described as a material that generally exhibits a band gap. In some non-limiting examples, the band gap may be formed between a highest occupied molecular orbital (HOMO) and a lowest unoccupied molecular orbital (LUMO) of the semiconductor material. Semiconductor materials may thus tend to exhibit electrical conductivity that is substantially no more than that of a conductive material (including without limitation, at least one of: a metal, and an alloy), but that is substantially at least as great as an insulating material (including without limitation, glass). In some non-limiting examples, the semiconductor material may comprise an organic semiconductor material. In some non-limiting examples, the semiconductor material may comprise an inorganic semiconductor material.

In some non-limiting examples, an optical gap of a material, including without limitation, a patterning material 211, may tend to correspond to the HOMO-LUMO gap of the material.

In some non-limiting examples, a material, including without limitation, a patterning material 211, having a substantially large/wide optical (HOMO-LUMO) gap may tend to exhibit substantially weak, including without limitation, substantially no, photoluminescence in at least one of: the deep B(lue) region of the visible spectrum, the near UV spectrum, the visible spectrum, and the NIR spectrum.

In some non-limiting examples, a coating, including without limitation, a patterning coating 110, comprised of a material, including without limitation, a patterning material 211, having a substantially weak, including without limitation, substantially no, photoluminescence/absorption in a wavelength range of one of at least about: 365 nm, and 460 nm, may tend to not act as either of: a photoluminescent, and an absorbing, coating and may have applicability in some scenarios calling for substantially high transparency in at least one of: the visible spectrum, and the NIR spectrum.

In some non-limiting examples, such material may tend to exhibit substantially low photoluminescence upon being subjected to EM radiation having a wavelength of about 365 nm, which is a common wavelength of the radiation source used in fluorescence microscopy. The presence of such materials, including without limitation, a patterning material 211, especially when deposited, by way of non-limiting example, as a thin film, may have reduced applicability in some scenarios calling for typical optical detection techniques, including without limitation, fluorescence microscopy. This may impose constraints in some scenarios in which such material may be selectively deposited, for example through an FMM, over part(s) of a substrate 10, as there may be some scenarios for determining, following the deposition of the material, the part(s) in which such materials are present.

In some non-limiting examples, a material having a substantially large HOMO-LUMO gap may have applicability in some scenarios calling for weak, including without limitation, substantially no, photoluminescence/absorption in a wavelength range of one of at least about: 365 nm, and 460 nm.

In some non-limiting examples, a material having a substantially small HOMO-LUMO gap may have applicability in some scenarios to detect a film of the material using optical techniques.

In some non-limiting examples, an optical gap of the patterning material 211 may be wider than a photon energy of the EM radiation emitted by the source, such that the patterning material 211 does not undergo photoexcitation when subjected to such EM radiation.

### Refractive Index and Extinction Coefficient

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may have a low refractive index.

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may have a refractive index for EM radiation at a wavelength of 550 nm that may be one of no more than about: 1.55, 1.5, 1.45, 1.43, 1.4, 1.39, 1.37, 1.35, 1.32, and 1.3.

In some non-limiting examples, the refractive index of the patterning coating 110 may be no more than about 1.7. In some non-limiting examples, the refractive index of the patterning coating 110 may be one of no more than about: 1.6, 1.5, 1.4, and 1.3. In some non-limiting examples, the refractive index of the patterning coating 110 may be one of between about: 1.2-1.6, 1.2-1.5, and 1.25-1.45. As further described in various non-limiting examples above, the patterning coating 110 exhibiting a substantially low refractive index may have application in some scenarios, to enhance at least one of: the optical properties, and performance, of the device, including without limitation, by enhancing outcoupling of EM radiation emitted by the opto-electronic device.

Without wishing to be bound by any particular theory, it has been observed that providing the patterning coating 110 having a substantially low refractive index may, at least in some devices 100, enhance transmission of external EM radiation through the second portion 102 thereof. In some non-limiting examples, devices 100 including an air gap therein, which may be arranged near to the patterning coating 110, may exhibit a substantially high transmittance when the patterning coating 110 has a substantially low refractive index relative to a similarly configured device in which such low-index patterning coating 110 was not provided.

In some non-limiting examples, a series of samples was fabricated to measure the refractive index at a wavelength of 550 nm for the coatings formed by some of the various example materials. The results of the measurement are summarized in Table 4 below:

TABLE 4

| Material | Refractive Index |
|---|---|
| HT211 | 1.76 |
| HT01 | 1.80 |
| TAZ | 1.69 |
| Balq | 1.69 |
| Liq | 1.64 |
| Example Material 2 | 1.72 |
| Example Material 3 | 1.37 |
| Example Material 5 | 1.38 |
| Example Material 7 | 1.3 |
| Example Material 8 | 1.37 |

Based on the foregoing measurement of refractive index in Table 4, and the previous observation regarding one of: the presence, and absence, of a substantially closed coating 140 of Ag in Table 4, it was found that materials that form a low refractive index coating, which in some non-limiting examples, may be those having a refractive index of one of no more than about: 1.4 and 1.38, may have applicability in some scenarios for forming the patterning coating 110 to substantially inhibit deposition of a deposited material 331 thereon, including without limitation, at least one of: a metal, and an alloy, including without limitation, at least one of: Yb, Ag, Mg, and an Ag-containing material, including without limitation, MgAg.

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may have a low refractive index.

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may have a refractive index for EM radiation at a wavelength of 550 nm that may be one of no more than about: 1.55, 1.5, 1.45, 1.43, 1.4, 1.39, 1.37, 1.35, 1.32, and 1.3.

In some non-limiting examples, at least one of: the patterning coating 110, and the patterning material 211, in some non-limiting examples, when deposited as a film/coating in a form, and under circumstances similar to the deposition of the patterning coating 110 within the device 100, may have a low refractive index.

In some non-limiting examples, at least one of: the patterning coating 110, and the patterning material 211, in some non-limiting examples, when deposited as a film/coating in a form, and under circumstances similar to the deposition of the patterning coating 110 within the device 100, may have a refractive index for EM radiation at a wavelength of 550 nm that may be one of no more than about: 1.55, 1.5, 1.45, 1.43, 1.4, 1.39, 1.37, 1.35, 1.32, and 1.3.

In some non-limiting examples, the patterning coating 110 may be at least one of: substantially transparent, and EM radiation-transmissive.

In some non-limiting examples, at least one of: the patterning coating 110, and the patterning material 211, in some non-limiting examples, when deposited as at least one of: a film, and a coating, in a form, and under similar circumstances to the deposition of the patterning coating 110 within the device 100, may have an extinction coefficient that may be no more than about 0.01 for photons at a wavelength that is one of at least about: 600 nm, 500 nm, 460 nm, 420 nm, and 410 nm.

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may have an extinction coefficient that may be one of at least about: 0.05, 0.1, 0.2, and 0.5 for EM radiation at a wavelength that is one of no more than about: 400 nm, 390 nm, 380 nm, and 370 nm.

In this way, at least one of: the patterning coating 110, and the patterning material 211, when deposited as at least one of: a film, and a coating, in a form, and under circumstances similar to the deposition of the patterning coating 110 within the device 100, may absorb EM radiation in the UVA spectrum incident upon the device 100, thereby reducing a likelihood that EM radiation in the UVA spectrum may impart constraints in terms of at least one of: device performance, device stability, device reliability, and device lifetime.

In some non-limiting examples, the patterning coating 110 may exhibit an extinction coefficient of one of no more than about: 0.1, 0.08, 0.05, 0.03, and 0.01 in the visible light spectrum.

### Photoluminescence, Absorption and Other Optical Effects

In some non-limiting examples, photoluminescence of at least one of: a coating, and a material may be observed through a photoexcitation process. In a photoexcitation process, at least one of: the coating, and the material, may be subjected to EM radiation emitted by a source, including without limitation, a UV lamp.

When the emitted EM radiation is absorbed by at least one of: the coating, and the material, the electrons thereof may be temporarily excited. Following excitation, at least one relaxation process may occur, including without limitation, at least one of: fluorescence and phosphorescence, in which EM radiation may be emitted from at least one of: the coating, and the material.

The EM radiation emitted from at least one of: the coating, and the material, during such process may be detected, for example, by a photodetector, to characterize the photoluminescence properties of at least one of: the coating, and the material.

As used herein, a wavelength of photoluminescence, in relation to at least one of: the coating, and the material, may generally refer to a wavelength of EM radiation emitted by such at least one of: the coating, and the material, as a result of relaxation of electrons from an excited state. As would be appreciated by a person having ordinary skill in the relevant art, a wavelength of light emitted by at least one of: the coating, and the material, as a result of the photoexcitation process may, in some non-limiting examples, be longer than a wavelength of radiation used to initiate photoexcitation. Photoluminescence may be detected using various techniques known in the art, including, without limitation, fluorescence microscopy.

In some non-limiting examples, the optical gap of the various coatings/materials may correspond to an energy gap of the coating/material from which EM radiation is one of: absorbed, and emitted, during the photoexcitation process.

In some non-limiting examples, photoluminescence may be detected by subjecting the coating/material to EM radiation having a wavelength corresponding to the UV spectrum, such as in some non-limiting examples, one of: UVA, and UVB. In some non-limiting examples, EM radiation for causing photoexcitation may have a wavelength of about 365 nm.

In some non-limiting examples, the patterning material 211 may not substantially exhibit photoluminescence at any wavelength corresponding to the visible spectrum.

In some non-limiting examples, the patterning material 211 may not exhibit photoluminescence upon being subjected to EM radiation having a wavelength of one of at least about: 300 nm, 320 nm, 350 nm, and 365 nm.

As used herein, at least one of: the coating, and the material, that is photoluminescent, may be one that exhibits photoluminescence at a wavelength when irradiated with an excitation radiation at a certain wavelength. In some non-limiting examples, at least one of: the coating, and the material, that is photoluminescent, may exhibit photoluminescence at a wavelength that exceeds about 365 nm, which is a wavelength of the radiation source frequently used in fluorescence microscopy, upon being irradiated with an excitation radiation having a wavelength of 365 nm.

At least one of: the coating, and the material, that is photoluminescent, may be detected on a substrate 10 using standard optical techniques including without limitation, fluorescence microscopy, which may establish the presence of such at least one of: the coating, and the material.

In some non-limiting examples, a coating, including without limitation, a patterning coating 110, may exhibit photoluminescence, including without limitation, by comprising a material that exhibits photoluminescence.

In some non-limiting examples, the presence of such patterning coating 110 may be detected (observed) using routine characterization techniques such as fluorescence microscopy upon deposition of the patterning coating 110.

In some non-limiting examples, a coating, including without limitation, a patterning coating 110, may exhibit photoluminescence at a wavelength corresponding to at least one of: the UV spectrum, and visible spectrum, including without limitation, by comprising a material that exhibits photoluminescence. In some non-limiting examples, photoluminescence may occur at a wavelength (range) corresponding to the UV spectrum, including, without limitation, one of: the UVA spectrum, and UVB spectrum. In some non-limiting examples, photoluminescence may occur at a wavelength (range) corresponding to the visible spectrum. In some non-limiting examples, photoluminescence may occur at a wavelength (range) corresponding to one of: deep B(lue) and near UV.

In some non-limiting examples, at least one of the materials of the patterning coating 110 that may exhibit photoluminescence may comprise at least one of: a conjugated bond, an aryl moiety, a donor-acceptor group, and a heavy metal complex.

In some non-limiting examples, a coating, including without limitation, a patterning coating 110, comprised of a material, including without limitation, a patterning material 211, having substantially weak to no photoluminescence (absorption) in a wavelength range of one of at least about: 365 nm, and 460 nm, may tend to not act as one of: a photoluminescent, and an absorbing, coating and may have applicability in some scenarios calling for substantially high transparency in at least one of: the visible spectrum, and the NIR spectrum.

In some non-limiting examples, such material may tend to exhibit substantially low photoluminescence upon being subjected to EM radiation having a wavelength of about 365 nm, which is a wavelength of the radiation source frequently used in fluorescence microscopy. The presence of such materials, including without limitation, a patterning material 211, especially when deposited, in some non-limiting examples, as a thin film, may have reduced applicability in some scenarios calling for typical optical detection techniques, including without limitation, fluorescence microscopy. This may impose constraints in some scenarios in which such material may be selectively deposited, for example through an FMM, over part(s) of a substrate 10, as there may be some scenarios for determining, following the deposition of the material, the part(s) in which such materials are present.

In some non-limiting examples, at least one of: the patterning coating 110, and the patterning material 211, in some non-limiting examples, when deposited as at least one of: a film, and a coating, in a form, and under circumstances similar to the deposition of the patterning coating 110 within the device 100, may not substantially attenuate EM radiation passing therethrough, in at least the visible spectrum.

In some non-limiting examples, at least one of: the patterning coating 110, and the patterning material 211, when deposited as at least one of: a film, and a coating, in a form, and under circumstances similar to the deposition of the patterning coating 110 within the device 100, may not substantially attenuate EM radiation passing therethrough, in one of: the IR spectrum, and the NIR spectrum.

In this way, at least one of: the patterning coating 110, and the patterning material 211, when deposited as at least one of: a film, and a coating, in a form, and under circumstances similar to the deposition of the patterning coating 110 within the device 100, may absorb EM radiation in the UVA spectrum incident upon the device 100, thereby reducing a likelihood that EM radiation in the UVA spectrum may impart constraints in terms of at least one of: device performance, device stability, device reliability, and device lifetime.

In some non-limiting examples, the patterning coating 110 may modify at least one of: at least one property, and at least one characteristic, of EM radiation (including without limitation, in the form of photons) emitted by the device 100. In some non-limiting examples, the patterning coating 110 may exhibit a degree of haze, causing emitted EM radiation to be scattered. In some non-limiting examples, the patterning coating 110 may comprise a crystalline material for causing EM radiation transmitted therethrough to be scattered. Such scattering of EM radiation may facilitate enhancement of the outcoupling of EM radiation from the device 100 in some non-limiting examples. In some non-limiting examples, the patterning coating 110 may initially be deposited as a substantially non-crystalline, including without limitation, substantially amorphous, coating, whereupon, after deposition thereof, the patterning coating 110 may become crystallized and thereafter serve as an optical coupling.

In some non-limiting examples, the patterning material 211 may exhibit insignificant, including without limitation, no detectable, absorption when subjected to EM radiation having a wavelength of one of at least about: 300 nm, 320 nm, 350 nm, and 365 nm.

In some non-limiting examples, the patterning coating 110 may not exhibit any substantial EM radiation absorption at any wavelength corresponding to the visible spectrum.

### Average Layer Thickness

In some non-limiting examples, an average layer thickness of the patterning coating 110 may be one of no more than about: 10 nm, 8 nm, 7 nm, 6 nm, and 5 nm.

### Weight

Without wishing to be bound by any particular theory, it may be postulated that, for compounds that are adapted to form surfaces with substantially low surface energy, there may be scenarios calling for, in at least some applications, the molecular weight of such compounds to be one of between about: 500-3,000 g/mol, 550-3,000 g/mol, 800-3,000 g/mol, 900-2,000 g/mol, 900-1,800 g/mol, and 900-1,600 g/mol.

In some non-limiting examples, the molecular weight of the compound of the at least one patterning material 211 may be no more than about 5,000 g/mol. In some non-limiting examples, the molecular weight of the compound may be one of no more than about: 4,500 g/mol, 4,000 g/mol, 3,800 g/mol, and 3,500 g/mol.

In some non-limiting examples, the molecular weight of the compound of the at least one patterning material 211 may be at least about 500 g/mol. In some non-limiting examples, the molecular weight of the compound may be one of at least about: 500 g/mol, 550 g/mol, 700 g/mol, 800 g/mol, 1,000 g/mol, 1200 g/mol, 1300 g/mol, 1,500 g/mol, 1,700 g/mol, 2,000 g/mol, 2,200 g/mol, and 2,500 g/mol.

In some non-limiting examples, the molecular weight of the compound of the at least one patterning material 211 may be one of between about: 800-3,000 g/mol, 900-2,000 g/mol, 900-1,800 g/mol, and 900-1,600 g/mol.

In some non-limiting examples, a percentage of the molar weight of such compounds, including without limitation, of the at least one patterning material 211, that may be attributable to the presence of F atoms, may be one of between about: 40-90%, 45-85%, 50-80%, 55-75%, and 60-75%. In some non-limiting examples, F atoms may constitute a majority of a molar weight of such compound.

In some non-limiting examples, a percentage of the molar weight of such compounds, including without limitation, of the at least one patterning material 211, that may be attributable to the presence of fluorocarbon moieties, may be one of at least about: 50%, 60%, 65%, 70%, 75%, 80%, and 85%. In some non-limiting examples, fluorocarbon moieties may constitute a majority of a molar weight of such compound. In some non-limiting examples, fluorocarbon moieties, including without limitation, those comprising at least one of: CF, CF₂, CF₃, and a CF₂H unit, may be moieties primarily constituting F and C atoms.

In some non-limiting examples, a quotient of: a number of F atoms contained in the compound/a number of C atoms contained in the compound, including without limitation, of the at least one patterning material 211, may be one of at least about: 0.5, 0.7, 1, 1.5, 2, and 2.5. In some non-limiting examples, a quotient of: a number of F atoms/a number of Si atoms may be one of no more than about: 5, 4, and 3.

In some non-limiting examples, a percentage of the molar weight of such compounds, including without limitation, of the at least one patterning material 211, that may be attributable to the presence of the heterocyclic moiety, may be one of no more than about: 60%, 50%, 45%, 40%, 35%, 30%, 25%, 20%, 15%, and 10%.

In some non-limiting examples, a percentage of the molar weight of such compounds, including without limitation, of the at least one patterning material 211, that may be attributable to the presence of the heterocyclic moiety, may be no more than a percentage of the molar weight of such compounds that may be attributable to at least one of: the fluorocarbon moieties, and F atoms, contained by such compound.

Without wishing to be limited by any particular theory, it may be postulated that the heterocyclic moiety may generally exhibit a substantially high surface tension. In some non-limiting examples, in some scenarios calling for a substantially low surface energy of the film/coating formed by the compound, a molecular structure of the compound comprising an increased proportion of low surface tension moieties, including without limitation, at least one of: fluorocarbon moieties and F atoms, may have applicability compared to a molecular structure of such compound comprising an increased proportion of high surface tension moieties, including without limitation, a heterocyclic moiety.

In some non-limiting examples, a quotient of: a number of F atoms contained in the compound/a number of N atoms contained in the compound, including without limitation, of the at least one patterning material 211, may be one of at least about: 2, 4, 5, 6, 8, 10, 14, 18, 20, and 24. In some non-limiting examples, such quotient may be calculated by only counting the number of F atoms contained in the F-containing moiety and dividing it by the number of N atoms contained in the heterocyclic moiety of the compound.

### Inter-Relationships Between Patterning Coating Attributes

Without wishing to be bound by any particular theory, it may be postulated that exposed layer surfaces 11 exhibiting low initial sticking probability with respect to the deposited material 331, including without limitation, at least one of: a metal, and an alloy, including without limitation, Yb, Ag, Mg, and an Ag-containing material, including without limitation, MgAg, may exhibit high transmittance. Without wishing to be bound by any particular theory, it may be postulated that exposed layer surfaces 11 exhibiting high sticking probability with respect to the deposited material 331, including without limitation, at least one of: a metal, and an alloy, including without limitation, Yb, Ag, Mg, and an Ag-containing material, including without limitation, MgAg, may exhibit low transmittance.

In some non-limiting examples, a material, including without limitation, a patterning material 211, may tend to have a substantially high initial sticking probability against deposition of a deposited material, including without limitation, at least one of: a metal, and an alloy, including without limitation, at least one of: Yb, Ag, Mg, and an Ag-containing material, including without limitation, MgAg, if the material has a substantially high surface energy.

In some non-limiting examples, a patterning material 211 that has a substantially low surface tension that is not unduly low, may have applicability in some scenarios calling for a substantially high melting point, including without limitation, between about 15-22 dynes/cm.

In some non-limiting examples, a material, including without limitation, a patterning material 211, having a surface tension that is substantially low, but not unduly low, may have applicability in some scenarios that call for a substantially high sublimation temperature.

In some non-limiting examples, a coating, including without limitation, a patterning coating 110, comprised of a material, including without limitation, a patterning material 211, having a substantially low surface energy and a substantially high sublimation temperature may have application in some scenarios calling for substantially high precision in the control of the average layer thickness of a film comprising such material.

Without wishing to be bound by any particular theory, it may be postulated that materials that form an exposed layer surface 11 having a surface energy of no more than about, in some non-limiting examples, 13 dynes/cm, may have reduced applicability as a patterning material 211 in some scenarios, as such materials may exhibit at least one of: substantially low adhesion to layer(s) surrounding such materials, a substantially low melting point, and a substantially low sublimation temperature.

In some non-limiting examples, a patterning coating 110 having a substantially low surface energy and a substantially high melting point may have applicability in some scenarios calling for high temperature reliability. In some non-limiting examples, there may be challenges in achieving such a combination from a single material given that in some non-limiting examples, a single material having a low surface energy may tend to exhibit a low melting point.

Without wishing to be bound by any particular theory, it may be postulated that such compounds, including without limitation, of at least one patterning material 211, may exhibit at least one property that may have applicability in some scenarios for forming at least one of: a coating, and layer, having at least one of: a substantially high melting point, in some non-limiting examples, of at least 100° C., a substantially low surface energy, and a substantially amorphous structure, when deposited, in some non-limiting examples, using vacuum-based thermal evaporation processes.

In some non-limiting examples, a coating, including without limitation, a patterning coating 110, having a substantially low surface energy, a substantially high cohesion energy, and a substantially high melting point may have applicability in some scenarios that call for substantially high reliability under various conditions. In some non-limiting examples, there may be challenges in achieving such a combination from a single material, given that, in some non-limiting examples, a unitary material having a substantially low surface energy may tend to exhibit a substantially low cohesion energy and a substantially low melting point.

In some non-limiting examples, a material, including without limitation, a patterning material 211, having a substantially low surface energy and a substantially high cohesion energy may have applicability in some scenarios that call for substantially high reliability under at least one of: sheer, and bending, stress. In some non-limiting examples, there may be challenges in achieving such a combination from a single material, given that, in some non-limiting examples, a thin film formed substantially of a single material having a substantially low surface energy may tend to exhibit a substantially low cohesion energy.

In some non-limiting examples, a material, including without limitation, a patterning material 211, having a substantially low surface energy may tend to exhibit at least one of: a substantially large, and substantially wide, optical gap. In some non-limiting examples, the optical gap of a material, including without limitation, a patterning material 211, may tend to correspond to the HOMO-LUMO gap of the material.

In general, a material with a low surface energy may exhibit at least one of: a large, and wide, optical gap which may, in some non-limiting examples, correspond to the HOMO-LUMO gap of the material.

It has also now been found that a patterning coating 110 formed by a compound exhibiting a substantially low surface energy may also exhibit a substantially low refractive index.

In some non-limiting examples, at least one of: the patterning coating 110, and the patterning material 211, may exhibit a surface energy of no more than about 25 dynes/cm and a refractive index of no more than about 1.45. In some non-limiting examples, at least one of: the patterning coating 110, and the patterning material 211, may comprise a material exhibiting a surface energy of no more than about 20 dynes/cm and a refractive index of no more than about 1.4.

In some non-limiting examples, a material, including without limitation, a patterning material 211, having a substantially low surface energy may have applicability in some scenarios calling for substantially weak to no, at least one of: photoluminescence, and absorption, in a wavelength range that is one of at least about: 365 nm and 460 nm.

In some non-limiting examples, a material, including without limitation, a patterning material 211, having at least one of: a substantially large, and substantially wide optical gap (and HOMO-LUMO gap) may tend to exhibit a substantially weak to no photoluminescence in at least one of: the deep B(lue) region of the visible spectrum, the near UV spectrum, the visible spectrum, and the NIR spectrum.

Without wishing to be bound by any particular theory, it may be postulated that, for compounds that are adapted to form surfaces with substantially low surface energy, there may be an aim, in at least some applications, for the molecular weight of such compounds to be one of between about: 1,500-5,000 g/mol, 1,500-4,500 g/mol, 1,700-4,500 g/mol, 2,000-4,000 g/mol, 2,200-4,000 g/mol, and 2,500-3,800 g/mol.

At least some materials with at least one of: one of: a large, and wide, optical gap, and HOMO-LUMO gap, may exhibit substantially weak to no photoluminescence in at least one of: the visible spectrum, the deep B(lue) region thereof, and the near UV spectrum. In some non-limiting examples, a material with a substantially small HOMO-LUMO gap may have applicability in applications to detect a film of the material using optical techniques. In some non-limiting examples, a material with higher surface energy may have applicability for applications to detect of a film of the material using optical techniques.

In some non-limiting examples, a material having a substantially large HOMO-LUMO gap may have applicability in some scenarios calling for weak to no at least one of: photoluminescence, and absorption, in a wavelength range of one of at least about: 365 nm, and 460 nm.

### Doping

In some non-limiting examples, the patterning coating 110 may provide, including without limitation, because of at least one of: the patterning material 211 used, and the deposition environment, at least one nucleation site for the deposited material 331.

In some non-limiting examples, the patterning coating 110 may be doped, including without limitation, by at least one of: covering, and supplementing, with another material that may act as at least one of: a seed, and heterogeneity, to act as such a nucleation site for the deposited material 331. In some non-limiting examples, such other material may comprise an NPC 520 material. In some non-limiting examples, such other material may comprise an organic material, including without limitation, at least one of: a polycyclic aromatic compound, and a material comprising a non-metallic element, including without limitation, at least one of: O, S, N, and C, whose presence might otherwise be a contaminant in at least one of: the source material, equipment used for deposition, and the vacuum chamber environment. In some non-limiting examples, such other material may be deposited in a layer thickness that is a fraction of a monolayer, to avoid forming a closed coating 140 thereof. Rather, the monomers of such other material may tend to be spaced apart in the lateral aspect so as form discrete nucleation sites for the deposited material.

### Plurality of Patterning Materials

In some non-limiting examples, forming a patterning coating 110 of a single patterning material 211 against the deposition of a deposited material 331, including without limitation, at least one of: a given metal, and a given alloy, including without limitation, at least one of: Yb, Ag, Mg, and Ag-containing materials, including without limitation, MgAg, that satisfied constraints of at least one material property selected from at least one of: initial sticking probability, transmittance, deposition contrast, surface energy, glass transition temperature, melting point, sublimation temperature, evaporation temperature, cohesion energy, optical gap, photoluminescence, refractive index, extinction coefficient, absorption, other optical effect, average layer thickness, molecular weight, and composition, for a given scenario, may impose challenges, given the substantially complex inter-relationships between the various material properties.

In some non-limiting examples, the patterning coating 110 may comprise a plurality of materials. In some non-limiting examples, the patterning coating 110 may comprise a first material and a second material.

In some non-limiting examples, at least one of the plurality of materials of the patterning coating 110 may serve as an NIC when deposited as a thin film.

In some non-limiting examples, at least one of the plurality of materials of the patterning coating 110 may serve as an NIC when deposited as a thin film, and another material thereof may form an NPC 520 when deposited as a thin film. In some non-limiting examples, the first material may form an NPC 520 when deposited as a thin film, and the second material may form an NIC when deposited as a thin film. In some non-limiting examples, the presence of the first material in the patterning coating 110 may result in an increased initial sticking probability thereof compared to cases in which the patterning coating 110 is formed of the second material and is substantially devoid of the first material.

In some non-limiting examples, at least one of the materials of the patterning coating 110 may be adapted to form a surface having a low surface energy when deposited as a thin film. In some non-limiting examples, the first material, when deposited as a thin film, may be adapted to form a surface having a lower surface energy than a surface provided by a thin film comprising the second material.

In some non-limiting examples, the patterning coating 110 may exhibit photoluminescence, including without limitation, by comprising a material which exhibits photoluminescence.

In some non-limiting examples, the first material may exhibit photoluminescence at a wavelength corresponding to the visible spectrum, and the second material may not exhibit substantial photoluminescence at any wavelength corresponding to the visible spectrum.

In some non-limiting examples, the second material may not substantially exhibit photoluminescence at any wavelength corresponding to the visible spectrum. In some non-limiting examples, the second material may not exhibit photoluminescence upon being subjected to EM radiation having a wavelength of one of at least about: 300 nm, 320 nm, 350 nm, and 365 nm. In some non-limiting examples, the second material may exhibit insignificant to no detectable absorption when subjected to such EM radiation.

In some non-limiting examples, the second optical gap of the second material may be wider than the photon energy of the EM radiation emitted by the source, such that the second material does not undergo photoexcitation when subjected to such EM radiation. However, in some non-limiting examples, the patterning coating 110 comprising such second material may nevertheless exhibit photoluminescence upon being subjected to EM radiation due to the first material exhibiting photoluminescence. In some non-limiting examples, the presence of the patterning coating 110 may be detected using routine characterization techniques such as fluorescence microscopy upon deposition of the patterning coating 110.

In some non-limiting examples, the first material may have a first optical gap, and the second material may have a second optical gap. In some non-limiting examples, the second optical gap may exceed the first optical gap. In some non-limiting examples, a difference between the first optical gap and the second optical gap may exceed one of about: 0.3 eV, 0.5 eV, 0.7 eV, 1 eV, 1.3 eV, 1.5 eV, 1.7 eV, 2 eV, 2.5 eV, and 3 eV.

In some non-limiting examples, the first optical gap may be one of no more than about: 4.1 eV, 3.5 eV, and 3.4 eV. In some non-limiting examples, the second optical gap may exceed one of about: 3.4 eV, 3.5 eV, 4.1 eV, 5 eV, and 6.2 eV.

In some non-limiting examples, at least one of: the first optical gap, and the second optical gap, may correspond to the HOMO-LUMO gap.

In some non-limiting examples, an optical gap of at least one of: the various coatings, and materials, including without limitation, at least one of: the first optical gap, and the second optical gap, may correspond to an energy gap of at least one of: the coating, and the material, from which EM radiation is at least one of: absorbed, and emitted, during the photoexcitation process.

In some non-limiting examples, a concentration, including without limitation by weight, of the first material in the patterning coating 110 may be no more than that of the second material in the patterning coating 110. In some non-limiting examples, the patterning coating 110 may comprise one of at least about: 0.1 wt. %, 0.2 wt. %, 0.5 wt. %, 0.8 wt. %, 1 wt. %, 3 wt. %, 5 wt. %, 8 wt. %, 10 wt. %, 15 wt. %, and 20 wt. %, of the first material. In some non-limiting examples, the patterning coating 110 may comprise one of no more than about: 50 wt. %, 40 wt. %, 30 wt. %, 25 wt. %, 20 wt. %, 15 wt. %, 10 wt. %, 8 wt. %, 5 wt. %, 3 wt. %, and 1 wt. %, of the first material. In some non-limiting examples, a remainder of the patterning coating 110 may be substantially comprised of the second material. In some non-limiting examples, the patterning coating 110 may comprise additional materials, including without limitation, at least one of: a third material, and a fourth material.

In some non-limiting examples, at least one of the materials of the patterning coating 110, including without limitation, the first material and the second material, may comprise at least one of: F, and Si. By way of non-limiting example, at least one of: the first material, and the second material, may comprise at least one of: F, and Si. In some further non-limiting examples, the first material may comprise at least one of: F, and Si, and the second material may comprise at least one of: F, and Si. In some non-limiting examples, the first material and the second material both may comprise F. In some non-limiting examples, the first material and the second material both may comprise Si. In some non-limiting examples, each of the first material and the second material may comprise at least one: F, and Si.

In some non-limiting examples, at least one material of the first material and the second material may comprise both F and Si. In some non-limiting examples, one of the first material and the second material may not comprise at least one of: F, and Si. In some non-limiting examples, the second material may comprise at least one of: F, and Si, and the first material may not comprise at least one of: F, and Si.

In some non-limiting examples, at least one of the materials of the patterning coating 110, including without limitation, the first material and the second material, may comprise a compound that comprises F. In some non-limiting examples, at least one of: the first material, and the second material, may comprise a compound that comprises F and a C atom. In some non-limiting examples, at least one of: the first material, and the second material, may comprise a compound that comprises F and C in an atomic ratio corresponding to a quotient of F/C of one of at least about: 0.5, 0.7, 1, 1.5, 2, and 2.5.

In some non-limiting examples, an atomic ratio of F to C may be determined by counting the F atoms present in the compound structure, and for C atoms, only counting the sp³ hybridized C atoms present in the compound structure. In some non-limiting examples, at least one of the materials of the patterning coating 110, including without limitation, the first material and the second material, may comprise a compound that comprises, as part of its molecular sub-structure, a moiety comprising F and C in an atomic ratio corresponding to a quotient of F/C of one of at least about: 1, 1.5, and 2.

In some non-limiting examples, at least one of the materials of the patterning coating 110, which for example, may be at least one of: the first material, and the second material, may comprise F, and at least one of the other materials of the patterning coating 110 may comprise a sp² carbon. In some non-limiting examples, at least one of the materials of the patterning coating 110, including without limitation, at least one of: the first material, and the second material, may comprise F, and at least one of the other materials of the patterning coating 110 may comprise a sp³ carbon. In some non-limiting examples, at least one of the materials of the patterning coating 110, including without limitation, at least one of: the first material, and the second material, may comprise F and a sp³ carbon, and at least one of the other materials of the patterning coating 110 may comprise a sp² carbon. In some non-limiting examples, at least one of the materials of the patterning coating 110, including without limitation, at least one of: the first material, and the second material, may comprise F and a sp³ carbon wherein all F bonded to a C may be bonded to a sp³ carbon, and at least one of the other materials of the patterning coating 110 may comprise a sp² carbon. In some non-limiting examples, at least one of the materials of the patterning coating 110, including without limitation, at least one of: the first material, and the second material, may comprise F and a sp³ carbon wherein all F bonded to C may be bonded to an sp³ carbon, and at least one of the other materials of the patterning coating 110 may comprise a sp² carbon and may not comprise F. By way of non-limiting example, in any of the foregoing non-limiting examples, “at least one of the materials of the patterning coating 110” may correspond to the second material, and the “at least one of the other materials of the patterning coating 110” may correspond to the first material.

Those having ordinary skill in the relevant art will appreciate that the presence of materials in a coating which comprises at least one of: F, sp² carbon, sp³ carbon, an aromatic hydrocarbon moiety, other functional groups, and other moieties, may be detected using various methods known in the art, including by way of non-limiting example, X-ray Photoelectron Spectroscopy (XPS).

In some non-limiting examples, at least one of the materials of the patterning coating 110, which by way of non-limiting example may be at least one of: the first material, and the second material, may comprise F, and at least one of the other materials of the patterning coating 110 may comprise an aromatic hydrocarbon moiety. In some non-limiting examples, at least one of the materials of the patterning coating 110, including without limitation, at least one of: the first material, and the second material, may comprise F, and at least one of the materials of the patterning coating 110 may not comprise an aromatic hydrocarbon moiety. In some non-limiting examples, at least one of the materials of the patterning coating 110, including without limitation, at least one of: the first material, and the second material, may comprise F and may not comprise an aromatic hydrocarbon moiety, and at least one of the other materials of the patterning coating 110 may comprise an aromatic hydrocarbon moiety. In some non-limiting examples, at least one of the materials of the patterning coating 110, including without limitation, at least one of: the first material, and the second material, may comprise F and may not comprise an aromatic hydrocarbon moiety, and at least one of the other materials of the patterning coating 110 may comprise an aromatic hydrocarbon moiety and may not comprise F. In some non-limiting examples, the aromatic hydrocarbon moiety may comprise at least one of: a substituted polycyclic aromatic hydrocarbon moiety, an unsubstituted polycyclic aromatic hydrocarbon moiety, a substituted phenyl moiety, and an unsubstituted phenyl moiety.

In some non-limiting examples, at least one of the materials of the patterning coating 110, including without limitation, at least one of: the first material, and the second material, may comprise F, and at least one of the other materials of the patterning coating 110 may comprise a polycyclic aromatic hydrocarbon moiety. In some non-limiting examples, at least one of the materials of the patterning coating 110, including without limitation, at least one of: the first material, and the second material, may comprise F, and at least one of the materials of the patterning coating 110 may not comprise a polycyclic aromatic hydrocarbon moiety. In some non-limiting examples, at least one of the materials of the patterning coating 110, including without limitation, at least one of: the first material, and the second material, may comprise F and may not comprise a polycyclic aromatic hydrocarbon moiety, and at least one of the other materials of the patterning coating 110 may comprise a polycyclic aromatic hydrocarbon moiety. In some non-limiting examples, at least one of the materials of the patterning coating 110, including without limitation, at least one of: the first material, and the second material, may comprise F and may not comprise a polycyclic aromatic hydrocarbon moiety, and at least one of the other materials of the patterning coating 110 may comprise a polycyclic aromatic hydrocarbon moiety and may not comprise F.

In some non-limiting examples, at least one of the materials of the patterning coating 110, including without limitation, the first material and the second material, may comprise an organic-inorganic hybrid material.

In some non-limiting examples, at least one of the materials of the patterning coating 110, including without limitation, the first material and the second material, may comprise an oligomer.

In some non-limiting examples, at least one of the materials of the patterning coating 110, including without limitation, the first material and the second material, may comprise a compound having a molecular structure comprising a backbone and at least one functional group bonded to the backbone. In some non-limiting examples, the backbone may be an inorganic moiety, and the at least one functional group may be an organic moiety.

In some non-limiting examples, such compound may have a molecular structure comprising a siloxane group. In some non-limiting examples, the siloxane group may be one of: a linear siloxane group, a branched siloxane group, and a cyclic siloxane group. In some non-limiting examples, the backbone may comprise a siloxane group. In some non-limiting examples, the backbone may comprise a siloxane group and at least one functional group comprising F. In some non-limiting examples, the at least one functional group comprising F may be a fluoroalkyl group. In some non-limiting examples, such compound may comprise fluoro-siloxanes, including without limitation, Example Material 6 and Example Material 9 (discussed below).

In some non-limiting examples, at least one of the materials of the patterning coating 110, including without limitation, at least one of: the first material, and the second material, may comprise at least one of: a fluorocarbon moiety and a siloxane moiety, and at least one of the other materials of the patterning coating 110 may comprise a polycyclic aromatic hydrocarbon moiety. In some non-limiting examples, at least one of the materials of the patterning coating 110, including without limitation, at least one of: the first material, and the second material, may comprise at least one of: a fluorocarbon moiety, and a siloxane moiety, and at least one of the materials of the patterning coating 110 may not comprise a polycyclic aromatic hydrocarbon moiety. In some non-limiting examples, at least one of the materials of the patterning coating 110, including without limitation, at least one of: the first material, and the second material, may comprise at least one of: a fluorocarbon moiety, and a siloxane moiety, and may not comprise a polycyclic aromatic hydrocarbon moiety, and at least one of the other materials of the patterning coating 110 may comprise a polycyclic aromatic hydrocarbon moiety. In some non-limiting examples, at least one of the materials of the patterning coating 110, including without limitation, at least one of: the first material, and the second material, may comprise at least one of: a fluorocarbon moiety, and a siloxane moiety, and may not comprise a polycyclic aromatic hydrocarbon moiety, and at least one of the other materials of the patterning coating 110 may comprise a polycyclic aromatic hydrocarbon moiety and may not comprise at least one of: a fluorocarbon moiety, and a siloxane moiety.

In some non-limiting examples, the compound may have a molecular structure comprising a silsesquioxane group. In some non-limiting examples, the silsesquioxane group may be a POSS. In some non-limiting examples, the backbone may comprise a silsesquioxane group. In some non-limiting examples, the backbone may comprise a silsesquioxane group and at least one functional group comprising F. In some non-limiting examples, the at least one functional group comprising F may be a fluoroalkyl group. In some non-limiting examples, such compound may comprise fluoro-silsesquioxane and fluoro-POSS, including without limitation, Example Material 8 (discussed below).

In some non-limiting examples, the compound may have a molecular structure comprising at least one of: a substituted aryl group, an unsubstituted aryl group, a substituted heteroaryl group, and an unsubstituted heteroaryl group. In some non-limiting examples, the aryl group may be at least one of: phenyl, and naphthyl. In some non-limiting examples, at least one C atom of an aryl group may be substituted by a heteroatom, which by way of non-limiting example may be at least one of: O, N, and S, to derive a heteroaryl group. In some non-limiting examples, the backbone may comprise at least one of: a substituted aryl group, an unsubstituted aryl group, a substituted heteroaryl group, and an unsubstituted heteroaryl group. In some non-limiting examples, the molecular structure of the compound may comprise: a backbone comprising at least one of: a substituted aryl group, an unsubstituted aryl group, a substituted heteroaryl group, and an unsubstituted heteroaryl group; and at least one functional group comprising F. In some non-limiting examples, the at least one functional group comprising F may be a fluoroalkyl group.

In some non-limiting examples, at least one of the materials of the patterning coating 110, including without limitation, at least one of: the first material, and the second material, may comprise F, and at least one of the other materials of the patterning coating 110 may comprise a phenyl moiety. In some non-limiting examples, at least one of the materials of the patterning coating 110, including without limitation, at least one of: the first material, and the second material, may comprise F, and at least one of the materials of the patterning coating 110 may not comprise a phenyl moiety. In some non-limiting examples, at least one of the materials of the patterning coating 110, including without limitation, at least one of: the first material, and the second material, may comprise F and may not comprise a phenyl moiety, and at least one of the other materials of the patterning coating 110 may comprise a phenyl moiety. In some non-limiting examples, at least one of the materials of the patterning coating 110, including without limitation, at least one of: the first material, and the second material, may comprise F and may not comprise a phenyl moiety, and at least one of the other materials of the patterning coating 110 may comprise a phenyl moiety and may not comprise F.

In some non-limiting examples, at least one of the materials of the patterning coating 110, including without limitation, at least one of: the first material, and the second material, may comprise at least one of: a fluorocarbon moiety and a siloxane moiety, and at least one of the other materials of the patterning coating 110 may comprise a phenyl moiety. In some non-limiting examples, at least one of the materials of the patterning coating 110, including without limitation, at least one of: the first material, and the second material, may comprise at least one of: a fluorocarbon moiety, and a siloxane moiety, and at least one of the materials of the patterning coating 110 may not comprise a phenyl moiety. In some non-limiting examples, at least one of the materials of the patterning coating 110, including without limitation, at least one of: the first material, and the second material, may comprise at least one of: a fluorocarbon moiety, and a siloxane moiety and may not comprise a phenyl moiety, and at least one of the other materials of the patterning coating 110 may comprise a phenyl moiety. In some non-limiting examples, at least one of the materials of the patterning coating 110, including without limitation, at least one of: the first material, and the second material, may comprise at least one of: a fluorocarbon moiety, and a siloxane moiety and may not comprise a phenyl moiety, and at least one of the other materials of the patterning coating 110 may comprise a phenyl moiety and may not comprise either of: a fluorocarbon moiety, and a siloxane moiety.

In some non-limiting examples, the compound may have a molecular structure comprising at least one of: a substituted hydrocarbon group, an unsubstituted hydrocarbon group, a linear hydrocarbon group, a branched hydrocarbon group, and a cyclic hydrocarbon group. In some non-limiting examples, at least one C atom of the hydrocarbon group may be substituted by a heteroatom, including without limitation, at least one of: O, N, and S.

In some non-limiting examples, the compound may have a molecular structure comprising a phosphazene group. In some non-limiting examples, the phosphazene group may be at least one of: a linear phosphazene group, a branched phosphazene group, and a cyclic phosphazene group. In some non-limiting examples, the backbone may comprise a phosphazene group. In some non-limiting examples, the backbone may comprise a phosphazene group and at least one functional group comprising F. In some non-limiting examples, the at least one functional group comprising F may be a fluoroalkyl group. In some non-limiting examples, such compound may comprise fluoro-phosphazenes, including without limitation, Example Material 4 (discussed below).

In some non-limiting examples, the compound may be a fluoropolymer. In some non-limiting examples, the compound may be a block copolymer comprising F. In some non-limiting examples, the compound may be an oligomer. In some non-limiting examples, the oligomer may be a fluorooligomer. In some non-limiting examples, the compound may be a block oligomer comprising F. In some non-limiting examples, the at least one of: fluoropolymers, and fluorooligomers, include ones having a molecular structure of at least one of: Example Material 3, Example Material 5, and Example Material 7.

In some non-limiting examples, the compound may be a metal complex. In some non-limiting examples, the metal complex may be an organo-metal complex. In some non-limiting examples, the organo-metal complex may comprise F. In some non-limiting examples, the organo-metal complex may comprise at least one ligand comprising F. In some non-limiting examples, the at least one ligand comprising F may comprise a fluoroalkyl group.

In general, at least one of: the molecular structures, and molecular compositions, of the materials of the patterning coating 110, including without limitation, at least one of: the first material, and the second material, may be different. In some non-limiting examples, the materials may be selected such that they possess at least one property which is one of: substantially similar to, and substantially different from, one another, including without limitation, at least one of: at least one of: a molecular structure of a monomer, a monomer backbone, and a functional group; a presence of a element in common; a similarity in molecular structure; a characteristic surface energy; a refractive index; a molecular weight; and a thermal property, including without limitation, at least one of: a melting temperature, a sublimation temperature, a glass transition temperature, and a thermal decomposition temperature.

A characteristic surface energy, as used herein, in some non-limiting examples, with respect to a material, may generally refer to a surface energy determined from such material. By way of non-limiting example, a characteristic surface energy may be measured from a surface formed by the material deposited in a thin film form. Various methods and theories for determining the surface energy of a solid are known. By way of non-limiting example, a surface energy may be determined based on a series of contact angle measurements, in which various liquids may be brought into contact with a surface of a solid to measure a contact angle between the liquid-vapor interface and the surface. In some non-limiting examples, a surface energy of a solid surface may be equal to the surface tension of a liquid with the highest surface tension that completely wets the surface. By way of non-limiting example, a Zisman plot may be used to determine a highest surface tension value that would result in complete wetting (i.e. contact angle of 0°) of the surface.

In some non-limiting examples, at least one of: the first material, and the second material, of the patterning coating 110 may be an oligomer.

In some non-limiting examples, the first material may comprise a first oligomer, and the second material may comprise a second oligomer. Each of the first oligomer and the second oligomer may comprise a plurality of monomers.

In some non-limiting examples, at least a fragment of the molecular structure of the at least one of the materials of the patterning coating 110, including without limitation, at least one of: the first material, and the second material, may be represented by Formula (I):

$${( Mon )}_{n} \qquad ( I )$$

where:
Mon represents a monomer, and
n is an integer of at least 2.

In some non-limiting examples, n may be an integer of one of between about: 2-100, 2-50, 3-20, 3-15, 3-10, and 3-7.

In some non-limiting examples, the molecular structure of the first material and the second material of the patterning coating 110 may each be independently represented by Formula (I). In some non-limiting examples, at least one of: the monomer, and n, of the first material may be different from that of the second material. In some non-limiting examples, n of the first material may be the same as n of the second material. In some non-limiting examples, n of the first material may be different from n of the second material. In some non-limiting examples, the first material and the second material may be oligomers.

In some non-limiting examples, the monomer may comprise at least one of: F, and Si.

In some non-limiting examples, the monomer may comprise a functional group. In some non-limiting examples, at least one functional group of the monomer may have a low surface tension. In some non-limiting examples, at least one functional group of the monomer may comprise at least one of: F, and Si. In some non-limiting examples, such functional group may comprise at least one of: a fluorocarbon group, and a siloxane group. In some non-limiting examples, the monomer may comprise a silsesquioxane group.

While some non-limiting examples have been described herein with reference to a first material and a second material, those having ordinary skill in the relevant art will appreciate that the patterning coating may further comprise at least one additional material, and descriptions regarding at least one of: the molecular structures, and properties, of at least one of: the first material, the second material, the first oligomer, and the second oligomer, may be applicable with respect to additional materials which may be contained in the patterning coating 110.

The surface tension attributable to a fragment of a molecular structure, including without limitation, at least one of: a monomer, a monomer backbone unit, a linker, and a functional group, may be determined using various known methods in the art, including without limitation, the use of a Parachor, such as may be further described, by way of non-limiting example, in “Conception and Significance of the Parachor”, Nature 196: 890-891. In some non-limiting examples, at least one functional group of the monomer may have a surface tension of one of no more than about: 25 dynes/cm, 21 dynes/cm, 20 dynes/cm, 19 dynes/cm, 18 dynes/cm, 17 dynes/cm, 16 dynes/cm, 15 dynes/cm, 14 dynes/cm, 13 dynes/cm, 12 dynes/cm, 11 dynes/cm, and 10 dynes/cm.

In some non-limiting examples, the monomer may comprise at least one of: a CF₂, and a CF₂H, moiety. In some non-limiting examples, the monomer may comprise at least one of: a CF₂, and a CF₃, moiety. In some non-limiting examples, the monomer may comprise a CH₂CF₃ moiety. In some non-limiting examples, the monomer may comprise at least one of: C, and O. In some non-limiting examples, the monomer may comprise a fluorocarbon monomer. In some non-limiting examples, the monomer may comprise at least one of: a vinyl fluoride moiety, a vinylidene fluoride moiety, a tetrafluoroethylene moiety, a chlorotrifluoroethylene moiety, a hexafluoropropylene moiety, and a fluorinated 1,3-dioxole moiety.

In some non-limiting examples, the monomer may comprise a monomer backbone and a functional group. In some non-limiting examples, the functional group may be bonded, one of: directly, and via a linker group, to the monomer backbone. In some non-limiting examples, the monomer may comprise the linker group, and the linker group may be bonded to the monomer backbone and to the functional group. In some non-limiting examples, the monomer may comprise a plurality of functional groups, which may be one of: the same, and different, from one another. In such examples, each functional group may be bonded, one of: directly, and via a linker group, to the monomer backbone. In some non-limiting examples, where a plurality of functional groups is present, a plurality of linker groups may also be present.

In some non-limiting examples, the molecular structure of at least one of the materials of the patterning coating 110, which may be at least one of: the first material, and the second material, may comprise a plurality of different monomers. In some non-limiting examples, such molecular structure may comprise monomer species that have different at least one of: molecular composition, and molecular structure. Non-limiting examples of such molecular structure include those represented by Formulae (II) and (III):

$${( {Mon}^{A} )}_{k} {( {Mon}^{B} )}_{m} \qquad ( II )$$

$${( {Mon}^{A} )}_{k} {( {Mon}^{B} )}_{m} {( {Mon}^{C} )}_{o} \qquad ( III )$$

where:
Mon^{A}, Mon^{B}, and Mon^{C} each represent a monomer specie, and
k, m, and o each represent an integer of at least 2.

In some non-limiting examples, k, m, and o each represent an integer of one of between about: 2-100, 2-50, 3-20, 3-15, 3-10, and 3-7. Those having ordinary skill in the relevant art will appreciate that various non-limiting examples and descriptions regarding monomer, Mon, may be applicable with respect to each of Mon^{A}, Mon^{B}, and Mon^{C}.

In some non-limiting examples, the monomer may be represented by Formula (IV):

$$M - {( L - {R}_{x} )}_{y} \qquad ( IV )$$

where:
M represents the monomer backbone unit,
L represents the linker group,
R represents the functional group,
x is an integer between 1 and 4, and
y is an integer between 1 and 3.

In some non-limiting examples, the linker group may be represented by at least one of: a single bond, O, N, NH, C, CH, CH₂, and S.

Various non-limiting examples of the functional group which have been described herein may apply with respect to R of Formula (IV). In some non-limiting examples, the functional group R may comprise an oligomer unit, and the oligomer unit may further comprise a plurality of functional group monomer units. In some non-limiting examples, a functional group monomer unit may be at least one of: CH₂, and CF₂. In some non-limiting examples, a functional group may comprise a CH₂CF₃ moiety. In some non-limiting examples, such functional group monomer units may be bonded together to form at least one of: an alkyl, and an fluoroalkyl, oligomer unit. In some non-limiting examples, the oligomer unit may further comprise a functional group terminal unit. In some non-limiting examples, the functional group terminal unit may be arranged at a terminal end of the oligomer unit and bonded to a functional group monomer unit. In some non-limiting examples, the terminal end at which the functional group terminal unit may be arranged may correspond to a fragment of the functional group that may be distal to the monomer backbone unit. In some non-limiting examples, the functional group terminal unit may comprise at least one of: CF₂H, and CF₃.

In some non-limiting examples, the monomer backbone unit M may have a high surface tension. In some non-limiting examples, the monomer backbone unit may have a higher surface tension than at least one of the functional group(s) R bonded thereto. In some non-limiting examples, the monomer backbone unit may have a higher surface tension than any functional group R bonded thereto.

In some non-limiting examples, the monomer backbone unit may have a surface tension of one of at least about: 25 dynes/cm, 30 dynes/cm, 40 dynes/cm, 50 dynes/cm, 75 dynes/cm, 100 dynes/cm, 150 dynes/cm, 200 dynes/cm, 250 dynes/cm, 500 dynes/cm, 1,000 dynes/cm, 1,500 dynes/cm, and 2,000 dynes/cm.

In some non-limiting examples, the monomer backbone unit may comprise at least one of: a heterocyclic moiety, and at least one atom forming the members of the heterocyclic moiety. In some non-limiting examples, the heterocyclic moiety may comprise an N atom.

In some non-limiting examples, the monomer backbone unit may comprise phosphorus (P) and N, including without limitation, a phosphazene, in which there is a double bond between P and N and may be represented as at least one of: “NP” and “N═P”. In some non-limiting examples, the monomer backbone unit may comprise Si and O, including without limitation, silsesquioxane, which may be represented as SiO_{3/2}.

In some non-limiting examples, at least a part of the molecular structure of the at least one of the materials of the patterning coating 110, including without limitation, at least one of: the first material, and the second material, is represented by Formula (V):

$${( NP - {( L - {R}_{x} )}_{y} )}_{n} \qquad ( V )$$

where:
NP represents the phosphazene monomer backbone unit,
L represents the linker group,
R represents the functional group,
x is an integer between 1 and 4,
y is an integer between 1 and 3, and
n is an integer of at least 2.

In some non-limiting examples, the molecular structure of at least one of: the first material, and the second material, may be represented by Formula (V). In some non-limiting examples, at least one of: the first material, and the second material, may be a cyclophosphazene. In some non-limiting examples, the molecular structure of the cyclophosphazene may be represented by Formula (V).

In some non-limiting examples, L may represent 0, x may be 1, and R may represent a fluoroalkyl group. In some non-limiting examples, at least a fragment of the molecular structure of the at least one material of the patterning coating 110, including without limitation, at least one of: the first material, and the second material, may be represented by Formula (VI):

$${( {NP ( {OR}_{f} )}_{2} )}_{n} \qquad ( VI )$$

where:
R_{f} represents the fluoroalkyl group, and
n is an integer between 3 and 7.

In some non-limiting examples, the fluoroalkyl group may comprise at least one of: a CF₂ group, a CF₂H group, CH₂CF₃ group, and a CF₃ group. In some non-limiting examples, the fluoroalkyl group may be represented by Formula (VII):

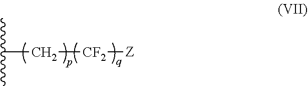

where:
p is an integer of 1 to 5;
q is an integer of 6 to 20; and
Z represents one of: H, and F.

In some non-limiting examples, p may be 1 and q may be an integer between 6 and 20.

In some non-limiting examples, the fluoroalkyl group R_{f} in Formula (VI) may be represented by Formula (VII).

In some non-limiting examples, at least a fragment of the molecular structure of at least one of the materials of the patterning coating 110, including without limitation, at least one of: the first material, and the second material, may be represented by Formula (VIII):

$${( {SiO}_{3 / 2} - ( L - R ) )}_{n} \qquad ( VIII )$$

where:
L represents the linker group,
R represents the functional group, and
n is an integer between 6 and 12.

In some non-limiting examples, L may represent the presence of at least one of: a single bond, O, substituted alkyl, and an unsubstituted alkyl. In some non-limiting examples, n may be one of: 8, 10, and 12. In some non-limiting examples R may comprise a functional group with low surface tension. In some non-limiting examples, R may comprise at least one of: an F-containing group, and a Si-containing group. In some non-limiting examples, R may comprise at least one of: a fluorocarbon group, and a siloxane-containing group. In some non-limiting examples, R may comprise at least one of: a CF₂ group, and a CF₂H group. In some non-limiting examples, R may comprise at least one of: a CF₂, and a CF₃, group. In some non-limiting examples, R may comprise a CH₂CF₃ group. In some non-limiting examples, the material represented by Formula (VIII) may be a polyoctahedral silsesquioxane.

In some non-limiting examples, at least a fragment of the molecular structure of at least one of the materials of the patterning coating 110, including without limitation, at least one of: the first material, and the second material, may be represented by Formula (IX):

$${( {SiO}_{3 / 2} - {R}_{f} )}_{n} \qquad ( IX )$$

where:
n is an integer of 6-12, and
R_{f} represents a fluoroalkyl group.

In some non-limiting examples n may be one of: 8, 10, and 12. In some non-limiting examples, R_{f} may comprise a functional group with low surface tension. In some non-limiting examples, R_{f} may comprise at least one of: a CF₂ moiety, and a CF₂H moiety. In some non-limiting examples, R_{f} may comprise at least one of: a CF₂, and a CF₃ moiety. In some non-limiting examples, R_{f} may comprise a CH₂CF₃ moiety. In some non-limiting examples, the material represented by Formula (IX) may be a polyoctahedral silsesquioxane.

In some non-limiting examples, the fluoroalkyl group, R_{f}, in Formula (IX) may be represented by Formula (VII).

In some non-limiting examples, at least a fragment of the molecular structure of at least one of the materials of the patterning coating 110, including without limitation, at least one of: the first material, and the second material, may be represented by Formula (X):

$${( {SiO}_{3 / 2} - {( {CH}_{2} )}_{x} ( {CF}_{3} ) )}_{n} \qquad ( X )$$

where:
x is an integer between 1 and 5, and
n is an integer between 6 and 12.

In some non-limiting examples, n may be one of: 8, 10, and 12.

In some non-limiting examples, the compound represented by Formula (X) may be a polyoctahedral silsesquioxane.

In some non-limiting examples, at least one of: the functional group R, and the fluoroalkyl group R_{f}, may be selected independently upon each occurrence of such group in any of the foregoing formulae. Those having ordinary skill in the relevant art will appreciate that any of the foregoing formulae may represent a sub-structure of the compound, and at least one of: additional groups, and additional moieties, may be present, which are not explicitly shown in the above formulae. Those having ordinary skill in the relevant art will appreciate that various formulae provided in the present application may represent at least one of: linear, branched, cyclic, cyclo-linear, and cross-linked, structures.

In some non-limiting examples, the patterning coating 110 may comprise at least one material represented by at least one of the following Formulae: (I), (II), (III), (IV), (V), (VI), (VIII), (IX), and (X), and at least one material exhibiting at least one of the following characteristics: includes an aromatic hydrocarbon moiety, includes an sp² carbon, includes a phenyl moiety, has a characteristic surface energy of at least about 20 dynes/cm, and exhibits photoluminescence, including without limitation, exhibiting photoluminescence at a wavelength of at least about 365 nm upon being irradiated by an excitation radiation having a wavelength of about 365 nm.

In some non-limiting examples, the patterning coating may comprise a third material that is different from the first material and the second material. In some non-limiting examples, the third material may comprise a monomer in common with at least one of: the first material, and the second material.

In some non-limiting examples, a difference in the sublimation temperature of the plurality of materials of the patterning coating 110, including, without limitation, a difference between the first material and the second material, may be one of no more than about: 5° C., 10° C., 15° C., 20° C., 30° C., 40° C., and 50° C. In some non-limiting examples, at least one of the materials of the patterning coating 110, including without limitation, at least one of: the first material, and the second material, may comprise at least one of: F, and Si, and the sublimation temperatures of the materials of the patterning coating 110 may differ by no more than one of about: 5° C., 10° C., 15° C., 20° C., 25° C., 40° C., and 50° C. In some non-limiting examples, at least one of the materials of the patterning coating 110, including without limitation, at least one of: the first material, and the second material, may comprise at least one of: a fluorocarbon moiety, and a siloxane moiety, and the sublimation temperatures of the materials of the patterning coating 110 may differ by one of no more than about: 5° C., 10° C., 15° C., 20° C., 25° C., 40° C., and 50° C.

In some non-limiting examples, a difference in a melting temperature of the plurality of materials of the patterning coating 110, including, without limitation, a difference between the first NIC material and the second NIC material, may be one of no more than about: 5° C., 10° C., 15° C., 20° C., 30° C., 40° C., and 50° C. In some non-limiting examples, at least one of the materials of the patterning coating 110, including without limitation, the first material, and the second material, may comprise at least one of: F, and Si, and the melting temperatures of the materials of the patterning coating 110 may differ by one of no more than about: 5° C., 10° C., 15° C., 20° C., 25° C., 40° C., and 50° C. In some non-limiting examples, at least one of the materials of the patterning coating 110, including without limitation, the first material, and the second material, may comprise at least one of: a fluorocarbon moiety, and a siloxane moiety, and the melting temperatures of the materials of the patterning coating 110 may differ by one of no more than about: 5° C., 10° C., 15° C., 20° C., 25° C., 40° C., and 50° C.

In some non-limiting examples, at least one of the materials of the patterning coating 110, including without limitation, at least one of: the first material, and the second material, may have a low characteristic surface energy. In some non-limiting examples, at least one of the materials of the patterning coating 110, including without limitation, the first material, and the second material, may have a low characteristic surface energy, and at least one of the materials of the patterning coating 110 may comprise at least one of: F, and Si. In some non-limiting examples, at least one of the materials of the patterning coating 110, including without limitation, at least one of: the first material, and the second material, may have a low characteristic surface energy, may comprise at least one of: F, and Si, and at least one other material of the patterning coating 110 may have a high characteristic surface energy. In some non-limiting examples, the presence of F and Si may be accounted for by the presence of a fluorocarbon moiety and a siloxane moiety, respectively. In some non-limiting examples, at least one of the materials, including without limitation, the second material, may have a low characteristic surface energy of one of between about: 10-20 dynes/cm, 12-20 dynes/cm, 15-20 dynes/cm, and 17-19 dynes/cm, and another material, including without limitation, the first material, may have a high characteristic surface energy of one of between about: 20-100 dynes/cm, 20-50 dynes/cm, and 25-45 dynes/cm. In some non-limiting examples, at least one of the materials may comprise at least one of: F, and Si. In some non-limiting examples, the second material may comprise at least one of: F, and Si.

In some non-limiting examples, at least one of the materials of the patterning coating 110, including without limitation, the second material, may have a low characteristic surface energy of no more than about 20 dynes/cm and may comprise at least one of: at least one of: F, and Si, and another material, including without limitation, the first material, may have a characteristic surface energy of at least about 20 dynes/cm.

In some non-limiting examples, at least one of the materials of the patterning coating 110, including without limitation, the second material, may have a low characteristic surface energy of no more than about 20 dynes/cm and may comprise at least one of: a fluorocarbon moiety, and a siloxane moiety, and another material of the patterning coating 110, including without limitation, the first material, may have a characteristic surface energy of at least about 20 dynes/cm.

In some non-limiting examples, the surface energy of each of the at least two materials of the patterning coating 110, including, without limitation, those of the first material and the second material, is one of no more about: 25 dynes/cm, 21 dynes/cm, 20 dynes/cm, 19 dynes/cm, 18 dynes/cm, 17 dynes/cm, 16 dynes/cm, 15 dynes/cm, 14 dynes/cm, 13 dynes/cm, 12 dynes/cm, 11 dynes/cm, and 10 dynes/cm.

In some non-limiting examples, a refractive index at a wavelength at least one of: 500 nm, and 460 nm, of at least one of the materials of the patterning coating 110, including without limitation, at least one of: the first material, and the second material, may be one of no more than about: 1.5, 1.45, 1.44, 1.43, 1.42, and 1.41. In some non-limiting examples, the patterning coating 110 may comprise at least one material that exhibits photoluminescence, and the patterning coating 110 may have a refractive index, at a wavelength of at least one of: 500 nm, and 460 nm, of one of no more than about: 1.5, 1.45, 1.44, 1.43, 1.42, and 1.41.

In some non-limiting examples, a molecular weight of at least one of the materials of the patterning coating 110, including without limitation, at least one of: the first material, and the second material, may be one of at least about: 750 g/mol, 1,000 g/mol, 1,500 g/mol, 2,000 g/mol, 2,500 g/mol, and 3,000 g/mol.

In some non-limiting examples, a molecular weight of at least one of the materials of the patterning coating 110, including without limitation, at least one of: the first material, and the second material, may be one of no more than about: 10,000 g/mol, 7,500 g/mol, and 5,000 g/mol.

In some non-limiting examples, the patterning coating 110 may comprise a plurality of materials exhibiting similar thermal properties, wherein at least one of the materials may exhibit photoluminescence. In some non-limiting examples, the patterning coating 110 may comprise a plurality of materials with similar thermal properties, wherein at least one of the materials may exhibit photoluminescence, and wherein at least one of the materials, may comprise at least one of: F, and Si. In some non-limiting examples, the patterning coating 110 may comprise a plurality of materials with similar thermal properties, including without limitation, at least one of: a melting temperature, and a sublimation temperature, of the materials, wherein at least one of the materials may exhibit photoluminescence at a wavelength of at least about 365 nm when excited by a radiation having an excitation wavelength of about 365 nm, and wherein at least one of the materials may comprise at least one of: F, and Si.

In some non-limiting examples, the patterning coating 110 may comprise a plurality of having at least one of: at least one element in common, and at least one sub-structure in common, wherein at least one of the materials may exhibit photoluminescence. In some non-limiting examples, at least one of the materials may comprise F and Si. In some non-limiting examples, the patterning coating 110 may comprise a plurality of materials with similar thermal properties, wherein at least one of the materials may exhibit photoluminescence at a wavelength that is at least about 365 nm when excited by a radiation having an excitation wavelength of about 365 nm, and wherein at least one of the materials may comprise at least one of: F, and Si. In some non-limiting examples, the at least one element in common may comprise at least one of: F, and Si. In some non-limiting examples, the at least one sub-structure in common may comprise at least one of: fluorocarbon, fluoroalkyl, and siloxyl.

In some non-limiting examples, a method for manufacturing an opto-electronic device 100 may comprise actions of: depositing a patterning coating on a first exposed layer surface 11 of the device 100 in a first portion 101 of a lateral aspect thereof; and depositing a deposited material 331 on a second exposed layer surface 11 of the device 100 in a second portion 102 of the lateral aspect thereof. An initial sticking probability against deposition of the deposited material 331 onto an exposed layer surface 11 of the patterning coating 110 in the first portion 101, may be substantially less than the initial sticking probability against deposition of the deposited material 331 onto an exposed layer surface 11 in the second portion 102, such that the exposed layer surface 11 of the patterning coating 110 in the first portion 101 may be substantially devoid of a closed coating 140 of the deposited material 331. The patterning coating 110 deposited on the first exposed layer surface 11 of the device 100 may comprises a first material and a second material.

In some non-limiting examples, depositing the patterning coating 110 on the first exposed layer surface 11 of the device 100 may comprise providing a mixture comprising a plurality of materials, and causing the mixture to be deposited onto the first exposed layer surface 11 of the device 100 to form the patterning coating 110 thereon. In some non-limiting examples, the mixture may comprise the first material and the second material. In some non-limiting examples, the first material and the second material may both be deposited onto the first exposed layer surface 11 to form the patterning coating 110 thereon.

In some non-limiting examples, the mixture comprising the plurality of materials may be deposited onto the first exposed layer surface 11 of the device 100 by a PVD process, including without limitation, thermal evaporation. In some non-limiting examples, the patterning coating 110 may be formed by evaporating the mixture from a single evaporation source and causing the mixture to be deposited on the first exposed layer surface 11 of the device 100. In some non-limiting examples, the mixture comprising, by way of non-limiting example, the first material and the second material, may be placed in a single evaporation source (crucible) to be heated under vacuum. Once the evaporation temperature of the materials is reached, a vapor flux generated therefrom may be directed towards the first exposed layer surface 11 of the device 100 to cause the deposition of the patterning coating 110 thereon.

In some non-limiting examples, the patterning coating 110 may be deposited by co-evaporation of the first material and the second material. In some non-limiting examples, the first material may be evaporated from a first evaporation source, and the second material may be concurrently evaporated from a second evaporation source such that the mixture may be formed in the vapor phase and may be co-deposited onto the first exposed layer surface 11 to provide the patterning coating 110 thereon.

In order to evaluate properties of certain example patterning coatings 110 comprising at least two materials, a series of samples were fabricated by depositing, in vacuo, an approximately 20 nm thick layer of an organic material that may be used as an HTL material, followed by depositing, over the organic material layer, a nucleation modifying coating having varying compositions as summarized in Table 5 below.

TABLE 5

| Sample Identifier | Composition of Nucleation Modifying Coating |
|---|---|
| Sample 1 | Patterning Material (15 nm) |
| Sample 2 | Patterning Material: PL Material 1 (0.5%, 15 nm) |
| Sample 3 | Patterning Material: PL Material 2 (0.5%, 15 nm) |
| Sample 4 | PL Material 1 (10 nm) |
| Sample 5 | PL Material 2 (10 nm) |
| Sample 6 | No nucleation modifying coating provided |

In the present example, the patterning material was selected such that, for example when deposited as a thin film, the patterning material exhibits a low initial sticking probability against deposition of the deposited material(s) 331, including without limitation, at least one of: Ag, and Yb.

In the present example, PL Material 1 and PL Material 2 were selected such that, by way of non-limiting example, when deposited as a thin film, each of PL Material 1 and PL Material 2 may exhibit photoluminescence detectable by standard optical measurement techniques including without limitation, fluorescence microscopy.

In Table 5, Sample 1 is a comparison sample in which the nucleation modifying coating was provided by depositing the Patterning Material. Sample 2 is an example sample in which the nucleation modifying coating was provided by co-depositing the Patterning Material and PL Material 1 together to form a coating comprising PL Material 1 in a concentration of 0.5 vol. %. Sample 3 is an example sample in which the nucleation modifying coating was provided by co-depositing the Patterning Material and PL Material 2 to form a coating comprising PL Material 2 in a concentration of 0.5 vol. %. Sample 4 is a comparison sample in which the nucleation modifying coating was provided by depositing PL Material 1. Sample 5 is a comparison sample in which the nucleation modifying coating was provided by depositing PL Material 2. Sample 6 is a comparison sample in which no nucleation modifying coating was provided over the organic material layer.

The photoluminescence (PL) response of each of Sample 1, Sample 2, Sample 3, and Sample 6 were measured. It was observed that the PL intensities of Sample 1 and Sample 6 were identical, thus indicating that the Patterning Material does not exhibit photoluminescence in the detected wavelength range. For each of Sample 2 and Sample 3, photoluminescence was detected in wavelengths of around 500 nm to about 600 nm.

Each of Samples 1 to 6 was then subjected to an open mask deposition of Yb, followed by Ag. Specifically, the surfaces of the nucleation modifying coatings formed by the above materials were subjected to an open mask deposition of Yb, followed by Ag. More specifically, each sample was subjected to a Yb vapor flux until a reference thickness of about 1 nm was reached, followed by an Ag vapor flux until a reference thickness of about 12 nm was reached. Once the samples were fabricated, optical transmission measurements were taken to determine the amount of at least one of: Yb, and Ag, deposited on the exposed layer surface 11 of the nucleation modifying coatings. Those having ordinary skill in the relevant art will appreciate that samples having little to no metal present thereon may be substantially transparent, while samples with metal deposited thereon, particularly as a closed coating 140, may generally exhibit a substantially lower light transmittance. Accordingly, the performance of various example coatings as a patterning coating 110 may be assessed by measuring the EM radiation transmission, which may directly correlate to an amount (thickness) of deposited material deposited thereon from deposition of either of both of Yb and Ag.

The reduction in optical transmittance as a function of wavelength of each of Sample 1, Sample 2, Sample 3, Sample 4, Sample 5, and Sample 6 were measured. Additionally, a reduction in optical transmittance at a wavelength of 600 nm after each sample was subjected to an Ag vapor flux was measured and summarized in Table 6 below.

TABLE 6

| Sample Identifier | Transmittance Reduction (%) at λ = 600 nm |
|---|---|
| Sample 1 | <1% |
| Sample 2 | <2% |
| Sample 3 | <1% |
| Sample 4 | 43% |
| Sample 5 | 47% |
| Sample 6 | 45% |

Specifically, the transmittance reduction (%) for each sample in Table 6 was determined by measuring the light transmission through the sample before and after the exposure to the Yb and Ag vapor flux and expressing the reduction in the EM radiation transmittance as a percentage.

As may be seen, Sample 1, Sample 2, and Sample 3 exhibited a substantially low transmittance reduction of less than 2%, and in the case of Samples 1 and 3, less than 1%. Accordingly, it may be observed that the nucleation modifying coatings provided for these samples acted as an NIC. By contrast, Sample 4, Sample 5, and Sample 6 each exhibited a transmittance reduction of 43%, 47%, and 45%, respectively. Accordingly, the nucleation modifying coatings provided for these samples did not act as an NIC but may have indeed acted as an NPC 520.

Moreover, it was found that Sample 1, in which the patterning coating 110 was comprised of substantially only the NIC Material, did not exhibit photoluminescence. However, Sample 2 and Sample 3 in which the patterning coating 110 comprised PL Material 1 and PL Material 2, respectively, in addition to the NIC material, were found to exhibit photoluminescence while also acting as an NIC by providing a surface with low initial sticking probability against the deposition of the deposited material 331.

### Deposited Layer

In some non-limiting examples, where the patterning coating 110 is restricted in its lateral extent to the first portion 101, in the second portion 102 of the lateral aspect of the device 100, a deposited layer 130 comprising a deposited material 331 may be disposed as a closed coating 140 on an exposed layer surface 11 of the underlying layer 210.

In some non-limiting examples, the deposited layer 130 may comprise a deposited material 331.

In some non-limiting examples, the deposited material 331 may comprise an element selected from at least one of: potassium (K), sodium (Na), lithium (Li), Ba, cesium (Cs), Yb, Ag, gold (Au), Cu, Al, Mg, Zn, Cd, tin (Sn), and yttrium (Y). In some non-limiting examples, the element may comprise at least one of: K, Na, Li, Ba, Cs, Yb, Ag, Au, Cu, Al, and Mg. In some non-limiting examples, the element may comprise at least one of: Cu, Ag, and Au. In some non-limiting examples, the element may be Cu. In some non-limiting examples, the element may be Al. In some non-limiting examples, the element may comprise at least one of: Mg, Zn, Cd, and Yb. In some non-limiting examples, the element may comprise at least one of: Mg, Ag, Al, Yb, and Li. In some non-limiting examples, the element may comprise at least one of: Mg, Ag, and Yb. In some non-limiting examples, the element may comprise at least one of: Mg, and Ag. In some non-limiting examples, the element may be Ag.

In some non-limiting examples, the deposited material 331 may comprise a pure metal. In some non-limiting examples, the deposited material 331 may be (substantially) pure Ag. In some non-limiting examples, the substantially pure Ag may have a purity of one of at least about: 95%, 99%, 99.9%, 99.99%, 99.999%, and 99.9995%. In some non-limiting examples, the deposited material 331 may be (substantially) pure Mg. In some non-limiting examples, the substantially pure Mg may have a purity of one of at least about: 95%, 99%, 99.9%, 99.99%, 99.999%, and 99.9995%.

In some non-limiting examples, the deposited material 331 may comprise an alloy. In some non-limiting examples, the alloy may be one of: an Ag-containing alloy, an Mg-containing alloy, and an AgMg-containing alloy. In some non-limiting examples, the AgMg-containing alloy may have an alloy composition that may range from about 1:10 (Ag:Mg) to about 10:1 by volume.

In some non-limiting examples, the deposited material 331 may comprise other metals in one of: in place of, and in combination with, Ag. In some non-limiting examples, the deposited material 331 may comprise an alloy of Ag with at least one other metal. In some non-limiting examples, the deposited material 331 may comprise an alloy of Ag with at least one of: Mg, and Yb. In some non-limiting examples, such alloy may be a binary alloy having a composition between about 5-95 vol. % Ag, with the remainder being the other metal. In some non-limiting examples, the deposited material 331 may comprise Ag and Mg. In some non-limiting examples, the deposited material 331 may comprise an Ag:Mg alloy having a composition between about 1:10-10:1 by volume. In some non-limiting examples, the deposited material 331 may comprise Ag and Yb. In some non-limiting examples, the deposited material 331 may comprise a Yb:Ag alloy having a composition between about 1:20-10:1 by volume. In some non-limiting examples, the deposited material 331 may comprise Mg and Yb. In some non-limiting examples, the deposited material 331 may comprise an Mg:Yb alloy. In some non-limiting examples, the deposited material 331 may comprise Ag, Mg, and Yb. In some non-limiting examples, the deposited layer 130 may comprise an Ag:Mg:Yb alloy.

In some non-limiting examples, the deposited layer 130 may comprise at least one additional element. In some non-limiting examples, such additional element may be a non-metallic element. In some non-limiting examples, the non-metallic element may be at least one of: O, S, N, and C. It will be appreciated by those having ordinary skill in the relevant art that, in some non-limiting examples, such additional element(s) may be incorporated into the deposited layer 130 as a contaminant, due to the presence of such additional element(s) in at least one of: the source material, equipment used for deposition, and the vacuum chamber environment. In some non-limiting examples, the concentration of such additional element(s) may be limited to be below a threshold concentration. In some non-limiting examples, such additional element(s) may form a compound together with other element(s) of the deposited layer 130. In some non-limiting examples, a concentration of the non-metallic element in the deposited material 331 may be one of no more than about: 1%, 0.1%, 0.01%, 0.001%, 0.0001%, 0.00001%, 0.000001%, and 0.0000001%. In some non-limiting examples, the deposited layer 130 may have a composition in which a combined amount of O and C therein may be one of no more than about: 10%, 5%, 1%, 0.1%, 0.01%, 0.001%, 0.0001%, 0.00001%, 0.000001%, and 0.0000001%.

It has now been found, that reducing a concentration of certain non-metallic elements in the deposited layer 130, particularly in cases wherein the deposited layer 130 may be substantially comprised of at least one of: metal(s), and metal alloy(s), may facilitate selective deposition of the deposited layer 130. Without wishing to be bound by any particular theory, it may be postulated that certain non-metallic elements, such as, in some non-limiting examples, at least one of: O, and C, when present in the vapor flux 332 of at least one of: the deposited layer 130, in the deposition chamber, and the environment, may be deposited onto the surface of the patterning coating 110 to act as nucleation sites for the metallic element(s) of the deposited layer 130. It may be postulated that reducing a concentration of such non-metallic elements that could act as nucleation sites may facilitate reducing an amount of deposited material 331 deposited on the exposed layer surface 11 of the patterning coating 110.

In some non-limiting examples, the deposited material 331 may be deposited on a metal-containing underlying layer 210. In some non-limiting examples, the deposited material 331 and the underlying layer 210 thereunder may comprise a metal in common.

In some non-limiting examples, the deposited layer 130 may comprise a plurality of layers of the deposited material 331. In some non-limiting examples, the deposited material 331 of a first one of the plurality of layers may be different from the deposited material 331 of a second one of the plurality of layers. In some non-limiting examples, the deposited layer 130 may comprise a multilayer coating. In some non-limiting examples, such multilayer coating may be one of: Yb/Ag, Yb/Mg, Yb/Mg:Ag, Yb/Yb:Ag, Yb/Ag/Mg, and Yb/Mg/Ag.

In some non-limiting examples, the deposited material 331 may comprise a metal having a bond dissociation energy, of one of no more than about: 300 kJ/mol, 200 kJ/mol, 165 kJ/mol, 150 kJ/mol, 100 kJ/mol, 50 kJ/mol, and 20 kJ/mol.

In some non-limiting examples, the deposited material 331 may comprise a metal having an electronegativity that is one of no more than about: 1.4, 1.3, and 1.2.

In some non-limiting examples, a sheet resistance of the deposited layer 130 may generally correspond to a sheet resistance of the deposited layer 130, measured in isolation from other components, layers, and parts of the device 100. In some non-limiting examples, the deposited layer 130 may be formed as a thin film. Accordingly, in some non-limiting examples, the characteristic sheet resistance for the deposited layer 130 may be determined based on at least one of: the composition, thickness, and morphology, of such thin film. In some non-limiting examples, the sheet resistance may be one of no more than about: 10Ω/□, 5Ω/□, 1Ω/□, 0.5Ω/□, 0.2Ω/□, and 0.1Ω/□.

In some non-limiting examples, the deposited layer 130 may be disposed in a pattern that may be defined by at least one region therein that is substantially devoid of a closed coating 140 of the deposited layer 130. In some non-limiting examples, the at least one region may separate the deposited layer 130 into a plurality of discrete fragments thereof. In some non-limiting examples, each discrete fragment of the deposited layer 130 may be a distinct second portion 102. In some non-limiting examples, the plurality of discrete fragments of the deposited layer 130 may be physically spaced apart from one another in the lateral aspect thereof. In some non-limiting examples, at least two of such plurality of discrete fragments of the deposited layer 130 may be electrically coupled. In some non-limiting examples, at least two of such plurality of discrete fragments of the deposited layer 130 may be each electrically coupled with a common conductive coating, including without limitation, the underlying layer 210, to allow the flow of electrical current between them. In some non-limiting examples, at least two of such plurality of discrete fragments of the deposited layer 130 may be electrically insulated from one another.

### Selective Deposition Using Patterning Coatings

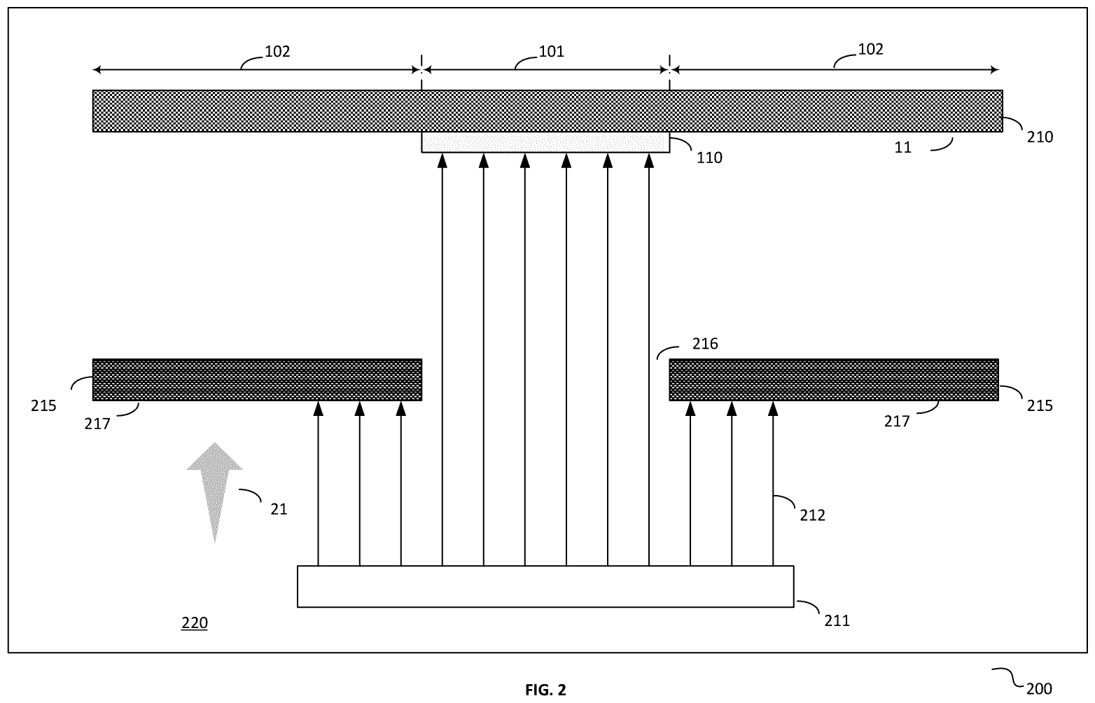

FIG. 2 is an example schematic diagram illustrating a non-limiting example of an evaporative deposition process, shown generally at 200, in a chamber 220, for selectively depositing a patterning coating 110 onto a first portion 101 of an exposed layer surface 11 of an underlying layer 210, including without limitation, a substrate 10.

In the process 200, a quantity of a patterning material 211 may be heated under vacuum, to evaporate (sublime) the patterning material 211. In some non-limiting examples, the patterning material 211 may comprise substantially (including without limitation, entirely), a material used to form the patterning coating 110. In some non-limiting examples, such material may comprise an organic material.

An evaporated flux 212 of the patterning material 211 may flow through the chamber 220, including in a direction indicated by arrow 21, toward the exposed layer surface 11. When the evaporated flux 212 is incident on the exposed layer surface 11, the patterning coating 110 may be formed thereon.

In some non-limiting examples, as shown in the figure for the process 200, the patterning coating 110 may be selectively deposited only onto a portion, in the example illustrated, the first portion 101, of the exposed layer surface 11 of the underlying layer 210, by the interposition, between the vapor flux 212 and the exposed layer surface 11 of the underlying layer 210, of a shadow mask 215, which in some non-limiting examples, may be an FMM. In some non-limiting examples, such a shadow mask 215 may, in some non-limiting examples, be used to form substantially small features, with a feature size on the order of (smaller than) tens of microns.

The shadow mask 215 may have at least one aperture 216 extending therethrough such that a part of the evaporated flux 212 passes through the aperture 216 and may be incident on the exposed layer surface 11 to form the patterning coating 110. Where the evaporated flux 212 does not pass through the aperture 216 but is incident on a surface 217 of the shadow mask 215, it is precluded from being disposed on the exposed layer surface 11 to form the patterning coating 110. In some non-limiting examples, the shadow mask 215 may be configured such that the evaporated flux 212 that passes through the aperture 216 may be incident on the first portion 101 but not the second portion 102. The second portion 102 of the exposed layer surface 11 may thus be substantially devoid of the patterning coating 110. In some non-limiting examples (not shown), the patterning material 211 that is incident on the shadow mask 215 may be deposited on the surface 217 thereof.

Accordingly, a patterned surface may be produced upon completion of the deposition of the patterning coating 110.

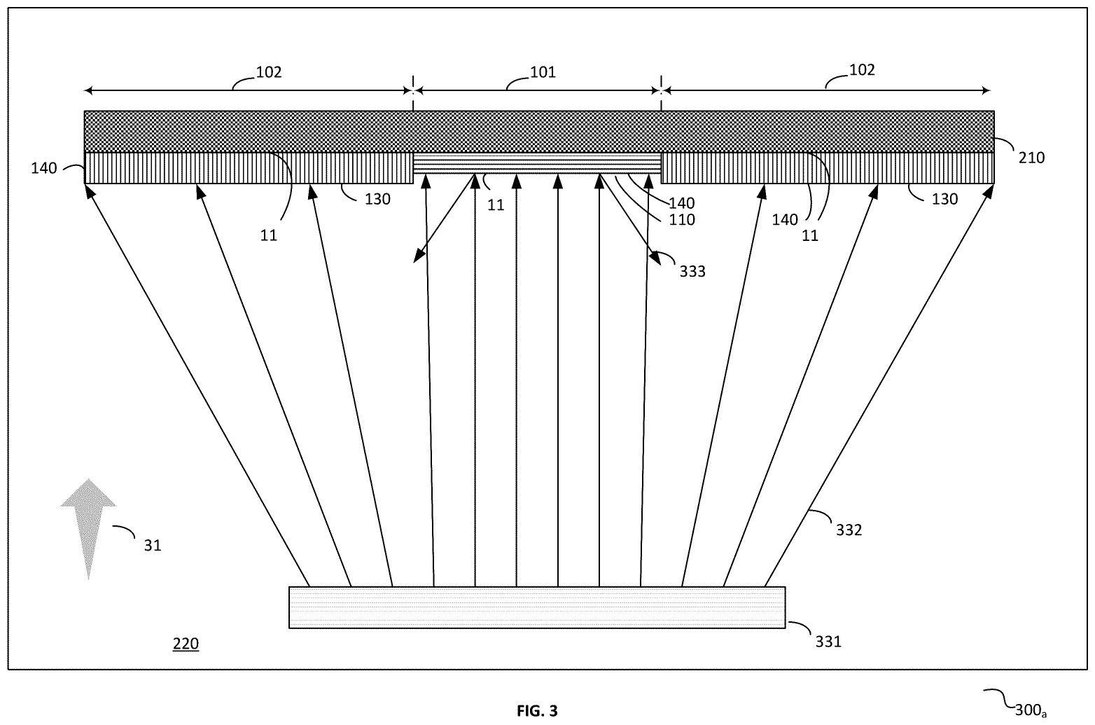

FIG. 3 is an example schematic diagram illustrating a non-limiting example of a result of an evaporative process, shown generally at 300ₐ, in a chamber 220, for selectively depositing a closed coating 140 of a deposited layer 130 onto the second portion 102 of an exposed layer surface 11 of the underlying layer 210 that is substantially devoid of the patterning coating 110 that was selectively deposited onto the first portion 101, including without limitation, by the evaporative process 200 of FIG. 2.

In some non-limiting examples, the deposited layer 130 may be comprised of a deposited material 331, in some non-limiting examples, comprising at least one metal. It will be appreciated by those having ordinary skill in the relevant art that in some non-limiting examples, a vaporization temperature of an organic material is low relative to the vaporization temperature of metals, such as may be employed as a deposited material 331.

Thus, in some non-limiting examples, there may be fewer constraints in employing a shadow mask 215 to selectively deposit a patterning coating 110 in a pattern, relative to directly patterning the deposited layer 130 using such shadow mask 215.

Once the patterning coating 110 has been deposited on the first portion 101 of the exposed layer surface 11 of the underlying layer 210, a closed coating 140 of the deposited material 331 may be deposited, on the second portion 102 of the exposed layer surface 11 that is substantially devoid of the patterning coating 110, as the deposited layer 130.

In the process 300ₐ, a quantity of the deposited material 331 may be heated under vacuum, to sublime the deposited material 331. In some non-limiting examples, the deposited material 331 may be comprised of substantially, including without limitation, entirely, a material used to form the deposited layer 130.

An evaporated flux 332 of the deposited material 331 may be directed inside the chamber 220, including in a direction indicated by arrow 31, toward the exposed layer surface 11 of the first portion 101 and of the second portion 102. When the evaporated flux 332 is incident on the second portion 102 of the exposed layer surface 11, a closed coating 140 of the deposited material 331 may be formed thereon as the deposited layer 130.

In some non-limiting examples, deposition of the deposited material 331 may be performed using one of: an open mask, and a mask-free, deposition process.

It will be appreciated by those having ordinary skill in the relevant art that, contrary to that of a shadow mask 215, the feature size of an open mask may be generally comparable to the size of a device 100 being manufactured.

It will be appreciated by those having ordinary skill in the relevant art that, in some non-limiting examples, the use of an open mask may be omitted. In some non-limiting examples, an open mask deposition process described herein may alternatively be conducted without the use of an open mask, such that an entire target exposed layer surface 11 may be exposed.

Indeed, as shown in FIG. 3, the evaporated flux 332 may be incident both on an exposed layer surface 11 of the patterning coating 110 across the first portion 101 as well as the exposed layer surface 11 of the underlying layer 210 across the second portion 102 that is substantially devoid of the patterning coating 110.

Since the exposed layer surface 11 of the patterning coating 110 in the first portion 101 may exhibit a substantially low initial sticking probability against the deposition of the deposited material 331 relative to the exposed layer surface 11 of the underlying layer 210 in the second portion 102, the deposited layer 130 may be selectively deposited substantially only on the exposed layer surface 11, of the underlying layer 210 in the second portion 102, that is substantially devoid of the patterning coating 110. By contrast, the evaporated flux 332 incident on the exposed layer surface 11 of the patterning coating 110 across the first portion 101 may tend to not be deposited (as shown 333), and the exposed layer surface 11 of the patterning coating 110 across the first portion 101 may be substantially devoid of a closed coating 140 of the deposited layer 130.

In some non-limiting examples, an initial deposition rate, of the evaporated flux 332 on the exposed layer surface 11 of the underlying layer 210 in the second portion 102, may exceed one of about: 200 times, 550 times, 900 times, 1,000 times, 1,500 times, 1,900 times, and 2,000 times an initial deposition rate of the evaporated flux 332 on the exposed layer surface 11 of the patterning coating 110 in the first portion 101.

Thus, the combination of the selective deposition of a patterning coating 110 in FIG. 2 using a shadow mask 215 and one of: the open mask, and a mask-free, deposition of the deposited material 331 may result in a version 300ₐ of the device 100 shown in FIG. 3.

After selective deposition of the patterning coating 110 across the first portion 101, a closed coating 140 of the deposited material 331 may be deposited over the device 300ₐ as the deposited layer 130, in some non-limiting examples, using one of: an open mask, and a mask-free, deposition process, but may remain substantially only within the second portion 102, which is substantially devoid of the patterning coating 110.

The patterning coating 110 may provide, within the first portion 101, an exposed layer surface 11 with a substantially low initial sticking probability, against the deposition of the deposited material 331, and that is substantially less than the initial sticking probability, against the deposition of the deposited material 331, of the exposed layer surface 11 of the underlying layer 210 of the device 300ₐ within the second portion 102.

Thus, the first portion 101 may be substantially devoid of a closed coating 140 of the deposited material 331.

While the present disclosure contemplates the patterned deposition of the patterning coating 110 by an evaporative deposition process, involving a shadow mask 215, those having ordinary skill in the relevant art will appreciate that, in some non-limiting examples, this may be achieved by any applicable deposition process, including without limitation, a micro-contact printing process.

While the present disclosure contemplates the patterning coating 110 being an NIC, those having ordinary skill in the relevant art will appreciate that, in some non-limiting examples, the patterning coating 110 may be an NPC 520. In such examples, the portion (such as, without limitation, the first portion 101) in which the NPC 520 has been deposited may, in some non-limiting examples, have a closed coating 140 of the deposited material 331, while the other portion (such as, without limitation, the second portion 102) may be substantially devoid of a closed coating 140 of the deposited material 331.

In some non-limiting examples, an average layer thickness of the patterning coating 110 and of the deposited layer 130 deposited thereafter may be varied according to a variety of parameters, including without limitation, a given application and given performance characteristics. In some non-limiting examples, the average layer thickness of the patterning coating 110 may be comparable to, including without limitation, substantially no more than, an average layer thickness of the deposited layer 130 deposited thereafter. Use of a substantially thin patterning coating 110 to achieve selective patterning of a deposited layer 130 may have applicability to provide flexible devices 100.

In some non-limiting examples, the device 300 may further comprise an NPC 520 disposed between the patterning coating 110 and the second electrode 640.

In some non-limiting examples, the patterning coating 110 may be formed concurrently with the at least one semiconducting layer(s) 630. In some non-limiting examples, at least one material used to form the patterning coating 110 may also be used to form the at least one semiconducting layer(s) 630 to reduce a number of stages for fabricating the device 100.

### Edge Effects

### Patterning Coating Transition Region

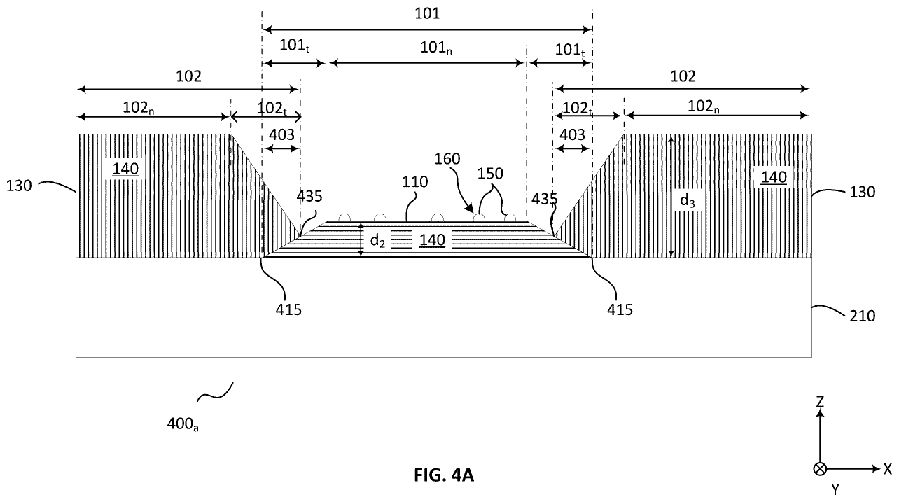

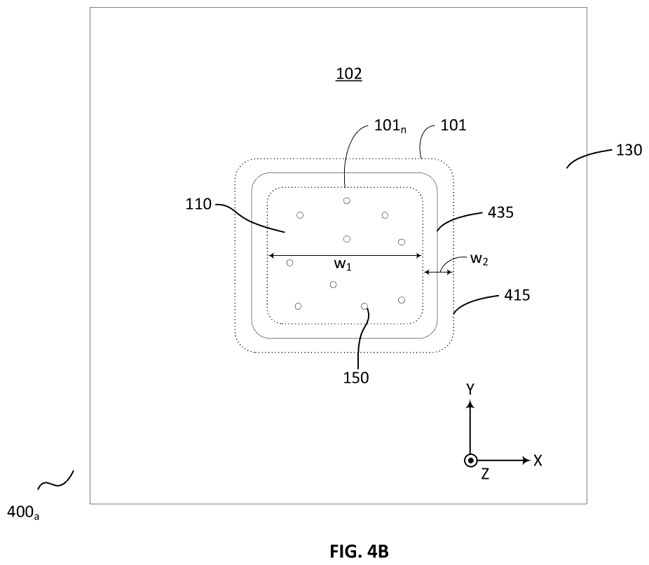

Turning to FIG. 4A, there may be shown a version 400ₐ of the device 100 of FIG. 1 that may show in exaggerated form, an interface between the patterning coating 110 in the first portion 101 and the deposited layer 130 in the second portion 102. FIG. 4B may show the device 400ₐ in plan.

As may be better seen in FIG. 4B, in some non-limiting examples, the patterning coating 110 in the first portion 101 may be surrounded on all sides by the deposited layer 130 in the second portion 102, such that the first portion 101 may have a boundary that is defined by the further edge 415 of the patterning coating 110 in the lateral aspect along each lateral axis. In some non-limiting examples, the patterning coating edge 415 in the lateral aspect may be defined by a perimeter of the first portion 101 in such aspect.

In some non-limiting examples, the first portion 101 may comprise at least one patterning coating transition region 101ₜ, in the lateral aspect, in which a thickness of the patterning coating 110 may transition from a maximum thickness to a reduced thickness. The extent of the first portion 101 that does not exhibit such a transition may be identified as a patterning coating non-transition part 101 of the first portion 101. In some non-limiting examples, the patterning coating 110 may form a substantially closed coating 140 in the patterning coating non-transition part 101 of the first portion 101.

In some non-limiting examples, the patterning coating transition region 101ₜ may extend, in the lateral aspect, between the patterning coating non-transition part 101 of the first portion 101 and the patterning coating edge 415.

In some non-limiting examples, in plan, the patterning coating transition region 101ₜ may extend along a perimeter of the patterning coating non-transition part 101 of the first portion 101.

In some non-limiting examples, along at least one lateral axis, the patterning coating non-transition part 101 may occupy the entirety of the first portion 101, such that there is no patterning coating transition region 101 between it and the second portion 102.

As illustrated in FIG. 4A, in some non-limiting examples, the patterning coating 110 may have an average film thickness d₂ in the patterning coating non-transition part 101ₙ of the first portion 101 that may be in a range of one of between about: 1-100 nm, 2-50 nm, 3-30 nm, 4-20 nm, 5-15 nm, 5-10 nm, and 1-10 nm. In some non-limiting examples, the average film thickness d₂ of the patterning coating 110 in the patterning coating non-transition part 101ₙ of the first portion 101 may be substantially the same (constant) thereacross. In some non-limiting examples, an average film thickness d₂ of the patterning coating 110 may remain, within the patterning coating non-transition part 101ₙ, within one of about: 95%, and 90%, of the average film thickness d₂ of the patterning coating 110.

In some non-limiting examples, the average film thickness d₂ may be between about 1-100 nm. In some non-limiting examples, the average film thickness d₂ may be one of no more than about: 80 nm, 60 nm, 50 nm, 40 nm, 30 nm, 20 nm, 15 nm, and 10 nm. In some non-limiting examples, the average film thickness d₂ of the patterning coating 110 may be one of at least about: 3 nm, 5 nm, and 8 nm.

In some non-limiting examples, the average film thickness d₂ of the patterning coating 110 in the patterning coating non-transition part 101 of the first portion 101 may be no more than about 10 nm. Without wishing to be bound by any particular theory, it has been found, that a non-zero average film thickness d₂ of the patterning coating 110 that is no more than about 10 nm may, at least in some non-limiting examples, provide certain advantages for achieving, in some non-limiting examples, enhanced patterning contrast of the deposited layer 130, relative to a patterning coating 110 having an average film thickness d₂ in the patterning coating non-transition part 101 of the first portion 101 of at least about 10 nm.

In some non-limiting examples, the patterning coating 110 may have a patterning coating thickness that decreases from a maximum to a minimum within the patterning coating transition region 101ₜ. In some non-limiting examples, the maximum may be proximate to a boundary between the patterning coating transition region 101ₜ and the patterning coating non-transition part 101 of the first portion 101. In some non-limiting examples, the minimum may be proximate to the patterning coating edge 415. In some non-limiting examples, the maximum may be the average film thickness d₂ in the patterning coating non-transition part 101 of the first portion 101. In some non-limiting examples, the maximum may be no more than one of about: 95%, and 90%, of the average film thickness d₂ in the patterning coating non-transition part 101 of the first portion 101. In some non-limiting examples, the minimum may be in a range of between about 0-0.1 nm.

In some non-limiting examples, a profile of the patterning coating thickness in the patterning coating transition region 101ₜ may be sloped. In some non-limiting examples, such profile may be tapered. In some non-limiting examples, the taper may follow one of: a linear, non-linear, parabolic, and exponential decaying, profile.

In some non-limiting examples, the patterning coating 110 may completely cover the underlying layer 210 in the patterning coating transition region 101ₜ. In some non-limiting examples, at least a part of the underlying layer 210 may be left uncovered by the patterning coating 110 in the patterning coating transition region 101ₜ. In some non-limiting examples, the patterning coating 110 may comprise a substantially closed coating 140 in at least one of: at least a part of the patterning coating transition region 101, and at least a part of the patterning coating non-transition part 101ₙ.

In some non-limiting examples, the patterning coating 110 may comprise a discontinuous layer 160 in at least one of: at least a part of the patterning coating transition region 101ₜ, and at least a part of the patterning coating non-transition part 101ₙ.

In some non-limiting examples, at least a part of the patterning coating 110 in the first portion 101 may be substantially devoid of a closed coating 140 of the deposited layer 130. In some non-limiting examples, at least a part of the exposed layer surface 11 of the first portion 101 may be substantially devoid of a closed coating 140 of one of: the deposited layer 130, and the deposited material 331.

In some non-limiting examples, along at least one lateral axis, including without limitation, the X-axis, the patterning coating non-transition part 101ₙ may have a width of w₁, and the patterning coating transition region 101ₜ may have a width of w₂. In some non-limiting examples, the patterning coating non-transition part 101 may have a cross-sectional area that, in some non-limiting examples, may be approximated by multiplying the average film thickness d₂ by the width w₁. In some non-limiting examples, the patterning coating transition region 101ₜ may have a cross-sectional area that, in some non-limiting examples, may be approximated by multiplying an average film thickness across the patterning coating transition region 101ₜ by the width w₁.

In some non-limiting examples, w₁ may exceed w₂. In some non-limiting examples, a quotient of w₁/w₂ may be one of at least about: 5, 10, 20, 50, 100, 500, 1,000, 1,500, 5,000, 10,000, 50,000, and 100,000.

In some non-limiting examples, at least one of w₁ and w₂ may exceed the average film thickness d₁ of the underlying layer 210.

In some non-limiting examples, at least one of w1 and w₂ may exceed d₂. In some non-limiting examples, both w₁ and w₂ may exceed d₂. In some non-limiting examples, w₁ and w₂ both may exceed d₁, and d₁ may exceed d₂.

### Deposited Layer Transition Region

As may be better seen in FIG. 4B, in some non-limiting examples, the patterning coating 110 in the first portion 101 may be surrounded by the deposited layer 130 in the second portion 102 such that the second portion 102 has a boundary that is defined by the further edge 435 of the deposited layer 130 in the lateral aspect along each lateral axis. In some non-limiting examples, the deposited layer edge 435 in the lateral aspect may be defined by a perimeter of the second portion 102 in such aspect.

In some non-limiting examples, the second portion 102 may comprise at least one deposited layer transition region 102ₜ, in the lateral aspect, in which a thickness of the deposited layer 130 may transition from a maximum thickness to a reduced thickness. The extent of the second portion 102 that does not exhibit such a transition may be identified as a deposited layer non-transition part 102ₙ of the second portion 102. In some non-limiting examples, the deposited layer 130 may form a substantially closed coating 140 in the deposited layer non-transition part 102ₙ of the second portion 102.

In some non-limiting examples, in plan, the deposited layer transition region 102ₜ may extend, in the lateral aspect, between the deposited layer non-transition part 102ₙ of the second portion 102 and the deposited layer edge 435.

In some non-limiting examples, in plan, the deposited layer transition region 102ₜ may extend along a perimeter of the deposited layer non-transition part 102ₙ of the second portion 102.

In some non-limiting examples, along at least one lateral axis, the deposited layer non-transition part 102ₙ of the second portion 102 may occupy the entirety of the second portion 102, such that there is no deposited layer transition region 102ₜ between it and the first portion 101.

As illustrated in FIG. 4A, in some non-limiting examples, the deposited layer 130 may have an average film thickness d₃ in the deposited layer non-transition part 102ₙ of the second portion 102 that may be in a range of one of between about: 1-500 nm, 5-200 nm, 5-40 nm, 10-30 nm, and 10-100 nm. In some non-limiting examples, d₃ may exceed one of about: 10 nm, 50 nm, and 100 nm. In some non-limiting examples, the average film thickness d₃ of the deposited layer 130 in the deposited layer non-transition part 102ₜ of the second portion 102 may be substantially the same (constant) thereacross.

In some non-limiting examples, d₃ may exceed the average film thickness d₁ of the underlying layer 210.

In some non-limiting examples, a quotient d₃/d₁ may be one of at least about: 1.5, 2, 5, 10, 20, 50, and 100. In some non-limiting examples, the quotient d₃/d₁ may be in a range of one of between about: 0.1-10, and 0.2-40.

In some non-limiting examples, d₃ may exceed an average film thickness d₂ of the patterning coating 110.

In some non-limiting examples, a quotient d₃/d₂ may be one of at least about: 1.5, 2, 5, 10, 20, 50, and 100. In some non-limiting examples, the quotient d₃/d₂ may be in a range of one of between about: 0.2-10, and 0.5-40.

In some non-limiting examples, d₃ may exceed d₂ and d₂ may exceed d₁. In some non-limiting examples, d₃ may exceed d₁ and d₁ may exceed d₂.

In some non-limiting examples, a quotient d₂/d₁ may be between one of about: 0.2-3, and 0.1-5.

In some non-limiting examples, along at least one lateral axis, including without limitation, the X-axis, the deposited layer non-transition part 102ₙ of the second portion 102 may have a width of w₃. In some non-limiting examples, the deposited layer non-transition part 102ₙ of the second portion 102 may have a cross-sectional area a₃ that, in some non-limiting examples, may be approximated by multiplying the average film thickness d₃ by the width w₃.

In some non-limiting examples, w₃ may exceed the width w₁ of the patterning coating non-transition part 101ₙ. In some non-limiting examples, w₁ may exceed w₃.

In some non-limiting examples, a quotient w₁/w₃ may be in a range of one of between about: 0.1-10, 0.2-5, 0.3-3, and 0.4-2. In some non-limiting examples, a quotient w₃/w₁ may be one of at least about: 1, 2, 3, and 4.

In some non-limiting examples, w₃ may exceed the average film thickness d₃ of the deposited layer 130.

In some non-limiting examples, a quotient w₃/d₃ may be one of at least about: 10, 50, 100, and 500. In some non-limiting examples, the quotient w₃/d₃ may be no more than about 100,000.

In some non-limiting examples, the deposited layer 130 may have a thickness that decreases from a maximum to a minimum within the deposited layer transition region 102ₜ. In some non-limiting examples, the maximum may be proximate to the boundary between the deposited layer transition region 102ₜ and the deposited layer non-transition part 102ₙ of the second portion 102. In some non-limiting examples, the minimum may be proximate to the deposited layer edge 435. In some non-limiting examples, the maximum may be the average film thickness d₃ in the deposited layer non-transition part 102ₙ of the second portion 102. In some non-limiting examples, the minimum may be in a range of between about 0-0.1 nm. In some non-limiting examples, the minimum may be the average film thickness d₃ in the deposited layer non-transition part 102ₙ of the second portion 102.

In some non-limiting examples, a profile of the thickness in the deposited layer transition region 102ₜ may be sloped. In some non-limiting examples, such profile may be tapered. In some non-limiting examples, the taper may follow one of: a linear, non-linear, parabolic, and exponential decaying, profile.

In some non-limiting examples, although not shown, the deposited layer 130 may completely cover the underlying layer 210 in the deposited layer transition region 102ₜ. In some non-limiting examples, the deposited layer 130 may comprise a substantially closed coating 140 in at least a part of the deposited layer transition region 102ₜ. In some non-limiting examples, at least a part of the underlying layer 210 may be uncovered by the deposited layer 130 in the deposited layer transition region 102ₜ.

In some non-limiting examples, the deposited layer 130 may comprise a discontinuous layer 160 in at least a part of the deposited layer transition region 102ₜ.

Those having ordinary skill in the relevant art will appreciate that, although not shown, the patterning material 211 may also be present to some extent at an interface between the deposited layer 130 and an underlying layer 210. Such material may be deposited as a result of a shadowing effect, in which a deposited pattern is not identical to a pattern of a mask and may, in some non-limiting examples, result in some evaporated patterning material 211 being deposited on a masked part of a target exposed layer surface 11. In some non-limiting examples, such material may form as at least one of: particle structures 150, and as a thin film having a thickness that may be substantially no more than an average thickness of the patterning coating 110.

### Overlap

In some non-limiting examples, although not shown, the deposited layer edge 435 may be spaced apart, in the lateral aspect from the patterning coating transition region 101ₜ of the first portion 101, such that there is no overlap between the first portion 101 and the second portion 102 in the lateral aspect.

In some non-limiting examples, at least a part of the first portion 101 and at least a part of the second portion 102 may overlap in the lateral aspect. Such overlap may be identified by an overlap portion 403, such as may be shown in some non-limiting examples in FIG. 4A, in which at least a part of the second portion 102 overlaps at least a part of the first portion 101.

In some non-limiting examples, although not shown, at least a part of the deposited layer transition region 102ₜ may be disposed over at least a part of the patterning coating transition region 101ₜ. In some non-limiting examples, at least a part of the patterning coating transition region 101ₜ may be substantially devoid of at least one of: the deposited layer 130, and the deposited material 331. In some non-limiting examples, the deposited material 331 may form a discontinuous layer 160 on an exposed layer surface 11 of at least a part of the patterning coating transition region 101ₜ.

In some non-limiting examples, although not shown, at least a part of the deposited layer transition region 102ₜ may be disposed over at least a part of the patterning coating non-transition part 101ₙ of the first portion 101.

Although not shown, those having ordinary skill in the relevant art will appreciate that, in some non-limiting examples, the overlap portion 403 may reflect a scenario in which at least a part of the first portion 101 overlaps at least a part of the second portion 102.

Thus, in some non-limiting examples, at least a part of the patterning coating transition region 101ₜ may be disposed over at least a part of the deposited layer transition region 102ₜ. In some non-limiting examples, at least a part of the deposited layer transition region 102ₜ may be substantially devoid of at least one of: the patterning coating 110, and the patterning material 211. In some non-limiting examples, the patterning material 211 may form a discontinuous layer 160 on an exposed layer surface of at least a part of the deposited layer transition region 102ₜ.

In some non-limiting examples, at least a part of the patterning coating transition region 101ₜ may be disposed over at least a part of the deposited layer non-transition part 102ₙ of the second portion 102.

In some non-limiting examples, the patterning coating edge 415 may be spaced apart, in the lateral aspect, from the deposited layer non-transition part 102ₙ of the second portion 102.

In some non-limiting examples, the deposited layer 130 may be formed as a single monolithic coating across both the deposited layer non-transition part 102ₙ and the deposited layer transition region 102ₜ of the second portion 102.

In some non-limiting examples, at least one deposited layer 130, including without limitation, an initial deposited layer 130, may provide, at least in part, the functionality of an EIL 639, in the emissive region 610. Non-limiting examples, of the deposited material 331 for forming such initial deposited layer 130 include Yb, which for example, may be about 1-3 nm in thickness.

### Edge Effects of Patterning Coatings and Deposited Layers

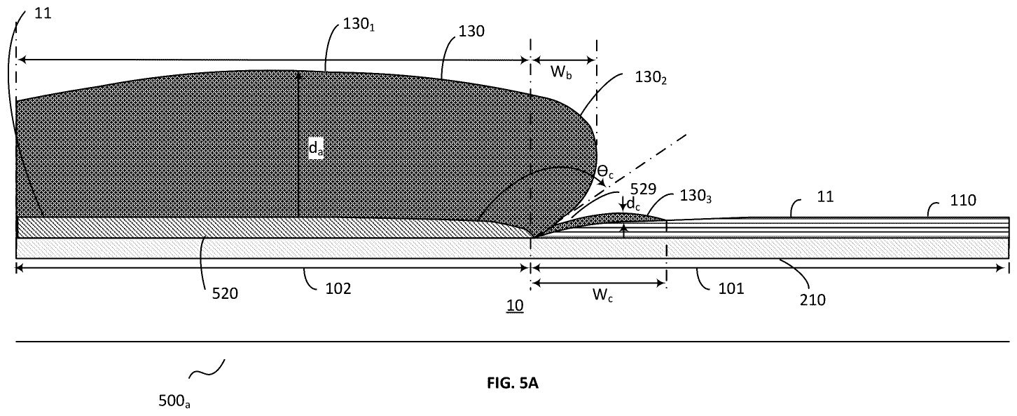

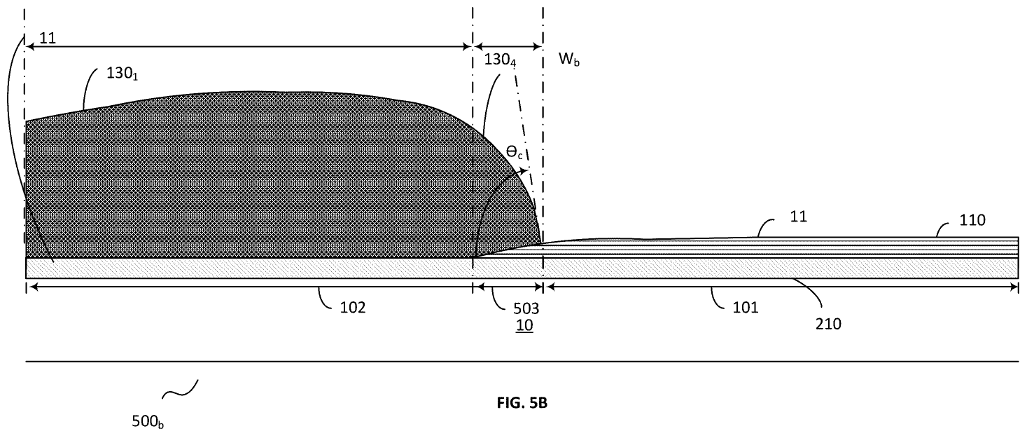

FIGS. 5A-5B describe various potential behaviours of patterning coatings 110 at a deposition interface with deposited layers 140.

Turning to FIG. 5A, there may be shown a first example of a part of an example version 500ₐ of the device 100 at a patterning coating deposition boundary. The device 500ₐ may comprise a substrate 10 having an exposed layer surface 11. A patterning coating 110 may be deposited over a first portion 101 of the exposed layer surface 11 of the underlying layer 210. A deposited layer 130 may be deposited over a second portion 102 of the exposed layer surface 11 of the underlying layer 210. As shown, by way of non-limiting example, the first portion 101 and the second portion 102 may be distinct and non-overlapping parts of the exposed layer surface 11.

The deposited layer 130 may comprise a first part 130₁ and a second part 130₂. As shown, by way of non-limiting example, the first part 130₁ of the deposited layer 130 may substantially cover the second portion 102 and the second part 130₂ of the deposited layer 130 may partially overlap (project over) a first part of the patterning coating 110.

In some non-limiting examples, since the patterning coating 110 may be formed such that its exposed layer surface 11 exhibits a substantially low initial sticking probability against deposition of the deposited material 331, there may be a gap 529 formed between the projecting second part 130₂ of the deposited layer 130 and the exposed layer surface 11 of the patterning coating 110. As a result, the second part 130₂ may not be in physical contact with the patterning coating 110 but may be spaced-apart therefrom by the gap 529 in a cross-sectional aspect. In some non-limiting examples, the first part 130₁ of the deposited layer 130 may be in physical contact with the patterning coating 110 at an interface (boundary) between the first portion 101 and the second portion 102.

In some non-limiting examples, the projecting second part 130₂ of the deposited layer 130 may extend laterally over the patterning coating 110 by a comparable extent as an average layer thickness dₐ of the first part 130₁ of the deposited layer 130. By way of non-limiting example, as shown, a width w_{b} of the second part 130₂ may be comparable to the average layer thickness dₐ of the first part 130₁. In some non-limiting examples, a ratio of a width w_{b} of the second part 130₂ by an average layer thickness dₐ of the first part 130₁ may be in a range of one of between about: 1:1-1:3, 1:1-1:1.5, and 1:1-1:2. While the average layer thickness dₐ may in some non-limiting examples be substantially uniform across the first part 130₁, in some non-limiting examples, the extent to which the second part 130₂ may project over the patterning coating 110 (namely w_{b}) may vary to some extent across different parts of the exposed layer surface 11.

In some non-limiting examples, the deposited layer 130 may be shown to include a third part 130₃ disposed between the second part 130₂ and the patterning coating 110. As shown, the second part 130₂ of the deposited layer 130 may extend laterally over and may be longitudinally spaced apart from the third part 130₃ of the deposited layer 130 and the third part 130₃ may be in physical contact with the exposed layer surface 11 of the patterning coating 110. An average layer thickness d_{c} of the third part 130₃ of the deposited layer 130 may be no more than, and in some non-limiting examples, substantially less than, the average layer thickness dₐ of the first part 130₁ thereof. In some non-limiting examples, a width we of the third part 130₃ may exceed the width w_{b} of the second part 130₂. In some non-limiting examples, the third part 130₃ may extend laterally to overlap the patterning coating 110 to a greater extent than the second part 130₂. In some non-limiting examples, a ratio of a width w_{c} of the third part 130₃ by an average layer thickness dₐ of the first part 130₁ may be in a range of one of between about: 1:2-3:1, and 1:1.2-2.5:1. While the average layer thickness dₐ may in some non-limiting examples be substantially uniform across the first part 130₁, in some non-limiting examples, the extent to which the third part 130₃ may project (overlap) with the patterning coating 110 (namely w_{c}) may vary to some extent across different parts of the exposed layer surface 11.

In some non-limiting examples, the average layer thickness d_{c} of the third part 130₃ may not exceed about 5% of the average layer thickness dₐ of the first part 130₁. By way of non-limiting example, d_{c} may be one of no more than about: 4%, 3%, 2%, 1%, and 0.5% of dₐ. Instead of (including without limitation, in addition to) the third part 130₃ being formed as a thin film, as shown, the deposited material 331 of the deposited layer 130 may form as particle structures 150 (not shown) on a part of the patterning coating 110. By way of non-limiting example, such particle structures 150 may comprise features that are physically separated from one another, such that they do not form a continuous layer.

In some non-limiting examples, as shown, an NPC 520 may be disposed between the substrate 10 and the deposited layer 130. The NPC 520 may be disposed between the first part 130₁ of the deposited layer 130 and the second portion 102 of the exposed layer surface 11 of the underlying layer 210. The NPC 520 is illustrated as being disposed on the second portion 102 and not on the first portion 101, where the patterning coating 110 has been deposited. The NPC 520 may be formed such that, at an interface (boundary) between the NPC 520 and the deposited layer 130, a surface of the NPC 520 may exhibit a substantially high initial sticking probability against deposition of the deposited material 331. As such, the presence of the NPC 520 may promote the formation (growth) of the deposited layer 130 during deposition.

In some non-limiting examples, although not shown, the NPC 520 may be disposed on both the first portion 101 and the second portion 102 of the substrate 10 and the underlying layer 210 may cover a part of the NPC 520 disposed on the first portion 101, and another part of the NPC 520 may be substantially devoid of the underlying layer 210 and of the patterning coating 110, and the deposited layer 130 may cover such part of the NPC 520.

Turning now to FIG. 5B, in some non-limiting examples, the first portion 101 of the substrate 10 may be coated with the patterning coating 110 and the second portion may be coated with the deposited layer 130. In some non-limiting examples, the deposited layer 130 may partially overlap a part of the patterning coating 110 in a third portion 503 of the substrate 10. In some non-limiting examples, although not shown, in addition to the first part 130₁ (and, if present, at least one of: the second part 130₂, and the third part 130₃), the deposited layer 130 may further comprise a fourth part 130₄ that may be disposed between the first part 130₁ and the second part 130₂ of the deposited layer 130 and in physical contact with the exposed layer surface 11 of the patterning coating 110. In some non-limiting examples, the fourth part 130₄ of the deposited layer 130 overlapping a subset of the patterning coating in the third portion 503 may be in physical contact with the exposed layer surface 11 thereof. In some non-limiting examples, the overlap in the third portion 503 may be formed as a result of lateral growth of the deposited layer 130 during one of: an open mask, and mask-free, deposition process. In some non-limiting examples, while the exposed layer surface 11 of the patterning coating 110 may exhibit a substantially low initial sticking probability against deposition of the deposited material 331, and thus a probability of the material nucleating on the exposed layer surface 11 may be low, as the deposited layer 130 grows in thickness, the deposited layer 130 may also grow laterally and may cover a subset of the patterning coating 110 as shown.

In some non-limiting examples, it has been observed that conducting one of: an open mask, and mask-free, deposition of the deposited layer 130 may result in the deposited layer 130 exhibiting a tapered cross-sectional profile proximate to an interface between the deposited layer 130 and the patterning coating 110.

In some non-limiting examples, an average layer thickness of the deposited layer 130 proximate to the interface may be less than an average film thickness d₃ of the deposited layer 130. While such tapered profile may be shown as being at least one of: curved, and arched, in some non-limiting examples, the profile may, in some non-limiting examples be substantially one of: linear, and non-linear. By way of non-limiting example, an average film thickness d₃ of the deposited layer 130 may decrease, without limitation, in a substantially at least one of: linear, exponential, and quadratic, fashion in a region proximate to the interface.

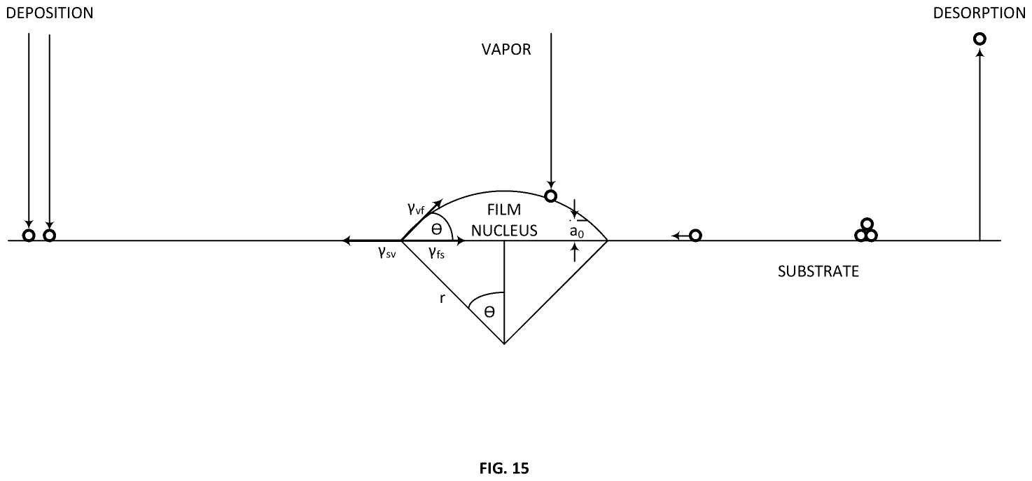

It has been observed that a contact angle θ_{c} of the deposited layer 130 proximate to the interface between the deposited layer 130 and the patterning coating 110 may vary, depending on properties of the patterning coating 110, such as an initial sticking probability. It may be further postulated that the contact angle θ (FIG. 15) of the nuclei may, in some non-limiting examples, dictate the thin film contact angle θ_{c} of the deposited layer 130 formed by deposition. Referring to FIG. 5B by way of non-limiting example, the contact angle θ_{c} may be determined by measuring a slope of a tangent of the deposited layer 130 proximate to the interface between the deposited layer 130 and the patterning coating 110. In some non-limiting examples, where the cross-sectional taper profile of the deposited layer 130 is substantially linear, the contact angle θ_{c} may be determined by measuring the slope of the deposited layer 130 proximate to the interface. As will be appreciated by those having ordinary skill in the relevant art, the contact angle θ_{c} may be generally measured relative to a non-zero angle of the underlying layer 210. In the present disclosure, for purposes of simplicity of illustration, the patterning coating 110 and the deposited layer 130 may be shown deposited on a planar surface. However, those having ordinary skill in the relevant art will appreciate that the patterning coating 110 and the deposited layer 130 may be deposited on non-planar surfaces.

In some non-limiting examples, as shown in FIG. 5A, the contact angle θ_{c} of the deposited layer 130 may exceed about 90° and, by way of non-limiting example, the deposited layer 130 may be shown as including a part 130₂ extending past the interface between the patterning coating 110 and the deposited layer 130 and may be spaced apart from the patterning coating 110 (and, in some non-limiting examples, the third part 130₃ of the deposited layer 130) by the gap 529. In such non-limiting scenario, the contact angle θ_{c} may, in some non-limiting examples, exceed 90°.

In some non-limiting examples, there may be scenarios calling for a deposited layer 130 exhibiting a substantially high contact angle θ_{c}. By way of non-limiting example, the contact angle θ_{c} may exceed one of about: 10°, 15°, 20°, 25°, 30°, 35°, 40°, 50°, 70°, 75°, and 80°. By way of non-limiting example, a deposited layer 130 having a substantially high contact angle θ_{c} may allow for creation of finely patterned features while maintaining a substantially high aspect ratio. By way of non-limiting example, there may be scenarios calling for a deposited layer 130 exhibiting a contact angle θ_{c} that exceeds about 90°. By way of non-limiting example, the contact angle θ_{c} may exceed one of about: 90°, 95°, 100°, 105°, 110° 120°, 130°, 135°, 140°, 145°, 150°, and 170°.

In some non-limiting examples, the deposited layer 130 may partially overlap a part of the patterning coating 110 in the third portion 503 of the substrate 10, which may be disposed between the first portion 101 and the second portion 102 thereof. As shown, the subset of the deposited layer 130 partially overlapping a subset of the patterning coating 110 may be in physical contact with the exposed layer surface 11 thereof. In some non-limiting examples, the overlap in the third portion 503 may be formed because of lateral growth of the deposited layer 130 during one of: an open mask, and mask-free, deposition process. In some non-limiting examples, while the exposed layer surface 11 of the patterning coating 110 may exhibit a substantially low initial sticking probability against deposition of the deposited material 331 and thus the probability of the deposited material 331 nucleating on the exposed layer surface 11 is low, as the deposited layer 130 grows in thickness, the deposited layer 130 may also grow laterally and may cover a subset of the patterning coating 110.

In some non-limiting examples, the contact angle θ_{c} of the deposited layer 130 may be measured at an edge thereof near the interface between it and the patterning coating 110, as shown. In FIG. 5A, the contact angle θ_{c} may exceed about 90°, which may in some non-limiting examples result in a subset, namely the second part 130₂, of the deposited layer 130 being spaced apart from the patterning coating 110 (and, in some non-limiting examples, the third part 130₃ of the deposited layer 130) by the gap 529.

### Particle Structure

An NP is a particle of matter whose predominant characteristic size is of nanometer (nm) scale, generally understood to be between about: 1-300 nm. At nm scale, NPs of a given material may possess unique properties (including without limitation, optical, chemical, physical, and electrical) relative to the same material in bulk form, including without limitation, an amount of absorption of EM radiation exhibited by such NPs at different wavelengths (ranges).

These properties may be exploited when a plurality of NPs is formed into a layer of a layered semiconductor device, including without limitation, an opto-electronic device, to improve its performance.

Current mechanisms for introducing such a layer of NPs into such a device have some drawbacks.

First, in some non-limiting examples, such NPs are formed into at least one of: a close-packed layer, and dispersed into a matrix material, of such device. Consequently, the thickness of such an NP layer may be much thicker than the characteristic size of the NPs themselves. The thickness of such NP layer may impart undesirable characteristics in terms of at least one of: device performance, device stability, device reliability, and device lifetime that may reduce, including without limitation, obviate, any perceived advantages provided by the unique properties of NPs.

Second, techniques to synthesize NPs, in and for use in such devices may introduce large amounts of at least one of: C, O, and S through various mechanisms.

In some non-limiting examples, wet chemical methods may be used to introduce NPs that have a precisely controlled at least one of: characteristic size, length, width, diameter, height, size distribution, shape, surface coverage, configuration, deposited density, dispersity, and composition into an opto-electronic device 600. However, such methods may, in some non-limiting examples, employ an organic capping group (such as the synthesis of citrate-capped Ag NPs) to stabilize the NPs, but such organic capping groups introduce at least one of: C, O, and S into the synthesized NPs.

Still further, in some non-limiting examples, an NP layer deposited from solution may comprise at least one of: C, O, and S, because of the solvents used in deposition.

Additionally, these elements may be introduced as contaminants during at least one of: the wet chemical process, and the deposition of the NP layer.

However introduced, the presence of a high amount of at least one of: C, O, and S, in the NP layer of such a device, may erode at least one of: the performance, stability, reliability, and lifetime, of such device.

Third, when depositing an NP layer from solution, as the employed solvents dry, the NP layer(s) may tend to have non-uniform properties at least one of: across the NP layer, and between different patterned regions of such layer. In some non-limiting examples, an edge of a given layer may be considerably at least one of: thicker and thinner, than an internal region of such layer, which disparities may adversely impact at least one of: the device performance, stability, reliability, and lifetime.

Fourth, while there are other methods (and processes) beyond wet chemical synthesis and solution deposition processes, of at least one of: synthesizing and depositing, NPs, including without limitation, a vacuum-based process such as, without limitation, PVD, such methods tend to provide poor control of the at least one of: characteristic size, length, width, diameter, height, size distribution, shape, surface coverage, configuration, deposited density, dispersity, and composition, of the NPs deposited thereby. In some non-limiting examples, in a PVD process, the NPs tend to form a close-packed film as their size increases. As a result, methods such as PVD are generally not well-suited to form a layer of large disperse NPs with low surface coverage. Rather, the poor control of at least one of: the characteristic size, length, width, diameter, height, size distribution, shape, surface coverage, configuration, deposited density, dispersity, and composition, imparted by such methods may result in poor at least one of: device performance, stability, reliability, and lifetime.

In some non-limiting examples, such as may be shown in FIG. 4A, there may be at least one particle, including without limitation, at least one of: a nanoparticle (NP), an island, a plate, a disconnected cluster, and a network (collectively particle structure 150) disposed on an exposed layer surface 11 of an underlying layer 210. In some non-limiting examples, the underlying layer 210 may be the patterning coating 110 in the first portion 101. In some non-limiting examples, the at least one particle structure 150 may be disposed on an exposed layer surface 11 of the patterning coating 110. In some non-limiting examples, there may be a plurality of such particle structures 150.

In some non-limiting examples, the at least one particle structure 150 may comprise a particle material. In some non-limiting examples, the particle material may be the same as the deposited material 331 in the deposited layer.

In some non-limiting examples, the particle material in the discontinuous layer 160 in the first portion 101, at least one of: the deposited material 331 in the deposited layer 130, and a material of which the underlying layer 210 thereunder may be comprised, may comprise a metal in common.

In some non-limiting examples, the particle material may comprise an element selected from at least one of: K, Na, Li, Ba, Cs, Yb, Ag, Au, Cu, Al, Mg, Zn, Cd, Sn, and Y. In some non-limiting examples, the element may comprise at least one of: K, Na, Li, Ba, Cs, Yb, Ag, Au, Cu, Al, and Mg. In some non-limiting examples, the element may comprise at least one of: Cu, Ag, and Au. In some non-limiting examples, the element may be Cu. In some non-limiting examples, the element may be Al. In some non-limiting examples, the element may comprise at least one of: Mg, Zn, Cd, and Yb. In some non-limiting examples, the element may comprise at least one of: Mg, Ag, Al, Yb, and Li. In some non-limiting examples, the element may comprise at least one of: Mg, Ag, and Yb. In some non-limiting examples, the element may comprise at least one of: Mg, and Ag. In some non-limiting examples, the element may be Ag.

In some non-limiting examples, the particle material may comprise a pure metal. In some non-limiting examples, the at least one particle structure 150 may be a pure metal. In some non-limiting examples, the at least one particle structure 150 may be (substantially) pure Ag. In some non-limiting examples, the substantially pure Ag may have a purity of one of about: 95%, 99%, 99.9%, 99.99%, 99.999%, and 99.9995%. In some non-limiting examples, the at least one particle structure 150 may be (substantially) pure Mg. In some non-limiting examples, the substantially pure Mg may have a purity of one of at least about: 95%, 99%, 99.9%, 99.99%, 99.999%, and 99.9995%.

In some non-limiting examples, the at least one particle structure 150 may comprise an alloy. In some non-limiting examples, the alloy may be at least one of: an Ag-containing alloy, an Mg-containing alloy, and an AgMg-containing alloy. In some non-limiting examples, the AgMg-containing alloy may have an alloy composition that may range from about 1:10 (Ag:Mg) to about 10:1 by volume.

In some non-limiting examples, the particle material may comprise other metals one of: in place of, and in combination with, Ag. In some non-limiting examples, the particle material may comprise an alloy of Ag with at least one other metal. In some non-limiting examples, the particle material may comprise an alloy of Ag with at least one of: Mg, and Yb. In some non-limiting examples, such alloy may be a binary alloy having a composition of between about: 5-95 vol. % Ag, with the remainder being the other metal. In some non-limiting examples, the particle material may comprise Ag and Mg. In some non-limiting examples, the particle material may comprise an Ag:Mg alloy having a composition of between about 1:10-10:1 by volume. In some non-limiting examples, the particle material may comprise Ag and Yb. In some non-limiting examples, the particle material may comprise a Yb:Ag alloy having a composition of between about 1:20-10:1 by volume. In some non-limiting examples, the particle material may comprise Mg and Yb. In some non-limiting examples, the particle material may comprise an Mg:Yb alloy. In some non-limiting examples, the particle material may comprise an Ag:Mg:Yb alloy.

In some non-limiting examples, the at least one particle structure 150 may comprise at least one additional element. In some non-limiting examples, such additional element may be a non-metallic element. In some non-limiting examples, the non-metallic material may be at least one of: O, S, N, and C. It will be appreciated by those having ordinary skill in the relevant art that, in some non-limiting examples, such additional element(s) may be incorporated into the at least one particle structure 150 as a contaminant, due to the presence of such additional element(s) in at least one of: the source material, equipment used for deposition, and the vacuum chamber environment. In some non-limiting examples, such additional element(s) may form a compound together with other element(s) of the at least one particle structure 150. In some non-limiting examples, a concentration of the non-metallic element in the particle material may be one of no more than about: 1%, 0.1%, 0.01%, 0.001%, 0.0001%, 0.00001%, 0.000001%, and 0.0000001%. In some non-limiting examples, the at least one particle structure 150 may have a composition in which a combined amount of O and C therein is one of no more than about: 10%, 5%, 1%, 0.1%, 0.01%, 0.001%, 0.0001%, 0.00001%, 0.000001%, and 0.0000001%.

The at least one particle structure 150 takes advantage of plasmonics, a branch of nanophotonics, which studies the resonant interaction of EM radiation with metals. Those having ordinary skill in the relevant art will appreciate that metal NPs may exhibit at least one of: localized surface plasmon (LSP) excitations, and coherent oscillations of free electrons, whose optical response may be tailored by varying at least one of: a characteristic size, size distribution, shape, surface coverage, configuration, deposited density, and composition, of the nanostructures. Such optical response, in respect of particle structures 150, may include absorption of EM radiation incident thereon, thereby reducing at least one of: reflection thereof, and shifting to one of: a lower, and higher, wavelength ((sub-) range) of the EM spectrum, including without limitation, (a sub-range of) the visible spectrum.

It has also been reported that arranging certain metal NPs near a medium having substantially low refractive index, may shift the absorption spectrum of such NPs to a lower wavelength (sub-) range (blue-shifted).

Accordingly, it may be further postulated that disposing particle material, in some non-limiting examples, as a discontinuous layer 160 of at least one particle structure 150 on an exposed layer surface 11 of an underlying layer 210, such that the at least one particle structure 150 is in physical contact with the underlying layer 210, may, in some non-limiting examples, favorably shift the absorption spectrum of the particle material, including without limitation, blue-shift, such that it does not substantially overlap with a wavelength range of the EM spectrum of EM radiation being at least one of: emitted by, and transmitted at least partially through, the device 100.

In some non-limiting examples, a peak absorption wavelength of the at least one particle structure 150 may be less than a peak wavelength of the EM radiation being at least one of: emitted by, and transmitted, at least partially through the device 100. In some non-limiting examples, the particle material may exhibit a peak absorption at a wavelength (range) that is one of no more than about: 470 nm, 460 nm, 455 nm, 450 nm, 445 nm, 440 nm, 430 nm, 420 nm, and 400 nm.

It has now been found, that providing particle material, including without limitation, in the form of at least one particle structure 150, including without limitation, those comprised of a metal, proximate to, including without limitation, within, at least one low(er)-index coating, may further impact at least one of: the absorption, and transmittance, of EM radiation passing through the device 100, including without limitation, in the first direction, in at least a wavelength (sub-) range of the EM spectrum, including without limitation, (a sub-range of) the visible spectrum, passing in the first direction from, including without limitation, through, the at least one low(er)-index layer(s) and the at least one particle structure(s) 150.

In some non-limiting examples, at least one of: absorption may be reduced, and transmittance may be facilitated, in at least a wavelength (sub-) range of the EM spectrum, including without limitation, (a sub-range of) the visible spectrum.

In some non-limiting examples, the absorption may be concentrated in an absorption spectrum that is a wavelength (sub-) range of the EM spectrum, including without limitation, (a sub-range of) the visible spectrum.

In some non-limiting examples, the absorption spectrum may be one of: blue-shifted, and shifted to a higher wavelength (sub-) range (red-shifted), including without limitation, to a wavelength (sub-) range of the EM spectrum, including without limitation, (a sub-range of) the visible spectrum, and to a wavelength (sub-) range of the EM spectrum that lies, at least in part, beyond the visible spectrum.

Those having ordinary skill in the relevant art will appreciate that in some non-limiting examples, a plurality of layers of at least one particle structure 150 may be disposed on one another, whether separated by additional layers, with varying lateral aspects and having different absorption spectra. In this fashion, the absorption of certain regions of the device may be tuned according to at least one desired absorption spectra.

In some non-limiting examples, the presence of the at least one particle structure 150, including without limitation, NPs, including without limitation, in a discontinuous layer 160, on an exposed layer surface 11 of the patterning coating 110 may affect some optical properties of the device 100.

In some non-limiting examples, such plurality of particle structures 150 may form a discontinuous layer 160.

Without wishing to be limited to any particular theory, it may be postulated that, while the formation of a closed coating 140 of the particle material may be substantially inhibited by the patterning coating 110, in some non-limiting examples, when the patterning coating 110 is exposed to deposition of the particle material thereon, some vapor monomers of the particle material may ultimately form at least one particle structure 150 of the particle material thereon.

In some non-limiting examples, at least some of the particle structures 150 may be disconnected from one another. In other words, in some non-limiting examples, the discontinuous layer 160 may comprise features, including particle structures 150, that may be physically separated from one another, such that the particle structures 150 do not form a closed coating 140. Accordingly, such discontinuous layer 160 may, in some non-limiting examples, thus comprise a thin disperse layer of deposited material 331 formed as particle structures 150, inserted at, including without limitation, substantially across, the lateral extent of, an interface between the patterning coating 110 and at least one overlying layer in the device 100.

In some non-limiting examples, at least one of the particle structures 150 of particle material may be in physical contact with an exposed layer surface 11 of the patterning coating 110. In some non-limiting examples, substantially all of the particle structures 150 of particle material may be in physical contact with the exposed layer surface 11 of the patterning coating 110.

Without wishing to be bound by any particular theory, it has been found, that the presence of such a thin, disperse discontinuous layer 160 of particle material, including without limitation, at least one particle structure 150, including without limitation, metal particle structures 150, on an exposed layer surface 11 of the patterning coating 110, may exhibit at least one varied characteristic and concomitantly, varied behaviour, including without limitation, optical effects and properties of the device 100, as discussed herein. In some non-limiting examples, such effects and properties may be controlled to some extent by judicious selection of at least one of: the characteristic size, size distribution, shape, surface coverage, configuration, deposited density, and dispersity, of the particle structures 150 on the patterning coating 110.

In some non-limiting examples, the particle structures 150 may be controllably selected so as to have at least one of: a characteristic size, length, width, diameter, height, size distribution, shape, surface coverage, configuration, deposited density, dispersity, and composition, to achieve an effect related to an optical response exhibited by the particle structures 150.

Those having ordinary skill in the relevant art will appreciate that, having regard to the mechanism by which materials are deposited, due to possible stacking, including without limitation, clustering, of at least one of: monomers, and atoms, at least one of: an actual size, height, weight, thickness, shape, profile, and spacing, of the at least one particle structure 150 may be, in some non-limiting examples, substantially non-uniform. Additionally, although the at least one particle structure 150 are illustrated as having a given profile, this is intended to be illustrative only, and not determinative of at least one of: a size, height, weight, thickness, shape, profile, and spacing, thereof.

In some non-limiting examples, the at least one particle structure 150 may have a characteristic dimension of no more than about 200 nm. In some non-limiting examples, the at least one particle structure 150 may have a characteristic diameter that may be one of between about: 1-200 nm, 1-160 nm, 1-100 nm, 1-50 nm, and 1-30 nm.

In some non-limiting examples, the at least one particle structure 150 may comprise discrete metal plasmonic islands (clusters).

In some non-limiting examples, the at least one particle structure 150 may comprise a particle material.

In some non-limiting examples, such particle structures 150 may be formed by depositing a scant amount, in some non-limiting examples, having an average layer thickness that may be on the order of one of: a few, and a fraction of one, angstrom(s), of a particle material on an exposed layer surface 11 of the underlying layer 210. In some non-limiting examples, the exposed layer surface 11 may be of an NPC 520.

In some non-limiting examples, the particle material may comprise at least one of: Ag, Yb, and Mg.

In some non-limiting examples, the formation of at least one of: the characteristic size, size distribution, shape, surface coverage, configuration, deposited density, and dispersity, of such discontinuous layer 160 may be controlled, in some non-limiting examples, by judicious selection of at least one of: at least one characteristic of the patterning material 211, an average film thickness d₂ of the patterning coating 110, the introduction of heterogeneities in at least one of: the patterning coating 110, and a deposition environment, including without limitation, a temperature, pressure, duration, deposition rate, and deposition process, for the patterning coating 110.

In some non-limiting examples, the formation of at least one of the characteristic size, size distribution, shape, surface coverage, configuration, deposited density, and dispersity, of such discontinuous layer 160 may be controlled, in some non-limiting examples, by judicious selection of at least one of: at least one characteristic of the particle material (which may be the deposited material 331), an extent to which the patterning coating 110 may be exposed to deposition of the particle material (which, in some non-limiting examples may be specified in terms of a thickness of the corresponding discontinuous layer 160), and a deposition environment, including without limitation, at least one of: a temperature, pressure, duration, deposition rate, and method of deposition for the particle material.

In some non-limiting examples, the discontinuous layer 160 may be deposited in a pattern across the lateral extent of the patterning coating 110.

In some non-limiting examples, the discontinuous layer 160 may be disposed in a pattern that may be defined by at least one region therein that is substantially devoid of the at least one particle structure 150.

In some non-limiting examples, the characteristics of such discontinuous layer 160 may be assessed, in some non-limiting examples, somewhat arbitrarily, according to at least one of several criteria, including without limitation, at least one of: a characteristic size, size distribution, shape, configuration, surface coverage, deposited distribution, dispersity, and a presence, and an extent of aggregation instances, of the particle material, formed on a part of the exposed layer surface 11 of the underlying layer 210.

In some non-limiting examples, an assessment of the discontinuous layer 160 according to such at least one criterion, may be performed on, including without limitation, by at least one of: measuring, and calculating, at least one attribute of the discontinuous layer 160, using a variety of imaging techniques, including without limitation, at least one of: transmission electron microscopy (TEM), atomic force microscopy (AFM), and scanning electron microscopy (SEM).

Those having ordinary skill in the relevant art will appreciate that such an assessment of the discontinuous layer 160 may depend, to at least one of: a greater, and lesser, extent, by the extent, of the exposed layer surface 11 under consideration, which in some non-limiting examples may comprise an area, including without limitation, a region thereof. In some non-limiting examples, the discontinuous layer 160 may be assessed across the entire extent, in at least one of: a first lateral aspect, and a second lateral aspect that is substantially transverse thereto, of the exposed layer surface 11. In some non-limiting examples, the discontinuous layer 160 may be assessed across an extent that comprises at least one observation window applied against (a part of) the discontinuous layer 160.

In some non-limiting examples, the at least one observation window may be located at at least one of: a perimeter, interior location, and grid coordinate, of the lateral aspect of the exposed layer surface 11. In some non-limiting examples, a plurality of the at least one observation windows may be used in assessing the discontinuous layer 160.

In some non-limiting examples, the observation window may correspond to a field of view of an imaging technique applied to assess the discontinuous layer 160, including without limitation, at least one of: TEM, AFM, and SEM. In some non-limiting examples, the observation window may correspond to a given level of magnification, including without limitation, one of: 2.00 μm, 1.00 μm, 500 nm, and 200 nm.

In some non-limiting examples, the assessment of the discontinuous layer 160, including without limitation, at least one observation window used, of the exposed layer surface 11 thereof, may involve at least one of: calculating, and measuring, by any number of mechanisms, including without limitation, at least one of: manual counting, and known estimation techniques, which may, in some non-limiting examples, comprise at least one of: curve, polygon, and shape, fitting techniques.

In some non-limiting examples, the assessment of the discontinuous layer 160, including without limitation, at least one observation window used, of the exposed layer surface 11 thereof, may involve at least one of: calculating, and measuring, at least one of: an average, median, mode, maximum, minimum, and other at least one of: probabilistic, statistical, and data, manipulation, of a value of the at least one of: calculation, and measurement.

In some non-limiting examples, one of the at least one criterion by which such discontinuous layer 160 may be assessed, may be a surface coverage of the particle material on such (part of the) discontinuous layer 160. In some non-limiting examples, the surface coverage may be represented by a (non-zero) percentage coverage by such particle material of such (part of the) discontinuous layer 160. In some non-limiting examples, the percentage coverage may be compared to a maximum threshold percentage coverage.

In some non-limiting examples, a (part of a) discontinuous layer 160 having a surface coverage that may be substantially no more than the maximum threshold percentage coverage, may result in a manifestation of different optical characteristics that may be imparted by such part of the discontinuous layer 160, to EM radiation passing therethrough, whether at least one of: transmitted entirely through the device 100, and emitted thereby, relative to EM radiation passing through a part of the discontinuous layer 160 having a surface coverage that substantially exceeds the maximum threshold percentage coverage.

In some non-limiting examples, one measure of a surface coverage of an amount of an electrically conductive material on a surface may be a (EM radiation) transmittance, since in some non-limiting examples, electrically conductive materials, including without limitation, metals, including without limitation: Ag, Mg, and Yb, may at least one of: attenuate, and absorb, EM radiation.

Those having ordinary skill in the relevant art will appreciate that in some non-limiting examples, surface coverage may be understood to encompass at least one of: particle size, and deposited density. Thus, in some non-limiting examples, a plurality of these three criteria may be positively correlated. Indeed, in some non-limiting examples, a criterion of low surface coverage may comprise some combination of a criterion of low deposited density with a criterion of low particle size.

In some non-limiting examples, one of the at least one criterion by which such discontinuous layer 160 may be assessed, may be a characteristic size of the constituent particle structures 150.

In some non-limiting examples, the at least one particle structure 150 of the discontinuous layer 160, may have a characteristic size that is no more than a maximum threshold size. Non-limiting examples of the characteristic size may include at least one of: height, width, length, and diameter.

In some non-limiting examples, substantially all of the particle structures 150 of the discontinuous layer 160 may have a characteristic size that lies within a specified range.

In some non-limiting examples, such characteristic size may be characterized by a characteristic length, which in some non-limiting examples, may be considered a maximum value of the characteristic size. In some non-limiting examples, such maximum value may extend along a major axis of the particle structure 150. In some non-limiting examples, the major axis may be understood to be a first dimension extending in a plane defined by the plurality of lateral axes. In some non-limiting examples, a characteristic width may be identified as a value of the characteristic size of the particle structure 150 that may extend along a minor axis of the particle structure 150. In some non-limiting examples, the minor axis may be understood to be a second dimension extending in the same plane but substantially transverse to the major axis.

In some non-limiting examples, the characteristic length of the at least one particle structure 150, along the first dimension, may be no more than the maximum threshold size.

In some non-limiting examples, the characteristic width of the at least one particle structure 150, along the second dimension, may be no more than the maximum threshold size.

In some non-limiting examples, a size of the constituent particle structures 150, in the (part of the) discontinuous layer 160, may be assessed by at least one of: calculating, and measuring a characteristic size of such at least one particle structure 150, including without limitation, at least one of: a mass, volume, length of a diameter, perimeter, major, and minor axis, thereof.

In some non-limiting examples, one of the at least one criterion by which such discontinuous layer 160 may be assessed, may be a deposited density thereof.

In some non-limiting examples, the characteristic size of the particle structure 150 may be compared to a maximum threshold size.

In some non-limiting examples, the deposited density of the particle structures 150 may be compared to a maximum threshold deposited density.

In some non-limiting examples, at least one of such criteria may be quantified by a numerical metric. In some non-limiting examples, such a metric may be a calculation of a dispersity D that describes the distribution of particle (area) sizes in a deposited layer 130 of particle structures 150, in which:

$$D = \frac{\overline{{S}_{s}}}{\overline{{S}_{n}}} \qquad ( 1 )$$

$$where :$$

$$\overline{{S}_{s}} = \frac{{\sum }_{i = 1}^{n} {S}_{i}^{2}}{{\sum }_{i = 1}^{n} {S}_{i}} , \overline{{S}_{n}} = \frac{{\sum }_{i = 1}^{n} {S}_{i}}{n} , \qquad ( 2 )$$

n is the number of particle structures 150 in a sample area,
Sᵢ is the (area) size of the i^{th} particle structure 150,
S̅ₙ is the number average of the particle (area) sizes and
S̅ₛ is the (area) size average of the particle (area) sizes.

Those having ordinary skill in the relevant art will appreciate that the dispersity is roughly analogous to a polydispersity index (PDI) and that these averages are roughly analogous to the concepts of number average molecular weight and weight average molecular weight familiar in organic chemistry, but applied to an (area) size, as opposed to a molecular weight of a sample particle structure 150.

Those having ordinary skill in the relevant will also appreciate that while the concept of dispersity may, in some non-limiting examples, be considered a three-dimensional volumetric concept, in some non-limiting examples, the dispersity may be considered to be a two-dimensional concept. As such, the concept of dispersity may be used in connection with viewing and analyzing two-dimensional images of the deposited layer 130, such as may be obtained by using a variety of imaging techniques, including without limitation, at least one of: TEM, AFM, and SEM. It is in such a two-dimensional context, that the equations set out above are defined.

In some non-limiting examples, at least one of: the dispersity, and the number average, of the particle (area) size and the (area) size average of the particle (area) size may involve a calculation of at least one of: the number average of the particle diameters and the (area) size average of the particle diameters:

$$\overline{{d}_{n}} = 2 \sqrt{\frac{\overline{{s}_{n}}}{π}} , \overline{{d}_{s}} = 2 \sqrt{\frac{\overline{{s}_{s}}}{π}} \qquad ( 3 )$$

In some non-limiting examples, the particle material, including without limitation as particle structures 150, of the at least one deposited layer 130, may be deposited by one of: an open mask, and mask-free, deposition process.

In some non-limiting examples, the particle structures 150 may have a substantially round shape. In some non-limiting examples, the particle structures 150 may have a substantially spherical shape.

For purposes of simplification, in some non-limiting examples, it may be assumed that a longitudinal extent of each particle structure 150 may be substantially the same (and, in any event, may not be directly measured from a plan view SEM image) so that the (area) size of the particle structure 150 may be represented as a two-dimensional area coverage along the pair of lateral axes. In the present disclosure, a reference to an (area) size may be understood to refer to such two-dimensional concept, and to be differentiated from a size (without the prefix “area”) that may be understood to refer to a one-dimensional concept, such as a linear dimension.

Indeed, in some early investigations, it appears that, in some non-limiting examples, the longitudinal extent, along the longitudinal axis, of such particle structures 150, may tend to be small relative to the lateral extent (along at least one of the lateral axes), such that the volumetric contribution of the longitudinal extent thereof may be much less than that of such lateral extent. In some non-limiting examples, this may be expressed by an aspect ratio (a ratio of a longitudinal extent to a lateral extent) that may be no more than 1. In some non-limiting examples, such aspect ratio may be one of about: 1:10, 1:20, 1:50, 1:75, and 1:300.

In this regard, the assumption set out above (that the longitudinal extent is substantially the same and can be ignored) to represent the particle structure 150 as a two-dimensional area coverage may be appropriate.

Those having ordinary skill in the relevant art will appreciate, having regard to the non-determinative nature of the deposition process, especially in the presence of at least one of: defects, and anomalies, on the exposed layer surface 11 of the underlying layer 210, including without limitation, heterogeneities, including without limitation, at least one of: a step edge, a chemical impurity, a bonding site, a kink, and a contaminant, thereon, and consequently the formation of particle structures 150 thereon, the non-uniform nature of coalescence thereof as the deposition process continues, and in view of the uncertainty in the at least one of: size, and position, of observation windows, as well as the intricacies and variability inherent in at least one of: the calculation, and measurement, of their characteristic size, spacing, deposited density, degree of aggregation, and the like, there may be considerable variability in terms of the features (topology) within observation windows.

In the present disclosure, for purposes of simplicity of illustration, certain details of particle materials, including without limitation, at least one of: thickness profiles, and edge profiles, of layer(s) have been omitted.

Those having ordinary skill in the relevant art will appreciate that certain metal NPs, whether as part of a discontinuous layer 160 of particle material, including without limitation, at least one particle structure 150, may exhibit at least one of: surface plasmon (SP) excitations, and coherent oscillations of free electrons, with the result that such NPs may one of: absorb, and scatter, light in a range of the EM spectrum, including without limitation, (a sub-range of) the visible spectrum. The optical response, including without limitation, at least one of: the (sub-) range of the EM spectrum over which absorption may be concentrated (absorption spectrum), refractive index, and extinction coefficient, of such one of: LSP excitations, and coherent oscillations, may be tailored by varying properties of such NPs, including without limitation, at least one of: a characteristic size, size distribution, shape, surface coverage, configuration, deposition density, dispersity, and property, including without limitation, at least one of: material, and degree of aggregation, of at least one of: the nanostructures, and a medium proximate thereto.

Such optical response, in respect of photon-absorbing coatings, may include absorption of photons incident thereon, thereby reducing reflection. In some non-limiting examples, the absorption may be concentrated in a range of the EM spectrum, including without limitation, (a sub-range of) the visible spectrum. While the at least one particle structure 150 may absorb EM radiation incident thereon from beyond the layered semiconductor device 100, thus reducing reflection, those having ordinary skill in the relevant art will appreciate that, in some non-limiting examples, the at least one particle structure 150 may absorb EM radiation incident thereon that is emitted by the device 100. In some non-limiting examples, employing a photon-absorbing layer as part of an opto-electronic device may reduce reliance on a polarizer therein.

It has been reported in Fusella et al., “Plasmonic enhancement of stability and brightness in organic light-emitting devices”, Nature 2020, 585, at 379-382, that the stability of an OLED device may be enhanced by incorporating an NP-based outcoupling layer above the cathode layer to extract energy from the plasmon modes. The NP-based outcoupling layer was fabricated by spin-casting cubic Ag NPs on top of an organic layer on top of a cathode. However, since most commercial OLED devices are fabricated using vacuum-based processing, spin-casting from solution may not constitute an appropriate mechanism for forming such an NP-based outcoupling layer above the cathode.

It has been discovered that such an NP-based outcoupling layer above the cathode may be fabricated in vacuum (and thus, may have applicability for use in a commercial OLED fabrication process), by depositing a metal particle material in a discontinuous layer 160 onto a patterning coating 110, which in some non-limiting examples, may at least one of: be, and be deposited on, the cathode. Such process may avoid the use of one of: solvents, and other wet chemicals, that may at least one of: cause damage to the OLED device, and may adversely impact device reliability.

In some non-limiting examples, the presence of such a discontinuous layer 160 of particle material, including without limitation, at least one particle structure 150, may contribute to enhanced extraction of at least one of: EM radiation, performance, stability, reliability, and lifetime of the device.

In some non-limiting examples, the existence, in a layered device 100, of at least one discontinuous layer 160, proximate to at least one of: the exposed layer surface 11 of a patterning coating 110, and, in some non-limiting examples, proximate to the interface of such patterning 110 with at least one overlying layer 170, may impart optical effects to EM signals, including without limitation, photons, that are one of: emitted by the device, and transmitted therethrough.

Those having ordinary skill in the relevant art will appreciate that, while a simplified model of the optical effects is presented herein, at least one of: other models, and other explanations, may be applicable.

In some non-limiting examples, the presence of such a discontinuous layer 160 of the particle material, including without limitation, at least one particle structure 150, may reduce (mitigate) crystallization of thin film coatings disposed adjacent in the longitudinal aspect, including without limitation, at least one of: the patterning coating 110, and at least one overlying layer 170, thereby stabilizing the property of the thin film(s) disposed adjacent thereto, and, in some non-limiting examples, reducing scattering. In some non-limiting examples, such thin film may comprise at least one layer of at least one of: an outcoupling, and an encapsulating coating (not shown) of the device, including without limitation, a capping layer (CPL).

In some non-limiting examples, the presence of such a discontinuous layer 160 of particle material, including without limitation, at least one particle structure 150, may provide an enhanced absorption in at least a part of the UV spectrum. In some non-limiting examples, controlling the characteristics of such particle structures 150, including without limitation, at least one of: characteristic size, size distribution, shape, surface coverage, configuration, deposited density, dispersity, particle material, and refractive index, of the particle structures 150, may facilitate controlling the degree of absorption, wavelength range and peak wavelength of the absorption spectrum, including in the UV spectrum. Enhanced absorption of EM radiation in at least a part of the UV spectrum may have applicability in some scenarios, for improving at least one of: device performance, stability, reliability, and lifetime.

In some non-limiting examples, the optical effects may be described in terms of its impact on at least one of: the transmission, and absorption wavelength spectrum, including at least one of: a wavelength range, and peak intensity thereof.

Additionally, while the model presented may suggest certain effects imparted on at least one of: the transmission, and absorption, of photons passing through such discontinuous layer 160, in some non-limiting examples, such effects may reflect local effects that may not be reflected on a broad, observable basis.

### Opto-Electronic Device

As shown in FIG. 1, the layers of the device 100 may comprise a substrate 10, and a patterning coating 110 disposed on an exposed layer surface 11 of at least a portion of the lateral aspect thereof. In some non-limiting examples, the patterning coating 110 may be limited in its lateral extent to the first portion 101 and a deposited layer 130 may be disposed as a closed coating 140 on an exposed layer surface 11 of the device 100 in a second portion 102 of its lateral aspect.

In some non-limiting examples, at least one particle structure 150 may be disposed as a discontinuous layer 160 on the exposed layer surface 11 of the patterning coating 110. In some non-limiting examples, although not shown, at least one of: the patterning coating 110, the deposited layer 130, and at least one particle structure 150, may be deposited on a layer (underlying layer 210) other than the substrate 10 including without limitation, an intervening layer between the substrate 10 and at least one of: the patterning coating 110, deposited layer 130, and the at least one particle structure 150. In some non-limiting examples, the underlying layer 210 may comprise at least one of: an orientation layer, and an organic supporting layer.

In some non-limiting examples, at least one of: the patterning coating 110, the deposited layer 130, and the at least one particle structure 150, may be covered by at least one overlying layer 170.

In some non-limiting examples, such overlying layer 170 may comprise at least one of: an encapsulation layer and an optical coating. Non-limiting examples of an encapsulation layer include a glass cap, a barrier film, a barrier adhesive, a barrier coating, an encapsulation layer, and a thin film encapsulation (TFE) layer, provided to encapsulate the device 100. Non-limiting examples of an optical coating include at least one of: an optical, and structural, coating, and at least one component thereof, including without limitation, a polarizer, a color filter, an anti-reflection coating, an anti-glare coating, cover glass, and an optically clear adhesive (OCA).

In some non-limiting examples, at least one of: a substantially thin patterning coating 110 in the first portion 101, and a deposited layer 130 in the second portion 102, may provide a substantially planar surface on which the overlying layer 170 may be deposited. In some non-limiting examples, providing such a substantially planar surface for application of such overlying layer 170 may increase adhesion thereof to such surface.

In some non-limiting examples, the optical coating may be used to modulate optical properties of EM radiation being at least one of: transmitted, emitted, and absorbed, by the device 100, including without limitation, plasmon modes. In some non-limiting examples, the optical coating may be used as at least one of: an optical filter, index-matching coating, optical outcoupling coating, scattering layer, diffraction grating, and parts thereof.

In some non-limiting examples, the optical coating may be used to modulate at least one optical microcavity effect in the device by, without limitation, tuning at least one of: the total optical path length, and the refractive index thereof. At least one optical property of the device may be affected by modulating at least one optical microcavity effect including without limitation, the output EM radiation, including without limitation, at least one of: an angular dependence of an intensity thereof, and a wavelength shift thereof. In some non-limiting examples, the optical coating may be a non-electrical component, that is, the optical coating may not be configured to at least one of: conduct, and transmit, electrical current during normal device operations.

In some non-limiting examples, the optical coating may be formed of any deposited material 331, and in some non-limiting examples, may employ any mechanism of depositing a deposited layer 130 as described herein.

FIG. 6 is a simplified block diagram from a longitudinal aspect, of an example opto-electronic device, which may be, in some non-limiting examples, an electro-luminescent device 600, according to the present disclosure. In some non-limiting examples, the device 600 may be an OLED.

The device 600 may comprise a substrate 10, upon which a frontplane 601, comprising a plurality of layers, respectively, a first electrode 620, at least one semiconducting layer 630, and a second electrode 640, are disposed. In some non-limiting examples, the frontplane 601 may provide mechanisms for at least one of: emission of EM radiation, including without limitation, photons, and manipulation of emitted EM radiation.

In some non-limiting examples, the deposited layer 130 and the underlying layer 210 may together form at least a part of at least one of: the first electrode 620, and the second electrode 640, of the device 600.

In some non-limiting examples, various coatings of such devices 600 may be formed by vacuum-based deposition processes.

In some non-limiting examples, the second electrode 640 may extend partially over the patterning coating 110 in a transition region 645.

In some non-limiting examples, at least one particle structure 150_{d} of a discontinuous layer 160 of a material of which the deposited layer 130 may be comprised (deposited material 331) may extend partially over the patterning coating 110, which may act as a particle structure patterning coating 110ₚ in the transition region 645. In some non-limiting examples, such discontinuous layer 160 may form at least a part of the second electrode 640.

In some non-limiting examples, the device 600 may be electrically coupled with a power source 604. When so coupled, the device 600 may emit EM radiation, including without limitation, photons, as described herein.

### Substrate

In some non-limiting examples, the substrate 10 may comprise a base substrate 615. In some non-limiting examples, the base substrate 615 may be formed of material suitable for use thereof, including without limitation, at least one of: an inorganic material, including without limitation, at least one of: Si, glass, metal (including without limitation, a metal foil), sapphire, and other inorganic material, and an organic material, including without limitation, a polymer, including without limitation, at least one of: a polyimide, and an Si-based polymer. In some non-limiting examples, the base substrate 615 may be one of: rigid, and flexible. In some non-limiting examples, the substrate 10 may be defined by at least one planar surface. In some non-limiting examples, the substrate 10 may have at least one exposed layer surface 11 that supports the remaining frontplane 601 components of the device 600, including without limitation, at least one of: the first electrode 620, the at least one semiconducting layer 630, and the second electrode 640.

In some non-limiting examples, such surface may be at least one of: an organic surface, and an inorganic surface.

In some non-limiting examples, the substrate 10 may comprise, in addition to the base substrate 615, at least one additional at least one of: organic, and inorganic, layer (not shown nor specifically described herein) supported on an exposed layer surface 11 of the base substrate 615.

In some non-limiting examples, such additional layers may comprise at least one organic layer, which may at least one of: comprise, replace, and supplement, at least one of the semiconducting layers 630.

In some non-limiting examples, such additional layers may comprise at least one inorganic layer, which may comprise, at least one electrode, which in some non-limiting examples, may at least one of: comprise, replace, and supplement, at least one of: the first electrode 620, and the second electrode 640.

### Backplane and TFT Structure(s) Embodied Therein

In some non-limiting examples, such additional layers may comprise a backplane 602. In some non-limiting examples, the backplane 602 may comprise at least one of: power circuitry, and switching elements for driving the device 600, including without limitation, at least one of: at least one electronic TFT structure 606, and at least one component thereof, that may be formed by a photolithography process.

In some non-limiting examples, the backplane 602 of the substrate 10 may comprise at least one electronic, including without limitation, an opto-electronic, component, including without limitation, one of: transistors, resistors, and capacitors, such as which may support the device 600 acting as one of: an active-matrix, and a passive matrix, device. In some non-limiting examples, such structures may be a thin-film transistor (TFT) structure 606.

Non-limiting examples of TFT structures 606 include one of: top-gate, bottom-gate, n-type and p-type TFT structures 606. In some non-limiting examples, the TFT structure 606 may incorporate one of: amorphous Si (a-Si), indium gallium zinc oxide (IGZO), and low-temperature polycrystalline Si (LTPS).

### First Electrode

The first electrode 620 may be deposited over the substrate 10. In some non-limiting examples, the first electrode 620 may be electrically coupled with at least one of: a terminal of the power source 604, and ground. In some non-limiting examples, the first electrode 620 may be so coupled through at least one driving circuit which in some non-limiting examples, may incorporate at least one TFT structure 606 in the backplane 602 of the substrate 10.

In some non-limiting examples, the first electrode 620 may comprise one of: an anode, and cathode. In some non-limiting examples, the first electrode 620 may be an anode.

In some non-limiting examples, the first electrode 620 may be formed by depositing at least one thin conductive film, over (a part of) the substrate 10. In some non-limiting examples, there may be a plurality of first electrodes 620, disposed in a spatial arrangement over a lateral aspect of the substrate 10. In some non-limiting examples, at least one of such at least one first electrodes 620 may be deposited over (a part of) a TFT insulating layer 607 disposed in a lateral aspect in a spatial arrangement. If so, in some non-limiting examples, at least one of such at least one first electrodes 620 may extend through an opening of the corresponding TFT insulating layer 607 to be electrically coupled with an electrode of the TFT structures 606 in the backplane 602.

In some non-limiting examples, at least one of: the at least one first electrode 620, and at least one thin film thereof, may comprise various materials, including without limitation, at least one metallic material, including without limitation, at least one of: Mg, aluminum (Al), calcium (Ca), Zn, Ag, Cd, barium (Ba), and Yb, including without limitation, alloys comprising any of such materials, at least one metal oxide, including without limitation, a TCO, including without limitation, ternary compositions such as, without limitation, at least one of: FTO, IZO, and ITO, in varying proportions, including without limitation, combinations of any plurality thereof in at least one layer, any at least one of which may be, without limitation, a thin film.

### Second Electrode

The second electrode 640 may be deposited over the at least one semiconducting layer 630. In some non-limiting examples, the second electrode 640 may be electrically coupled with at least one of: a terminal of the power source 604, and ground. In some non-limiting examples, the second electrode 640 may be so coupled through at least one driving circuit, which in some non-limiting examples, may incorporate at least one TFT structure 606 in the backplane 602 of the substrate 10.

In some non-limiting examples, the second electrode 640 may comprise one of: an anode, and a cathode. In some non-limiting examples, the second electrode 640 may be a cathode.

In some non-limiting examples, the second electrode 640 may be formed by depositing a deposited layer 130, in some non-limiting examples, as at least one thin film, over (a part of) the at least one semiconducting layer 630. In some non-limiting examples, there may be a plurality of second electrodes 640, disposed in a spatial arrangement over a lateral aspect of the at least one semiconducting layer 630.

In some non-limiting examples, the at least one second electrode 640 may comprise various materials, including without limitation, at least one metallic material, including without limitation, at least one of: Mg, Al, Ca, Zn, Ag, Cd, Ba, and Yb, including without limitation, alloys comprising at least one of: any of such materials, at least one metal oxide, including without limitation, a TCO, including without limitation, ternary compositions such as, without limitation, at least one of: FTO, IZO, and ITO, including without limitation, in varying proportions, zinc oxide (ZnO), and other oxides comprising at least one of: In, and Zn, in at least one layer, and at least one non-metallic material, any of which may be, without limitation, a thin conductive film. In some non-limiting examples, for a Mg:Ag alloy, such alloy composition may range between about 1:9-9:1 by volume.

In some non-limiting examples, the deposition of the second electrode 640 may be performed using one of: an open mask, and a mask-free, deposition process.

In some non-limiting examples, the second electrode 640 may comprise a plurality of such coatings. In some non-limiting examples, such coatings may be distinct coatings disposed on top of one another.

In some non-limiting examples, the second electrode 640 may comprise a Yb/Ag bi-layer coating. In some non-limiting examples, such bi-layer coating may be formed by depositing a Yb coating, followed by an Ag coating. In some non-limiting examples, a thickness of such Ag coating may exceed a thickness of the Yb coating.

In some non-limiting examples, the second electrode 640 may be a multi-coating electrode 640 comprising a plurality of one of: a metallic coating, and an oxide coating.

In some non-limiting examples, the second electrode 640 may comprise a fullerene and Mg.

In some non-limiting examples, such coating may be formed by depositing a fullerene coating followed by an Mg coating. In some non-limiting examples, a fullerene may be dispersed within the Mg coating to form a fullerene-containing Mg alloy coating. Non-limiting examples of such coatings are described in at least one of: United States Patent Application Publication No. 2015/0287846 published 8 Oct. 2015, and in PCT International Application No. PCT/IB2017/054970 filed 15 Aug. 2017 and published as WO2018/033860 on 22 Feb. 2018.

### Semiconducting Layer

In some non-limiting examples, the at least one semiconducting layer 630 may comprise a plurality of layers 631, 633, 635, 637, 639, any of which may be disposed, in some non-limiting examples, in a thin film, in a stacked configuration, which may include, without limitation, at least one of: a hole injection layer (HIL) 631, a hole transport layer (HTL) 633, an emissive layer (EML) 635, an electron transport layer (ETL) 637, and an electron injection layer (EIL) 639.

In some non-limiting examples, the at least one semiconducting layer 630 may form a “tandem” structure comprising a plurality of EMLs 635. In some non-limiting examples, such tandem structure may also comprise at least one charge generation layer (CGL).

Those having ordinary skill in the relevant art will readily appreciate that the structure of the device 600 may be varied by one of: omitting, and combining, at least one of the semiconductor layers 631, 633, 635, 637, 639.

In some non-limiting examples, any of the layers 631, 633, 635, 637, 639 of the at least one semiconducting layer 630 may comprise any number of sub-layers. In some non-limiting examples, any of such layers 631, 633, 635, 637, 639, including without limitation, sub-layer(s) thereof may comprise various ones of: a mixture, and a composition gradient. In some non-limiting examples, although not shown, the device 600 may comprise at least one layer comprising one of: an inorganic, and an organometallic, material, and may not be necessarily limited to devices comprised solely of organic materials. By way of non-limiting example, the device 600 may comprise at least one quantum dot (QD).

In some non-limiting examples, the HIL 631 may be formed using a hole injection material, which may, in some non-limiting examples, facilitate injection of holes by the anode.

In some non-limiting examples, the HTL 633 may be formed using a hole transport material, which may, in some non-limiting examples, exhibit high hole mobility.

In some non-limiting examples, the ETL 637 may be formed using an electron transport material, which may, in some non-limiting examples, exhibit high electron mobility.

In some non-limiting examples, the EIL 639 may be formed using an electron injection material, which may, in some non-limiting examples, facilitate injection of electrons by the cathode.

In some non-limiting examples, the at least one EML 635 may be formed, by way of non-limiting example, by doping a host material with at least one emitter material. In some non-limiting examples, the emitter material may be at least one of: a fluorescent emitter material, a phosphorescent emitter material, and a thermally activated delayed fluorescence (TADF) emitter material.

In some non-limiting examples, the emitter material may be one of a R(ed) emitter material, a G(reen) emitter material, and a B(lue) emitter material, that is, an emitter material that facilitates the emission of respectively, R(ed), G(reen), and B(lue) EM radiation.

In some non-limiting examples, the device 600 may be an OLED in which the at least one semiconducting layer 630 may comprise at least one EML 635 interposed between conductive thin film electrodes 620, 640, whereby, when a potential difference is applied across them, holes may be injected into the at least one semiconducting layer 630 through the anode and electrons may be injected into the at least one semiconducting layer 630 through the cathode, to migrate toward the at least one EML 635 and combine to emit EM radiation in the form of photons.

In some non-limiting examples, the device 600 may be an electro-luminescent QD device in which the at least one semiconducting layer 630 may comprise an active layer comprising at least one QD. When current is provided by the power source 604 to the first electrode 620 and second electrode 640, EM radiation, including without limitation, in the form of photons, may be emitted from the active layer comprising the at least one semiconducting layer 630 between them.

In some non-limiting examples, including where the device 600 comprises a lighting panel, an entire lateral aspect of the device 600 may correspond to a single emissive element. As such, the substantially planar cross-sectional profile shown in FIG. 6 may extend substantially along the entire lateral aspect of the device 600, such that EM radiation is emitted from the device 600 substantially along the entirety of the lateral extent thereof. In some non-limiting examples, such single emissive element may be driven by a single driving circuit of the device 600.

In some non-limiting examples, including where the device 600 comprises a display module, the lateral aspect of the device 600 may be sub-divided into a plurality of emissive regions 610 of the device 600, in which the longitudinal aspect of the device structure 600, within each of the emissive region(s) 610, may cause EM radiation to be emitted therefrom when energized.

Those having ordinary skill in the relevant art will readily appreciate that the structure of the device 600 may be varied by the introduction of at least one additional layer (not shown) at appropriate position(s) within the at least one semiconducting layer 630 stack, including without limitation, at least one of: a hole blocking layer (HBL) (not shown), an electron blocking layer (EBL) (not shown), a charge transport layer (CTL) (not shown), and a charge injection layer (CIL) (not shown).

In some non-limiting examples, the patterning coating 110 may be formed concurrently with the at least one semiconducting layer(s) 630. In some non-limiting examples, at least one material used to form the patterning coating 110 may also be used to form the at least one semiconducting layer(s) 630. In some non-limiting examples, the ETL 637 of the at least one semiconducting layer 630 may be a patterning coating 110 that may be deposited in the first portion 101 and the second portion 102 during the deposition of the at least one semiconducting layer 630. The EIL 639 may then be selectively deposited in the emissive region 610 of the second portion 102 over the ETL 637, such that the exposed layer surface 11 of the ETL 637 in the first portion 101 may be substantially devoid of the EIL 639. The exposed layer surface 11 of the EIL 639 in the emissive region 610 and the exposed layer surface of the ETL 637, which acts as the patterning coating 110, may then be exposed to a vapor flux 332 of the deposited material 331 to form a closed coating 140 of the deposited layer 130 on the EIL 639 in the second portion 102, and a discontinuous layer 160 of the deposited material 331 on the ETL 637 in the first portion 101. In such non-limiting example, several stages for fabricating the device 600 may be reduced.

### Emissive Region(s)

In some non-limiting examples, including where the OLED device 600 may comprise a display module, the lateral aspect of the device 600 may be sub-divided into a plurality of emissive regions 610 of the device 600, in which the longitudinal aspect of the device 600 structure, within each of the emissive region(s) 610, may cause EM radiation to be emitted therefrom when energized.

In some non-limiting examples, an individual emissive region 610 may have an associated pair of electrodes 620, 640, one of which may act as an anode and the other of which may act as a cathode, and at least one semiconducting layer 630 between them. Such an emissive region 610 may emit EM radiation at a given wavelength spectrum and may correspond to one of: a pixel 1015, and a sub-pixel 616 thereof. In some non-limiting examples, a plurality of sub-pixels 616, each corresponding to and emitting EM radiation of a different wavelength (range) may collectively form a pixel 1015.

In some non-limiting examples, the wavelength spectrum may correspond to a colour in, without limitation, the visible spectrum. The EM radiation at a first wavelength (range) emitted by a first sub-pixel 616 of a pixel 1015 may perform differently than the EM radiation at a second wavelength (range) emitted by a second sub-pixel 616 thereof because of the different wavelength (range) involved.

In some non-limiting examples, an active region 608 of an individual emissive region 610 may comprise at least one semiconducting layer 630. In some non-limiting examples, the active region 608 may be defined to be bounded, in the longitudinal aspect, by the first electrode 620 and the second electrode 640, and to be confined, in the lateral aspect, to an emissive region 610 defined by presence of each of the first electrode 620, the second electrode 640, and the at least one semiconducting layer 630 therebetween, that is, the first electrode 620, the second electrode 640, and the at least one semiconducting layer 630 therebetween, overlap laterally. In some non-limiting examples, the active region 608 may be substantially devoid of the compound comprising a heterocyclic moiety and at least one fluorine (F)-containing moiety.

Those having ordinary skill in the relevant art will appreciate that the lateral aspect of the emissive region 610, and thus the lateral boundaries of the active region 608, may not correspond to the entire lateral aspect of at least one of the first electrode 620 and the second electrode 640. Rather, the lateral aspect of the emissive region 610 may be substantially no more than the lateral extent of either of the first electrode 620 and the second electrode 640. In some non-limiting examples, at least one of: parts of the first electrode 620 may be covered by at least one pixel definition layer (PDL) 609, and parts of the second electrode 640 may not be disposed on the at least one semiconducting layer 630, with the result, in at least one scenario, that the emissive region 610 may be laterally constrained.

In some non-limiting examples, at least one of the various layers, including without limitation, the first electrode 620, the second electrode 640, and at least one semiconducting layer therebetween (“emissive region layers”) may be deposited by deposition of a corresponding constituent emissive region layer material.

In some non-limiting examples, some of the at least one semiconducting layers 630 may be laid out in a desired pattern by vapor deposition of the corresponding emissive region layer material through a fine metal mask (FMM) having apertures corresponding to the desired locations where the emissive region layer material is to be deposited. In some non-limiting examples, a plurality of the emissive region layers may be laid out in a similar pattern, including without limitation, by depositing the respective emissive region layer material thereof in their respective deposition stages using an FMM.

In some non-limiting examples, as discussed herein, the emissive region layer material corresponding to at least one of the first electrode 620 and the second electrode 640, including without limitation, the second electrode 640, may be deposited by prior deposition of a patterning coating 110 by vapor deposition of a patterning material through an FMM having apertures corresponding to the desired locations where the patterning coating 110 is to be deposited and thereafter depositing the emissive region layer material using one of: an open mask, and mask-free deposition process.

In some non-limiting examples, the patterning coating 110 may be adapted to impact a propensity of a vapor flux 332 of a deposited material 331 of which the emissive region layer material may be comprised, to be deposited thereon, including without limitation, an initial sticking probability against the deposition of the deposited material 331 that is no more than an initial sticking probability against the deposition of the deposited material 331 of the exposed layer surface 11 of the at least one semiconducting layer 630.

Where the layout of the emissive region layers is not identical, a given emissive region may be defined by overlaying the layouts of each emissive region layer thereof and selecting the intersection thereof, such that the emissive region 610 corresponds to the lateral aspect of the device 600 wherein each of the emissive region layers overlap.

In some non-limiting examples, in a longitudinal aspect, the configuration of each emissive region 610 may, in some non-limiting examples, be defined by the introduction of at least one PDL 609. In some non-limiting examples, the PDLs 609 may comprise an insulating at least one of: organic, and inorganic, material.

In some non-limiting examples, the first electrode 620 may be disposed over an exposed layer surface 11 of the device 600, in some non-limiting examples, within at least a part of the lateral aspect of the emissive region 610. In some non-limiting examples, at least within the lateral aspect of the emissive region 610 of the (sub-) pixel(s) 1015/616, the exposed layer surface 11, may, at the time of deposition of the first electrode 620, comprise the TFT insulating layer 607 of the various TFT structures 606 that make up the driving circuit for the emissive region 610 corresponding to a single display (sub-) pixel 1015/616.

In some non-limiting examples, the TFT insulating layer 607 may be formed with an opening extending therethrough to permit the first electrode 620 to be electrically coupled with a TFT electrode including, without limitation, a TFT drain electrode.

Those having ordinary skill in the relevant art will appreciate that the driving circuit may comprise a plurality of TFT structures 606. In FIG. 6, for purposes of simplicity of illustration, only one TFT structure 606 may be shown, but it will be appreciated by those having ordinary skill in the relevant art, that such TFT structure 606 may be representative of at least one of: such plurality thereof, and at least one component thereof, that comprise the driving circuit.

In some non-limiting examples, an extremity of the first electrode 620 may be covered by at least one PDL 609 such that a part of the at least one PDL 609 may be interposed between the first electrode 620 and the at least one semiconducting layer 630, such that such extremity of the first electrode 620 may lie beyond the active region 608 of the associated emissive region 610.

In some non-limiting examples, part(s) of the second electrode 640 may not be disposed directly on the at least one semiconducting layer 630, such that the emissive region 610 may be laterally constrained thereby.

In some non-limiting examples, the at least one semiconducting layer 630 (including without limitation, at least one of: layers 631, 633, 635, 637, 639 thereof) may be deposited over the exposed layer surface 11 of the device 600, including at least a part of the lateral aspect of such emissive region 610 of the (sub-) pixel(s) 1015/616. In some non-limiting examples, at least within the lateral aspect of the emissive region 610 of the (sub-) pixel(s) 1015/616, such exposed layer surface 11, may, at the time of deposition of such at least one semiconducting layer 630 comprise the first electrode 620.

In some non-limiting examples, the at least one semiconducting layer 630 may also extend beyond the lateral aspect of the emissive region 610 of the (sub-) pixel(s) 1015/616 and at least partially within the lateral aspects of the surrounding non-emissive region(s) 611. In some non-limiting examples, such exposed layer surface 11 of such surrounding non-emissive region(s) 611 may, at the time of deposition of the at least one semiconducting layer 630, comprise the PDL(s) 609.

In some non-limiting examples, the second electrode 640 may be disposed over an exposed layer surface 11 of the device 600, including at least a part of the lateral aspect of the emissive region 610 of the (sub-) pixel(s) 1015/616. In some non-limiting examples, at least within the lateral aspect of the emissive region 610 of the (sub-) pixel(s) 1015/616, such exposed layer surface 11, may, at the time of deposition of the second electrode 620, comprise the at least one semiconducting layer 630.

In some non-limiting examples, the second electrode 640 may also extend beyond the lateral aspect of the emissive region 610 of the (sub-) pixel(s) 1015/616 and at least partially within the lateral aspects of the surrounding non-emissive region(s) 611. In some non-limiting examples, an exposed layer surface 11 of such surrounding non-emissive region(s) 611 may, at the time of deposition of the second electrode 640, comprise the PDL(s) 609.

In some non-limiting examples, the second electrode 640 may extend throughout a substantial part, including without limitation, substantially all, of the lateral aspects of the surrounding non-emissive region(s) 611.

In some non-limiting examples, individual emissive regions 610 of the device 600 may be laid out in a lateral pattern. In some non-limiting examples, the pattern may extend along a first lateral direction. In some non-limiting examples, the pattern may also extend along a second lateral direction, which in some non-limiting examples, may extend at an angle relative to the first lateral direction. In some non-limiting examples, the second lateral direction may be substantially normal to the first lateral direction. In some non-limiting examples, the pattern may have a number of elements in such pattern, each element being characterized by at least one feature thereof, including without limitation, at least one of: a wavelength of EM radiation emitted by the emissive region 610 thereof, a shape of such emissive region 610, a dimension (along at least one of: the first, and second, lateral direction(s)), an orientation (relative to at least one of: the first, and second, lateral direction(s)), and a spacing (relative to at least one of: the first, and second, lateral direction(s)) from a previous element in the pattern. In some non-limiting examples, the pattern may repeat in at least one of: the first, and second, lateral direction(s).

In some non-limiting examples, each individual emissive region 610 of the device 600 may be associated with, and driven by, a corresponding driving circuit within the backplane 602 of the device 600, for driving an OLED structure for the associated emissive region 610. In some non-limiting examples, including without limitation, where the emissive regions 610 may be laid out in a regular pattern extending in both the first (row) lateral direction and the second (column) lateral direction, there may be a signal line in the backplane 602, corresponding to each row of emissive regions 610 extending in the first lateral direction and a signal line, corresponding to each column of emissive regions 610 extending in the second lateral direction. In such a non-limiting configuration, a signal on a row selection line may energize the respective gates of the switching TFT structure(s) 606 electrically coupled therewith and a signal on a data line may energize the respective sources of the switching TFT structure(s) 606 electrically coupled therewith, such that a signal on a row selection line/data line pair may electrically couple and energise, by the positive terminal of the power source 604, the anode of the OLED structure of the emissive region 610 associated with such pair, causing the emission of a photon therefrom, the cathode thereof being electrically coupled with the negative terminal of the power source 604.

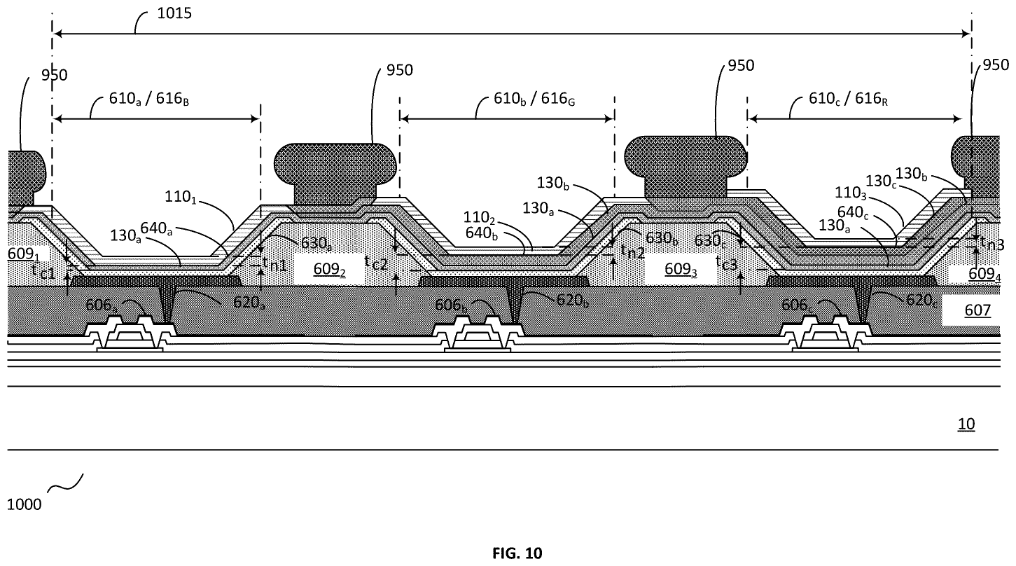

In some non-limiting examples, a single display pixel 1015 (FIG. 10) may comprise three sub-pixels 616, which in some non-limiting examples, may correspond respectively to a single sub-pixel 616 of each of three colours, including without limitation, at least one of: a R(ed) sub-pixel 616_{R}, a G(reen) sub-pixel 616_{G}, and a B(lue) sub-pixel 616_{B}. In some non-limiting examples, a single display pixel 1015 may comprise four sub-pixels 616, each corresponding respectively to a single sub-pixel 616 of each of two colours, including without limitation, a R(ed) sub-pixel 616_{R}, and a B(lue) sub-pixel 616_{B}, and two sub-pixels 616 of a third colour, including without limitation, a G(reen) sub-pixel 616_{G}. In some non-limiting examples, a single display pixel 1015 may comprise four sub-pixels 616, which in some non-limiting examples, may correspond respectively to a single sub-pixel 616 of each of three colours, including without limitation, at least one of: a R(ed) sub-pixel 616_{R}, a G(reen) sub-pixel 616_{G}, and a B(lue) sub-pixel 616_{B}, and a fourth W(hite) sub-pixel 616_{W}.

In some non-limiting examples, the emission spectrum of the EM radiation emitted by a given (sub-) pixel 1015/616 may correspond to the colour by which the (sub-) pixel 1015/616 may be denoted. In some non-limiting examples, the wavelength of the EM radiation may not correspond to such colour, but further processing may be performed, in a manner apparent to those having ordinary skill in the relevant art, to transform the wavelength to one that does so correspond.

In some non-limiting examples, the emission spectrum of the EM radiation emitted by a given (sub-) pixel 1015/616, corresponding to the colour by which the (sub-) pixel 1015/616 may be denoted, may be related to at least one of: the structure and composition of the at least one semiconducting layer 630 extending between the first electrode 620 and the second electrode 640 thereof, including without limitation, the at least one EML 635. In some non-limiting examples, the at least one EML 635 of the at least one semiconducting layer 630 may be tuned to facilitate the emission of EM radiation having an emission spectrum corresponding to the colour by which the (sub-) pixel 1015/616 may be denoted. In some non-limiting examples, the EML 635 of a R(ed) sub-pixel 616_{R} may comprise a R(ed) EML material, including without limitation, a host material doped with a R(ed) emitter material. In some non-limiting examples, the EML 635 of a G(reen) sub-pixel 616^{G} may comprise a G(reen) EML material, including without limitation, a host material doped with a G(reen) emitter material. In some non-limiting examples, the EML 635 of a B(lue) sub-pixel 616_{B} may comprise B(lue) EML material, including without limitation, a host material doped with a B(lue) emitter material.

In some non-limiting examples, at least one characteristic of at least one of the at least one semiconducting layer 630, including without limitation, the HIL 631, the HTL 633, the EML 635, the ETL 637, and the EIL 639, including without limitation, a presence thereof, an absence thereof, a thickness thereof, a composition thereof, and an order thereof, in the longitudinal aspect, may be selected to facilitate emission therefrom of EM radiation having a wavelength spectrum corresponding to the colour by which a given sub-pixel 616 may be denoted, including without limitation, at least one of: R(ed), G(reen), and B(lue).

In some non-limiting examples, emission of EM radiation having a wavelength spectrum corresponding to a plurality of colours selected from: R(ed), G(reen), and B(lue) may facilitate emission of EM radiation having a wavelength spectrum corresponding to a different colour, including without limitation W(hite) (R+G+B), Y(ellow) (R+G), C(yan) (G+B), and M(agenta) (B+R), according to the additive colour model.

In some non-limiting examples, the exposed layer surface 11 of the device 100 may be exposed to a vapor flux 332 of a deposited material 331, including without limitation, in one of: an open mask, and mask-free, deposition process.

In some non-limiting examples, in at least a part of the emissive region 610, the at least one semiconducting layer 630 may be deposited over the exposed layer surface 11 of the device 600, which in some non-limiting examples, comprise the first electrode 620.

In some non-limiting examples, the exposed layer surface 11 of the device 600, which may, in some non-limiting examples, comprise the at least one semiconducting layer 630, may be exposed to a vapor flux 212 of the patterning material 211, including without limitation, using a shadow mask 215, to form a patterning coating 110 in the first portion 101. Whether a shadow mask 215 is employed, the patterning coating 110 may be restricted, in its lateral aspect, substantially to a signal-transmissive region 612.

In some non-limiting examples, a lateral aspect of at least one emissive region 610 may extend across and include at least one TFT structure 606 associated therewith for driving the emissive region 610 along data and scan lines (not shown), which, in some non-limiting examples, may be formed of at least one of: Cu, and a TCO.

In some non-limiting examples, the (sub-) pixels 1015/616 may be disposed in a side-by-side arrangement. In some non-limiting examples, a (colour) order of the sub-pixels 616 of a first pixel 1015 may be the same as a (colour) order of the sub-pixels 616 of a second pixel 1015. In some non-limiting examples, a (colour) order of the sub-pixels 616 of a first pixel 1015 may be different from a (colour) order of the sub-pixels 616 of a second pixel 1015.

In some non-limiting examples, the sub-pixels 616 of adjacent pixels 1015 may be aligned in at least one of a row, column, and array arrangement.

In some non-limiting examples, a first at least one of a row and a column of aligned sub-pixels 616 of adjacent pixels 1015 may comprise sub-pixels 616 of one of: a same, and a different, colour.

In some non-limiting examples, a first at least one of a row and a column of aligned sub-pixels 616 of adjacent pixels 1015 may be aligned with at least one of: a second, and a third, at least one of: a row, and a column, of aligned sub-pixels 616 of adjacent pixels.

In some non-limiting examples, a first at least one of: a row, and a column, of aligned sub-pixels 616 of adjacent pixels 1015 may be one of: offset from, and mis-aligned with, at least one of: a second, and a third, at least one of: a row, and a column, of aligned sub-pixels 616 of adjacent pixels 1015.

In some non-limiting examples, the sub-pixels 616 of adjacent pixels 1015 of such at least one of: first, second, and third, at least one of: a row, and a column, may be arranged such that corresponding sub-pixels 616 of each of the at least one of: first, second, and third, at least one of: a row, and a column, may be of a same colour.

In some non-limiting examples, the sub-pixels 616 of adjacent pixels 1015 of such at least one of: first, second, and third, at least one of: a row, and a column, may be arranged such that corresponding sub-pixels 616 of each of the at least one of: first, second and third, at least one of: a row, and a column, may be of different colours.

In some non-limiting examples, in the at least one signal-exchanging part 703 (FIG. 7) of a display panel 700 (FIG. 3), at least one signal-transmissive region 612 may be disposed between a plurality of emissive regions 610. In some non-limiting examples, the at least one signal-transmissive region 612 may be disposed between adjacent (sub-) pixels 1015/616. In some non-limiting examples, the adjacent sub-pixels 616 surrounding the at least one signal-transmissive region 612 may form part of a same pixel 1015. In some non-limiting examples, the adjacent sub-pixels 616 surrounding the at least one signal-transmissive region 612 may be associated with different pixels 1015.

In some non-limiting examples, a region that may be substantially devoid of a closed coating 140 of a second electrode material (“cathode-free region”), including without limitation, the at least one signal-transmissive region 612, in some non-limiting examples, may exhibit different opto-electronic characteristics from other regions, including without limitation, the at least one emissive region 610. In some non-limiting examples, such cathode-free regions may nevertheless comprise some second electrode material, including without limitation, in the form of a discontinuous layer 160 of one of: at least one particle structure 150, and at least one instance of such particle structures 150.

In some non-limiting examples, this may be achieved by laser ablation of the second electrode material. However, in some non-limiting examples, laser ablation may create a debris cloud, which may impact the vapour deposition process.

In some non-limiting examples, this may be achieved by disposing a patterning coating 110, which may, in some non-limiting examples, be an NIC, using an FMM, in a pattern on an exposed layer surface 11 of the at least one semiconducting layer 630 prior to depositing a deposited material 331 for forming the second electrode 640 thereon.

In some non-limiting examples, the patterning coating 110 may be adapted to impact a propensity of a vapor flux 332 of the deposited material 331 to be deposited thereon, including without limitation, an initial sticking probability against the deposition of the deposited material 331 that is no more than an initial sticking probability against the deposition of the deposited material 331 of the exposed layer surface 11 of the at least one semiconducting layer 630.

In some non-limiting examples, the patterning coating 110 may be deposited in a pattern that may correspond to the first portion 101 of a lateral aspect, including without limitation, of at least some of the signal-transmissive regions 612.

In some non-limiting examples, the patterning coating 110 may be deposited in a plurality of stages, each using a different FMM defining a different pattern within the first portion 101, that respectively correspond to a different subset of the signal-transmissive regions 612.

In some non-limiting examples, the display panel 700 may, subsequent to (all of the stages of) the deposition of the patterning coating 110, be subjected to a vapor flux 332 of the deposited material 331, in one of: an open mask. and mask-free, deposition process, to form the second electrode 640 for each of the emissive regions 610 corresponding to a (sub-) pixel 1015/616 in at least the second portion 102 of the lateral aspect, but not in the first portion 101 of the lateral aspect.

In some non-limiting examples, although not shown, the overlying layer 170 may be arranged above at least one of: the second electrode 640, and the patterning coating 110. In some non-limiting examples, although not shown, the overlying layer 170 may be deposited at least partially across the lateral extent of the opto-electronic device 600, in some non-limiting examples, covering the second electrode 640 in the second portion 102, and, in some non-limiting examples, at least partially covering the at least one particle structure 150 and forming an interface with the patterning coating 110 at the exposed layer surface 11 thereof in the first portion 101.

### Non-Emissive Regions

In some non-limiting examples, the various emissive regions 610 of the device 600 may be substantially surrounded and separated by, in at least one lateral direction, at least one non-emissive region 611, in which at least one of: the structure, and configuration, along the longitudinal aspect, of the device 600 shown, without limitation, may be varied, to substantially inhibit EM radiation to be emitted therefrom.

In some non-limiting examples, the non-emissive regions 611 may comprise those regions in the lateral aspect, that are substantially devoid of an emissive region 610.

In some non-limiting examples, the longitudinal topology of the various layers of the at least one semiconducting layer 630 may be varied to define at least one emissive region 610, surrounded (at least in one lateral direction) by at least one non-emissive region 611.

A non-limiting example of an implementation of the longitudinal aspect of the device 600 as applied to an emissive region 610 corresponding to a single display (sub-) pixel 1015/616 of the device 600 will now be described. While features of such implementation are shown to be specific to the emissive region 610, those having ordinary skill in the relevant art will appreciate that in some non-limiting examples, more than one emissive region 610 may encompass features in common.

In some non-limiting examples, the lateral aspects of the surrounding non-emissive region(s) 611 may be characterized by the presence of a corresponding PDL 609.

In some non-limiting examples, a thickness of the PDL 609 may increase from a minimum, where it covers the extremity of the first electrode 620, to a maximum beyond the lateral extent of the first electrode 620. In some non-limiting examples, the change in thickness of the at least one PDL 609 may define a valley shape centered about the emissive region 610. In some non-limiting examples, the valley shape may constrain the field of view (FOV) of the EM radiation emitted by the emissive region 610.

While the PDL(s) 609 have been generally illustrated as having a linearly sloped surface to form a valley-shaped configuration that define the emissive region(s) 610 surrounded thereby, those having ordinary skill in the relevant art will appreciate that in some non-limiting examples, at least one of: the shape, aspect ratio, thickness, width, and configuration of such PDL(s) 609 may be varied. By way of non-limiting example, a PDL 609 may be formed with one of: a substantially steep part and a more gradually sloped part. In some non-limiting examples, such PDL(s) 609 may be configured to extend substantially normally away from a surface on which it is deposited, that may cover at least one edge of the first electrode 620. In some non-limiting examples, such PDL(s) 609 may be configured to have deposited thereon at least one semiconducting layer 630 by a solution-processing technology, including without limitation, by printing, including without limitation, ink-jet printing.

In some non-limiting examples, the PDLs 609 may be deposited substantially over the TFT insulating layer 607, although, as shown, in some non-limiting examples, the PDLs 609 may also extend over at least a part of the deposited first electrode 620, including without limitation, its outer edges.

In some non-limiting examples, the lateral extent of at least one of the non-emissive regions 611 may be at least, and in some non-limiting examples, exceed, including without limitation, be a multiple of, the lateral extent of the emissive region 610 interposed therebetween.

In some non-limiting examples, a thickness of at least one PDL 609 in at least one signal-transmissive region 612, in some non-limiting examples, of at least one non-emissive region 611, interposed between adjacent emissive regions 610, in some non-limiting examples, at least in a region laterally spaced apart therefrom, and in some non-limiting examples; although not shown, of the TFT insulating layer 607, may be reduced in order to enhance at least one of: a transmittivity, and a transmittivity angle, relative to and through the layers of a display panel 700, to facilitate transmission of EM radiation therethrough.

### Patterning

In some non-limiting examples, with reference to FIG. 1, in some non-limiting examples, a patterning coating 110, comprising a patterning material 211, which in some non-limiting examples, may be an NIC material, may be disposed, in some non-limiting examples, as a closed coating 140, on an exposed layer surface 11 of an underlying layer 210, including without limitation, a substrate 10, of the device 100, in some non-limiting examples, restricted in lateral extent by selective deposition, including without limitation, using a shadow mask 215 such as, without limitation, an FMM, including without limitation, to the first portion 101.

Thus, in some non-limiting examples, in the second portion 102 of the device 100, the exposed layer surface 11 of the underlying layer 210 of the device 100, may be substantially devoid of a closed coating 140 of the patterning coating 110.

In some non-limiting examples, with reference to FIG. 1, in some non-limiting examples, a patterning coating 110, comprising a patterning material 211, which in some non-limiting examples, may be an NIC material, may be disposed, in some non-limiting examples, as a closed coating 140, on an exposed layer surface 11 of an underlying layer 210, including without limitation, a substrate 10, of the device 100, in some non-limiting examples, restricted in lateral extent by selective deposition, including without limitation, using a shadow mask 215 such as, without limitation, an FMM, including without limitation, to the first portion 101.

Thus, in some non-limiting examples, in the second portion 102 of the device 100, the exposed layer surface 11 of the underlying layer 210 of the device 100, may be substantially devoid of a closed coating 140 of the patterning coating 110.

### Display Panel and User Device

Turning now to FIG. 7, there is shown a cross-sectional view of an example layered device, such as a display panel 700. In some non-limiting examples, the display panel 700 may comprise a plurality of layers deposited on a substrate 10, culminating with an outermost layer that forms a face 701 thereof. In some non-limiting examples, the display panel 700 may be a version of the device 600.

The face 701 of the display panel 700 may extend across a lateral aspect thereof, substantially along a plane defined by the lateral axes.

In some non-limiting examples, the face 701, and indeed, the entire display panel 700, may act as a face of a user device 710 through which at least one EM signal 731 may be exchanged therethrough at a non-zero angle relative to the plane of the face 701. In some non-limiting examples, the user device 710 may be a computing device, such as, without limitation, a smartphone, a tablet, a laptop, an e-reader, and some other electronic device, such as a monitor, a television set, and a smart device, including without limitation, an automotive display, windshield, a household appliance, and a medical, commercial, and industrial device.

In some non-limiting examples, the face 701 may correspond to, and in some non-limiting examples, mate with, at least one of: a body 720, and an opening 721 therewithin, within which at least one under-display component 730 may be housed.

In some non-limiting examples, the at least one under-display component 730 may be formed, including without limitation, at least one of: integrally, and as an assembled module, with the display panel 700 on a surface thereof opposite to the face 701.

In some non-limiting examples, at least one aperture 722 may be formed in the display panel 700 to allow for the exchange of at least one EM signal 731 through the face 701 of the display panel 700, at a non-zero angle to the plane defined by the lateral axes, including without limitation, concomitantly, the layers of the display panel 700, including without limitation, the face 701 of the display panel 700.

In some non-limiting examples, the at least one aperture 722 may be understood to comprise one of: the absence, and reduction in at least one of: thickness, and capacity, of a substantially opaque coating otherwise disposed across the display panel 700. In some non-limiting examples, the at least one aperture 722 may be embodied as a signal-transmissive region 612 as described herein.

However the at least one aperture 722 is embodied, the at least one EM signal 731 may pass therethrough such that it passes through the face 701. As a result, the at least one EM signal 731 may be considered to exclude any EM radiation that may extend along the plane defined by the lateral axes, including without limitation, any electric current that may be conducted across at least one particle structure 150 laterally across the display panel 700.

Further, those having ordinary skill in the relevant art will appreciate that the at least one EM signal 731 may be differentiated from EM radiation per se, including without limitation, one of: electric current, and an electric field generated thereby, in that the at least one EM signal 731 may convey, either one of: alone, and in conjunction with other EM signals 731, some information content, including without limitation, an identifier by which the at least one EM signal 731 may be distinguished from other EM signals 731. In some non-limiting examples, the information content may be conveyed by at least one of: specifying, altering, and modulating, at least one of: the wavelength, frequency, phase, timing, bandwidth, resistance, capacitance, impedance, conductance, and other characteristic of the at least one EM signal 731.

In some non-limiting examples, the at least one EM signal 731 passing through the at least one aperture 722 of the display panel 700 may comprise at least one photon and, in some non-limiting examples, may have a wavelength spectrum that lies, without limitation, within at least one of: the visible spectrum, the IR spectrum, and the NIR spectrum. In some non-limiting examples, the at least one EM signal 731 passing through the at least one aperture 722 of the display panel 700 may have a wavelength that lies, without limitation, within at least one of: the IR, and NIR spectrum.

In some non-limiting examples, the at least one EM signal 731 passing through the at least one aperture 722 of the display panel 700 may comprise ambient light incident thereon.

In some non-limiting examples, the at least one EM signal 731 exchanged through the at least one aperture 722 of the display panel 700 may be at least one of: transmitted, and received, by the at least one under-display component 730.

In some non-limiting examples, the at least one under-display component 730 may have a size that is at least a single signal-transmissive region 612, but may underlie not only a plurality thereof, but also at least one emissive region 610 extending therebetween. Similarly, in some non-limiting examples, the at least one under-display component 730 may have a size that is at least a single one of the at least one apertures 622.

In some non-limiting examples, the at least one under-display component 730 may comprise a receiver 730ᵣ, adapted to receive and process at least one received EM signal 731ᵣ, passing through the at least one aperture 722 from beyond the user device 710. Non-limiting examples of such receiver 730ᵣ include an under-display camera (UDC), and a sensor, including without limitation, IR sensor/detector, an NIR sensor/detector, a LIDAR sensing module, a fingerprint sensing module, an optical sensing module, an IR (proximity) sensing module, an iris recognition sensing module, and a facial recognition sensing module, including without limitation, a part thereof.

In some non-limiting examples, the at least one under-display component 730 may comprise a transmitter 730ₜ adapted to emit at least one transmitted EM signal 73Iₜ passing through the at least one aperture 722 beyond the user device 710. Non-limiting examples, of such transmitter 730ₜ include a source of EM radiation, including without limitation, a built-in flash, a flashlight, an IR emitter, a NIR emitter, a LIDAR sensing module, a fingerprint sensing module, an optical sensing module, an IR (proximity sensing module, an iris recognition sensing module, and a facial recognition sensing module, including without limitation, a part thereof.

In some non-limiting examples, the at least one received EM signal 731ᵣ may include at least a fragment of the at least one transmitted EM signal 73Iₜ which is one of: reflected off, and otherwise returned by, an external surface to the user device 710, including without limitation, a user 70.

In some non-limiting examples, the at least one EM signal 731 passing through the at least one aperture 722 of the display panel 700 beyond the user device 710, including without limitation, those transmitted EM signals 731ₜ emitted by the at least one under-display component 730 that may comprise a transmitter 730ₜ, may emanate from the display panel 700, and pass back as received EM signals 731ᵣ through the at least aperture 722 of the display panel 700 to at least one under-display component 730 that may comprise a receiver 730ᵣ.

In some non-limiting examples, the under-display component 730 may comprise an IR emitter and an IR sensor. In some non-limiting examples, such under-display component 730 may comprise, as one of: a part, component, and module, thereof: at least one of: a dot-matrix projector, a time-of-flight (ToF) sensor module, which may operate as one of: a direct ToF, and an indirect ToF, sensor, a vertical cavity surface-emitting laser (VCSEL), flood illuminator, NIR imager, folded optics, and a diffractive grating.

In some non-limiting examples, there may be a plurality of under-display components 730 within the user device 710, a first one of which may comprise a transmitter 730ₜ for emitting at least one transmitted EM signal 731ₜ to pass through the at least one aperture 722, beyond the user device 710, and a second one of which may comprise a receiver 730ᵣ, for receiving at least one received EM signal 731ᵣ. In some non-limiting examples, such transmitter 730ₜ and receiver 730ᵣ may be embodied in a single under-display component 730.

In some non-limiting examples, the display panel 700 may comprise at least one signal-exchanging part 703 and at least one display part 707.

In some non-limiting examples, the at least one display part 707 may comprise a plurality of emissive regions 610. In some non-limiting examples, the emissive regions 610 in the at least one display part 707 may correspond to (sub-) pixels 1015/616 of the display panel 700.

In some non-limiting examples, the at least one signal-exchanging part 703 may comprise a plurality of emissive regions 610 and a plurality of signal-transmissive regions 612. In some non-limiting examples, the emissive regions 610 in the at least one signal-exchanging part 703 may correspond to (sub-) pixels 1015/616 of the display panel 700.

In some non-limiting examples, the at least one display part 707 may be adjacent to, and in some non-limiting examples, separated by, at least one signal-exchanging part 703.

In some non-limiting examples, the at least one signal-exchanging part 703 may be positioned proximate to an extremity of the display panel 700, including without limitation, at least one of: an edge, and a corner, thereof. In some non-limiting examples, the at least one signal-exchanging part 703 may be positioned substantially centrally within the lateral aspect of the display panel 700.

In some non-limiting examples, the at least one display part 707 may substantially surround, including without limitation, in conjunction with at least one other display part 707, the at least one signal-exchanging part 703. In some non-limiting examples, the at least one signal-exchanging part 703 may be positioned proximate to an extremity of the display panel 700. In some non-limiting examples, the at least one signal-exchanging part 703 may be positioned proximate to an extremity and configured such that the at least one display part(s) 707 do(es) not completely surround the at least one signal-exchanging part 703.

In some non-limiting examples, a pixel density of the at least one emissive region 610 of the at least one signal-exchanging part 703 may be substantially the same as a pixel density of the at least one emissive region 610 of the at least one display part 707 proximate thereto, at least in an area thereof that is substantially proximate to the at least one signal-exchanging part 703. In some non-limiting examples, the pixel density of the display panel 700 may be substantially uniform thereacross. In at least some applications, there may be scenarios calling for the at least one signal-exchanging part 703 and the at least one display part 707 to have substantially the same pixel density, including without limitation, so that a resolution of the display panel 700 may be substantially the same across both the at least one signal-exchanging part 703 and the at least one display part 707 thereof.

Those having ordinary skill in the relevant art will appreciate that there may be scenarios calling for the layout of (sub-) pixels 1015/616 in the signal-exchanging part 703 of the display panel 700 to resemble, to some extent, the layout thereof in the display part 707 of the display panel 700, including without limitation, a size, shape, (colour) order, and configuration of (sub-) pixels 1015/616, and wherein a spacing between adjacent (sub-) pixels 1015/616 (“pitch”) in the signal-exchanging part 703 is one of: the same, and an integer multiple thereof, of a pitch thereof in the display part 707.

Having said this, examples in the present disclosure may have applicability in scenarios in which the layout of (sub-) pixels 1015/616 in the signal-exchanging part 703 may be substantially different than the layout thereof in the display part 707 of the display panel 700.

In some non-limiting examples, the display panel 700 may further comprise at least one transition region (not shown) between the at least one signal-exchanging part 703 and the at least one display part 707, wherein the configuration of at least one of: the emissive regions 610, and the signal-transmissive regions 612 therein, may differ from those of at least one of: the at least one signal-exchanging part 703, and the at least one display part 707. In some non-limiting examples, such transition region may be omitted such that the emissive regions 610 may be provided in a substantially continuous repeating pattern across both the at least one signal-exchanging part 703 and the at least one display part 707.

In some non-limiting examples, the at least one signal-exchanging part 703 may have a polygonal contour, including without limitation, at least one of a substantially square, and rectangular, configuration.

In some non-limiting examples, the at least one signal-exchanging part 703 may have a curved contour, including without limitation, at least one of a substantially circular, oval, and elliptical, configuration.

In some non-limiting examples, the signal-transmissive regions 612 in the at least one signal-exchanging part 703 may be configured to allow EM signals having a wavelength (range) corresponding to the IR spectrum to pass through the entirety of a cross-sectional aspect thereof.

In some non-limiting examples, the at least one signal-exchanging part 703 may have a reduced number of, including without limitation, be substantially devoid of, backplane components, including without limitation, TFT structures 606, including without limitation, metal trace lines, capacitors, and other EM radiation-absorbing element, including without limitation, opaque elements, the presence of which may otherwise interfere with the capture of the EM radiation by the at least one under-display component 730, including without limitation, the capture of an image by a camera.

In some non-limiting examples, the user device 710 may house at least one transmitter 730ₜ for transmitting at least one transmitted EM signal 731ₜ through at least one first signal-transmissive region 612 in, and in some non-limiting examples, substantially corresponding to, a first signal-exchanging part 703, beyond the face 701. In some non-limiting examples, the user device 710 may house at least one receiver 730ᵣ for receiving at least one received EM signal 731ᵣ through at least one second signal-transmissive region 612 in, and in some non-limiting examples, substantially corresponding to, a second signal-exchanging part 703, from beyond the face 701. In some non-limiting examples, the at least one received EM signal 731ᵣ may be the same as the at least one transmitted EM signal 731ₜ, reflected off an external surface, including without limitation, a user 70, including without limitation, for biometric authentication thereof.

In some non-limiting examples, at least one of: the at least one transmitter 730ₜ, and the at least one receiver 730ₜ, may be arranged behind the corresponding at least one signal-exchanging part 703, such that IR signals may be at least one of: emitted, and received, respectively, by passing through the at least one signal-exchanging part 703 of the display panel 700. In some non-limiting examples, the at least one transmitter 730ₜ and the at least one receiver 730ᵣ may both be arranged behind a single signal-exchanging part 703, which in some non-limiting examples, may be elongated along at least one configuration axis, such that it extends across both the at least one transmitter 730ₜ and the at least one receiver 730ᵣ.

In some non-limiting examples, the display panel 700 may further comprise a non-display part (not shown), which in some non-limiting examples, may be substantially devoid of any emissive regions 610. In some non-limiting examples, the user device 710 may house an under-display component 730, including without limitation, a camera, arranged within the non-display part.

In some non-limiting examples, the non-display part may be arranged adjacent to, and in some non-limiting examples, between a plurality of signal-exchanging parts 703 corresponding to a plurality of under-display components 730, including without limitation, a transmitter 730ₜ and a receiver 730ᵣ.

In some non-limiting examples, the non-display part may comprise a through-hole part (not shown), which in some non-limiting examples, may be arranged to overlap the camera. In some non-limiting examples, the display panel 700 may, in the through-hole part, be substantially devoid of any of at least one of: a layer, coating, and component, that may otherwise be present in at least one of: the at least one signal-exchanging part 703, and the at least one display part 707, including without limitation, a component of at least one of: the backplane 602, and the frontplane 601, the presence of which may otherwise interfere with the capture of an image by the camera. In some non-limiting examples, an overlying layer 170, including without limitation, at least one of: a polarizer, and one of: a cover glass, and a glass cap, of the display panel 700, may extend substantially across the at least one signal-exchanging part 703, the at least one display part 707, and the non-display part, such that it may extend substantially across the display panel 700. In some non-limiting examples, the through-hole part may be substantially devoid of a polarizer in order to enhance the transmission of EM radiation therethrough.

In some non-limiting examples, the non-display part may comprise a non-through-hole part, which in some non-limiting examples, may be arranged between the through-hole part and an adjacent signal-exchanging part 703 in a lateral aspect. In some non-limiting examples, the non-through-hole part may surround at least a part of a perimeter of the through-hole part. In some non-limiting examples, the user device 710 may comprise additional ones of at least one of: a module, component, and sensor, in a part of the user device 710 corresponding to the non-through-hole part of the display panel 700.

In some non-limiting examples, the emissive regions 610 in the at least one signal-exchanging part 703 may be electrically coupled with at least one TFT structure located in the non-through-hole part of the non-display part. That is, in some non-limiting examples, the TFT structures 606 for actuating the (sub-) pixels 1015/616 in the at least one signal-exchanging part 703 may be relocated outside the at least one signal-exchanging part 703 and within the non-through-hole part of the display panel 700, such that a substantially high transmission of EM radiation, in at least one of: the IR spectrum, and the NIR spectrum, may be directed through the non-emissive regions 611 within the at least one signal-exchanging part 703. In some non-limiting examples, the TFT structures 606 in the non-through-hold part may be electrically coupled with (sub-) pixels 1015/616 in the at least one signal-exchanging part 703 via conductive trace(s). In some non-limiting examples, at least one of the transmitter 730ₜ and the receiver 730ᵣ may be arranged to be proximate to the non-through-hole part in the lateral aspect, such that a distance over which electrical current travels between the TFT structures 606 and the (sub-) pixels 1015/616 associated therewith, may be reduced.

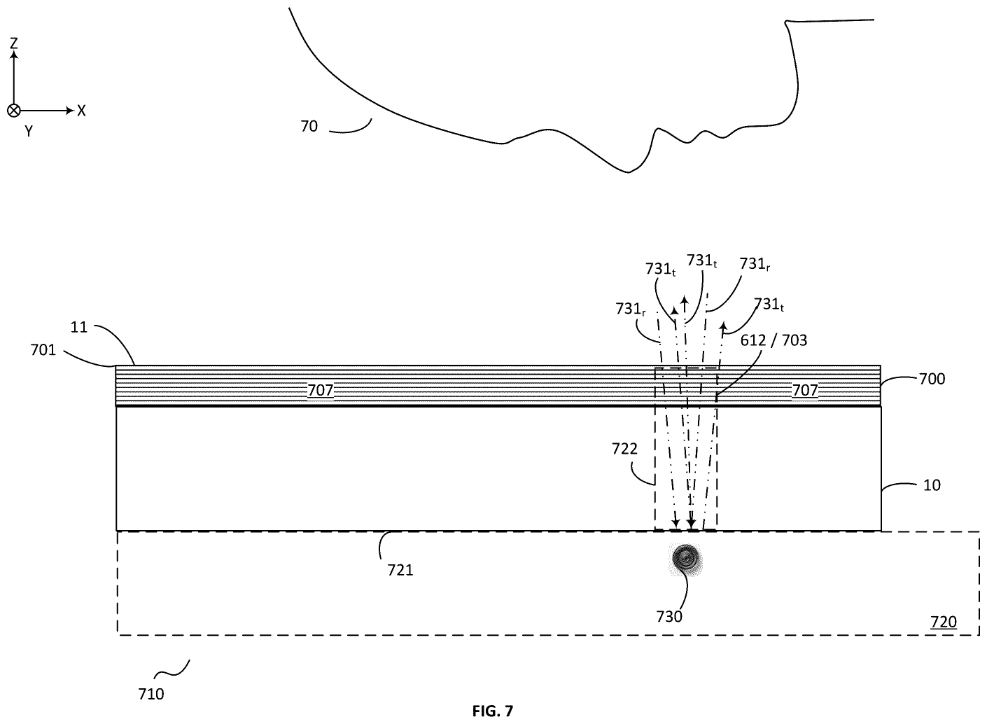

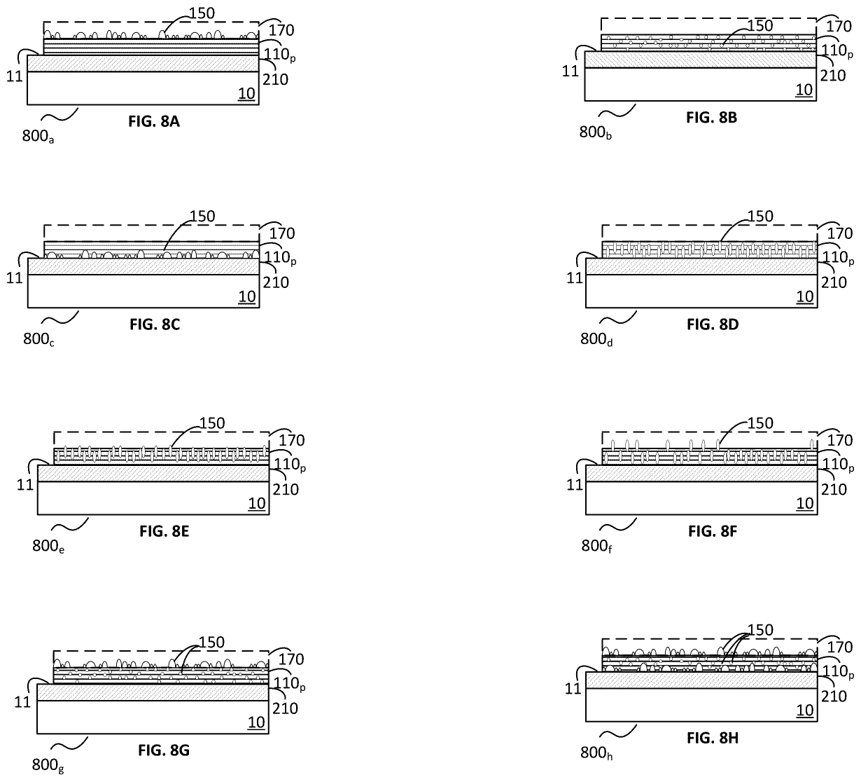

FIGS. 8A-8H illustrate non-limiting examples of possible interactions between the particle structure patterning coating 110ₚ and the at least one particle structure 150 in contact therewith.

Thus, as shown in FIGS. 8A-8H, the particle material may be in physical contact with the patterning material 211, including without limitation, as shown in the various figures, being one of: deposited thereon, and being substantially surrounded thereby.

In FIG. 8A, the particle material may be in physical contact with the particle structure patterning coating 110ₚ in that it is deposited thereon.

In FIG. 8B, the particle material may be substantially surrounded by the particle structure patterning coating 110ₚ. In some non-limiting examples, the at least one particle structure 150 may be distributed throughout at least one of: the lateral, and longitudinal, extent of the particle structure patterning coating 110ₚ.

In some non-limiting examples, the distribution of the at least one particle structure 150 throughout the particle structure patterning coating 110ₚ may be achieved by causing the particle structure patterning coating 110ₚ to be at least one of: deposited, and to remain, in a substantially viscous state at the time of deposition of the particle material thereon, such that the at least one particle structure 150 may tend to penetrate (settle) within the particle structure patterning coating 110ₚ.

In some non-limiting examples, the viscous state of the particle structure patterning coating 110ₚ may be achieved in a number of manners, including without limitation, conditions during deposition of the patterning material 211, including without limitation, at least one of: a time, temperature, and pressure, of the deposition environment thereof, a composition of the patterning material 211, a characteristic of the patterning material 211, including without limitation, a melting point, a freezing temperature, a sublimation temperature, a viscosity, and a surface energy, thereof, conditions during deposition of the particle material, including without limitation, at least one of: a time, temperature, and pressure, of the deposition environment thereof, a composition of the particle material, and a characteristic of the particle material, including without limitation, a melting point, a freezing temperature, a sublimation temperature, a viscosity, and a surface energy thereof.

In some non-limiting examples, the distribution of the at least one particle structure 150 throughout the particle structure patterning coating 110ₚ may be achieved through the presence of small apertures, including without limitation, at least one of: pin-holes, tears, and cracks, therein. Those having ordinary skill in the relevant art will appreciate that such apertures may be formed during the deposition of a thin film of the patterning structure patterning coating 110ₚ, using various techniques and processes, including without limitation, those described herein, due to inherent variability in the deposition process, and in some non-limiting examples, to the existence of impurities in at least one of the particle material and the exposed layer surface 11 of the patterning material 211.

In FIG. 8C, the particle material of which the at least one particle structure 150 may be comprised may settle at a bottom of the particle structure patterning coating 110ₚ such that it is effectively disposed on the exposed layer surface 11 of the underlying layer 210.

In some non-limiting examples, the distribution of the at least one particle structure 150 at a bottom of the particle structure patterning coating 110ₚ may be achieved by causing the particle structure patterning coating 110ₚ to be at least one of: deposited, and to remain, in a substantially viscous state at the time of deposition of the particle material thereon, such that the at least one particle structure 150 may tend to settle to the bottom of the particle structure patterning coating 110ₚ. In some non-limiting examples, the viscosity of the patterning material 211 used in FIG. 8C may be no more than the viscosity of the patterning material 211 used in FIG. 8B, allowing the at least one particle structure 150 to settle further within the particle structure patterning coating 110ₚ, eventually descending to the bottom thereof.

In FIGS. 8D-8F, a shape of the at least one particle structure 150 is shown as being longitudinally elongated relative to a shape of the at least one particle structure 150 of FIG. 8B.

In some non-limiting examples, the longitudinally elongated shape of the at least one particle structure 150 may be achieved in a number of manners, including without limitation, conditions during deposition of the patterning material 211, including without limitation, at least one of: a time, temperature, and pressure, of the deposition environment thereof, a composition of the patterning material 211, a characteristic of the patterning material 211, including without limitation, a melting point, a freezing temperature, a sublimation temperature, a viscosity, and a surface energy thereof, conditions during deposition of the particle material, including without limitation, a time, temperature, and pressure, of the deposition environment thereof, a composition of the particle material, and a characteristic of the particle material, including without limitation, a melting point, a freezing temperature, a sublimation temperature, a viscosity, and a surface energy thereof, that may tend to facilitate the deposition of such longitudinally elongated particle structures 150.

In FIG. 8D, the longitudinally elongated particle structures 150 are shown to remain substantially entirely within the particle structure patterning coating 110ₚ. By contrast, in FIG. 8E, at least one of the longitudinally elongated particle structures 150 may be shown to protrude at least partially beyond the exposed layer surface 11 of the particle structure patterning coating 110ₚ. Further, in FIG. 8F, at least one of the longitudinally elongated particle structures 150 may be shown to protrude substantially beyond the exposed layer surface 11 of the particle structure patterning coating 110ₚ, to the extent that such protruding particle structures 150 may begin to be considered to be substantially deposited on the exposed layer surface 11 of the particle structure patterning coating 110ₚ.

Thus, as shown in FIG. 8G, there may be a scenario in which at least one particle structure 150 may be deposited on the exposed layer surface 11 of the particle structure patterning coating 110ₚ and at least one particle structure 150 may settle within the particle structure patterning coating 110ₚ. Although the at least one particle structure 150 shown within the particle structure patterning coating 110ₚ is shown as having a shape such as is shown in FIG. 8B, those having ordinary skill in the relevant art will appreciate that, although not shown, such particle structures 150 may have a longitudinally elongated shape such as is shown in FIGS. 8D-8F.

Further, FIG. 8H shows a scenario in which at least one particle structure 150 may be deposited on the exposed layer surface 11 of the particle structure patterning coating 110ₚ, at least one particle structure 150 may penetrate (settle within) the particle structure patterning coating 110ₚ, and at least one particle structure 150 may settle to the bottom of the particle structure patterning coating 110ₚ.

### Auxiliary Electrode

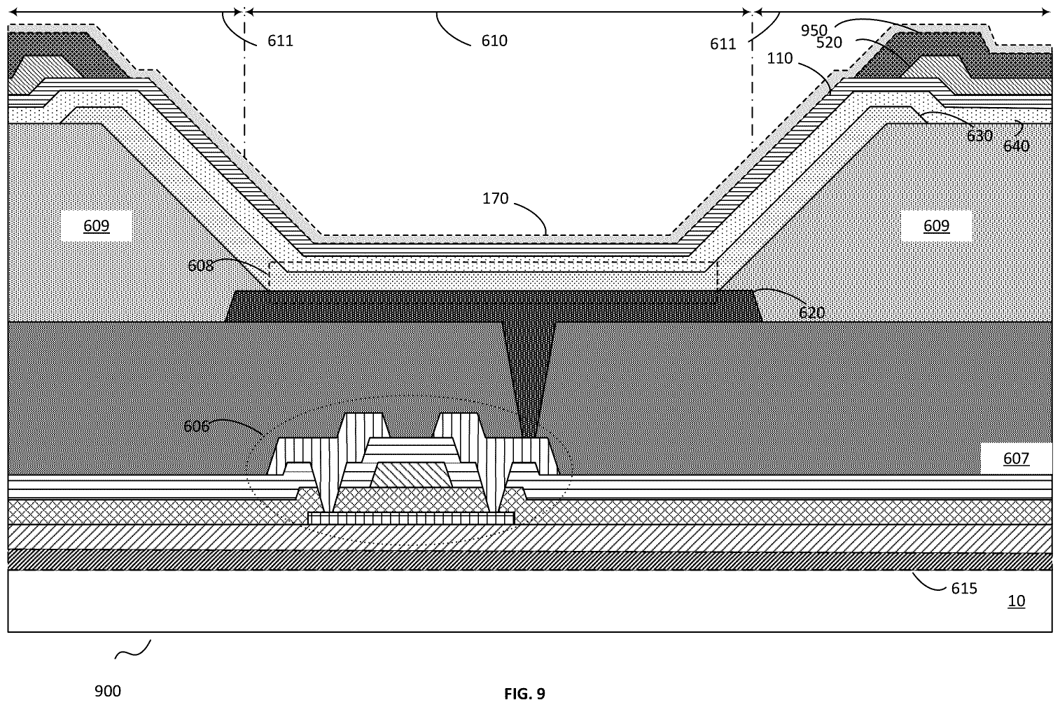

Those having ordinary skill in the relevant art will appreciate that the process of depositing a deposited layer 130 to form the second electrode 640 may, in some non-limiting examples, be used in similar fashion to form an auxiliary electrode 950 (FIG. 9) for the device 600.

In some non-limiting examples, particularly in a top-emission device 600, the second electrode 640 may be formed by depositing a substantially thin conductive film layer in order, in some non-limiting examples, to reduce optical interference (including, without limitation, at least one of: attenuation, reflections, and diffusion) related to the presence of the second electrode 640.

In some non-limiting examples, particularly in at least one of: a bottom-emission, and double-sided emission, device 600, the second electrode 640 may be formed as a substantially thick conductive layer without substantially affecting optical characteristics of such a device 600. Nevertheless, even in such scenarios, the second electrode 640 may nevertheless be formed as a substantially thin conductive film layer, in some non-limiting examples, so that the device 600 may be substantially transmissive relative to EM radiation incident on an external surface thereof, such that a substantial part of such externally-incident EM radiation may be transmitted through the device 600, in addition to the emission of EM radiation generated internally within the device 600 as disclosed herein.

In some non-limiting examples, a device 600 having at least one electrode 620, 640 with a high sheet resistance may create a large current resistance (IR) drop when coupled with the power source 604, in operation. In some non-limiting examples, such an IR drop may be compensated for, to some extent, by increasing a level of the power source 604. However, in some non-limiting examples, increasing the level of the power source 604 to compensate for the IR drop due to high sheet resistance, for at least one (sub-) pixel 1015/616 may call for increasing the level of a voltage to be supplied to other components to maintain effective operation of the device 600.

In some non-limiting examples, as discussed elsewhere, a reduced thickness of the second electrode 640, may generally increase a sheet resistance of the second electrode 640, which may, in some non-limiting examples, reduce at least one of: the performance, and efficiency, of the device 600. By providing the auxiliary electrode 950 that may be electrically coupled with the second electrode 640, the sheet resistance and thus, the IR drop associated with the second electrode 640, may, in some non-limiting examples, be decreased.

In some non-limiting examples, to reduce power supply demands for a device 600 without significantly impacting an ability to make an electrode 620, 640 substantially thin, an auxiliary electrode 950 may be formed on the device 600 to allow current to be carried more effectively to various emissive region(s) 610 of the device 600, while at the same time, reducing the sheet resistance and its associated IR drop of the transmissive electrode 620, 640.

In some non-limiting examples, a sheet resistance specification, for a common electrode 620, 640 of a display device 600, may vary according to several parameters, including without limitation, at least one of: a (panel) size of the device 600, and a tolerance for voltage variation across the device 600. In some non-limiting examples, the sheet resistance specification may increase (that is, a lower sheet resistance is specified) as the panel size increases. In some non-limiting examples, the sheet resistance specification may increase as the tolerance for voltage variation decreases.

In some non-limiting examples, a sheet resistance specification may be used to derive an example thickness of an auxiliary electrode 950 to comply with such specification for various panel sizes.

In some non-limiting examples, the auxiliary electrode 950 may be electrically coupled with the second electrode 640 to reduce a sheet resistance thereof. In some non-limiting examples, the auxiliary electrode 950 may be in physical contact, including without limitation, being deposited over at least a part thereof, with the second electrode 640 to reduce a sheet resistance thereof. In some non-limiting examples, the auxiliary electrode 950 may not be in physical contact with the second electrode 640 but may be electrically coupled with the second electrode 640 by several well-understood mechanisms. In some non-limiting examples, the presence of a substantially thin film (in some non-limiting examples, of up to about 50 nm) of a patterning coating 110 extending between and separating the auxiliary electrode 950 and the second electrode 640, may still allow a current to pass therethrough, thus allowing a sheet resistance of the second electrode 640 to be reduced.

The auxiliary electrode 950 may be electrically conductive. In some non-limiting examples, the auxiliary electrode 950 may be formed by at least one of: a metal, and a metal oxide. Non-limiting examples of such metals include Cu, Al, molybdenum (Mo), and Ag. In some non-limiting examples, the auxiliary electrode 950 may comprise a multi-layer metallic structure, including without limitation, one formed by Mo/Al/Mo. Non-limiting examples of such metal oxides include ITO, ZnO, IZO, and other oxides comprising In, and Zn. In some non-limiting examples, the auxiliary electrode 950 may comprise a multi-layer structure formed by a combination of at least one metal and at least one metal oxide, including without limitation, Ag/ITO, Mo/ITO, ITO/Ag/ITO, and ITO/Mo/ITO. In some non-limiting examples, the auxiliary electrode 950 comprises a plurality of such electrically conductive materials.

Because of the nucleation-inhibiting properties of those portions 101 where the patterning coating 110 was disposed, the deposited material 331 disposed in the first portion 101 may tend to not remain, resulting in a pattern of selective deposition of the deposited layer 130, that may correspond substantially to at least one second portion 102, leaving the first portion 101 substantially devoid of a closed coating 140 of the deposited layer 130.

In other words, the deposited layer 130 that may form the auxiliary electrode 950 may be selectively deposited substantially only on a second portion 102 comprising those regions of the at least one semiconducting layer 630, that surround but do not occupy the first portion 101.

In some non-limiting examples, selectively depositing the auxiliary electrode 950 to cover only certain portions 102 of the lateral aspect of the device 600, while other portions 101 thereof remain uncovered, may one of: control, and reduce, optical interference related to the presence of the auxiliary electrode 950.

In some non-limiting examples, the auxiliary electrode 950 may be selectively deposited in a pattern that may not be readily detected by the naked eye from a typical viewing distance.

In some non-limiting examples, the auxiliary electrode 950 may be formed in devices other than OLED devices, including for decreasing an effective resistance of the electrodes of such devices.

Turning now to FIG. 9, there may be shown an example version 900 of the device 600, which may encompass the device shown in cross-sectional view in FIG. 6, but with additional deposition steps that are described herein.

The device 900 may show a patterning coating 110 deposited over the exposed layer surface 11 of the underlying layer 210, in the figure, the second electrode 640.

The patterning coating 110 may provide an exposed layer surface 11 with a substantially low initial sticking probability against deposition of a deposited material 331 to be thereafter deposited as a deposited layer 130 to form an auxiliary electrode 950.

In some non-limiting examples, after deposition of the patterning coating 110, an NPC 520 may be selectively deposited over the exposed layer surface 11 of the underlying layer 210, in the figure, the patterning coating 110.

In some non-limiting examples, the NPC 520 may be disposed between the auxiliary electrode 950 and the second electrode 640.

In some non-limiting examples, the NPC 520 may be selectively deposited using a shadow mask 215, in a second portion 102 of the lateral aspect of the device 900.

The NPC 520 may provide an exposed layer surface 11 with a substantially high initial sticking probability against deposition of a deposited material 331 to be thereafter deposited as a deposited layer 130 to form an auxiliary electrode 950.

After selective deposition of the NPC 520, the deposited material 331 may be deposited over the device 900 but may remain substantially where the patterning coating 110 has been overlaid with the NPC 520, to form the auxiliary electrode 950, that is, substantially only the second portion 102.

In some non-limiting examples, the deposited layer 130 may be deposited using one of: an open mask, and a mask-free, deposition process.

### Transparent OLED

Because the OLED device 600 may emit EM radiation through at least one of: the first electrode 620 (in the case of one of: a bottom-emission, and a double-sided emission, device), as well as the substrate 10, and the second electrode 640 (in the case of one of: a top-emission, and double-sided emission, device), there may be an aim to make at least one of: the first electrode 620, and the second electrode 640, substantially EM radiation-(light-)transmissive (“transmissive”), in some non-limiting examples, at least across a substantial part of the lateral aspect of the emissive region(s) 610 of the device 600. In the present disclosure, such a transmissive element, including without limitation, an electrode 620, 640, at least one of: a material from which such element may be formed, and a property thereof, may comprise at least one of: an element, material, and property thereof, that is one of: substantially transmissive (“transparent”), and, in some non-limiting examples, partially transmissive (“semi-transparent”), in some non-limiting examples, in at least one wavelength range.

A variety of mechanisms may be adopted to impart transmissive properties to the device 600, at least across a substantial part of the lateral aspect of the emissive region(s) 610 thereof.

In some non-limiting examples, including without limitation, where the device 600 is at least one of: a bottom-emission, and a double-sided emission, device, the TFT structure(s) 606 of the driving circuit associated with an emissive region 610 of a (sub-) pixel 1015/616, which may at least partially reduce the transmissivity of the surrounding substrate 10, may be located within the lateral aspect of the surrounding non-emissive region(s) 611 to avoid impacting the transmissive properties of the substrate 10 within the lateral aspect of the emissive region 610.

In some non-limiting examples, where the device 600 is a double-sided emission device, in respect of the lateral aspect of an emissive region 610 of a (sub-) pixel 1015/616, a first one of the electrodes 620, 640 may be made substantially transmissive, including without limitation, by at least one of the mechanisms disclosed herein, in respect of the lateral aspect of neighbouring (sub-) pixel(s) 1015/616, a second one of the electrodes 620, 640 may be made substantially transmissive, including without limitation, by at least one of the mechanisms disclosed herein. Thus, the lateral aspect of a first emissive region 610 of a (sub-) pixel 1015/616 may be made substantially top-emitting while the lateral aspect of a second emissive region 610 of a neighbouring (sub-) pixel 1015/616 may be made substantially bottom-emitting, such that a subset of the (sub-) pixel(s) 1015/616 may be substantially top-emitting and a subset of the (sub-) pixel(s) 1015/616 may be substantially bottom-emitting, in an alternating (sub-) pixel 1015/616 sequence, while only a single electrode 620, 640 of each (sub-) pixel 1015/616 may be made substantially transmissive.

In some non-limiting examples, a mechanism to make an electrode 620, 640, in the case of at least one of: a bottom-emission device, and a double-sided emission device, the first electrode 620, and in the case of at least one of: a top-emission device, and a double-sided emission device, the second electrode 640, transmissive, may be to form such electrode 620, 640 of a transmissive thin film.

In some non-limiting examples, an electrically conductive deposited layer 130, in a thin film, including without limitation, those formed by depositing a thin conductive film layer of at least one of: a metal, including without limitation, Ag, Al, and a metallic alloy, including without limitation, at least one of: an Mg:Ag alloy, and a Yb:Ag alloy, may exhibit transmissive characteristics. In some non-limiting examples, the alloy may comprise a composition ranging from between about 1:9-9:1 by volume. In some non-limiting examples, the electrode 620, 640 may be formed of a plurality of thin conductive film layers of any combination of deposited layers 130, any at least one of which may be comprised of at least one of: TCOs, thin metal films, and thin metallic alloy films.

In some non-limiting examples, especially in the case of such thin conductive films, a substantially thin layer thickness may be up to substantially a few tens of nm to contribute to enhanced transmissive qualities but also favorable optical properties (including without limitation, reduced microcavity effects) for use in an OLED device 600.

Thus, in some non-limiting examples, an average layer thickness of the second electrode 640 may be no more than about 40 nm, including without limitation, one of between about: 5-30 nm, 10-25 nm, and 15-25 nm.

In some non-limiting examples, a reduction in the thickness of an electrode 620, 640 to promote transmissive qualities may be accompanied by an increase in the sheet resistance of the electrode 620, 640.

In some non-limiting examples, the auxiliary electrode 950 may be electrically coupled with the second electrode 640 to reduce a sheet resistance of thin, and concomitantly, (substantially) transmissive, second electrode 640.

In some non-limiting examples, the auxiliary electrode 950 may not be substantially transmissive but may be electrically coupled with the second electrode 640, including without limitation, by deposition of a conductive deposited layer 130 therebetween, to reduce an effective sheet resistance of the second electrode 640.

In some non-limiting examples, such auxiliary electrode 950 may be one of: positioned, and shaped, in at least one of: a lateral aspect, and longitudinal aspect, to not interfere with the emission of photons from the lateral aspect of the emissive region 610 of a (sub-) pixel 1015/616.

In some non-limiting examples, a mechanism to make at least one of: the first electrode 620, and the second electrode 640, may be to form such electrode 620, 640 in a pattern across at least one of: at least a part of the lateral aspect of the emissive region(s) 610 thereof, and in some non-limiting examples, across at least a part of the lateral aspect of the non-emissive region(s) 611 surrounding them. In some non-limiting examples, such mechanism may be employed to form the auxiliary electrode 950 in one of: a position, and shape, in at least one of: a lateral aspect, and longitudinal aspect to not interfere with the emission of photons from the lateral aspect of the emissive region 610 of a (sub-) pixel 1015/616, as discussed above.

In some non-limiting examples, the device 600 may be configured such that it may be substantially devoid of a conductive oxide material in an optical path of EM radiation emitted by the device 600. In some non-limiting examples, in the lateral aspect of at least one emissive region 610 corresponding to a (sub-) pixel 1015/616, at least one of the coatings deposited after the at least one semiconducting layer 630, including without limitation, at least one of: the second electrode 640, the patterning coating 110, and any other coatings deposited thereon, may be substantially devoid of any conductive oxide material. In some non-limiting examples, being substantially devoid of any conductive oxide material may reduce at least one of: absorption, and reflection, of EM radiation emitted by the device 600. In some non-limiting examples, conductive oxide materials, including without limitation, at least one of: ITO, and IZO, may absorb EM radiation in at least the B(lue) region of the visible spectrum, which may, in generally, reduce at least one of: efficiency, and performance, of the device 600.

In some non-limiting examples, a combination of these mechanisms may be employed.

Additionally, in some non-limiting examples, in addition to rendering at least one of the first electrode 620, the second electrode 640, and the auxiliary electrode 950, substantially transmissive across at least across a substantial part of the lateral aspect of the emissive region 610 corresponding to the (sub-) pixel(s) 1015/616 of the device 600, to allow EM radiation to be emitted substantially across the lateral aspect thereof, there may be an aim to make at least one of the lateral aspect(s) of the surrounding non-emissive region(s) 611 of the device 600 substantially transmissive in both the bottom and top directions, to render the device 600 substantially transmissive relative to EM radiation incident on an external surface thereof, such that a substantial part of such externally-incident EM radiation may be transmitted through the device 600, in addition to the emission (in at least one of: a top-emission, bottom-emission, and double-sided emission) of EM radiation generated internally within the device 600 as disclosed herein.

In some non-limiting examples, the signal-transmissive region 612 of the device 600 may remain substantially devoid of any materials that may substantially affect the transmission of EM radiation therethrough, including without limitation, EM signals, including without limitation, in at least one of: the IR, and the NIR, spectrum. In some non-limiting examples, the TFT structure(s) 606 and the first electrode 620 may be positioned, in a longitudinal aspect, below the (sub-) pixel 1015/616 corresponding thereto, and together with the auxiliary electrode 950, may lie beyond the signal-transmissive region 612. As a result, these components may not impede, including without limitation, attenuate EM radiation, including without limitation, light, from being transmitted through the signal-transmissive region 612. In some non-limiting examples, such arrangement may allow a viewer viewing the device 600 from a typical viewing distance to see through the device 600, in some non-limiting examples, when all the (sub-) pixel(s) 1015/616 may not be emitting, thus creating a transparent device 600.

In some non-limiting examples, a patterning coating 110 may be selectively deposited over first portion(s) 101 of the device 600, comprising a signal-transmissive region 612.

In some non-limiting examples, at least one particle structure 150 may be disposed on an exposed layer surface 11 within the signal-transmissive region 612, to facilitate absorption of EM radiation therein in at least a part of the visible spectrum, while allowing EM signals having a wavelength in at least a part of at least one of: the IR, and NIR, spectrum to be exchanged through the device in the signal-transmissive region 612.

Those having ordinary skill in the relevant art will appreciate that in some non-limiting examples, various other coatings, including without limitation those forming at least one of: the at least one semiconducting layer(s) 630, and the second electrode 640, may cover a part of the signal-transmissive region 612, especially if such coatings are substantially transparent. In some non-limiting examples, the PDL(s) 609 may have a reduced thickness, including without limitation, by forming a well therein, which in some non-limiting examples may be similar to the well defined for emissive region(s) 610, to further facilitate transmission of EM radiation through the signal-transmissive region 612.

In some non-limiting examples, the signal-transmissive region 612 of the device 600 may remain substantially devoid of any materials that may substantially inhibit the transmission of EM radiation, including without limitation, EM signals, including without limitation, in at least one of: the IR spectrum, and the NIR spectrum, therethrough. In some non-limiting examples, at least one of: the TFT structure 606, and the first electrode 620, may be positioned, in a longitudinal aspect below the (sub-) pixel 1015/616 corresponding thereto and beyond the signal-transmissive region 612. As a result, these components may not impede, including without limitation, attenuate, EM radiation from being transmitted through the signal-transmissive region 612. In some non-limiting examples, such arrangement may allow a viewer viewing the device 600 from a typical viewing distance to see through the device 600, in some non-limiting examples, when the (sub-) pixel(s) 1015/616 are not emitting, thus creating a transparent AMOLED device 600.

In some non-limiting examples, such arrangement may also allow at least one of: an IR emitter 730ₑ, and an IR detector 730_{d}, to be arranged behind the device 600 such that EM signals, including without limitation, in at least one of: the IR, and NIR, spectrum, to be exchanged through the device 600 by such under-display components 730.

In some non-limiting examples, as discussed herein, the patterning coating 110 may be formed concurrently with the at least one semiconducting layer(s) 630. In some non-limiting examples, at least one material used to form the patterning coating 110 may also be used to form the at least one semiconducting layer(s) 630. In such non-limiting example, several stages for fabricating the device 600 may be reduced, which may, in some non-limiting examples, facilitate making the signal-transmissive region 612 (substantially) transmissive.

Turning now to FIG. 10, there is shown an example cross-sectional view of a fragment of an example version 1000 of the opto-electronic device 600 according to the present disclosure. In the fragment shown, emissive regions 610 corresponding to each of three sub-pixels 616, of a single pixel 1015, are shown, which in some non-limiting examples, may correspond to a B(lue) sub-pixel 616_{B}, a G(reen) sub-pixel 616_{G}, and a R(ed) sub-pixel 616_{R}. In some non-limiting examples, each sub-pixel 616 may have a first electrode 620, with which an associated TFT structure 606 may be electrically coupled, a second electrode 640, and at least one semiconducting layer 630 deposited between the first electrode 620 and the second electrode 640.

In some non-limiting examples, the at least one semiconducting layer 630 may comprise at least one R(ed) EML material within at least the lateral aspect of the R(ed) sub-pixel 616_{R}. In some non-limiting examples, the at least one semiconducting layer 630 may comprise at least one G(reen) EML material within at least the lateral aspect of the G(reen) sub-pixel 616_{G}. In some non-limiting examples, the at least one semiconducting layer 630 may comprise at least one B(lue) EML material within at least the lateral aspect of the B(lue) sub-pixel 616_{B}.

In some non-limiting examples, at least one characteristic of at least one of the at least one semiconducting layer 630, including without limitation, at least one of: the HIL 631, HTL 633, EML 635, ETL 637, and EIL 639, including without limitation, a presence thereof, an absence thereof, a thickness thereof, a composition thereof, and an order thereof, in the longitudinal aspect, may be varied within at least a lateral aspect of one of the sub-pixels 616, to facilitate emission therefrom of EM radiation having a wavelength spectrum corresponding to the colour by which such sub-pixel 616 may be denoted, including without limitation, at least one of: R(ed), G(reen), and B(lue), such that such at least one characteristic may be varied across substantially its entire lateral extent.

In some non-limiting examples, neighboring sub-pixels 616 may be separated by a non-emissive region 611 having a corresponding PDL 609, that covers at least a part of an extremity of the corresponding first electrodes 620. In some non-limiting examples, although not shown, the PDL 609 may be truncated in at least one of: a lateral aspect, and a longitudinal aspect. In some non-limiting examples, truncation of the PDL 609 in the lateral aspect may cause the lateral extent of the neighboring emissive regions 610 to be at least, and in some non-limiting examples, exceed, including without limitation, be a multiple of, the lateral extent of the non-emissive region 611 interposed therebetween.

In some non-limiting examples, although not shown, at least one PDL 609 between neighboring emissive regions 610 may be truncated to a greater extent than shown, until the emissive regions 610 may be considered to be substantially immediately adjacent to one another, substantially without a non-emissive region 611 therebetween.

In some non-limiting examples, although not shown, neighboring emissive regions 610 may not have a PDL 609 interposed therebetween, although, in such scenario, alternative measures may be called for to electrically isolate a first electrode 620 corresponding to a first emissive region 610 from a first electrode 620 corresponding to a second emissive region 610 immediately adjacent thereto.

In some non-limiting examples, the at least one semiconducting layer 630 may extend across substantially the lateral extent of each of the first electrodes 620 and across substantially the lateral extent of each of the non-emissive regions 611 corresponding to the PDLs 609 separating them. In some non-limiting examples, the at least one semiconducting layer 630 may extend across substantially the entire lateral aspect of the device 1000.

### Selective Deposition to Modulate Electrode Thickness Over Emissive Region(s)

In some non-limiting examples, the output, including without limitation, the emission spectrum, of a given (sub-) pixel 1015/616 may be impacted, according to at least one of: its associated color, and wavelength range, including without limitation, by at least one of: controlling, modulating, and tuning, optical microcavity effects, including without limitation, at least one of: an emission spectrum, a(n) (luminous) intensity, and an angular distribution of at least one of: a brightness, and a color shift, of emitted light in each emissive region 610 corresponding each (sub-) pixel 1015/616.

Some factors that may impact an observed microcavity effect in a device 600 include, without limitation, a total path length (which in some non-limiting examples may correspond to a total thickness (in the longitudinal aspect) of the device 600 through which EM radiation emitted therefrom will travel before being outcoupled) and the refractive indices of various layers and coatings.

Since the wavelength of (sub-) pixels 1015/616 of different colours may be different, the optical characteristics of such (sub-) pixels 1015/616 may differ, especially if a common electrode 620, 640 having a substantially uniform thickness profile may be employed for (sub-) pixels 1015/616 of different colours.

In some non-limiting examples, a separation distance between the pair of electrodes 620, 640 within an emissive region 610 corresponding to a (sub-) pixel 1015/616, may be varied to reflect a (half-) integer multiple of a wavelength range associated with an emitted colour of the (sub-) pixel 1015/616.

In some non-limiting examples, such tuning may be achieved, at least in part, by varying the thickness of the at least one semiconducting layer 630 extending between the electrodes 620, 640.

In some non-limiting examples, where (substantially all) the at least one semiconducting layer 630 comprise(s) a common layer extending across all of the (sub-) pixels 1015/616, such measures may be incomplete.

In some non-limiting examples, irrespective of whether a thickness of the at least one semiconducting layer 630 may be varied, at least one of: across the device 600, and as between (sub-) pixels 1015/616 thereof, the separation distance between the pair of electrodes 620, 640 within an emissive region 610 corresponding to a (sub-) pixel 1015/616 may be further varied by modulating the thickness of an electrode 620, 640 in, and across a lateral aspect of emissive region(s) 610 of such (sub-) pixel 1015/616.

The second electrode 640 used in such devices 600 may in some non-limiting examples, be a common electrode 620, 640 coating a plurality of (sub-) pixels 1015/616. In some non-limiting examples, such common electrode 620, 640 may be a substantially thin conductive film having a substantially uniform thickness across the device 600. When a common electrode 620, 640 having a substantially uniform thickness may be provided as the second electrode 640 in a device 600, the optical performance of the device 600 may not be readily be fine-tuned according to an emission spectrum associated with each (sub-) pixel 1015/616.

In some non-limiting examples, modulating a thickness of an electrode 620, 640 in and across a lateral aspect of emissive region(s) 610 of a (sub-) pixel 1015/616 may impact the microcavity effect observable. In some non-limiting examples, such impact may be attributable to a change in the total optical path length.

In some non-limiting examples, modulating a thickness of an electrode 620, 640 in and across a lateral aspect of emissive region(s) 610 of a (sub-) pixel 1015/616 may impact the microcavity effect observable. In some non-limiting examples, such impact may be attributable to a change in the total optical path length.

In some non-limiting examples, a change in a thickness of the electrode 620, 640 may also change the refractive index of EM radiation passing therethrough, in some non-limiting examples, in addition to a change in the total optical path length. In some non-limiting examples, this may be particularly the case where the electrode 620, 640 may be formed of at least one deposited layer 130.

Thus, in some non-limiting examples, the presence of optical interfaces created by a plurality of thin-film coatings with different refractive indices, such as may in some non-limiting examples be used to construct opto-electronic devices including without limitation devices 600, may create different optical microcavity effects for (sub-) pixels 1015/616 of different colours.

In some non-limiting examples, selective deposition of at least one deposited layer 130 through deposition of at least one patterning coating 110, including without limitation, at least one of: an NIC, and an NPC 520, in the lateral aspects of emissive region(s) 610 corresponding to different (sub-) pixel(s) 1015/616, may allow the thickness of at least one electrode 620, 640, of each (sub-) pixel 1015/616 to be varied, and concomitantly, for the optical microcavity effect in each emissive region 610 corresponding thereto, to be at least one of: controlled, and modulated, to optimize desirable optical microcavity effects on a (sub-) pixel 1015/616 basis.

The thickness of the at least one electrode 620, 640 may be varied by independently modulating at least one of: an average layer thickness, and a number, of the deposited layer(s) 130, disposed in each emissive region 610 of the (sub-) pixel(s) 1015/616. By way of non-limiting example, the average layer thickness of a second electrode 640 disposed over, and corresponding to, a B(lue) sub-pixel 616_{B} may be no more than the average layer thickness of a second electrode 640 disposed over, and corresponding to, a G(reen) sub-pixel 616_{G}, and the average layer thickness of a second electrode 640 disposed over, and corresponding to, a G(reen) sub-pixel 616_{G} may be no more than the average layer thickness of a second electrode 640 disposed over, and corresponding to, a R(ed) sub-pixel 616_{R}.

Turning now to FIG. 10, in some non-limiting examples, including without limitation, in versions 1000 of an OLED display device 600 there may be deposited layer(s) 130 of varying average layer thickness selectively deposited for emissive region(s) 610 corresponding to sub-pixel(s) 616, having different emission spectra. In some non-limiting examples, a first emissive region 610ₐ may correspond to a (sub-) pixel 1015/616 configured to emit EM radiation of a first at least one of: a wavelength, and an emission spectrum. In some non-limiting examples, a device 1000 may comprise a second emissive region 610_{b} that may correspond to a (sub-) pixel 1015/616 configured to emit EM radiation of a second at least one of: a wavelength, and an emission spectrum. In some non-limiting examples, a device 1000 may comprise a third emissive region 610_{c} that may correspond to a (sub-) pixel 1015/616 configured to emit EM radiation of a third at least one of: a wavelength, and an emission spectrum.

In some non-limiting examples, the first wavelength may be one of: no more than, greater than, and equal to, at least one of: the second wavelength, and the third wavelength. In some non-limiting examples, the second wavelength may be one of: no more than, greater than, and equal to, at least one of: the first wavelength, and the third wavelength. In some non-limiting examples, the third wavelength may be one of: no more than, greater than, and equal to, at least one of: the first wavelength, and the second wavelength.

As shown by way of non-limiting example in FIG. 10, there may be deposited layer(s) 130 of varying at least one of: number, and average layer thickness, selectively deposited for various emissive region(s) 610 corresponding to various (sub-) pixel(s) 1015/616, in some non-limiting examples, in a version 1000 of device 600, having different emission spectra. In some non-limiting examples, the device 1000 may comprise a first emissive region 610ₐ corresponding to a sub-pixel 616^{B} configured to emit EM radiation of at least one of: a first wavelength, and emission spectrum, which in some non-limiting examples, may be associated with a B(lue) emitted colour. In some non-limiting examples, the device 1000 may comprise a second emissive region 610_{b} corresponding to a sub-pixel 616_{G} configured to emit EM radiation of at least one of: a second wavelength, and emission spectrum, which in some non-limiting examples, may be associated with a G(reen) emitted colour. In some non-limiting examples, the device 1000 may comprise a third emissive region 610_{c} corresponding to a sub-pixel 616_{R} configured to emit EM radiation of at least one of: a third wavelength, and emission spectrum, which in some non-limiting examples, may be associated with a R(ed) emitted colour.

In some non-limiting examples, the first wavelength may be one of: equal to, at least, and no more than, at least one of: the second wavelength, and the third wavelength. In some non-limiting examples, the second wavelength may be one of: equal to, at least, and no more than, at least one of: the first wavelength, and the third wavelength. In some non-limiting examples, the third wavelength may be one of: equal to, at least, and no more than, at least one of: the first wavelength, and the second wavelength.

In some non-limiting examples, although not shown, the device 1000 may comprise at least one additional emissive region 610 that may in some non-limiting examples be configured to emit EM radiation having at least one of: a wavelength, and emission spectrum, that may be substantially identical to at least one of: the first emissive region 610ₐ, the second emissive region 610_{b}, and the third emissive region 610_{c}, including without limitation, the second emissive region 610_{b}.

In some non-limiting examples, the device 1000 may also comprise any number of emissive regions 610, and (sub-) pixel(s) 1015/616 thereof.

In some non-limiting examples, the plurality of sub-pixels 616 may correspond to a single pixel 1015. In some non-limiting examples, the device 1000 may comprise a plurality of pixels 1015, wherein each pixel 1015 comprises a plurality of sub-pixel(s) 616.

Those having ordinary skill in the relevant art will appreciate that the specific arrangement of (sub-) pixel(s) 1015/616 may be varied depending on the device design. In some non-limiting examples, the sub-pixel(s) 616 may be arranged according to known arrangement schemes, including without limitation, RGB side-by-side, diamond, and PenTile®.

In some non-limiting examples, the device 1000 may be shown as comprising a substrate 10, and a plurality of emissive regions 610, each having a corresponding at least one TFT structure 606, covered by at least one TFT insulating layer 607, and a corresponding first electrode 620, formed on an exposed layer surface 11 of the TFT insulating layer 607.

In some non-limiting examples, the substrate 10 may comprise the base substrate 615.

In some non-limiting examples, each at least one TFT structure 606 may be longitudinally aligned below and within the lateral extent of its corresponding emissive region 610, for driving the corresponding (sub-) pixel 1015/616 and electrically coupled with its associated first electrode 620.

In some non-limiting examples, neighboring first electrodes 620 may be separated by a non-emissive region 611 having a corresponding PDL 609, formed over the TFT insulating layer 607, that may, in some non-limiting examples, cover at least a part of an extremity of the corresponding first electrodes 200.

In the present disclosure, each of the various emissive region layers of the device 600, including without limitation, at least one of: the first electrode 620, the second electrode 640, and the at least one semiconducting layer 630 therebetween, may be formed by depositing a respective constituent emissive region layer material in a desired pattern in a manufacturing process. In some non-limiting examples, such deposition may take place in a deposition process, in combination with a shadow mask 215, which may, in some non-limiting examples, may be one of: an open mask, and an FMM, having apertures to achieve such desired pattern by at least one of: masking, and precluding deposition of, the emissive region layer material on certain parts of an exposed layer surface of an underlying material exposed thereto.

The device 1000 may be shown as comprising a substrate 10, a TFT insulating layer 607 and a plurality of first electrodes 620, formed on an exposed layer surface 11 of the TFT insulating layer 607.

In some non-limiting examples, the substrate 10 may comprise the base substrate 615 (not shown for purposes of simplicity of illustration), and in some non-limiting examples, at least one TFT structure 606 corresponding to, and for driving, a corresponding emissive region 610, each having a corresponding (sub-) pixel 1015/616, positioned substantially thereunder and electrically coupled with its associated first electrode 620. PDL(s) 609 may be formed over the substrate 10, to define emissive region(s) 610. In some non-limiting examples, the PDL(s) 609 may cover edges of their respective first electrode 620.

In some non-limiting examples, at least one semiconducting layer 630 may be deposited over exposed region(s) of the first electrodes 210 corresponding to the emissive region 610 of each (sub-) pixel 1015/616 and, in some non-limiting examples, at least parts of corresponding at least one of: non-emissive regions 611, and corresponding PDLs 609, interposed therebetween.

In some non-limiting examples, a first deposited layer 130ₐ may be deposited over the exposed layer surface 11 of the at least one semiconducting layer(s) 630. In some non-limiting examples, such deposition may be effected by exposing the entire exposed layer surface 11 of the device 1000 to a vapor flux 332 of deposited material 331, using one of: an open mask, and a mask-free, deposition process, to deposit the first deposited layer 130ₐ over the at least one semiconducting layer(s) 630 to form a first layer of a second electrode 640 for a first emissive region 610ₐ so that such second electrode 640 is designated as a second electrode 640ₐ. Such second electrode 640ₐ may have a first thickness t_{c1} in the first emissive region 610ₐ. In some non-limiting examples, the first thickness t_{c1} may correspond to a thickness of the first deposited layer 130ₐ.

In some non-limiting examples, a first patterning coating 110₁ may be selectively deposited over first portions 101 of the device 1000, comprising the first emissive region 610ₐ.

In some non-limiting examples, the patterning coating 110i may be selectively deposited using a shadow mask 215 that may also have been used to deposit the at least one semiconducting layer 630ₐ of the first emissive region 610ₐ to reduce a number of stages for fabricating the device 1000.

In some non-limiting examples, a second deposited layer 130_{b} may be deposited over an exposed layer surface 11 of the device 1000 that is substantially devoid of the patterning coating 110, namely the exposed layer surface 11 of the first deposited layer 130ₐ in both of the second emissive region 610_{b}, and the third emissive region 610_{c} and, in some non-limiting examples, at least part(s) of the non-emissive region(s) 611 interposed therebetween, in which the PDLs 609 (if any) may lie. In some non-limiting examples, such deposition may be effected by exposing the entire exposed layer surface 11 of the device 1000 to a vapor flux 332 of deposited material 331, using one of: an open mask, and a mask-free deposition process, to deposit the second deposited layer 130_{b} over the first deposited layer 130ₐ to the extent that it is substantially devoid of the first patterning coating 110₁, such that the second deposited layer 130_{b} may be deposited on the second portion(s) 102 of the first deposited layer 130ₐ that are substantially devoid of the first patterning coating 110₁ to form a second layer of a second electrode 640 for the second emissive region 610_{b}, so that such second electrode 640 may be designated as a second electrode 640_{b}. Such second electrode 640_{b} may have a second thickness t_{c2} in the second emissive region 610_{b}. In some non-limiting examples, the second thickness t_{c2} may correspond to a combined average layer thickness of the first deposited layer 130ₐ and of the second deposited layer 130_{b} and may, in some non-limiting examples, be at least the first thickness t_{c1}.

In some non-limiting examples, a second patterning coating 110₂ may be selectively deposited over further first portions 101 of the device 1000, comprising the second emissive region 610_{b}.

In some non-limiting examples, a third deposited layer 130_{c} may be deposited over an exposed layer surface 11 of the device 1000, namely the exposed layer surface 11 of the second deposited layer 130_{b} in the third emissive region 610_{c}. In some non-limiting examples, such deposition may be effected by exposing the entire exposed layer surface 11 of the device 1000 to a vapor flux 332 of deposited material 331, In some non-limiting examples, the third deposited layer 130_{c} may be deposited using one of: an open mask, and a mask-free, deposition process, to deposit the third deposited layer 130_{c} over the second deposited layer 130_{b} to the extent that it is substantially devoid of any of: the first patterning coating 110₁, and the second patterning coating 110₂ to form a third layer of a second electrode 640 for the third emissive region 610_{c}, so that such second electrode 640 may be designated as a second electrode 640_{c}. Such second electrode 640_{c} may have a third thickness t_{c3} in the third emissive region 610_{c}. In some non-limiting examples, the third thickness t_{c3} may correspond to a combined average layer thickness of the first deposited layer 130ₐ, the second deposited layer 130_{b}, and the third deposited layer 130_{c} and may, in some non-limiting examples, be at least one of: the first thickness t_{c1}, and the second thickness t_{c2}.

In some non-limiting examples, a third patterning coating 110₃ may be selectively deposited over additional first portions 101 of the device 1000, comprising the third emissive region 610_{c}.

In some non-limiting examples, at least one auxiliary electrode 950 may be disposed in the non-emissive region(s) 611 of the device 1000 between neighbouring emissive regions 610 thereof and in some non-limiting examples, over the PDLs 609. In some non-limiting examples, the deposited layer 130 used to deposit the at least one auxiliary electrode 950 may be deposited using one of: an open mask, and a mask-free, deposition process, to deposit a deposited material 331 over the first deposited layer 130ₐ, the second deposited layer 130_{b}, and the third deposited layer 130_{c}, to the extent that it is substantially devoid of any of: the first patterning coating, 110₁, the second patterning coating 110₂, and the third patterning coating 110₃ to form the at least one auxiliary electrode 950. In some non-limiting examples, each of the at least one auxiliary electrodes 950 may be electrically coupled with a respective at least one of the second electrodes 640.

In some non-limiting examples, at least one of: the first deposited layer 130ₐ, the second deposited layer 130_{b}, and the third deposited layer 130_{c} may be at least one of: transmissive, and substantially transparent, in at least a part of the visible spectrum. Thus, in some non-limiting examples, at least one of: the second deposited layer 130_{b}, and the third deposited layer 130_{c} (and any additional deposited layer(s) 130 (not shown) may be disposed on top of the first deposited layer 130ₐ to form a multi-coating electrode 620, 640 that may also be at least one of: transmissive, and substantially transparent, in at least a part of the visible spectrum. In some non-limiting examples, the transmittance of at least one of: at least one of: the first deposited layer 130ₐ, the second deposited layer 130_{b}, and the third deposited layer 130_{c}, (and any additional deposited layer(s) 130), and the multi-coating electrode 620, 640 formed thereby, may exceed one of about: 30%, 40% 45%, 50%, 60%, 70%, 75%, and 80% in at least a part of the visible spectrum.

In some non-limiting examples, an average layer thickness of at least one of: the first deposited layer 130ₐ, the second deposited layer 130_{b}, and the third deposited layer 130_{c} may be made substantially thin to maintain a substantially high transmittance. In some non-limiting examples, an average layer thickness of the first deposited layer 130ₐ may be one of between about: 5-30 nm, 8-25 nm, and 10-20 nm. In some non-limiting examples, an average layer thickness of the second deposited layer 130_{b} may be one of between about: 1-25 nm, 1-20 nm, 1-15 nm, 1-10 nm, and 3-6 nm. In some non-limiting examples, an average layer thickness of the third deposited layer 130_{c} may be one of between about: 1-25 nm, 1-20 nm, 1-15 nm, 1-10 nm, and 3-6 nm. In some non-limiting examples, a thickness of a multi-coating electrode formed by a combination of the first deposited layer 130ₐ, the second deposited layer 130_{b}, and the third deposited layer 130_{c}, (and any additional deposited layer(s) 130) may be one of between about: 6-35 nm, 10-30 nm, 10-25 nm, and 12-18 nm.

The thickness of the at least one electrode 620, 640 may be varied to an even greater extent by independently modulating the average layer thickness, and a number, of at least one of: the patterning coating 110, and an NPC 520, deposited in part(s) of each emissive region 610 of the (sub-) pixel(s) 616.

In some non-limiting examples, an average layer thickness of at least one of: the first patterning coating 110₁, the second patterning coating 110₂, and the third patterning coating 110₃ disposed in at least one of: the first emissive region 610ₐ, the second emissive region 610_{b}, and the third emissive region 610_{c} respectively, may be varied according to at least one of: a colour, and emission spectrum of EM radiation, emitted by each emissive region 610. In some non-limiting examples, the first patterning coating 110i may have a first patterning coating thickness tₙ₁. In some non-limiting examples, the second patterning coating 110₂ may have a second patterning coating thickness tₙ₂. In some non-limiting examples, the third patterning coating 110₃ may have a third patterning coating thickness tₙ₃. In some non-limiting examples, at least one of: the first patterning coating thickness tₙ₁, the second patterning coating thickness tₙ₂, and the third patterning coating thickness tₙ₃, may be substantially the same. In some non-limiting examples, at least one of: the first patterning coating thickness tₙ₁, the second patterning coating thickness tₙ₂, and the third patterning coating thickness tₙ₃, may be different from one another.

In some non-limiting examples, an average layer thickness of the first deposited layer 130ₐ may exceed an average layer thickness of at least one of: the second deposited layer 130_{b}, and the third deposited layer 130_{c}. In some non-limiting examples, the average layer thickness of the second deposited layer 130_{b} may exceed the average layer thickness of at least one of: the first deposited layer 130ₐ, and the third deposited layer 130_{c}. In some non-limiting examples, the average layer thickness of the third deposited layer 130_{c} may exceed the average layer thickness of at least one of: the first deposited layer 130ₐ, and the second deposited layer 130_{b}. In some non-limiting examples, the average layer thickness of the first deposited layer 130ₐ, the average layer thickness of the second deposited layer 130_{b}, and the average layer thickness of the third deposited layer 130_{c}, may be substantially the same.

In some non-limiting examples, at least one deposited material 331 used to form the first deposited layer 130ₐ may be substantially the same as at least one deposited material 331 used to form at least one of: the second deposited layer 130_{b}, and the third deposited layer 130_{c}. In some non-limiting examples, such at least one deposited material 331 may be substantially as described herein in respect of at least one of: the first electrode 620, the second electrode 640, the auxiliary electrode 950, and a deposited layer 130 thereof.

In some non-limiting examples, at least one of: the first emissive region 610ₐ, the second emissive region 610_{b}, and the third emissive region 610_{c} may be substantially devoid of a closed coating 140 of the deposited material 331 used to form the at least one auxiliary electrode 950.

In some non-limiting examples, at least one of the first deposited layer 130ₐ, the second deposited layer 130_{b}, and the third deposited layer 130_{c}, may be at least one of: transmissive, and substantially transparent, in at least a part of the visible spectrum. Thus, in some non-limiting examples, at least one of: the second deposited layer 130_{b}, and the third deposited layer 130_{c} (and any additional deposited layer(s) 130) may be disposed on top of the first deposited layer 130ₐ to form a multi-coating electrode 620, 640, 950 that may also be at least one of: transmissive, and substantially transparent, in at least a part of the visible spectrum. In some non-limiting examples, the transmittance of any of the at least one of: the first deposited layer 130ₐ, the second deposited layer 130_{b}, the third deposited layer 130_{c}, any additional deposited layer(s) 130, and the multi-coating electrode 620, 640, 950, may exceed one of about: 30%, 40% 45%, 50%, 60%, 70%, 75%, and 80% in at least a part of the visible spectrum.

In some non-limiting examples, an average layer thickness of at least one of: the first deposited layer 130ₐ, the second deposited layer 130_{b}, and the third deposited layer 130_{c}, may be made substantially thin to maintain a substantially high transmittance. In some non-limiting examples, an average layer thickness of the first deposited layer 130ₐ may be one of between about: 5-30 nm, 8-25 nm, and 10-20 nm. In some non-limiting examples, an average layer thickness of the second deposited layer 130_{b} may be one of between about: 1-25 nm, 1-20 nm, 1-15 nm, 1-10 nm, and 3-6 nm. In some non-limiting examples, an average layer thickness of the third deposited layer 130_{c} may be one of between about: 1-25 nm, 1-20 nm, 1-15 nm, 1-10 nm, and 3-6 nm. In some non-limiting examples, a thickness of a multi-coating electrode formed by a combination of a plurality of: the first deposited layer 130ₐ, the second deposited layer 130_{b}, the third deposited layer 130_{c}, and any additional deposited layer(s) 130, may be one of between about: 6-35 nm, 10-30 nm, 10-25 nm, and 12-18 nm.

In some non-limiting examples, a thickness of the at least one auxiliary electrode 950 may exceed an average layer thickness of at least one of: the first deposited layer 130ₐ, the second deposited layer 130_{b}, the third deposited layer 130_{c}, and a common electrode. In some non-limiting examples, the thickness of the at least one auxiliary electrode 950 may be one of about: 50 nm, 80 nm, 100 nm, 150 nm, 200 nm, 300 nm, 400 nm, 500 nm, 700 nm, 800 nm, 1 μm, 1.2 μm, 1.5 μm, 2 μm, 2.5 μm, and 3 μm.

In some non-limiting examples, the at least one auxiliary electrode 950 may be substantially at least one of: non-transparent, and opaque. However, since the at least one auxiliary electrode 950 may be, in some non-limiting examples, provided in a non-emissive region 611 of the device 1000, the at least one auxiliary electrode 950 may not contribute to significant optical interference. In some non-limiting examples, the transmittance of the at least one auxiliary electrode 950 may be one of no more than about: 50%, 70%, 80%, 85%, 90%, and 95% in at least a part of the visible spectrum.

In some non-limiting examples, the at least one auxiliary electrode 950 may absorb EM radiation in at least a part of the visible spectrum.

### Conductive Coating for Electrically Coupling an Electrode to an Auxiliary Electrode

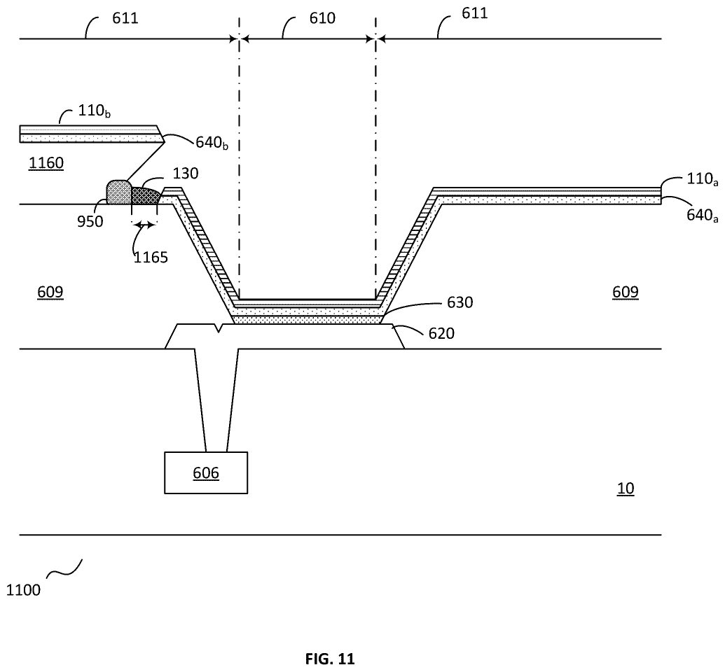

Turning to FIG. 11, there may be shown a cross-sectional view of an example version 1100 of an OLED device 600. The device 1100 may comprise in a lateral aspect, an emissive region 610 and an adjacent non-emissive region 611.

In some non-limiting examples, the emissive region 610 may correspond to a (sub-) pixel 1015/616 of the device 1100. The emissive region 610 may have a substrate 10, a first electrode 620, a second electrode 640 and at least one semiconducting layer 630 arranged therebetween.

The first electrode 620 may be disposed on an exposed layer surface 11 of the substrate 10. The substrate 10 may comprise a TFT structure 606, that may be electrically coupled with the first electrode 620. At least one of: the edges, and perimeter, of the first electrode 620 may generally be covered by at least one PDL 609.

The non-emissive region 611 may have an auxiliary electrode 950 and a first part of the non-emissive region 611 may have a projection 1160 arranged to project over a lateral aspect of the auxiliary electrode 950. The projection 1160 may extend laterally to provide a shaded region 1165. In some non-limiting examples, the projection 1160 may be recessed proximate to the auxiliary electrode 950 on at least one side to provide the shaded region 1165. As shown, the shaded region 1165 may in some non-limiting examples, correspond to a region on a surface of the PDL 609 that may overlap with a lateral projection of the projection 1160. The non-emissive region 611 may further comprise a deposited layer 130 disposed in the shaded region 1165. The deposited layer 130 may electrically couple the auxiliary electrode 950 with the second electrode 640.

A patterning coating 110ₐ may be disposed in the emissive region 610 over the exposed layer surface 11 of the second electrode 640. In some non-limiting examples, an exposed layer surface 11 of the projection 1160 may be coated with a residual thin conductive film from deposition of a thin conductive film to form a second electrode 640. In some non-limiting examples, an exposed layer surface 11 of the residual thin conductive film may be coated with a residual patterning coating 110_{b} from deposition of the patterning coating 110.

However, because of the lateral projection of the projection 1160 over the shaded region 1165, the shaded region 1165 may be substantially devoid of patterning coating 110. Thus, when a deposited layer 130 may be deposited on the device 1100 after deposition of the patterning coating 110, the deposited layer 130 may at least one of: be deposited on, and migrate to, the shaded region 1165 to couple the auxiliary electrode 950 with the second electrode 640.

Those having ordinary skill in the relevant art will appreciate that a non-limiting example has been shown in FIG. 11 and that various modifications may be apparent. In some non-limiting examples, the projection 1160 may provide a shaded region 1165 along at least two of its sides. In some non-limiting examples, the projection 1160 may be omitted and the auxiliary electrode 950 may comprise a recessed portion that may define the shaded region 1165. In some non-limiting examples, the auxiliary electrode 950 and the deposited layer 130 may be disposed directly on a surface of the substrate 10, instead of the PDL 609.

### Partition and Recess

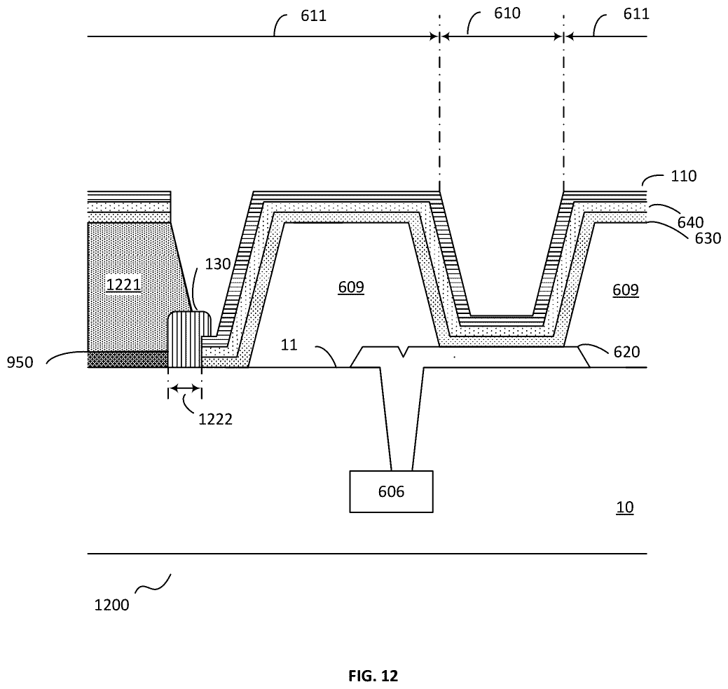

Turning to FIG. 12, there may be shown a cross-sectional view of an example version 1200 of an OLED device 600. The device 1200 may comprise a substrate 10 having an exposed layer surface 11. The substrate 10 may comprise at least one TFT structure 606. In some non-limiting examples, the at least one TFT structure 606 may be formed by depositing and patterning a series of thin films when fabricating the substrate 10, in some non-limiting examples, as described herein.

The device 1200 may comprise, in a lateral aspect, an emissive region 610 having an associated lateral aspect and at least one adjacent non-emissive region 611, each having an associated lateral aspect. The exposed layer surface 11 of the substrate 10 in the emissive region 610 may be provided with a first electrode 620, that may be electrically coupled with the at least one TFT structure 606. A PDL 609 may be provided on the exposed layer surface 11, such that the PDL 609 covers the exposed layer surface 11 as well as at least one of: an edge, and perimeter, of the first electrode 620. The PDL 609 may, in some non-limiting examples, be provided in the lateral aspect of the non-emissive region 611. The PDL 609 may define a valley-shaped configuration that may provide an opening that generally may correspond to the lateral aspect of the emissive region 610 through which a layer surface of the first electrode 620 may be exposed. In some non-limiting examples, the device 1200 may comprise a plurality of such openings defined by the PDLs 609, each of which may correspond to a (sub-) pixel 1015/616 region of the device 1200.

As shown, in some non-limiting examples, a partition 1221 may be provided on the exposed layer surface 11 in the lateral aspect of a non-emissive region 611 and, as described herein, may define a shaded region 1165, such as a recessed region 1222. In some non-limiting examples, the recessed region 1222 may be formed by an edge of a lower section of the partition 1221 being at least one of: recessed, staggered, and offset, with respect to an edge of an upper section of the partition 1221 that may project beyond the recessed region 1222.

In some non-limiting examples, the lateral aspect of the emissive region 610 may comprise at least one semiconducting layer 630 disposed over the first electrode 620, a second electrode 640, disposed over the at least one semiconducting layer 630, and a patterning coating 110 disposed over the second electrode 640. In some non-limiting examples, the at least one semiconducting layer 630, the second electrode 640 and the patterning coating 110 may extend laterally to cover at least the lateral aspect of a part of at least one adjacent non-emissive region 611. In some non-limiting examples, as shown, the at least one semiconducting layer 630, the second electrode 640 and the patterning coating 110 may be disposed on at least a part of at least one PDL 609 and at least a part of the partition 1221. Thus, as shown, the lateral aspect of the emissive region 610, the lateral aspect of a part of at least one adjacent non-emissive region 611, a part of at least one PDL 609, and at least a part of the partition 1221, together may make up a first portion 101, in which the second electrode 640 may lie between the patterning coating 110 and the at least one semiconducting layer 630.

An auxiliary electrode 950 may be disposed proximate to, including without limitation, within, the recessed region 1222 and a deposited layer 130 may be arranged to electrically couple the auxiliary electrode 950 with the second electrode 640. Thus, as shown, in some non-limiting examples, the recessed region 1222 may comprise a second portion 102, in which the deposited layer 130 is disposed on the exposed layer surface 11.

In some non-limiting examples, in depositing the deposited layer 130, at least a part of the evaporated flux 332 of the deposited material 331 may be directed at a non-normal angle relative to a lateral plane of the exposed layer surface 11. In some non-limiting examples, at least a part of the evaporated flux 332 may be incident on the device 1200 at a non-zero angle of incidence that is, relative to such lateral plane of the exposed layer surface 11, one of no more than about: 90°, 85°, 80°, 75°, 70°, 60°, and 50°. By directing an evaporated flux 332 of a deposited material 331, including at least a part thereof incident at a non-normal angle, at least one exposed layer surface 11 of, including without limitation, in, the recessed region 1222 may be exposed to such evaporated flux 332.

In some non-limiting examples, a likelihood of such evaporated flux 332 being precluded from being incident onto at least one exposed layer surface 11 of, including without limitation, in, the recessed region 1222 due to the presence of the partition 1221, may be reduced since at least a part of such evaporated flux 332 may be flowed at a non-normal angle of incidence.

In some non-limiting examples, at least a part of such evaporated flux 332 may be non-collimated. In some non-limiting examples, at least a part of such evaporated flux 332 may be generated by an evaporation source that is at least one of: a point, linear, and surface, source.

In some non-limiting examples, the device 1200 may be displaced during deposition of the deposited layer 130. In some non-limiting examples, at least one of: the device 1200, and the substrate 10 thereof, including without limitation, any layer(s) deposited thereon, may be subjected to a displacement that is angular, in an aspect that is at least one of: lateral, and substantially parallel, to the longitudinal aspect.

In some non-limiting examples, the device 1200 may be rotated about an axis that is substantially normal to the lateral plane of the exposed layer surface 11 while being subjected to the evaporated flux 332.

In some non-limiting examples, at least a part of such evaporated flux 332 may be directed toward the exposed layer surface 11 of the device 1200 in a direction that is substantially normal to the lateral plane of the exposed layer surface 11.

Without wishing to be bound by a particular theory, it may be postulated that the deposited material 331 may nevertheless be deposited within the recessed region 1222 due to at least one of: lateral migration, and desorption, of adatoms adsorbed onto the exposed layer surface 11 of the patterning coating 110. In some non-limiting examples, it may be postulated that any adatoms adsorbed onto the exposed layer surface 11 of the patterning coating 110 may tend to at least one of: migrate, and desorb, from such exposed layer surface 11 due to thermodynamic properties of the exposed layer surface 11 that may not have applicability for forming a stable nucleus. In some non-limiting examples, it may be postulated that at least some of the adatoms at least one of: migrating, and desorbing, off such exposed layer surface 11 may be re-deposited onto the surfaces in the recessed region 1222 to form the deposited layer 130.

In some non-limiting examples, the deposited layer 130 may be formed such that the deposited layer 130 may be electrically coupled with both the auxiliary electrode 950 and the second electrode 640. In some non-limiting examples, the deposited layer 130 may be in physical contact with at least one of the auxiliary electrode 950, and the second electrode 640. In some non-limiting examples, an intermediate layer may be present between the deposited layer 130 and at least one of: the auxiliary electrode 950, and the second electrode 640. However, in such example, such intermediate layer may not substantially preclude the deposited layer 130 from being electrically coupled with the at least one of: the auxiliary electrode 950, and the second electrode 640. In some non-limiting examples, such intermediate layer may be substantially thin and be such as to permit electrical coupling therethrough. In some non-limiting examples, a sheet resistance of the deposited layer 130 may be no more than a sheet resistance of the second electrode 640.

As shown in FIG. 12, the recessed region 1222 may be substantially devoid of the second electrode 640. In some non-limiting examples, during the deposition of the second electrode 640, the recessed region 1222 may be masked by the partition 1221, such that the evaporated flux 332 of the deposited material 331 for forming the second electrode 640 may be substantially precluded from being incident on at least one exposed layer surface 11 of, including without limitation, in, the recessed region 1222. In some non-limiting examples, at least a part of the evaporated flux 332 of the deposited material 331 for forming the second electrode 640 may be incident on at least one exposed layer surface 11 of, including without limitation, in, the recessed region 1222, such that the second electrode 640 may extend to cover at least a part of the recessed region 1222.

In some non-limiting examples, at least one of: the auxiliary electrode 950, the deposited layer 130, and the partition 1221, may be selectively provided in certain region(s) of an OLED display panel 700. In some non-limiting examples, any of these features may be provided proximate to at least one edge of such display panel 700 for electrically coupling at least one element of the frontplane 601, including without limitation, the second electrode 640, with at least one element of the backplane 602. In some non-limiting examples, providing such features proximate to such edges may facilitate supplying and distributing electrical current to the second electrode 640 from an auxiliary electrode 950 located proximate to such edges. In some non-limiting examples, such configuration may facilitate reducing a bezel size of the display panel 700.

In some non-limiting examples, at least one of: the auxiliary electrode 950, the deposited layer 130, and the partition 1221, may be omitted from certain regions(s) of such display panel 700. In some non-limiting examples, such features may be omitted from parts of the display panel 700, including without limitation, where a substantially high pixel density may be provided, other than proximate to at least one edge thereof.

### Aperture in Non-Emissive Region

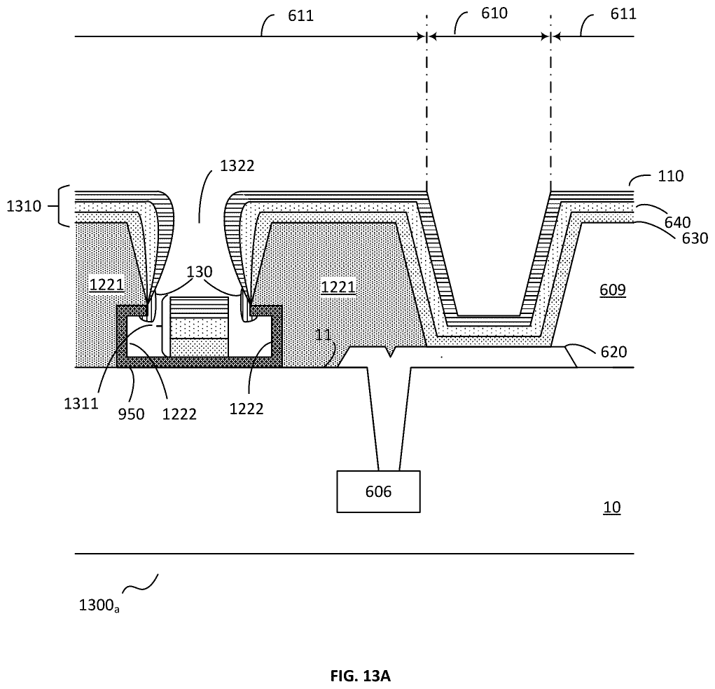

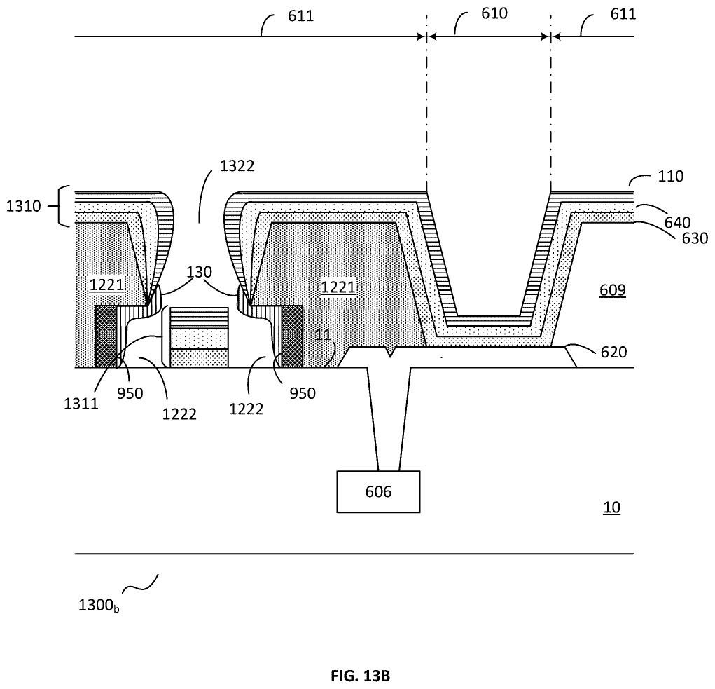

Turning now to FIG. 13A, there may be shown a cross-sectional view of an example version 1300ₐ of an OLED device 600. The device 1300ₐ may differ from the device 1200 in that a pair of partitions 1221 in the non-emissive region 611 may be disposed in a facing arrangement to define a shaded region 1165, such as an aperture 1322, therebetween. As shown, in some non-limiting examples, at least one of the partitions 1221 may function as a PDL 609 that covers at least an edge of the first electrode 620 and that defines at least one emissive region 610. In some non-limiting examples, at least one of the partitions 1221 may be provided separately from a PDL 609.

A shaded region 1165, such as the recessed region 1222, may be defined by at least one of the partitions 1221. In some non-limiting examples, the recessed region 1222 may be provided in a part of the aperture 1322 proximate to the substrate 10. In some non-limiting examples, the aperture 1322, when viewed in plan, may be substantially elliptical. In some non-limiting examples, the recessed region 1222, when viewed in plan, may be substantially annular and surround the aperture 1322.

In some non-limiting examples, the recessed region 1222 may be substantially devoid of materials for forming each of the layers of at least one of: a device stack 1310, and of a residual device stack 1311.

In these figures, a device stack 1310 may be shown comprising the at least one semiconducting layer 630, the second electrode 640 and the patterning coating 110 deposited on an upper section of the partition 1221.

In these figures, a residual device stack 1311 may be shown comprising the at least one semiconducting layer 630, the second electrode 640 and the patterning coating 110 deposited on the substrate 10 beyond the partition 1221 and recessed region 1222. From comparison with FIG. 12, it may be seen that the residual device stack 1311 may, in some non-limiting examples, correspond to the semiconductor layer 630, second electrode 640 and the patterning coating 110 as it approaches the recessed region 1222 proximate to a lip of the partition 1221. In some non-limiting examples, the residual device stack 1311 may be formed when one of: an open mask, and a mask-free, deposition process is used to deposit various materials of the device stack 1310.

In some non-limiting examples, the residual device stack 1311 may be disposed within the aperture 1322. In some non-limiting examples, evaporated materials for forming each of the layers of the device stack 1310 may be deposited within the aperture 1322 to form the residual device stack 1311 therein.

In some non-limiting examples, the auxiliary electrode 950 may be arranged such that at least a part thereof is disposed within the recessed region 1222. As shown, in some non-limiting examples, the auxiliary electrode 950 may be arranged within the aperture 1322, such that the residual device stack 1311 is deposited onto a surface of the auxiliary electrode 950.

A deposited layer 130 may be disposed within the aperture 1322 for electrically coupling the second electrode 640 with the auxiliary electrode 950. In some non-limiting examples, at least a part of the deposited layer 130 may be disposed within the recessed region 1222.

Turning now to FIG. 13B, there may be shown a cross-sectional view of a further version 1300_{b} of an OLED device 600. As shown, the auxiliary electrode 950 may be arranged to form at least a part of a side of the partition 1221. As such, the auxiliary electrode 950 may be substantially annular, when viewed in plan view, and may surround the aperture 1322. As shown, in some non-limiting examples, the residual device stack 1311 may be deposited onto an exposed layer surface 11 of the substrate 10.

In some non-limiting examples, the partition 1221 may comprise an NPC 520. In some non-limiting examples, the auxiliary electrode 950 may act as an NPC 520.

In some non-limiting examples, the NPC 520 may be provided by the second electrode 640, including without limitation, at least one of: a portion, layer, and material thereof. In some non-limiting examples, the second electrode 640 may extend laterally to cover the exposed layer surface 11 arranged in the shaded region 1165. In some non-limiting examples, the second electrode 640 may comprise a lower layer thereof and a second layer thereof, wherein the second layer thereof may be deposited on the lower layer thereof. In some non-limiting examples, the lower layer of the second electrode 640 may comprise an oxide such as, without limitation, ITO, IZO, and ZnO. In some non-limiting examples, the upper layer of the second electrode 640 may comprise a metal such as, without limitation, at least one of Ag, Mg, Mg:Ag, Yb/Ag, other alkali metals, and other alkali earth metals.

In some non-limiting examples, the lower layer of the second electrode 640 may extend laterally to cover a surface of the shaded region 1165, such that it forms the NPC 520. In some non-limiting examples, at least one surface defining the shaded region 1165 may be treated to form the NPC 520. In some non-limiting examples, such NPC 520 may be formed by at least one of: chemical, and physical, treatment, including without limitation, subjecting the surface(s) of the shaded region 1165 to at least one of: a plasma, UV, and UV-ozone treatment.

Without wishing to be bound to any particular theory, it may be postulated that such treatment may at least one of: chemically, and physically, alter such surface(s) to modify at least one property thereof. In some non-limiting examples, such treatment of the surface(s) may increase at least one of: a concentration of at least one of: C—O, and C—OH, bonds on such surface(s), a roughness of such surface(s), and a concentration of certain species, including without limitation, functional groups, including without limitation, at least one of: halogens, nitrogen-containing functional groups, and oxygen-containing functional groups, to thereafter act as an NPC 520.

### Diffraction Reduction

It has been discovered that, in some non-limiting examples, the at least one EM signal 731 passing through the at least one signal-transmissive region 612 may be impacted by a diffraction characteristic of a diffraction pattern imposed by a shape of the at least one signal-transmissive region 612.

At least in some non-limiting examples, a display panel 700 that causes at least one EM signal 731 to pass through the at least one signal-transmissive region 612 that is shaped to exhibit a distinctive and non-uniform diffraction pattern, may interfere with the capture of at least one of: an image, and an EM radiation pattern represented thereby.

In some non-limiting examples, such diffraction pattern may interfere with an ability to facilitate mitigating interference by such diffraction pattern, that is, to permit an under-display component 730 to be able to one of: accurately receive and process such pattern, even with the application of optical post-processing techniques, and to allow a viewer of such pattern through such display panel 700 to discern information contained therein.

In some non-limiting examples, at least one of: a distinctive, and non-uniform, diffraction pattern may result from a shape of the at least one signal-transmissive region 612 that may cause distinct, including without limitation, angularly separated, diffraction spikes in the diffraction pattern.

In some non-limiting examples, a first diffraction spike may be distinguished from a second proximate diffraction spike by simple observation, such that a total number of diffraction spikes along a full angular revolution may be counted. However, in some non-limiting examples, especially where the number of diffraction spikes is large, it may be more difficult to identify individual diffraction spikes. In such circumstances, the distortion effect of the resulting diffraction pattern may in fact facilitate mitigation of the interference caused thereby, since the distortion effect tends to be at least one of: blurred, and distributed more evenly. Such at least one of: blurring and more even distribution, of the distortion effect may, in some non-limiting examples, be more amenable to mitigation, including without limitation, by optical post-processing techniques, in order to recover the original image (information) contained therein.

In some non-limiting examples, an ability to facilitate mitigation of the interference caused by the diffraction pattern may increase as the number of diffraction spikes increases.

In some non-limiting examples, a distinctive and non-uniform diffraction pattern may result from a shape of the at least one signal-transmissive region 612 that at least one of: increases a length of a pattern boundary within the diffraction pattern between region(s) of high intensity of EM radiation and region(s) of low intensity of EM radiation as a function of a pattern circumference of the diffraction pattern, and that reduces a ratio of the pattern circumference relative to the length of the pattern boundary thereof.

Without wishing to be bound by any specific theory, it may be postulated that display panels 700 having closed boundaries of signal-transmissive regions 612 defined by a corresponding signal-transmissive region 612 that are polygonal may exhibit a distinctive and non-uniform diffraction pattern that may adversely impact an ability to facilitate mitigation of interference caused by the diffraction pattern, relative to a display panel 700 having closed boundaries of signal-transmissive regions 612 defined by a corresponding signal-transmissive region 612 that is non-polygonal.

In the present disclosure, the term “polygonal” may refer generally to at least one of: shapes, figures, closed boundaries, and perimeters, formed by a finite number of linear segments and the term “non-polygonal” may refer generally to at least one of: shapes, figures, closed boundaries, and perimeters, that are not polygonal. In some non-limiting examples, a closed boundary formed by a finite number of linear segments and at least one non-linear (curved) segment may be considered non-polygonal.

Without wishing to be bound by a particular theory, it may be postulated that when a closed boundary of an EM radiation signal-transmissive region 612 defined by a corresponding signal-transmissive region 612 comprises at least one non-linear (curved) segment, EM signals incident thereon and transmitted therethrough may exhibit a less distinctive (more uniform) diffraction pattern that facilitates mitigation of interference caused by the diffraction pattern.

In some non-limiting examples, a display panel 700 having a closed boundary of the EM radiation signal-transmissive regions 612 defined by a corresponding signal-transmissive region 612 that is substantially elliptical, including without limitation, circular may further facilitate mitigation of interference caused by the diffraction pattern.

In some non-limiting examples, a signal-transmissive region 612 may be defined by a finite plurality of convex rounded segments. In some non-limiting examples, at least some of these segments coincide at a concave notch (peak).

### Removal of Selective Coating

In some non-limiting examples, the patterning coating 110 may be removed after deposition of the deposited layer 130, such that at least a part of a previously exposed layer surface 11 of an underlying layer 210 of a device 600, covered by the patterning coating 110 may become exposed once again. In some non-limiting examples, the patterning coating 110 may be selectively removed by at least one of: etching, dissolving the patterning coating 110, and by employing at least one of: plasma, and solvent, processing techniques that do not substantially affect, including without limitation, erode, the deposited layer 130.

In some non-limiting examples, at an initial deposition stage, a patterning coating 110 may have been selectively deposited on a first portion 101 of an exposed layer surface 11 of an underlying layer 210, including without limitation, the substrate 10.

In some non-limiting examples, at a further deposition stage, a deposited layer 130 may be deposited on the exposed layer surface 11 of the underlying layer 210, that is, on both the exposed layer surface 11 of the patterning coating 110 where the patterning coating 110 may have been deposited during the initial deposition stage, as well as the exposed layer surface 11 of the substrate 10 where that patterning coating 110 may not have been deposited during the initial deposition stage. Because of the nucleation-inhibiting properties of the first portion 101 where the patterning coating 110 may have been disposed, the deposited layer 130 disposed thereon may tend to not remain, resulting in a pattern of selective deposition of the deposited layer 130, that may correspond to a second portion 102, leaving the first portion 101 substantially devoid of the deposited layer 130.

In some non-limiting examples, at a final deposition stage, the patterning coating 110 may have been removed from the first portion 101 of the exposed layer surface 11 of the substrate 10, such that the deposited layer 130 deposited during the further deposition stage may remain on the substrate 10 and regions of the substrate 10 on which the patterning coating 110 may have been deposited during the initial deposition stage may now be exposed (uncovered).

In some non-limiting examples, the removal of the patterning coating 110 in the final deposition stage may be effected by exposing the device 600 to at least one of: a solvent, and a plasma that etches away (reacts with) the patterning coating 110 without substantially impacting the deposited layer 130.

### Thin Film Formation

The formation of thin films during vapor deposition on an exposed layer surface 11 of an underlying layer 210 may involve processes of nucleation and growth.

During initial stages of film formation, a sufficient number of vapor monomers, which in some non-limiting examples may be at least one of: molecules, and atoms of a deposited material 331 in vapor form) may condense from a vapor phase to form initial nuclei on the exposed layer surface 11 presented of an underlying layer 210. As vapor monomers may impinge on such surface, at least one of: a characteristic size, and deposited density, of these initial nuclei may increase to form small particle structures 150. Non-limiting examples of a dimension to which such characteristic size refers may include at least one of: a height, width, length, and diameter, of such particle structure 150.

After reaching a saturation island density, adjacent particle structures 150 may start to coalesce, increasing an average characteristic size of such particle structures 150, while decreasing a deposited density thereof.

With continued vapor deposition of monomers, coalescence of adjacent particle structures 150 may continue until a substantially closed coating 140 may eventually be deposited on an exposed layer surface 11 of an underlying layer 210. The behaviour, including optical effects caused thereby, of such closed coatings 140 may be generally substantially uniform, and consistent.

There may be at least three basic growth modes for the formation of thin films, in some non-limiting examples, culminating in a closed coating 140: 1) island (Volmer-Weber), 2) layer-by-layer (Frank-van der Merwe), and 3) Stranski-Krastanov.

Island growth may occur when stale clusters of monomers nucleate on an exposed layer surface 11 and grow to form discrete islands. This growth mode may occur when the interaction between the monomers is stronger than that between the monomers and the surface.

The nucleation rate may describe how many nuclei of a given size (where the free energy does not push a cluster of such nuclei to one of: grow, and shrink) (“critical nuclei”) may be formed on a surface per unit time. During initial stages of film formation, it may be unlikely that nuclei will grow from direct impingement of monomers on the surface, since the deposited density of nuclei is low, and thus the nuclei may cover a substantially small fraction of the surface (e.g., there are large gaps/spaces between neighboring nuclei). Therefore, the rate at which critical nuclei may grow may depend on the rate at which adatoms (e.g., adsorbed monomers) on the surface migrate and attach to nearby nuclei.

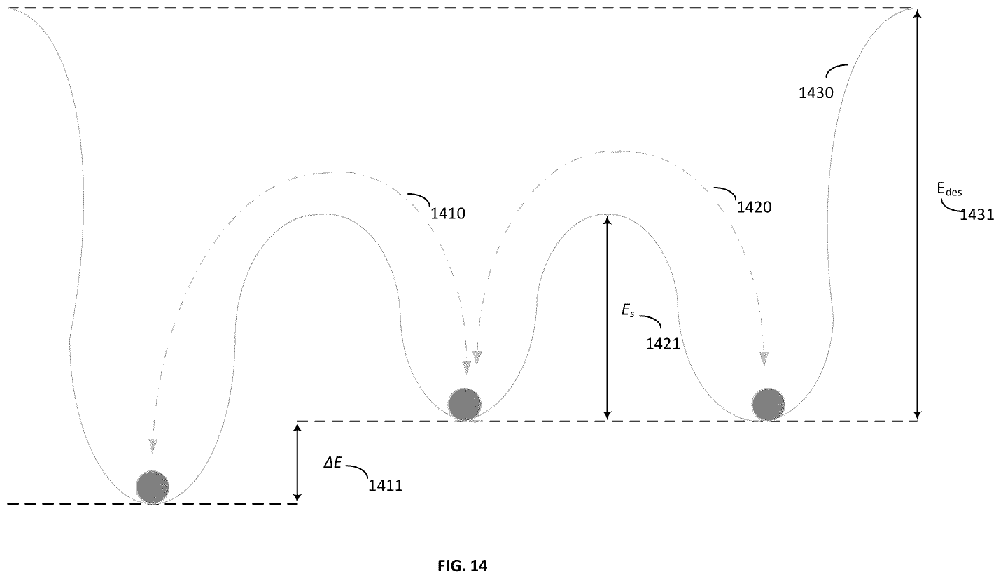

An example of an energy profile of an adatom adsorbed onto an exposed layer surface 11 of an underlying layer 210 is illustrated in FIG. 14. Specifically, FIG. 14 may illustrate example qualitative energy profiles corresponding to: an adatom escaping from a local low energy site (1410); diffusion of the adatom on the exposed layer surface 11 (1420); and desorption of the adatom (1430).

In 1410, the local low energy site may be any site on the exposed layer surface 11 of an underlying layer 210, onto which an adatom will be at a lower energy. In some non-limiting examples, the nucleation site may comprise at least one of: a defect, and an anomaly, on the exposed layer surface 11, including without limitation, at least one of: a ledge, a step edge, a chemical impurity, a bonding site, and a kink (“heterogeneity”).

Sites of substrate heterogeneity may increase an energy involved to desorb the adatom from the surface E_{des} 1431, leading to a higher deposited density of nuclei observed at such sites. Also, impurities, including without limitation, contamination, on a surface may also increase E_{des} 1431, leading to a higher deposited density of nuclei. For vapor deposition processes, conducted under high vacuum conditions, the type and deposited density of contaminants on a surface may be affected by a vacuum pressure and a composition of residual gases that make up that pressure.

Once the adatom is trapped at the local low energy site, there may, in some non-limiting examples, be an energy barrier before surface diffusion takes place. Such energy barrier may be represented as ΔE 1411 in FIG. 14. In some non-limiting examples, if the energy barrier ΔE 1411 to escape the local low energy site is substantially large, the site may act as a nucleation site.

In 1420, the adatom may diffuse on the exposed layer surface 11. In some non-limiting examples, in the case of localized absorbates, adatoms may tend to oscillate near a minimum of the surface potential and migrate to various neighboring sites until the adatom is either one of: desorbed, and is incorporated into growing islands 150 formed by at least one of: a cluster of adatoms, and a growing film. In FIG. 14, the activation energy associated with surface diffusion of adatoms may be represented as Eₛ 1421.

In 1430, the activation energy associated with desorption of the adatom from the surface may be represented as E_{des} 1431. Those having ordinary skill in the relevant art will appreciate that any adatoms that are not desorbed may remain on the exposed layer surface 11. In some non-limiting examples, such adatoms may diffuse on the exposed layer surface 11, become part of a cluster of adatoms that at least one of: form islands 150 on the exposed layer surface 11, and be incorporated as part of a growing coating.

After adsorption of an adatom on a surface, the adatom may one of: desorb from the surface, and may migrate some distance on the surface before either desorbing, interacting with other adatoms to one of: form a small cluster, attach to a growing nucleus. An average amount of time that an adatom may remain on the surface after initial adsorption may be given by Equation (4):

$${τ}_{s} = \frac{1}{v} exp ( \frac{{E}_{des}}{k T} ) \qquad ( 4 )$$

In the above Equation (4):
v is a vibrational frequency of the adatom on the surface,
k is the Boltzmann constant, and
T is temperature.

From Equation (4) it may be noted that the lower the value of E_{des} 1431, the easier it may be for the adatom to desorb from the surface, and hence the shorter the time the adatom may remain on the surface. A mean distance an adatom can diffuse may be given by Equation (5):

$$X = {a}_{0} exp ( \frac{{E}_{des} - {E}_{s}}{2 kT} ) \qquad ( 5 )$$

where:
α₀ is a lattice constant.

For at least one of: low values of E_{des} 1431, and high values of Eₛ 1421, the adatom may diffuse a shorter distance before desorbing, and hence may be less likely to at least one of: attach to growing nuclei, and interact with another one of: adatom, and cluster of adatoms.

During initial stages of formation of a deposited layer of particle structures 150, adsorbed adatoms may interact to form particle structures 150, with a critical concentration of particle structures 150 per unit area being given by Equation (6):

$$\frac{{N}_{i}}{{n}_{0}} = {\vert \frac{{N}_{1}}{{n}_{0}} \vert}^{i} exp ( \frac{{E}_{i}}{k T} ) \qquad ( 6 )$$

where:
Eᵢ is an energy involved to dissociate a critical cluster comprising I adatoms into separate adatoms,
n₀ is a total deposited density of adsorption sites, and
N₁ is a monomer deposited density given by Equation (7):

$${N}_{1} = \dot{R} {τ}_{s} \qquad ( 7 )$$

where:
\dot{R} is a vapor impingement rate.

In some non-limiting examples, i may depend on a crystal structure of a material being deposited and may determine a critical size of particle structures 150 to form a stable nucleus.

A critical monomer supply rate for growing particle structures 150 may be given by the rate of vapor impingement and an average area over which an adatom can diffuse before desorbing:

$$\dot{R} {X}^{2} = {α}_{0}^{2} exp ( \frac{{E}_{des} - {E}_{s}}{k T} ) \qquad ( 8 )$$

The critical nucleation rate may thus be given by the combination of the above equations to form Equation (9):

$${\dot{N}}_{i} = \dot{R} {α}_{0}^{2} {{n}_{0} ( \frac{R}{v {n}_{0}} )}^{i} exp ( \frac{( i + 1 ) {E}_{des} - {E}_{s} + {E}_{i}}{k T} ) \qquad ( 9 )$$

From Equation (9), it may be noted that the critical nucleation rate may be suppressed for surfaces that have a low desorption energy for adsorbed adatoms, a high activation energy for diffusion of an adatom, are at least one of: at high temperatures, and are subjected to vapor impingement rates.

Under high vacuum conditions, a flux of molecules that may impinge on a surface (per cm²-sec) may be given by Equation (10):

$$ϕ = 3.513 \times 1 {0}^{22} \frac{P}{M T} \qquad ( 10 )$$

where:
P is pressure, and
M is molecular weight.

Therefore, a higher partial pressure of a reactive gas, such as H₂O, may lead to a higher deposited density of contamination on a surface during vapor deposition, leading to an increase in E_{des} 1431 and hence a higher deposited density of nuclei.

In the present disclosure, “nucleation-inhibiting” may refer to at least one of: a coating, material, and a layer thereof, that may have a surface that exhibits an initial sticking probability against deposition of a deposited material 331 thereon, that may be close to 0, including without limitation, less than about 0.3, such that the deposition of the deposited material 331 on such surface may be inhibited.

In the present disclosure, “nucleation-promoting” may refer to at least one of: a coating, material, and a layer thereof, that has a surface that exhibits an initial sticking probability against deposition of a deposited material 331 thereon, that may be close to 1, including without limitation, greater than about 0.7, such that the deposition of the deposited material 331 on such surface may be facilitated.

Without wishing to be bound by a particular theory, it may be postulated that the shapes and sizes of such nuclei and the subsequent growth of such nuclei into islands and thereafter into a thin film may depend upon various factors, including without limitation, interfacial tensions between at least one of: the vapor, the surface, and the condensed film nuclei.

One measure of at least one of: a nucleation-inhibiting, and nucleation-promoting, property of a surface may be the initial sticking probability of the surface against the deposition of a given deposited material 331.

In some non-limiting examples, the sticking probability S may be given by Equation (11):

$$S = \frac{{N}_{a d s}}{{N}_{t o t a l}} \qquad ( 11 )$$

where:
N_{ads} is a number of adatoms that remain on an exposed layer surface 11 (that is, are incorporated into a film), and
Nₜₒₜₐₗ is a total number of impinging monomers on the surface.

A sticking probability S equal to 1 may indicate that all monomers that impinge on the surface are adsorbed and subsequently incorporated into a growing film. A sticking probability S equal to 0 may indicate that all monomers that impinge on the surface are desorbed and subsequently no film may be formed on the surface.

A sticking probability S of a deposited material 331 on various surfaces may be evaluated using various techniques of measuring the sticking probability S, including without limitation, a dual quartz crystal microbalance (QCM) technique as described by Walker et al., J. Phys. Chem. C 2007, 111, 765 (2006).

As the deposited density of a deposited material 331 may increase (e.g., increasing average film thickness), a sticking probability S may change.

An initial sticking probability S₀ may therefore be specified as a sticking probability S of a surface prior to the formation of any significant number of critical nuclei. One measure of an initial sticking probability S₀ may involve a sticking probability S of a surface against the deposition of a deposited material 331 during an initial stage of deposition thereof, where an average film thickness of the deposited material 331 across the surface is at, including without limitation, below, a threshold value. In the description of some non-limiting examples a threshold value for an initial sticking probability may be specified as, in some non-limiting examples, 1 nm. An average sticking probability S may then be given by Equation (12):

$$\overline{S} = {S}_{0} ( 1 - {A}_{n u c} ) + {S}_{n u c} ( {A}_{n u c} ) \qquad ( 12 )$$

where:
S_{nuc} is a sticking probability S of an area covered by particle structures 150, and
A_{nuc} is a percentage of an area of a substrate surface covered by particle structures 150.

In some non-limiting examples, a low initial sticking probability may increase with increasing average film thickness. This may be understood based on a difference in sticking probability between an area of an exposed layer surface 11 with no particle structures 150, in some non-limiting examples, a bare substrate 10, and an area with a high deposited density. In some non-limiting examples, a monomer that may impinge on a surface of a particle structure 150 may have a sticking probability that may approach 1.

Based on the energy profiles 1410, 1420, 1430 shown in FIG. 14, it may be postulated that materials that exhibit at least one of: substantially low activation energy for desorption (E_{des} 1431), and substantially high activation energy for surface diffusion (Eₛ 1421), may be deposited as a patterning coating 110, and may have applicability for use in various applications.

Without wishing to be bound by a particular theory, it may be postulated that, in some non-limiting examples, the relationship between various interfacial tensions present during nucleation and growth may be dictated according to Young's equation in capillarity theory (Equation (13)):

$${γ}_{s v} = {γ}_{fs} + {γ}_{vf} cos θ \qquad ( 13 )$$

where:
γₛᵥ (FIG. 15) corresponds to the interfacial tension between the substrate 10 and vapor,
γ_{fs} (FIG. 15) corresponds to the interfacial tension between the deposited material 331 and the substrate 10,
γ_{vf} (FIG. 15) corresponds to the interfacial tension between the vapor flux and the film, and
θ is the film nucleus contact angle.

FIG. 15 may illustrate the relationship between the various parameters represented in this equation.

On the basis of Young's equation (Equation (13)), it may be derived that, for island growth, the film nucleus contact angle may exceed 0 and therefore: γₛᵥ<γ_{fs}+γ_{vf}.

For layer growth, where the deposited material 331 may “wet” the substrate 10, the nucleus contact angle θ may be equal to 0, and therefore: γₛᵥ=γ_{fs}+γ_{vf}.

For Stranski-Krastanov growth, where the strain energy per unit area of the film overgrowth may be large with respect to the interfacial tension between the vapor 532 and the deposited material 331: γₛᵥ>γ_{fs}+γ_{vf}.

Without wishing to be bound by any particular theory, it may be postulated that the nucleation and growth mode of a deposited material 331 at an interface between the patterning coating 110 and the exposed layer surface 11 of the substrate 10, may follow the island growth model, where θ>0.

Particularly in cases where the patterning coating 110 may exhibit a substantially low initial sticking probability (in some non-limiting examples, under the conditions identified in the dual QCM technique described by Walker et al.) against deposition of the deposited material 331, there may be a substantially high thin film contact angle of the deposited material 331.

On the contrary, when a deposited material 331 may be selectively deposited on an exposed layer surface 11 without the use of a patterning coating 110, in some non-limiting examples, by employing a shadow mask 215, the nucleation and growth mode of such deposited material 331 may differ. In some non-limiting examples, it has been observed that a coating formed using a shadow mask 215 patterning process may, at least in some non-limiting examples, exhibit a substantially low thin film contact angle of no more than about 10°.

It has now been found, that in some non-limiting examples, a patterning coating 110 (including without limitation, the patterning material 211 of which it is comprised) may exhibit a substantially low critical surface tension.

Those having ordinary skill in the relevant art will appreciate that a “surface energy” of at least one of: a coating, layer, and a material constituting such at least one of: a coating, and layer, may generally correspond to a critical surface tension of the at least one of: coating, layer, and material. According to some models of surface energy, the critical surface tension of a surface may correspond substantially to the surface energy of such surface.

Generally, a material with a low surface energy may exhibit low intermolecular forces. Generally, a material with low intermolecular forces may readily one of: crystallize, and undergo other phase transformation, at a lower temperature in comparison to another material with high intermolecular forces. In at least some applications, a material that may readily one of: crystallize, and undergo other phase transformations, at substantially low temperatures may be detrimental to at least one of: the long-term performance, stability, reliability, and lifetime, of the device.

Without wishing to be bound by a particular theory, it may be postulated that certain low energy surfaces may exhibit substantially low initial sticking probabilities and may thus have applicability for forming the patterning coating 110.

Without wishing to be bound by any particular theory, it may be postulated that, especially for low surface energy surfaces, the critical surface tension may be positively correlated with the surface energy. In some non-limiting examples, a surface exhibiting a substantially low critical surface tension may also exhibit a substantially low surface energy, and a surface exhibiting a substantially high critical surface tension may also exhibit a substantially high surface energy.

In reference to Young's equation (Equation (13)), a lower surface energy may result in a greater contact angle, while also lowering the γₛᵥ, thus enhancing the likelihood of such surface having low wettability and low initial sticking probability with respect to the deposited material 331.

The critical surface tension values, in various non-limiting examples, herein may correspond to such values measured at around normal temperature and pressure (NTP), which in some non-limiting examples, may correspond to a temperature of 20° C., and an absolute pressure of 1 atm. In some non-limiting examples, the critical surface tension of a surface may be determined according to the Zisman method, as further detailed in Zisman, W. A., “Advances in Chemistry” 43 (1964), p. 1-51.

In some non-limiting examples, the exposed layer surface 11 of the patterning coating 110 may exhibit a critical surface tension of one of no more than about: 20 dynes/cm, 19 dynes/cm, 18 dynes/cm, 17 dynes/cm, 16 dynes/cm, 15 dynes/cm, 13 dynes/cm, 12 dynes/cm, and 11 dynes/cm.

In some non-limiting examples, the exposed layer surface 11 of the patterning coating 110 may exhibit a critical surface tension of one of at least about: 6 dynes/cm, 7 dynes/cm, 8 dynes/cm, 9 dynes/cm, and 10 dynes/cm.

Those having ordinary skill in the relevant art will appreciate that various methods and theories for determining the surface energy of a solid may be known. In some non-limiting examples, the surface energy may be calculated (derived) based on a series of measurements of contact angle, in which various liquids are brought into contact with a surface of a solid to measure the contact angle between the liquid-vapor interface and the surface. In some non-limiting examples, the surface energy of a solid surface may be equal to the surface tension of a liquid with the highest surface tension that completely wets the surface. In some non-limiting examples, a Zisman plot may be used to determine the highest surface tension value that would result in a contact angle of 0° with the surface. According to some theories of surface energy, various types of interactions between solid surfaces and liquids may be considered in determining the surface energy of the solid. In some non-limiting examples, according to some theories, including without limitation, at least one of: the Owens/Wendt theory, and Fowkes' theory, the surface energy may comprise a dispersive component and a non-dispersive (“polar”) component.

Without wishing to be bound by a particular theory, it may be postulated that, in some non-limiting examples, the contact angle of a coating of deposited material 331 may be determined, based at least partially on the properties (including, without limitation, initial sticking probability) of the patterning coating 110 onto which the deposited material 331 is deposited. Accordingly, patterning materials 211 that allow selective deposition of deposited materials 331 exhibiting substantially high contact angles may provide some benefit.

Those having ordinary skill in the relevant art will appreciate that various methods may be used to measure a contact angle θ, including without limitation, at least one of: the static, and dynamic, sessile drop method and the pendant drop method.

In some non-limiting examples, the activation energy for desorption (E_{des} 1431) (in some non-limiting examples, at a temperature T of about 300K) may be one of no more than about: 2 times, 1.5 times, 1.3 times, 1.2 times, 1.0 times, 0.8 times, and 0.5 times, the thermal energy. In some non-limiting examples, the activation energy for surface diffusion (Eₛ 1421) (in some non-limiting examples, at a temperature of about 300K) may exceed one of about: 1.0 times, 1.5 times, 1.8 times, 2 times, 3 times, 5 times, 7 times, and 10 times the thermal energy.

Without wishing to be bound by a particular theory, it may be postulated that, during thin film nucleation and growth of a deposited material 331 proximate to an interface between the exposed layer surface 11 of the underlying layer 210 and the patterning coating 110, a substantially high contact angle between the edge of the deposited material 331 and the underlying layer 210 may be observed due to the inhibition of nucleation of the solid surface of the deposited material 331 by the patterning coating 110. Such nucleation inhibiting property may be driven by minimization of surface energy between the underlying layer 210, thin film vapor and the patterning coating 110.

One measure of at least one of: a nucleation-inhibiting, and nucleation-promoting, property of a surface may be an initial deposition rate of a given (electrically conductive) deposited material 331, on the surface, relative to an initial deposition rate of the same deposited material 331 on a reference surface, where both surfaces are subjected to, (including without limitation, exposed to) an evaporation flux of the deposited material 331.

### Computer Device for Performing Method Actions

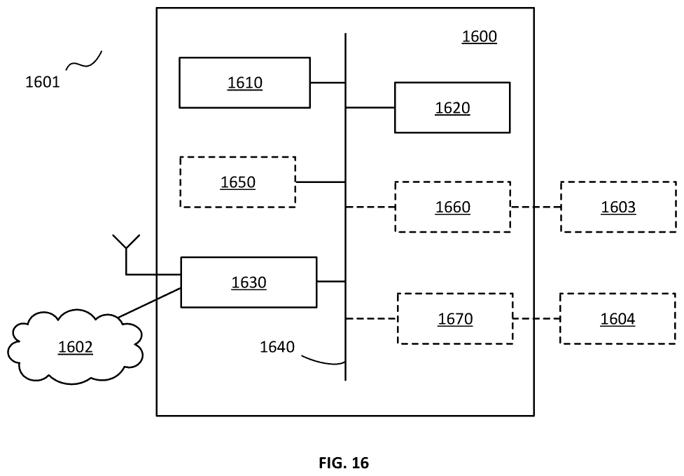

FIG. 16 is a simplified block diagram of a computing device 1600 illustrated within a computing and communications environment 1601, according to an example, that may be used for implementing the devices and methods disclosed herein.

In some non-limiting examples, the device 1600 may comprise a processor 1610, a memory 1620, a network interface 1630, and a bus 1640. In some non-limiting examples, the device 1600 may comprise a storage unit 1650, a video adapter 1660 and a peripheral interface 1670.

In some non-limiting examples, the device 1600 may utilize one of: all of the components shown, and only a subset thereof, and levels of integration may vary from device to device.

In some non-limiting examples, the device 1600 may comprise a plurality of instances of a component.

In some non-limiting examples, the processor 1610 may comprise a central processing unit (CPU), which in some non-limiting examples, may be one of: a single core processor, a multiple core processor, and a plurality of processors for parallel processing, and in some non-limiting examples, may comprise at least one of: a general-purpose processor, a dedicated application-specific specialized processor, including without limitation, a multiprocessor, a microcontroller, a reduced instruction set computer (RISC), a digital signal processor (DSP), a graphics processing unit (GPU), and the like, and a shared-purpose processor. In some non-limiting examples, the processor 1610 may comprise at least one of: dedicated hardware, and hardware capable of executing software. In some non-limiting examples, the processor 1610 may be part of a circuit, including without limitation, an integrated circuit. In some non-limiting examples, at least one other component of the device 1600 may be embodied in the circuit. In some non-limiting examples, the circuit may be one of: an application-specific integrated circuit (ASIC), and a floating-point gate array (FPGA).

In some non-limiting examples, the processor 1610 may control the general operation of the device 1600, in some non-limiting examples, by sending at least one of: data, and control signals, to at least one of: the memory 1620, the network interface 1630, the storage unit 1650, the video adapter 1660, and the peripheral interface 1670, and by retrieving at least one of: data, and instructions, from at least one of: the memory 1620, and the storage unit 1650, to execute methods disclosed herein. In some non-limiting examples, such instructions may be executed in at least one of: simultaneous, serial, and distributed fashion, by at least one processor 1610.

In some non-limiting examples, the processor 1610 may execute a sequence of one of: machine-readable, and machine-executable, instructions, which may be embodied in one of: a program, and software. In some non-limiting examples, the program may be stored in one of: the memory 1620, and the storage unit 1650. In some non-limiting examples, the program may be retrieved from one of: the memory 1620, and the storage unit 1650, and stored in the memory 1620 for ready access, and execution, by the processor 1610. In some non-limiting examples, the program may be directed to the processor 1610, which may subsequently configure the processor 1610 to implement methods of the present disclosure. Non-limiting examples of operations performed by the processor 1610 include at least one of: fetch, decode, execute, and writeback.

In some non-limiting examples, the program may be one of: pre-compiled, and configured for use with a machine having a processor adapted to execute the instructions and may be compiled during run-time. In some non-limiting examples, the program may be supplied in a programming language that may be selected to enable the instructions to execute in one of: a pre-compiled, interpreted, and an as-compiled, fashion.

However configured, the hardware of the processor 1610 may be configured so as to be capable of operating with sufficient software, processing power, memory resources, and network throughput capability, to handle any workload placed upon it.

In some non-limiting examples, the memory 1620 may be a storage device configured to store data, programs, in the form of one of: machine-readable, and machine-executable, instructions, and other information accessible within the device 1600, along the bus 1640.

In some non-limiting examples, the memory 1620 may comprise any type of transitory and non-transitory memory, including without limitation, at least one of: persistent, non-persistent, and volatile storage, including without limitation, system memory, readable by the processor 1610, including without limitation, semiconductor memory devices, including without limitation, random access memory (RAM), static RAM (SRAM), dynamic RAM (DRAM), synchronous DRAM (SDRAM), read-only memory (ROM), programmable ROM (PROM), erasable PROM (EPROM), and electrically erasable PROM (EEPROM), and at least one buffer circuit including without limitation, at least one of: latches and flip flops. In some non-limiting examples, the memory 1620 may comprise a plurality of types of memory, including without limitation, ROM for use at boot-up, and DRAM for program and data storage for use while executing programs.

In some non-limiting examples, the network interface 1630 may allow the device 1600 to communicate with remote entities, across at least one of: a telecommunications network, and a data network (network) 1602, including without limitation, at least one of: the Internet, an intranet, including without limitation, one in communication with the Internet, and an extranet, including without limitation, one in communication with the Internet, and may comprise at least one of: a network adapter, a wired network interface, including without limitation, a local area network (LAN) card, including without limitation, an ethernet card, a token ring card, and a fiber distributed data interface (FDDI) card, and a wireless network interface, including without limitation, a WIFI network interface, a modem, a modem bank, and a wireless LAN (WLAN) card, and a radio access network (RAN) interface, including without limitation, a radio transceiver card, to connect to other devices over a radio link.

In some non-limiting examples, the network 1602 may comprise at least one computer server, which may, in some non-limiting examples, comprise a device 1600, and which, in some non-limiting examples, may enable distributed computing, including without limitation, cloud computing. In some non-limiting examples, the network 1602, with the aid of the device 1600, may implement a peer-to-peer network, which may enable devices coupled with the device 1600, to behave as one of: a client, and a server.

In some non-limiting examples, the device 1600 may be a stand-alone device, while in some non-limiting examples, the device 1600 may be resident within a data centre. In some non-limiting examples, a data centre, as will be apparent to those having ordinary skill in the relevant art, may be a collection of computing resources (in some non-limiting examples, in the form of services) that may be used as a collective computing and storage resource. In some non-limiting examples, within a data centre, a plurality of services may be coupled together to provide a computing resource pool upon which virtualized entities may be instantiated. In some non-limiting examples, data centres may be coupled with each other to form networks comprising pooled computing and storage resources coupled with each other by connectivity resources. In some non-limiting examples, the connectivity resources may take the form of physical connections, including without limitation, Ethernet and optical communication links, and in some non-limiting examples, may comprise wireless communication channels as well. In some non-limiting examples, if a plurality of different data centres are coupled by a plurality of different communication channels, the links may be combined using any number of techniques, including without limitation, the formation of link aggregation groups (LAGs).

In some non-limiting examples, at least some of the computing, storage, and connectivity resources (along with other resources within the network 1602) may be divided between different sub-networks, in some cases in the form of a resource slice. In some non-limiting examples, if the resources across a number of connected at least one of: data centres, and collections of nodes, are sliced, different network slices may be created.

The device 1600 may, in some non-limiting examples, be schematically thought of, and described, in terms of a number of functional units, each of which has been described in the present disclosure.

In some non-limiting examples, the device 1600 may communicate with at least one remote device 1600, through the network 1602. In some non-limiting examples, the remote device 1600 may access the device 1600, via the network 1602.

In some non-limiting examples, the bus 1640 may couple the components of the device 1600 to facilitate the exchange of data, programs, and other information, within the device 1600 between components thereof. The bus 1640 may comprise at least one type of bus architecture, including without limitation, a memory bus, a memory controller, a peripheral bus, a video bus, and a motherboard.

In some non-limiting examples, the storage unit 1650 may be one of: a storage device that may, in some non-limiting examples, comprise at least one of: a solid-state memory device, a FLASH memory device, a solid-state drive, a hard disk drive, a magnetic disk drive, a magneto-optical disk, an optical memory, and an optical disk drive, and a data repository, for storing at least one of: data, including without limitation, user data, including without limitation, at least one of: user preferences, and user programs, and files, including without limitation, at least one of: drivers, libraries, and saved programs.

In some non-limiting examples, the storage unit 1650 may be distinguished from the memory 1620 in that it may perform storage tasks compatible with at least one of: higher latency, and lower volatility. In some no-limiting examples, the storage unit 1650 may be integrated with a heterogeneous memory 1620. In some non-limiting examples, the storage unit 1650 may be external to, and remote from, the device 1600, and accessible through use of the network interface 1630.

In some non-limiting examples, the video adapter 1660, including without limitation, an electronic display adapter, may provide interfaces to couple the device 1600 to external input and output (I/O) devices, including without limitation, one of: a display 1603, a monitor, a liquid crystal display (LCD), and a light-emitting diode (LED), coupled therewith.

In some non-limiting examples, the display 1603 may comprise a user interface (UI) 1604, including without limitation, a graphical user interface (GUI), and a web-based UI, for managing and organizing at least one of: inputs provided to, and outputs generated by the display 1603, including without limitation, at least one of: results, and solutions to the problems described herein.

In some non-limiting examples, the peripheral interface, including without limitation, at least one of: a parallel interface, and a serial interface, including without limitation, a universal serial bus (USB) interface, may be coupled with other I/O devices 1604, including without limitation, an input part of the display 1603, a touch screen, a printer, a keyboard, a keypad, a switch, a dial, a mouse, a trackball, a track pad, a biometric recognition (and input) device, a card reader, a paper tape reader, a camera, a sensor, a peripheral device, and a memory 1620, coupled therewith.

In some non-limiting examples, the device 1600 may be embodied as at least (part of) one of: a personal computer (PC), a desktop computer, a computer workstation, a mini computer, a mainframe computer, a laptop, and a mobile electronic device, including without limitation, a tablet (slate) PC (including without limitation, at least one of: Apple® iPad and Samsung® Galaxy Tab), a mobile telephone (including without limitation, a smartphone (including without limitation, at least one of: Apple® iPhone, Android-enabled device, and Blackberry© device), an e-reader, and a personal digital assistant).

Other components, as well as related functionality, of the device 1600, may have been omitted in order not to obscure the concepts presented herein.

In general terms each functional unit of the present disclosure may be implemented in at least one of: hardware, software, and firmware, as the context dictates. In some non-limiting examples, the processor 1610 may thus be arranged to fetch instructions from at least one of: the memory 1620, and the storage unit 1650, as provided by a functional unit of the present disclosure, to execute these instructions, thereby performing any of at least one of: an action, and an operation, as were described herein.

Aspects of the systems and methods provided herein, including without limitation, the device 1600, may be embodied in programming. Various aspects of the technology may be thought of as one of: “products”, and “articles of manufacture”, in some non-limiting examples, in the form of at least one of: machine-executable instructions, including without limitation, processor-executable instructions, and associated data, that is one of: carried on, and embodied in, a type of machine-readable medium.

In some non-limiting examples, “storage”-type media may include at least one of: the tangible memory of the device 1600, including without limitation, the processor 1610, and associated modules thereof, including without limitation, at least one of: various semiconductor memories, tape drives, and disk drives, of at least one of the memory 1620, and the storage unit 1650, which may provide non-transitory storage at any time for the software programming. In some non-limiting examples, one of: all, and parts, of the software may at times be communicated through the network 1602. In some non-limiting examples, such communications may enable loading of the software from one computer, including without limitation, the device 1600, including without limitation, a processor 1610 thereof, into another computer, including without limitation, a processor 1610 thereof, including without limitation, from one of: a management server, and a host computer, into the computer platform of an application server.

In some non-limiting examples, “storage”-type media that may bear the software elements of at least one functional unit of the present disclosure, may include at least one of: optical, electrical, and electromagnetic (EM) signals, including without limitation, such signals, including without limitation, waves, used across physical interfaces between local devices, through at least one of: wired, including without limitation a baseband signal, and optical, landline networks, and over various air-links, including without limitation, a signal embodied in a carrier wave. The physical elements that carry such signals, including without limitation, at least one of: the wired links, including without limitation, electrical conductors, including without limitation, coaxial cables, and waveguides, wireless links, including without limitation, those propagating through at least one of: the air, and free space, and optical links, including without limitation, optical media, including without limitation, optical fibre, also may be considered as “storage”-type media bearing the software.

As used herein, unless expressly restricted to non-transitory, tangible “storage” media, terms, including without limitation, one of: “computer-readable medium”, and “machine-readable medium” may refer to any medium that participates in providing instructions to a processor 1610 for execution. Such signals, including without limitation, other types of signals, including without limitation, those currently used and hereafter developed, referred to herein as the transmission medium, may be generated according to several well-known methods.

In some non-limiting examples, the information contained in such signals may be ordered according to different sequences, with applicability for at least one of: processing, and generating the information, and receiving the information.

In some non-limiting examples, a machine-readable medium, including without limitation, computer-executable code, may take many forms, including without limitation, at least one of: a tangible storage medium, a carrier wave medium, and a physical transmission medium.

In some non-limiting examples, non-volatile storage media may comprise one of: optical, and magnetic, disks, including without limitation, any of the storage devices 1620, 1650 in any device(s) 1600, including without limitation, one that may be used to implement the databases and at least some other associated components shown in the drawings.

In some non-limiting examples, volatile storage media may comprise dynamic memory, including without limitation, main memory 1620 of such a computer system 1600.

In some non-limiting examples, tangible transmission media may comprise at least one of: coaxial cables, copper wire, and fiber optics, including without limitation, the wires that comprise a bus 1640 within a computer system 1600.

In some non-limiting examples, carrier-wave transmission media may take the form of one of: electric signals, electromagnetic signals, acoustic waves, and light waves, including without limitation, those generated during radio frequency (RF) and infrared (IR) data communication.

Non-limiting example forms of computer-readable media include at least one of: a floppy disk, a flexible disk, a hard disk, a magnetic tape, any other magnetic medium, a CD-ROM, a DVD, a DVD-ROM, any other optical medium, punch cards, paper tape, any other physical storage medium with patterns of holes, a RAM, a ROM, a PROM, an EPROM, an EEPROM, a FLASH-EPROM, any other one of: a memory chip, and cartridge, a carrier wave transporting one of: data, and instructions, one of: cables, and links, transporting such a carrier wave, and any other medium from which a computer system 1600 may read one of: programming code, and data. In some non-limiting examples, many of these forms of computer-readable media may be involved in carrying at least one sequence of at least one instruction to a processor 1610 for execution.

### Definitions

In some non-limiting examples, the opto-electronic device may be an electro-luminescent device. In some non-limiting examples, the electro-luminescent device may be an organic light-emitting diode (OLED) device. In some non-limiting examples, the electro-luminescent device may be part of an electronic device. In some non-limiting examples, the electro-luminescent device may be an OLED lighting panel, including without limitation, a module thereof, including without limitation, an OLED display, including without limitation, a module thereof, of a computing device, such as a smartphone, a tablet, a laptop, an e-reader, a monitor, and a television set.

In some non-limiting examples, the opto-electronic device may be an organic photo-voltaic (OPV) device that converts photons into electricity. In some non-limiting examples, the opto-electronic device may be an electro-luminescent QD device.

In the present disclosure, unless specifically indicated to the contrary, reference will be made to OLED devices, with the understanding that such disclosure could, in some examples, equally be made applicable to other opto-electronic devices, including without limitation, at least one of: an OPV, and QD device, in a manner apparent to those having ordinary skill in the relevant art.

The structure of such devices may be described from each of two aspects, namely from at least one of: a longitudinal aspect, and from a lateral (plan view) aspect.

In the present disclosure, a directional convention may be followed, extending substantially normally to the lateral aspect described above, in which the substrate may be the “bottom” of the device, and the layers may be disposed on “top” of the substrate. Following such convention, the second electrode may be at the top of the device shown, even if (as may be the case in some examples, including without limitation, during a manufacturing process, in which at least one layers may be introduced by means of a vapor deposition process), the substrate may be physically inverted, such that the top surface, in which one of the layers, such as, without limitation, the first electrode, may be disposed, may be physically below the substrate, to allow the deposition material (not shown) to move upward and be deposited upon the top surface thereof as a thin film.

In the context of introducing the longitudinal aspect herein, the components of such devices may be shown in substantially planar lateral strata. Those having ordinary skill in the relevant art will appreciate that such substantially planar representation may be for purposes of illustration only, and that across a lateral extent of such a device, there may be localized substantially planar strata of different thicknesses and dimension, including, in some non-limiting examples, the substantially complete absence of a layer(s) separated by non-planar transition regions (including lateral gaps and even discontinuities). Thus, while for illustrative purposes, the device may be shown below in its longitudinal aspect as a substantially stratified structure, in the plan view aspect discussed below, such device may illustrate a diverse topography to define features, each of which may substantially exhibit the stratified profile discussed in the longitudinal aspect.

In the present disclosure, the terms “layer” and “strata” may be used interchangeably to refer to similar concepts.

The thickness of each layer shown in the figures may be illustrative only and not necessarily representative of a thickness relative to another layer.

In the present disclosure, a first layer may be said to be deposited on an exposed layer surface of a second layer to form a layer interface therebetween. Those having ordinary skill in the relevant art will appreciate that at the time of deposition of the first layer, the material from which the first layer will be comprised is deposited on a surface of the second layer that is one of: “presented”, and “exposed”, in that there is substantially no material deposited thereon, such that it is available to accept deposition thereon of the material from which the first layer will be composed.

Accordingly, as used herein, the surface of the second layer presented, at the time of deposition, for deposition thereon of the material from which the first layer will be composed, may be said to be an “exposed layer surface” of the second layer, even if, in a device in which deposition has proceeded further, including without limitation, to completion, such surface may no longer be “exposed”, because of the deposition thereon of the material from which the first layer may be composed.

Those having ordinary skill in the relevant art will appreciate that a third layer may be said to be deposited on an exposed layer surface of the first layer to form a layer interface therein. Thus, after deposition of the first layer onto the exposed layer surface of the second layer, and after deposition of the third layer onto the exposed layer surface of the first layer, the first layer may be said to extend between the second layer and the third layer, and concomitantly, the first layer may be said to extend between the layer interface between the first layer and the second layer, and the layer interface between the third layer and the first layer.

For purposes of simplicity of description, in the present disclosure, a combination of a plurality of elements in a single layer may be denoted by a colon “:”, while a plurality of (combination(s) of) elements comprising a plurality of layers in a multi-layer coating may be denoted by separating two such layers by a slash “/”. In some non-limiting examples, the layer after the slash may be deposited at least one of: after, and on, the layer preceding the slash.

For purposes of illustration, an exposed layer surface of an underlying layer, onto which at least one of: a coating, layer, and material, may be deposited, may be understood to be a surface of such underlying layer that may be presented for deposition of at least one of: the coating, layer, and material, thereon, at the time of deposition.

Those having ordinary skill in the relevant art will appreciate that when one of: a component, a layer, a region, and a portion thereof, is referred to as being at least one of: “formed”, “disposed”, and “deposited” on, and “deposited” over another underlying at least one of: a material, component, layer, region, and/portion, such at least one of: formation, disposition, and deposition, may be one of: directly, and indirectly, on an exposed layer surface (at the time of such at least one of: formation, disposition, and deposition) of such underlying at least one of: material, component, layer, region, and portion, with the potential of intervening at least one of: material(s), component(s), layer(s), region(s), and portion(s) therebetween.

In the present disclosure, the terms “overlap”, and “overlapping” may refer generally to a plurality of at least one of: layers, and structures, arranged to intersect a cross-sectional axis extending substantially normally away from a surface onto which such at least one of: layers, and structures, may be disposed.

While the present disclosure discusses thin film formation, in reference to at least one layer (coating), in terms of vapor deposition, those having ordinary skill in the relevant art will appreciate that, in some non-limiting examples, various components of the device may be selectively deposited using a wide variety of techniques, including without limitation, evaporation (including without limitation, at least one of: thermal, and electron beam, evaporation), photolithography, printing (including without limitation, ink jet, and vapor jet, printing, reel-to-reel printing, and micro-contact transfer printing), PVD (including without limitation, sputtering), chemical vapor deposition (CVD) (including without limitation, at least one of: plasma-enhanced CVD (PECVD), and organic vapor phase deposition (OVPD)), laser annealing, laser-induced thermal imaging (LITI) patterning, atomic-layer deposition (ALD), coating (including without limitation, spin-coating, di coating, line coating, and spray coating) (collectively “deposition process”).

Some processes may be used in combination with a shadow mask, which may, in some non-limiting examples, may be one of: an open mask, and FMM, during deposition of any of various at least one of: layers, and coatings, to achieve various patterns by at least one of: masking, and precluding deposition of, a deposited material on certain parts of a surface of an underlying layer exposed thereto.

In the present disclosure, the terms “evaporation”, and “sublimation” may be used interchangeably to refer generally to deposition processes in which a source material is converted into a vapor, including without limitation, by heating, to be deposited onto a target surface in, without limitation, a solid state. As will be understood, an evaporation deposition process may be a type of PVD process where at least one source material is sublimed under a low pressure (including without limitation, a vacuum) environment to form vapor monomers, and deposited on a target surface through de-sublimation of the at least one evaporated source material. A variety of different evaporation sources may be used for heating a source material, and, as such, it will be appreciated by those having ordinary skill in the relevant art, that the source material may be heated in various ways. In some non-limiting examples, the source material may be heated by at least one of: an electric filament, electron beam, inductive heating, and by resistive heating. In some non-limiting examples, the source material may be loaded into at least one of: a heated crucible, a heated boat, a Knudsen cell (which may be an effusion evaporator source), and any other type of evaporation source.

In some non-limiting examples, a deposition source material may be a mixture. In some non-limiting examples, at least one component of a mixture of a deposition source material may not be deposited during the deposition process (in some non-limiting examples, be deposited in a substantially small amount compared to other components of such mixture).

In the present disclosure, a reference to at least one of: a layer thickness, a film thickness, and an average one of: layer, and film, thickness, of a material, irrespective of the mechanism of deposition thereof, may refer to an amount of the material deposited on a target exposed layer surface, which corresponds to an amount of the material to cover the target surface with a uniformly thick layer of the material having the referenced layer thickness. In some non-limiting examples, depositing a layer thickness of 10 nm of material may indicate that an amount of the material deposited on the surface may correspond to an amount of the material to form a uniformly thick layer of the material that may be 10 nm thick. It will be appreciated that, having regard to the mechanism by which thin films are formed discussed above, in some non-limiting examples, due to possible at least one of: stacking, and clustering, of monomers, an actual thickness of the deposited material may be non-uniform. In some non-limiting examples, depositing a layer thickness of 10 nm may yield one of: some parts of the deposited material having an actual thickness greater than 10 nm, and other parts of the deposited material having an actual thickness of no more than 10 nm. A certain layer thickness of a material deposited on a surface may thus correspond, in some non-limiting examples, to an average thickness of the deposited material across the target surface.

In the present disclosure, a reference to a reference layer thickness may refer to a layer thickness of the deposited material (such as Mg), that may be deposited on a reference surface exhibiting one of: a high initial sticking probability, and initial sticking coefficient, (that is, a surface having an initial sticking probability that is about 1.0). The reference layer thickness may not indicate an actual thickness of the deposited material deposited on a target surface (such as, without limitation, a surface of a patterning coating). Rather, the reference layer thickness may refer to a layer thickness of the deposited material that would be deposited on a reference surface, in some non-limiting examples, a surface of a quartz crystal, positioned inside a deposition chamber for monitoring a deposition rate and the reference layer thickness, upon subjecting the target surface and the reference surface to identical vapor flux of the deposited material for the same deposition period. Those having ordinary skill in the relevant art will appreciate that in the event that the target surface and the reference surface are not subjected to identical vapor flux simultaneously during deposition, an appropriate tooling factor may be used to determine (monitor) the reference layer thickness.

In the present disclosure, a reference deposition rate may refer to a rate at which a layer of the deposited material would grow on the reference surface, if it were identically positioned and configured within a deposition chamber as the sample surface.

In the present disclosure, a reference to depositing a number X of monolayers of material may refer to depositing an amount of the material to cover a given area of an exposed layer surface with X single layer(s) of constituent monomers of the material, such as, without limitation, in a closed coating.

In the present disclosure, a reference to depositing a fraction of a monolayer of a material may refer to depositing an amount of the material to cover such fraction of a given area of an exposed layer surface with a single layer of constituent monomers of the material. Those having ordinary skill in the relevant art will appreciate that due to, in some non-limiting examples, possible at least one of: stacking, and clustering, of monomers, an actual local thickness of a deposited material across a given area of a surface may be non-uniform. In some non-limiting examples, depositing 1 monolayer of a material may result in some local regions of the given area of the surface being uncovered by the material, while other local regions of the given area of the surface may have multiple at least one of: atomic, and molecular, layers deposited thereon.

In the present disclosure, a target surface (including without limitation, target region(s) thereof) may be considered to be at least one of: “substantially devoid of”, “substantially free of”, and “substantially uncovered by”, a material if there may be a substantial absence of the material on the target surface as determined by any applicable determination mechanism.

In the present disclosure, the terms “sticking probability” and “sticking coefficient” may be used interchangeably.

In the present disclosure, the term “nucleation” may reference a nucleation stage of a thin film formation process, in which monomers in a vapor phase condense onto a surface to form nuclei.

In the present disclosure, in some non-limiting examples, as the context dictates, the terms “patterning coating” and “patterning material” may be used interchangeably to refer to similar concepts, and references to a patterning coating herein, in the context of being selectively deposited to pattern a deposited layer may, in some non-limiting examples, be applicable to a patterning material in the context of selective deposition thereof to pattern at least one of: a deposited material, and an electrode coating material.

Similarly, in some non-limiting examples, as the context dictates, the term “patterning coating” and “patterning material” may be used interchangeably to refer to similar concepts, and reference to an NPC herein, in the context of being selectively deposited to pattern a deposited layer may, in some non-limiting examples, be applicable to an NPC in the context of selective deposition thereof to pattern at least one of: a deposited material, and an electrode coating.

While a patterning material may be one of: nucleation-inhibiting, and nucleation-promoting, in the present disclosure, unless the context dictates otherwise, a reference herein to a patterning material is intended to be a reference to an NIC.

In some non-limiting examples, reference to a patterning coating may signify a coating having a specific composition as described herein.

In the present disclosure, the terms “deposited layer”, “conductive coating”, and “electrode coating” may be used interchangeably to refer to similar concepts and references to a deposited layer herein, in the context of being patterned by selective deposition of at least one of: a patterning coating, and an NPC, may, in some non-limiting examples, be applicable to a deposited layer in the context of being patterned by selective deposition of a patterning material. In some non-limiting examples, reference to an electrode coating may signify a coating having a specific composition as described herein. Similarly, in the present disclosure, the terms “deposited layer material”, “deposited material”, “conductive coating material”, and “electrode coating material” may be used interchangeably to refer to similar concepts and references to a deposited material herein.

In the present disclosure, as used herein, molecular formulae showing fragment(s) of a compound may comprise at least one bond connected to symbols, including without limitation, an asterisk symbol (denoted “*”), and those denoted

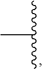

which symbols may be used to indicate the bonds to another atom (not shown) of the compound to which such fragment(s) may be attached.

In the present disclosure, it will be appreciated by those having ordinary skill in the relevant art that an organic material may comprise, without limitation, a wide variety of organic at least one of: molecules, and polymers. Further, it will be appreciated by those having ordinary skill in the relevant art that organic materials that are doped with various inorganic substances, including without limitation, elements, and inorganic compounds, may still be considered organic materials. Still further, it will be appreciated by those having ordinary skill in the relevant art that various organic materials may be used, and that the processes described herein are generally applicable to an entire range of such organic materials. Still further, it will be appreciated by those having ordinary skill in the relevant art that organic materials that comprise at least one of: metals, and other organic elements, may still be considered as organic materials. Still further, it will be appreciated by those having ordinary skill in the relevant art that various organic materials may be at least one of: molecules, oligomers, and polymers.

An organic opto-electronic device may encompass any opto-electronic device where at least one active layers (strata) thereof are formed primarily of an organic (carbon-containing) material, and more specifically, an organic semiconductor material.

In the present disclosure, the term “organic-inorganic hybrid material”, as used herein, may generally refer to a material that comprises both an organic component and an inorganic component. In some non-limiting examples, such organic-inorganic hybrid material may comprise an organic-inorganic hybrid compound that comprises an organic moiety and an inorganic moiety. In some non-limiting examples, such organic-inorganic hybrid compounds may include those in which an inorganic scaffold may be functionalized with at least one organic functional group.

Non-limiting examples of such organic-inorganic hybrid materials include those comprising at least one of: a siloxane group, a silsesquioxane group, a polyhedral oligomeric silsesquioxane (POSS) group, a phosphazene group, and a metal complex.

In the present disclosure, a semiconductor material may be described as a material that generally exhibits a band gap. In some non-limiting examples, the band gap may be formed between a highest occupied molecular orbital (HOMO) and a lowest unoccupied molecular orbital (LUMO) of the semiconductor material. Semiconductor materials thus generally exhibit electrical conductivity that is no more than that of a conductive material (including without limitation, a metal), but that is greater than that of an insulating material (including without limitation, a glass). In some non-limiting examples, the semiconductor material may comprise an organic semiconductor material. In some non-limiting examples, the semiconductor material may comprise an inorganic semiconductor material.

As used herein, an oligomer may generally refer to a material which includes at least two monomer (units). As would be appreciated by a person skilled in the art, an oligomer may differ from a polymer in at least one aspect, including, without limitation: (1) the number of monomer units contained therein; (2) the molecular weight; and (3) other material properties (characteristics). In some non-limiting examples, further description of polymers and oligomers may be found in Naka K. (2014) Monomers, Oligomers, Polymers, and Macromolecules (Overview), and in Kobayashi S., Mullen K. (eds.) Encyclopedia of Polymeric Nanomaterials, Springer, Berlin, Heidelberg.

One of: an oligomer, and a polymer, may generally include monomer units that may be chemically bonded together to form a molecule. Such monomer units may be substantially identical to one another such that one of: the molecule is primarily formed by repeating monomer units, and the molecule may include a plurality of different monomer units. Additionally, the molecule may include at least one terminal unit, which may be different from the monomer units of the molecule. One of: an oligomer, and a polymer, may be at least one of: linear, branched, cyclic, cyclo-linear, and cross-linked. One of: an oligomer, and a polymer, may include a plurality of different monomer units which are arranged in a repeating pattern, including without limitation, in alternating blocks, of different monomer units.

In the present disclosure, the term “semiconducting layer(s)” may be used interchangeably with “organic layer(s)” since the layers in an OLED device may in some non-limiting examples, may comprise organic semiconducting materials.

In the present disclosure, an inorganic substance may refer to a substance that primarily includes an inorganic material. In the present disclosure, an inorganic material may comprise any material that is not considered to be an organic material, including without limitation, metals, glasses, and minerals.

In the present disclosure, the term “aperture ratio”, as used herein, generally refers to a percentage of area within a (part of a) display panel, in plan, occupied by, including without limitation, attributed to, at least one feature present in such (part of a) display panel.

In the present disclosure, the terms “EM radiation”, “photon”, and “light” may be used interchangeably to refer to similar concepts. In the present disclosure, EM radiation may have a wavelength that lies in at least one of: the visible spectrum, infrared (IR) region (IR spectrum), near IR region (NIR spectrum), ultraviolet (UV) region (UV spectrum), UVA region (UVA spectrum) (which may correspond to a wavelength range between about 315-400 nm) thereof, and UVB region (UVB spectrum) (which may correspond to a wavelength between about 280-315 nm) thereof.

In the present disclosure, the term “visible spectrum” as used herein, generally refers to at least one wavelength in the visible part of the EM spectrum.

As would be appreciated by those having ordinary skill in the relevant art, such visible part may correspond to any wavelength between about 380-740 nm. In general, electro-luminescent devices may be configured to at least one of: emit, and transmit, EM radiation having wavelengths in a range of between about 425-725 nm, and more specifically, in some non-limiting examples, EM radiation having peak emission wavelengths of 456 nm, 528 nm, and 624 nm, corresponding to B(lue), G(reen), and R(ed) sub-pixels, respectively. Accordingly, in the context of such electro-luminescent devices, the visible part may refer to any wavelength that is one of: between about 425-725 nm, and between about 456-624 nm. EM radiation having a wavelength in the visible spectrum may, in some non-limiting examples, also be referred to as “visible light” herein.

In the present disclosure, the term “emission spectrum” as used herein, generally refers to an electroluminescence spectrum of light emitted by an opto-electronic device. In some non-limiting examples, an emission spectrum may be detected using an optical instrument, such as, in some non-limiting examples, a spectrophotometer, which may measure an intensity of EM radiation across a wavelength range.

In the present disclosure, the term “onset wavelength”, as used herein, may generally refer to a lowest wavelength at which an emission is detected within an emission spectrum.

In the present disclosure, the term “peak wavelength”, as used herein, may generally refer to a wavelength at which a maximum luminous intensity is detected within an emission spectrum.

In some non-limiting examples, the onset wavelength may be less than the peak wavelength. In some non-limiting examples, the onset wavelength λₒₙₛₑₜ may correspond to a wavelength at which a luminous intensity is one of no more than about: 10%, 5%, 3%, 1%, 0.5%, 0.1%, and 0.01%, of the luminous intensity at the peak wavelength.

In some non-limiting examples, an emission spectrum that lies in the R(ed) part of the visible spectrum may be characterized by a peak wavelength that may lie in a wavelength range of about 600-640 nm and in some non-limiting examples, may be substantially about 620 nm.

In some non-limiting examples, an emission spectrum that lies in the G(reen) part of the visible spectrum may be characterized by a peak wavelength that may lie in a wavelength range of about 510-540 nm and in some non-limiting examples, may be substantially about 530 nm.

In some non-limiting examples, an emission spectrum that lies in the B(lue) part of the visible spectrum may be characterized by a peak wavelength λₘₐₓ that may lie in a wavelength range of about 450-460 nm and in some non-limiting examples, may be substantially about 455 nm.

In the present disclosure, the term “IR signal” as used herein, may generally refer to EM radiation having a wavelength in an IR subset (IR spectrum) of the EM spectrum. An IR signal may, in some non-limiting examples, have a wavelength corresponding to a near-infrared (NIR) subset (NIR spectrum) thereof. In some non-limiting examples, an NIR signal may have a wavelength of one of between about: 750-1400 nm, 750-1300 nm, 800-1300 nm, 800-1200 nm, 850-1300 nm, and 900-1300 nm.

In the present disclosure, the term “absorption spectrum”, as used herein, may generally refer to a wavelength (sub-) range of the EM spectrum over which absorption may be concentrated.

In the present disclosure, the terms “absorption edge”, “absorption discontinuity”, and “absorption limit” as used herein, may generally refer to a sharp discontinuity in the absorption spectrum of a substance. In some non-limiting examples, an absorption edge may tend to occur at wavelengths where the energy of absorbed EM radiation may correspond to at least one of: an electronic transition, and ionization potential.

In the present disclosure, the term “extinction coefficient” as used herein, may generally refer to a degree to which an EM coefficient may be attenuated when propagating through a material. In some non-limiting examples, the extinction coefficient may be understood to correspond to the imaginary component k of a complex refractive index. In some non-limiting examples, the extinction coefficient of a material may be measured by a variety of methods, including without limitation, by ellipsometry.

In the present disclosure, the terms “refractive index”, and “index”, as used herein to describe a medium, may refer to a value calculated from a ratio of the speed of light in such medium relative to the speed of light in a vacuum. In the present disclosure, particularly when used to describe the properties of substantially transparent materials, including without limitation, thin film layers (coatings), the terms may correspond to the real part, n, in the expression N=n+ik, in which N may represent the complex refractive index and k may represent the extinction coefficient.

As would be appreciated by those having ordinary skill in the relevant art, substantially transparent materials, including without limitation, thin film layers (coatings), may generally exhibit a substantially low extinction coefficient value in the visible spectrum, and therefore the imaginary component of the expression may have a negligible contribution to the complex refractive index. On the other hand, light-transmissive electrodes formed, for example, by a metallic thin film, may exhibit a substantially low refractive index value and a substantially high extinction coefficient value in the visible spectrum. Accordingly, the complex refractive index, N, of such thin films may be dictated primarily by its imaginary component k.

In the present disclosure, unless the context dictates otherwise, reference without specificity to a refractive index may be intended to be a reference to the real part n of the complex refractive index N.

In some non-limiting examples, there may be a generally positive correlation between refractive index and transmittance, in other words, a generally negative correlation between refractive index and absorption. In some non-limiting examples, the absorption edge of a substance may correspond to a wavelength at which the extinction coefficient approaches 0.

In the present disclosure, the concept of a pixel may be discussed on conjunction with the concept of at least one sub-pixel thereof. For simplicity of description only, such composite concept may be referenced herein as a “(sub-) pixel” and such term may be understood to suggest at least one of: a pixel, and at least one sub-pixel thereof, unless the context dictates otherwise.

In some nonlimiting examples, one measure of an amount of a material on a surface may be a percentage coverage of the surface by such material. In some non-limiting examples, surface coverage may be assessed using a variety of imaging techniques, including without limitation, at least one of: TEM, AFM, and SEM.

In the present disclosure, the terms “particle”, “island”, and “cluster” may be used interchangeably to refer to similar concepts.

In the present disclosure, for purposes of simplicity of description, the terms “coating film”, “closed coating”, and “closed film”, as used herein, may refer to a thin film structure (coating) of a deposited material used for a deposited layer, in which a relevant part of a surface may be substantially coated thereby, such that such surface may be not substantially exposed by (through) the coating film deposited thereon.

In the present disclosure, unless the context dictates otherwise, reference without specificity to a thin film may be intended to be a reference to a substantially closed coating.

In some non-limiting examples, a closed coating, in some non-limiting examples, of at least one of: a deposited layer, and a deposited material, may be disposed to cover a part of an underlying layer, such that, within such part, one of no more than about: 40%, 30%, 25%, 20%, 15%, 10%, 5%, 3%, and 1% of the underlying layer therewithin may be exposed by (through), the closed coating.

Those having ordinary skill in the relevant art will appreciate that a closed coating may be patterned using various techniques and processes, including without limitation, those described herein, to deliberately leave a part of the exposed layer surface of the underlying layer to be exposed after deposition of the closed coating. In the present disclosure, such patterned films may nevertheless be considered to constitute a closed coating, if, in some non-limiting examples, the thin film (coating) that is deposited, within the context of such patterning, and between such deliberately exposed parts of the exposed layer surface of the underlying layer, itself substantially comprises a closed coating.

Those having ordinary skill in the relevant art will appreciate that, due to inherent variability in the deposition process, and in some non-limiting examples, to the existence of impurities in at least one of the deposited materials, in some non-limiting examples, the deposited material, and the exposed layer surface of the underlying layer, deposition of a thin film, using various techniques and processes, including without limitation, those described herein, may nevertheless result in the formation of small apertures, including without limitation, at least one of: pin-holes, tears, and cracks, therein. In the present disclosure, such thin films may nevertheless be considered to constitute a closed coating, if, in some non-limiting examples, the thin film (coating) that is deposited substantially comprises a closed coating and meets any specified percentage coverage criterion set out, despite the presence of such apertures.

In the present disclosure, for purposes of simplicity of description, the term “discontinuous layer” as used herein, may refer to a thin film structure (coating) of a material used for a deposited layer, in which a relevant part of a surface coated thereby, may be neither substantially devoid of such material, nor forms a closed coating thereof. In some non-limiting examples, a discontinuous layer of a deposited material may manifest as a plurality of discrete islands disposed on such surface.

In the present disclosure, for purposes of simplicity of description, the result of deposition of vapor monomers onto an exposed layer surface of an underlying layer, that has not (yet) reached a stage where a closed coating has been formed, may be referred to as a “intermediate stage layer”. In some non-limiting examples, such an intermediate stage layer may reflect that the deposition process has not been completed, in which such an intermediate stage layer may be considered as an interim stage of formation of a closed coating. In some non-limiting examples, an intermediate stage layer may be the result of a completed deposition process, and thus constitute a final stage of formation in and of itself.

In some non-limiting examples, an intermediate stage layer may more closely resemble a thin film than a discontinuous layer but may have apertures (gaps) in the surface coverage, including without limitation, at least one of: a dendritic projection, and a dendritic recess. In some non-limiting examples, such an intermediate stage layer may comprise a fraction of a single monolayer of the deposited material such that it does not form a closed coating.

In the present disclosure, for purposes of simplicity of description, the term “dendritic”, with respect to a coating, including without limitation, the deposited layer, may refer to feature(s) that resemble a branched structure when viewed in a lateral aspect. In some non-limiting examples, the deposited layer may comprise at least one of: a dendritic projection, and a dendritic recess. In some non-limiting examples, a dendritic projection may correspond to a part of the deposited layer that exhibits a branched structure comprising a plurality of short projections that are physically connected and extend substantially outwardly. In some non-limiting examples, a dendritic recess may correspond to a branched structure of at least one of: gaps, openings, and uncovered parts, of the deposited layer that are physically connected and extend substantially outwardly. In some non-limiting examples, a dendritic recess may correspond to, including without limitation, a mirror image (inverse pattern) to the pattern of a dendritic projection. In some non-limiting examples, at least one of: a dendritic projection, and a dendritic recess may have a configuration that exhibits, (mimics) at least one of: a fractal pattern, a mesh, a web, and an interdigitated structure.

In some non-limiting examples, sheet resistance may be a property of at least one of: a component, layer, and part, that may alter a characteristic of an electric current passing through at least one of: such component, layer, and part. In some non-limiting examples, a sheet resistance of a coating may generally correspond to a characteristic sheet resistance of the coating, measured (determined) in isolation from other at least one of: components, layers, and parts, of the device.

In the present disclosure, a deposited density may refer to a distribution, within a region, which in some non-limiting examples may comprise at least one of: an area, and a volume, of a deposited material therein. Those having ordinary skill in the relevant art will appreciate that such deposited density may be unrelated to a density of mass (material) within a particle structure itself that may comprise such deposited material. In the present disclosure, unless the context dictates otherwise, reference to a (deposited) density, may be intended to be a reference to a distribution of such deposited material, including without limitation, as at least one particle, within an area.

In some non-limiting examples, a bond dissociation energy of a metal may correspond to a standard-state enthalpy change measured at 298 K from the breaking of a bond of a diatomic molecule formed by two identical atoms of the metal. Bond dissociation energies may, in some non-limiting examples, be determined based on known literature including without limitation, Luo, Yu-Ran, “Bond Dissociation Energies” (2010).

Without wishing to be bound by a particular theory, it is postulated that providing an NPC may facilitate deposition of the deposited layer onto certain surfaces.

Non-limiting examples of materials having applicability for forming an NPC may comprise without limitation, at least one metal, including without limitation, alkali metals, alkaline earth metals, transition metals, post-transition metals, metal fluorides, metal oxides, and fullerene.

Non-limiting examples of such materials include Ca, Ag, Mg, Yb, ITO, IZO, ZnO, ytterbium fluoride (YbF₃), magnesium fluoride (MgF₂), and cesium fluoride (CsF).

In the present disclosure, the term “fullerene” may refer generally to a material including carbon molecules. Non-limiting examples of fullerene molecules include carbon cage molecules, including without limitation, a three-dimensional skeleton that includes multiple carbon atoms that form a closed shell, and which may be, without limitation, (semi-)spherical in shape. In some non-limiting examples, a fullerene molecule may be designated as Cₙ, where n may be an integer corresponding to several carbon atoms included in a carbon skeleton of the fullerene molecule. Non-limiting examples of fullerene molecules include Cₙ, where n may be in the range of 50 to 250, such as, without limitation, C₆₀, C₇₀, C₇₂, C₇₄, C₇₆, C₇₈, C₈₀, C₈₂, and C₈₄. Additional non-limiting examples of fullerene molecules include carbon molecules in at least one of: a tube, and a cylindrical shape, including without limitation, single-walled carbon nanotubes, and multi-walled carbon nanotubes.

Based on findings and experimental observations, it may be postulated that nucleation promoting materials, including without limitation, fullerenes, metals, including without limitation, at least one of: Ag, and Yb, and metal oxides, including without limitation, ITO, and IZO, as discussed further herein, may act as nucleation sites for the deposition of a deposited layer, including without limitation Mg.

In some non-limiting examples, applicable materials for use to form an NPC, may include those exhibiting (characterized) as having an initial sticking probability for a material of a deposited layer of one of at least about: 0.4, 0.5, 0.6, 0.7, 0.75, 0.8, 0.9, 0.93, 0.95, 0.98, and 0.99.

In some non-limiting examples, in scenarios where Mg is deposited using without limitation, an evaporation process on a fullerene-treated surface, in some non-limiting examples, the fullerene molecules may act as nucleation sites that may promote formation of stable nuclei for Mg deposition.

In some non-limiting examples, no more than a monolayer of an NPC, including without limitation, fullerene, may be provided on the treated surface to act as nucleation sites for deposition of Mg.

In some non-limiting examples, treating a surface by depositing several monolayers of an NPC thereon may result in a higher number of nucleation sites and accordingly, a higher initial sticking probability.

Those having ordinary skill in the relevant art will appreciate than an amount of material, including without limitation, fullerene, deposited on a surface, may be one of: more, and less than, one monolayer. In some non-limiting examples, such surface may be treated by depositing one of about: 0.1, 1, 10, and more monolayers of at least one of: a nucleation promoting, and a nucleation inhibiting, material.

In some non-limiting examples, an average layer thickness of the NPC deposited on an exposed layer surface of underlying layer(s) may be one of between about: 1-5 nm, and 1-3 nm.

Where features and aspects of the present disclosure may be described in terms of Markush groups, it will be appreciated by those having ordinary skill in the relevant art that the present disclosure may also be thereby described in terms of any individual member of sub-group of members of such Markush group.

### Terminology

References in the singular form may include the plural and vice versa, unless otherwise noted.

As used herein, relational terms, such as “first” and “second”, and numbering devices such as “a”, “b” and the like, may be used solely to distinguish one entity/element from another entity/element, without necessarily requiring/implying any physical/logical relationship/order between such entities/elements.

The terms “including” and “comprising” may be used expansively and in an open-ended fashion, and thus should be interpreted to mean “including, but not limited to”. The terms “example” and “exemplary” may be used simply to identify instances for illustrative purposes and should not be interpreted as limiting the scope of the invention to the stated instances. In some non-limiting examples, the term “exemplary” should not be interpreted to denote/confer any laudatory, beneficial, and other quality to the expression with which it is used, whether in terms of design, performance and otherwise.

Further, the term “critical”, especially when used in the expressions “critical nuclei”, “critical nucleation rate”, “critical concentration”, “critical cluster”, “critical monomer”, “critical particle structure size”, and “critical surface tension” may be a term familiar to those having ordinary skill in the relevant art, including as relating to/being in a state in which a measurement/point at which some at least one of: quality, property and phenomenon undergoes a definite change. As such, the term “critical” should not be interpreted to denote/confer any significance/importance to the expression with which it is used, whether in terms of design, performance, and otherwise.

The term “common”, especially when used in the expressions “common electrode”, “common conductive coating”, and “common layer” may be intended to mean an electrode, conductive coating, and layer, as the case may be, that is one of: deposited as, and acts as it was deposited as, a single continuous single structure.

The terms “couple” and “communicate” in any form may be intended to mean either one of: a direct, and indirect, connection through some one of: an interface, device, intermediate component, connection, whether optically, electrically, mechanically, chemically, and otherwise.

The terms “on” and “over”, when used in reference to a first component relative to another component, and at least one of: “covering” and which “covers” another component, may encompass situations where the first component is directly on (including without limitation, in physical contact with) the other component, as well as cases where at least one intervening component is positioned between the first component and the other component.

Directional terms such as “upward”, “downward”, “left” and “right” may be used to refer to directions in the drawings to which reference is made unless otherwise stated. Similarly, words such as “inward” and “outward” may be used to refer to directions toward and away from, respectively, the geometric center of the device, area, volume and designated parts thereof. Moreover, all dimensions described herein may be intended solely to be by way of example of purposes of illustrating certain examples and may not be intended to limit the scope of the disclosure to any examples that may depart from such dimensions as may be specified.

As used herein, the terms “substantially”, “substantial”, “approximately”, and “about” may be used to denote and account for small variations. When used in conjunction with an event/circumstance, such terms may refer to instances in which the event/circumstance occurs precisely, as well as instances in which the event/circumstance occurs to a close approximation. In some non-limiting examples, when used in conjunction with a numerical value, such terms may refer to a range of variation of no more than about ±10% of such numerical value, such as at least one of no more than about: ±5%, ±4%, ±3%, ±2%, ±1%, ±0.5%, ±0.1%, and ±0.05%.

As used herein, the phrase “consisting substantially of” may be understood to include those elements specifically recited and any additional elements that do not materially affect the basic and novel characteristics of the described technology, while the phrase “consisting of” without the use of any modifier, may exclude any element not specifically recited.

Whenever the term “at least” precedes the first numerical value in a series of a plurality numerical values, the term “at least” may apply to each of the numerical values in that series of numerical values. In some non-limiting examples, at least one of: 1, 2, and 3 may be equivalent to at least one of: at least 1, at least 2, and at least 3.

Whenever the term “no more than” precedes the first numerical value in a series of a plurality of numerical values, the term “no more than” may apply to each of the numerical values in that series of numerical values. In some non-limiting examples, no more than: 3, 2, and 1 may be equivalent to no more than 3, no more than 2, and no more than 1.

Certain examples herein contemplate numerical ranges. When ranges are present, the ranges may include the range endpoints. Additionally, every sub-range and value within the range may be present as if explicitly written out. The terms “about” and “approximately” may mean within an acceptable error range for the particular value, which will depend in part on how the value is measured (determined), including without limitation, the limitations of the measurement system. In some non-limiting examples, “about” may mean within one of: 1, and more than 1, standard deviation, per the practice in the relevant art. In some non-limiting examples, “about” may mean a range of one of no more than about: 20%, 10%, 5%, and 1% of a given value. Where particular values are described in the application and claims, unless otherwise stated the term “about” meaning within an acceptable error range for the particular value may be assumed.

As will be understood by those having ordinary skill in the relevant art, for any and all purposes, particularly in terms of providing a written description, all ranges disclosed herein may also encompass any and all possible sub-ranges, and combinations of sub-ranges thereof. Any listed range may be easily recognized as sufficiently describing, /enabling the same range being broken down at least into equal fractions thereof, including without limitation, halves, thirds, quarters, fifths, tenths etc. As a non-limiting example, each range discussed herein may be readily be broken down into a lower third, middle third, and upper third, etc.

As will be understood by those having ordinary skill in the relevant art, for any and all purposes, particularly in terms of providing a written description, all values/ranges disclosed herein that are described in terms of at least one decimal value, should be interpreted as encompassing a value/range that includes rounding error as would be understood by those having ordinary skill in the art, as determined based on the number of significant digits expressed by such decimal value. For greater certainty, the presence/absence of any additional decimal value, in the present disclosure, the same paragraph, and even the same sentence, as the first decimal value, which may have a greater/lesser number of significant digits than the first decimal value, should not be used to limit the value/range encompassed by such first decimal value, in any fashion that limits the value/range so encompassed, to a value/range that is no more than one that includes rounding error based on the number of significant digits expressed thereby.

As will also be understood by those having ordinary skill in the relevant art, all language, /terminology such as “up to”, “at least”, “at least”, “no more than”, “no more than”, and the like, may include, /refer the recited range(s) and may also refer to ranges that may be subsequently broken down into sub-ranges as discussed herein.

As will be understood by those having ordinary skill in the relevant art, a range may include each individual member of the recited range.

### General

The purpose of the Abstract is to enable the relevant patent office and the public generally, and specifically, persons of ordinary skill in the art who are not familiar with patent/legal terms/phraseology, to quickly determine from a cursory inspection, the nature of the technical disclosure. The Abstract is neither intended to define the scope of this disclosure, nor is it intended to be limiting as to the scope of this disclosure in any way.

All publications, patents, and patent applications mentioned in this specification are herein incorporated by reference to the same extent as if each individual one of: a publication, patent, and patent application, was specifically and individually indicated to be incorporated by reference. To the extent publications, patents, and patent applications incorporated by reference contradict the disclosure contained in the specification, the specification is intended to one of: supersede, and take precedence over, any such contradictory material.

Incorporation by reference is expressly limited to the technical aspects of the materials, systems, and methods described in the mentioned publications, patents, and patent applications and may not extend to any lexicographical definitions from the publications, patents, and patent applications. Any lexicographical definition appearing in the publications, patents, and patent applications that is not also expressly repeated in the instant disclosure should not be treated as such and should not be read as defining any terms appearing in the accompanying claims.

The structure, manufacture and use of the presently disclosed examples have been discussed above. The specific examples discussed are merely illustrative of specific ways to make and use the concepts disclosed herein, and do not limit the scope of the present disclosure. Rather, the general principles set forth herein are merely illustrative of the scope of the present disclosure.

It should be appreciated that the present disclosure, which is described by the claims and not by the implementation details provided, and which can be modified by varying, omitting, adding, replacing, and in the absence of, any element(s), at least one of: limitation(s) with alternatives, and equivalent functional elements, whether specifically disclosed herein, will be apparent to those having ordinary skill in the relevant art, and may be made to the examples disclosed herein, and may provide many applicable inventive concepts that may be embodied in a wide variety of specific contexts, without straying from the present disclosure.

In some non-limiting examples, features, techniques, systems, sub-systems and methods described and illustrated in at least one of the above-described examples, whether described and illustrated as discrete/separate, may be combined/integrated in another system without departing from the scope of the present disclosure, to create alternative examples comprised of a (sub-)combination of features that may not be explicitly described above, including without limitation, where certain features may be omitted/not implemented. Features having applicability for such combinations and sub-combinations would be readily apparent to persons skilled in the art upon review of the present application as a whole. Other examples of changes, substitutions, and alterations are easily ascertainable and could be made without departing from the spirit and scope disclosed herein.

All statements herein reciting principles, aspects, and examples of the disclosure, as well as specific examples thereof, are intended to encompass both structural and functional equivalents thereof and to cover and embrace all applicable changes in technology. Additionally, it is intended that such equivalents include both currently known equivalents as well as equivalents developed in the future, i.e., any elements developed that perform the same function, regardless of structure.

### Clauses

The present disclosure includes, without limitation, the following clauses:

The device according to at least one clause herein wherein the patterning coating comprises a patterning material.

The device according to at least one clause herein, wherein an initial sticking probability against deposition of the deposited material of the patterning coating is no more than an initial sticking probability against deposition of the deposited material of the exposed layer surface.

The device according to at least one clause herein, wherein the patterning coating is substantially devoid of a closed coating of the deposited material.

The device according to at least one clause herein, wherein at least one of the patterning coating and the patterning material has an initial sticking probability against deposition of the deposited material that is one of no more than about: 0.3, 0.2, 0.15, 0.1, 0.08, 0.05, 0.03, 0.02, 0.01, 0.008, 0.005, 0.003, 0.001, 0.0008, 0.0005, 0.0003, and 0.0001.

The device according to at least one clause herein, wherein at least one of the patterning coating and the patterning material has an initial sticking probability against deposition of at least one of silver (Ag) and magnesium (Mg) that is one of no more than about: 0.3, 0.2, 0.15, 0.1, 0.08, 0.05, 0.03, 0.02, 0.01, 0.008, 0.005, 0.003, 0.001, 0.0008, 0.0005, 0.0003, and 0.0001.

The device according to at least one clause herein, wherein at least one of the patterning coating and the patterning material has an initial sticking probability against deposition of the deposited material of one of between about: 0.15-0.0001, 0.1-0.0003, 0.08-0.0005, 0.08-0.0008, 0.05-0.001, 0.03-0.0001, 0.03-0.0003, 0.03-0.0005, 0.03-0.0008, 0.03-0.001, 0.03-0.005, 0.03-0.008, 0.03-0.01, 0.02-0.0001, 0.02-0.0003, 0.02-0.0005, 0.02-0.0008, 0.02-0.001, 0.02-0.005, 0.02-0.008, 0.02-0.01, 0.01-0.0001, 0.01-0.0003, 0.01-0.0005, 0.01-0.0008, 0.01-0.001, 0.01-0.005, 0.01-0.008, 0.008-0.0001, 0.008-0.0003, 0.008-0.0005, 0.008-0.0008, 0.008-0.001, 0.008-0.005, 0.005-0.0001, 0.005-0.0003, 0.005-0.0005, 0.005-0.0008, and 0.005-0.001.

The device according to at least one clause herein, wherein at least one of the patterning coating and the patterning material has an initial sticking probability against deposition of the deposited material that is no more than a threshold value that is one of about: 0.3, 0.2, 0.18, 0.15, 0.13, 0.1, 0.08, 0.05, 0.03, 0.02, 0.01, 0.008, 0.005, 0.003, and 0.001.

The device according to at least one clause herein, wherein at least one of the patterning coating and the patterning material has an initial sticking probability against the deposition of one of: Ag, Mg, ytterbium (Yb), cadmium (Cd), and zinc (Zn), that is no more than the threshold value.

The device according to at least one clause herein, wherein the threshold value has a first threshold value against the deposition of a first deposited material and a second threshold value against the deposition of a second deposited material.

The device according to at least one clause herein, wherein the first deposited material is Ag and the second deposited material is Mg.

The device according to at least one clause herein, wherein the first deposited material is Ag and the second deposited material is Yb.

The device according to at least one clause herein, wherein the first deposited material is Yb and the second deposited material is Mg.

The device according to at least one clause herein, wherein the first threshold value exceeds the second threshold value.

The device according to at least one clause herein, wherein at least one of the patterning coating and the patterning material has a transmittance for EM radiation of at least a threshold transmittance value after being subjected to a vapor flux of the deposited material.

The device according to at least one clause herein, wherein the threshold transmittance value is measured at a wavelength in the visible spectrum.

The device according to at least one clause herein, wherein the threshold transmittance value is one of at least about 60%, 65%, 70%, 75%, 80%, 85%, and 90% of incident EM power transmitted therethrough.

The device according to at least one clause herein, wherein at least one of the patterning coating and the patterning material has a surface energy of one of no more than about: 24 dynes/cm, 22 dynes/cm, 20 dynes/cm, 18 dynes/cm, 16 dynes/cm, 15 dynes/cm, 13 dynes/cm, 12 dynes/cm, and 11 dynes/cm.

The device according to at least one clause herein, wherein at least one of the patterning coating and the patterning material has a surface energy that is one of at least about: 6 dynes/cm, 7 dynes/cm, and 8 dynes/cm.

The device according to at least one clause herein, wherein at least one of the patterning coating and the patterning material has a surface energy that is one of between about: 10-20 dynes/cm, and 13-19 dynes/cm.

The device according to at least one clause herein, wherein at least one of the patterning coating and the patterning material has a refractive index for EM radiation at a wavelength of 550 nm that is one of no more than about: 1.55, 1.5, 1.45, 1.43, 1.4, 1.39, 1.37, 1.35, 1.32, and 1.3

The device according to at least one clause herein, wherein at least one of the patterning coating and the patterning material has an extinction coefficient that is no more than about 0.01 for photons at a wavelength that exceeds one of about: 600 nm, 500 nm, 460 nm, 420 nm, and 410 nm.

The device according to at least one clause herein, wherein at least one of the patterning coating and the patterning material has an extinction coefficient that is one of at least about: 0.05, 0.1, 0.2, 0.5 for EM radiation at a wavelength shorter than one of at least about: 400 nm, 390 nm, 380 nm, and 370 nm.

The device according to at least one clause herein, wherein at least one of the patterning coating and the patterning material has a glass transition temperature that is that is one of: one of at least about: 300° C., 150° C., 130° C., 120° C., and 100° C., and one of no more than about: 30° C., 0° C., −30° C., and −50° C.

The device according to at least one clause herein, wherein the patterning material has a sublimation temperature of one of between about: 100-320° C., 120-300° C., 140-280° C., and 150-250° C.

The device according to at least one clause herein, wherein at least one of the patterning coating and the patterning material comprises at least one of a fluorine atom and a silicon atom.

The device according to at least one clause herein, wherein the patterning coating comprises fluorine and carbon.

The device according to at least one clause herein, wherein an atomic ratio of a quotient of fluorine by carbon is one of about: 1, 1.5, and 2.

The device according to at least one clause herein, wherein the patterning coating comprises an oligomer.

The device according to at least one clause herein, wherein the patterning coating comprises a compound having a molecular structure comprising a backbone and at least one functional group bonded thereto.

The device according to at least one clause herein, wherein the compound comprises at least one of: a siloxane group, a silsesquioxane group, an aryl group, a heteroaryl group, a fluoroalkyl group, a hydrocarbon group, a phosphazene group, a fluoropolymer, and a metal complex.

The device according to at least one clause herein, wherein a molecular weight of the compound is one of no more than about: 5,000 g/mol, 4,500 g/mol, 4,000 g/mol, 3,800 g/mol, and 3,500 g/mol.

The device according to at least one clause herein, wherein the molecular weight is about: 1,500 g/mol, 1,700 g/mol, 2,000 g/mol, 2,200 g/mol, and 2,500 g/mol.

The device according to at least one clause herein, wherein the molecular weight is one of between about: 1,500-5,000 g/mol, 1,500-4,500 g/mol, 1,700-4,500 g/mol, 2,000-4,000 g/mol, 2,200-4,000 g/mol, and 2,500-3,800 g/mol.

The device according to at least one clause herein, wherein a percentage of a molar weight of the compound that is attributable to a presence of fluorine atoms, is one of between about: 40-90%, 45-85%, 50-80%, 55-75%, and 60-75%.

The device according to at least one clause herein, wherein fluorine atoms comprise a majority of the molar weight of the compound.

The device according to at least one clause herein, wherein the patterning material comprises an organic-inorganic hybrid material.

The device according to at least one clause herein, wherein the patterning coating has at least one nucleation site for the deposited material.

The device according to at least one clause herein, wherein the patterning coating is supplemented with a seed material that acts as a nucleation site for the deposited material.

The device according to at least one clause herein, wherein the seed material comprises at least one of: a nucleation promoting coating (NPC) material, an organic material, a polycyclic aromatic compound, and a material comprising a non-metallic element selected from one of oxygen (O), sulfur (S), nitrogen (N), I carbon (C).

The device according to at least one clause herein, wherein the patterning coating acts as an optical coating.

The device according to at least one clause herein, wherein the patterning coating modifies at least one of a property and a characteristic of EM radiation emitted by the device.

The device according to at least one clause herein, wherein the patterning coating comprises a crystalline material.

The device according to at least one clause herein, wherein the patterning coating is deposited as a non-crystalline material and becomes crystallized after deposition.

The device according to at least one clause herein, wherein the deposited layer comprises a deposited material.

The device according to at least one clause herein, wherein the deposited material comprises an element selected from at least one of: potassium (K), sodium (Na), lithium (Li), barium (Ba), cesium (Cs), ytterbium (Yb), silver (Ag), gold (Au), copper (Cu), aluminum (Al), magnesium (Mg), zinc (Zn), cadmium (Cd), tin (Sn), nickel (Ni), and yttrium (Y).

The device according to at least one clause herein, wherein the deposited material comprises a pure metal.

The device according to at least one clause herein, wherein the deposited material is selected from one of pure Ag and substantially pure Ag.

The device according to at least one clause herein, wherein the substantially pure Ag has a purity of one of at least about: 95%, 99%, 99.9%, 99.99%, 99.999%, and 99.9995%.

The device according to at least one clause herein, wherein the deposited material is selected from one of pure Mg and substantially pure Mg.

The device according to at least one clause herein, wherein the substantially pure Mg has a purity of one of at least about: 95%, 99%, 99.9%, 99.99%, 99.999%, and 99.9995%.

The device according to at least one clause herein, wherein the deposited material comprises an alloy.

The device according to at least one clause herein, wherein the deposited material comprises at least one of: an Ag-containing alloy, an Mg-containing alloy, and an AgMg-containing alloy.

The device according to at least one clause herein, wherein the AgMg-containing alloy has an alloy composition that ranges from 1:10 (Ag:Mg) to about 10:1 by volume.

The device according to at least one clause herein, wherein the deposited material comprises at least one metal other than Ag.

The device according to at least one clause herein, wherein the deposited material comprises an alloy of Ag with at least one metal.

The device according to at least one clause herein, wherein the at least one metal is selected from at least one of Mg and Yb.

The device according to at least one clause herein, wherein the alloy is a binary alloy having a composition between about 5-95 vol. % Ag.

The device according to at least one clause herein, wherein the alloy comprises a Yb:Ag alloy having a composition between about 1:20-10:1 by volume.

The device according to at least one clause herein, wherein the deposited material comprises an Mg:Yb alloy.

The device according to at least one clause herein, wherein the deposited material comprises an Ag:Mg:Yb alloy.

The device according to at least one clause herein, wherein the deposited layer comprises at least one additional element.

The device according to at least one clause herein, wherein the at least one additional element is a non-metallic element.

The device according to at least one clause herein, wherein the non-metallic element is selected from at least one of O, S, N, and C.

The device according to at least one clause herein, wherein a concentration of the non-metallic element is one of no more than about: 1%, 0.1%, 0.01%, 0.001%, 0.0001%, 0.00001%, 0.000001%, and 0.0000001%.

The device according to at least one clause herein, wherein the deposited layer has a composition in which a combined amount of O and C is one of no more than about: 10%, 5%, 1%, 0.1%, 0.01%, 0.001%, 0.0001%, 0.00001%, 0.000001%, and 0.0000001%.

The device according to at least one clause herein, wherein the non-metallic element acts as a nucleation site for the deposited material on the NIC.

The device according to at least one clause herein, wherein the deposited material and the underlying layer comprise a metal in common.

The device according to at least one clause herein, the deposited layer comprises a plurality of layers of the deposited material.

The device according to at least one clause herein, a deposited material of a first one of the plurality of layers is different from a deposited material of a second one of the plurality of layers.

The device according to at least one clause herein, wherein the deposited layer comprises a multilayer coating.

The device according to at least one clause herein, wherein the multilayer coating is one of: Yb/Ag, Yb/Mg, Yb/Mg:Ag, Yb/Yb:Ag, Yb/Ag/Mg, and Yb/Mg/Ag.

The device according to at least one clause herein, wherein the deposited material comprises a metal having a bond dissociation energy of one of no more than about: 300 kJ/mol, 200 kJ/mol, 165 kJ/mol, 150 kJ/mol, 100 kJ/mol, 50 kJ/mol, and 20 kJ/mol.

The device according to at least one clause herein, wherein the deposited material comprises a metal having an electronegativity of one of no more than about: 1.4, 1.3, and 1.2.

The device according to at least one clause herein, wherein a sheet resistance of the deposited layer is one of no more than about: 10Ω/□, 5Ω/□, 1Ω/□, 0.5Ω/□, 0.2Ω/□, and 0.1Ω/□.

The device according to at least one clause herein, wherein the deposited layer is disposed in a pattern defined by at least one region therein that is substantially devoid of a closed coating thereof.

The device according to at least one clause herein, wherein the at least one region separates the deposited layer into a plurality of discrete fragments thereof.

The device according to at least one clause herein, wherein at least two discrete fragments are electrically coupled.

The device according to at least one clause herein, wherein the patterning coating has a boundary defined by a patterning coating edge.

The device according to at least one clause herein, wherein the patterning coating comprises at least one patterning coating transition region and a patterning coating non-transition part.

The device according to at least one clause herein, wherein the at least one patterning coating transition region transitions from a maximum thickness to a reduced thickness.

The device according to at least one clause herein, wherein the at least one patterning coating transition region extends between the patterning coating non-transition part and the patterning coating edge.

The device according to at least one clause herein, wherein the patterning coating has an average film thickness in the patterning coating non-transition part that is in a range of one of between about: 1-100 nm, 2-50 nm, 3-30 nm, 4-20 nm, 5-15 nm, 5-10 nm, and 1-10 nm.

The device according to at least one clause herein, wherein a thickness of the patterning coating in the patterning coating non-transition part is within one of about: 95%, and 90% of the average film thickness of the NIC.

The device according to at least one clause herein, wherein the average film thickness is one of no more than about: 80 nm, 60 nm, 50 nm, 40 nm, 30 nm, 20 nm, 15 nm, and 10 nm.

The device according to at least one clause herein, wherein the average film thickness exceeds one of about: 3 nm, 5 nm, and 8 nm.

The device according to at least one clause herein, wherein the average film thickness is no more than about 10 nm.

The device according to at least one clause herein, wherein the patterning coating has a patterning coating thickness that decreases from a maximum to a minimum within the patterning coating transition region.

The device according to at least one clause herein, wherein the maximum is proximate to a boundary between the patterning coating transition region and the patterning coating non-transition part.

The device according to at least one clause herein, wherein the maximum is a percentage of the average film thickness that is one of about: 100%, 95%, and 90%.

The device according to at least one clause herein, wherein the minimum is proximate to the patterning coating edge.

The device according to at least one clause herein, wherein the minimum is in a range of between about: 0-0.1 nm.

The device according to at least one clause herein, wherein a profile of the patterning coating thickness is one of sloped, tapered, and defined by a gradient.

The device according to at least one clause herein, wherein the tapered profile follows one of a linear, non-linear, parabolic, and exponential decaying profile.

The device according to at least one clause herein, wherein a non-transition width along a lateral axis of the patterning coating non-transition region exceeds a transition width along the axis of the patterning coating transition region.

The device according to at least one clause herein, wherein a quotient of the non-transition width by the transition width is one of at least about: 5, 10, 20, 50, 100, 500, 1,000, 1,500, 5,000, 10,000, 50,000, and 100,000.

The device according to at least one clause herein, wherein at least one of the non-transition width and the transition width exceeds an average film thickness of the underlying layer.

The device according to at least one clause herein, wherein at least one of the non-transition width and the transition width exceeds the average film thickness of the patterning coating.

The device according to at least one clause herein, wherein the average film thickness of the underlying layer exceeds the average film thickness of the patterning coating.

The device according to at least one clause herein, wherein the deposited layer has a boundary defined by a deposited layer edge.

The device according to at least one clause herein, wherein the deposited layer comprises at least one deposited layer transition region and a deposited layer non-transition part.

The device according to at least one clause herein, wherein the at least one deposited layer transition region transitions from a maximum thickness to a reduced thickness.

The device according to at least one clause herein, wherein the at least one deposited layer transition region extends between the deposited layer non-transition part and the deposited layer edge.

The device according to at least one clause herein, wherein the deposited layer has an average film thickness in the deposited layer non-transition part that is in a range of one of between about: 1-500 nm, 5-200 nm, 5-40 nm, 10-30 nm, and 10-100 nm.

The device according to at least one clause herein, wherein the average film thickness exceeds one of about: 10 nm, 50 nm, and 100 nm.

The device according to at least one clause herein, wherein the average film thickness of is substantially constant thereacross.

The device according to at least one clause herein, wherein the average film thickness exceeds an average film thickness of the underlying layer.

The device according to at least one clause herein, wherein a quotient of the average film thickness of the deposited layer by the average film thickness of the underlying layer is one of at least about: 1.5, 2, 5, 10, 20, 50, and 100.

The device according to at least one clause herein, wherein the quotient is in a range of one of between about: 0.1-10, and 0.2-40.

The device according to at least one clause herein, wherein the average film thickness of the deposited layer exceeds an average film thickness of the patterning coating.

The device according to at least one clause herein, wherein a quotient of the average film thickness of the deposited layer by the average film thickness of the patterning coating is one of at least about: 1.5, 2, 5, 10, 20, 50, and 100.

The device according to at least one clause herein, wherein the quotient is in a range of one of between about: 0.2-10, and 0.5-40.

The device according to at least one clause herein, wherein a deposited layer non-transition width along a lateral axis of the deposited layer non-transition part exceeds a patterning coating non-transition width along the axis of the patterning coating non-transition part.

The device according to at least one clause herein, wherein a quotient of the patterning coating non-transition width by the deposited layer non-transition width is one of between about: 0.1-10, 0.2-5, 0.3-3, and 0.4-2.

The device according to at least one clause herein, wherein a quotient of the deposited layer non-transition width by the patterning coating non-transition width is one of at least: 1, 2, 3, and 4.

The device according to at least one clause herein, wherein the deposited layer non-transition width exceeds the average film thickness of the deposited layer.

The device according to at least one clause herein, wherein a quotient of the deposited layer non-transition width by the average film thickness is at least one of about: 10, 50, 100, and 500.

The device according to at least one clause herein, wherein the quotient is no more than about 100,000.

The device according to at least one clause herein, wherein the deposited layer has a deposited layer thickness that decreases from a maximum to a minimum within the deposited layer transition region.

The device according to at least one clause herein, wherein the maximum is proximate to a boundary between the deposited layer transition region and the deposited layer non-transition part.

The device according to at least one clause herein, wherein the maximum is the average film thickness.

The device according to at least one clause herein, wherein the minimum is proximate to the deposited layer edge.

The device according to at least one clause herein, wherein the minimum is in a range of between about: 0-0.1 nm.

The device according to at least one clause herein, wherein the minimum is the average film thickness.

The device according to at least one clause herein, wherein a profile of the deposited layer thickness is one of sloped, tapered, and defined by a gradient.

The device according to at least one clause herein, wherein the tapered profile follows one of a linear, non-linear, parabolic, and exponential decaying profile.

The device according to at least one clause herein, wherein the deposited layer comprises a discontinuous layer in at least a part of the deposited layer transition region.

The device according to at least one clause herein, wherein the deposited layer overlaps the patterning coating in an overlap portion.

The device according to at least one clause herein, wherein the patterning coating overlaps the deposited layer in an overlap portion.

The device according to at least one clause herein, further comprising at least one particle structure disposed on an exposed layer surface of an underlying layer.

The device according to at least one clause herein, wherein the underlying layer is the patterning coating.

The device according to at least one clause herein, wherein the at least one particle structure comprises a particle material.

The device according to at least one clause herein, wherein the particle material is the same as the deposited material.

The device according to at least one clause herein, wherein at least two of the particle material, the deposited material, and a material of which the underlying layer is comprised, comprises a metal in common.

The device according to at least one clause herein, wherein the particle material comprises an element selected from at least one of: potassium (K), sodium (Na), lithium (Li), barium (Ba), cesium (Cs), ytterbium (Yb), silver (Ag), gold (Au), copper (Cu), aluminum (Al), magnesium (Mg), zinc (Zn), cadmium (Cd), tin (Sn), nickel (Ni), and yttrium (Y).

The device according to at least one clause herein, wherein the particle material comprises a pure metal.

The device according to at least one clause herein, wherein the particle material is selected from one of pure Ag and substantially pure Ag.

The device according to at least one clause herein, wherein the substantially pure Ag has a purity of one of at least about: 95%, 99%, 99.9%, 99.99%, 99.999%, and 99.9995%.

The device according to at least one clause herein, wherein the particle material is selected from one of pure Mg and substantially pure Mg.

The device according to at least one clause herein, wherein the substantially pure Mg has a purity of one of at least about: 95%, 99%, 99.9%, 99.99%, 99.999%, and 99.9995%.

The device according to at least one clause herein, wherein the particle material comprises an alloy.

The device according to at least one clause herein, wherein the particle material comprises at least one of: an Ag-containing alloy, an Mg-containing alloy, and an AgMg-containing alloy.

The device according to at least one clause herein, wherein the AgMg-containing alloy has an alloy composition that ranges from 1:10 (Ag:Mg) to about 10:1 by volume.

The device according to at least one clause herein, wherein the particle material comprises at least one metal other than Ag.

The device according to at least one clause herein, wherein the particle material comprises an alloy of Ag with at least one metal.

The device according to at least one clause herein, wherein the at least one metal is selected from at least one of Mg and Yb.

The device according to at least one clause herein, wherein the alloy is a binary alloy having a composition between about 5-95 vol. % Ag.

The device according to at least one clause herein, wherein the alloy comprises a Yb:Ag alloy having a composition between about 1:20-10:1 by volume.

The device according to at least one clause herein, wherein the particle material comprises an Mg:Yb alloy.

The device according to at least one clause herein, wherein the particle material comprises an Ag:Mg:Yb alloy.

The device according to at least one clause herein, wherein the at least one particle structure comprises at least one additional element.

The device according to at least one clause herein, wherein the at least one additional element is a non-metallic element.

The device according to at least one clause herein, wherein the non-metallic element is selected from at least one of O, S, N, and C.

The device according to at least one clause herein, wherein a concentration of the non-metallic element is one of no more than about: 1%, 0.1%, 0.01%, 0.001%, 0.0001%, 0.00001%, 0.000001%, and 0.0000001%.

The device according to at least one clause herein, wherein the at least one particle structure has a composition in which a combined amount of O and C is one of no more than about: 10%, 5%, 1%, 0.1%, 0.01%, 0.001%, 0.0001%, 0.00001%, 0.000001%, and 0.0000001%.

The device according to at least one clause herein, wherein the at least one particle is disposed at an interface between the patterning coating and at least one overlying layer in the device.

The device according to at least one clause herein, wherein the at least one particle is in physical contact with an exposed layer surface of the patterning coating.

The device according to at least one clause herein, wherein the at least one particle structure affects at least one optical property of the device.

The device according to at least one clause herein, wherein the at least one optical property is controlled by selection of at least one property of the at least one particle structure selected from at least one of: a characteristic size, a length, a width, a diameter, a height, a size distribution, a shape, a surface coverage, a configuration, a deposited density, a dispersity, and a composition.

The device according to at least one clause herein, wherein the at least one property of the at least one particle structure is controlled by selection of at least one of: at least one characteristic of the patterning material, an average film thickness of the patterning coating, at least one heterogeneity in the patterning coating, and a deposition environment for the patterning coating, selected from at least one of a temperature, pressure, duration, deposition rate, and deposition process.

The device according to at least one clause herein, wherein the at least one property of the at least one particle structure is controlled by selection of at least one of: at least one characteristic of the particle material, an extent to which the patterning coating is exposed to deposition of the particle material, a thickness of the discontinuous layer, and a deposition environment for the particle material, selected from at least one of a temperature, pressure, duration, deposition rate, and deposition process.

The device according to at least one clause herein, wherein the at least one particle structures are disconnected from one another.

The device according to at least one clause herein, wherein the at least one particle structure forms a discontinuous layer.

The device according to at least one clause herein, wherein the discontinuous layer is disposed in a pattern defined by at least one region therein that is substantially devoid of the at least one particle structure.

The device according to at least one clause herein, wherein a characteristic of the discontinuous layer is determined by an assessment according to at least one criterion selected from one of: a characteristic size, length, width, diameter, height, size distribution, shape, configuration, surface coverage, deposited distribution, dispersity, presence of aggregation instances, and extent of such aggregation instances.

The device according to at least one clause herein, wherein the assessment is performed by determining at least one attribute of the discontinuous layer by an applied imaging technique selected from one of: electron microscopy, atomic force microscopy, and scanning electron microscopy.

The device according to at least one clause herein, wherein the assessment is performed across an extent defined by at least one observation window.

The device according to at least one clause herein, wherein the at least one observation window is located at one of: a perimeter, interior location, and grid coordinate of the lateral aspect.

The device according to at least one clause herein, wherein the observation window corresponds to a field of view of the applied imaging technique.

The device according to at least one clause herein, wherein the observation window corresponds to a magnification level selected from one of: 2.00 μm, 1.00 μm, 500 nm, and 200 nm.

The device according to at least one clause herein, wherein the assessment incorporates at least one of: manual counting, curve fitting, polygon fitting, shape fitting, and an estimation technique.

The device according to at least one clause herein, wherein the assessment incorporates a manipulation selected from one of: an average, median, mode, maximum, minimum, probabilistic, statistical, and data calculation.

The device according to at least one clause herein, wherein the characteristic size is determined from at least one of: a mass, volume, diameter, perimeter, major axis, and minor axis of the at least one particle structure.

The device according to at least one clause herein, wherein the dispersity is determined from:

$$D = \frac{\overline{{S}_{s}}}{\overline{{S}_{n}}}$$

$$where :$$

$$\overline{{S}_{s}} = \frac{{\sum }_{i = 1}^{n} {S}_{i}^{2}}{{\sum }_{i = 1}^{n} {S}_{i}} , \overline{{S}_{n}} = \frac{{\sum }_{i = 1}^{n} {S}_{i}}{n} ,$$

n is the number of particles in a sample area,
Sᵢ is the (area) size of the i^{th} particle,
S̅ₙ is the number average of the particle (area) sizes; and
S̅ₛ is the (area) size average of the particle (area) sizes.

Accordingly, the specification and the examples disclosed therein are to be considered illustrative only, with a true scope of the disclosure being disclosed by the following numbered claims:

What is claimed is:

1. A layered semiconductor device comprising a compound, the compound comprising a heterocyclic moiety and at least one fluorine (F)-containing moiety each bonded thereto, wherein the heterocyclic moiety comprises nitrogen (N).

2. The device of claim 1, wherein the compound comprises a plurality of F-containing moieties.

3. The device of claim 1, wherein the heterocyclic moiety is a heterocyclic moiety of between about 5-24 members.

4. The device of claim 1, wherein the F-containing moiety is bonded to the heterocyclic moiety by a linker moiety.

5. The device of claim 1, wherein a molecular structure of the compound is represented by one of: Formula (HC-1), and Formula (HC-2): 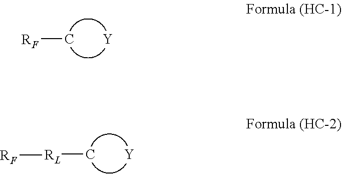
wherein:
Y represents a member comprising at least one N atom of the heterocyclic moiety;
R_{F} represents the F-containing moiety; and
R_{L} represents a linker moiety.

6. The device of claim 5, wherein Y represents a member of between about 4-17 atoms.

7. The device of claim 1, wherein the F-containing moiety comprises at least one of: a substituted fluoroalkyl, an unsubstituted fluoroalkyl, a substituted fluoroalkoxy, an unsubstituted fluoroalkoxy, a substituted fluoroalkylsiloxy, an unsubstituted fluoroalkylsiloxy, a substituted fluorocycloalkyl, an unsubstituted fluorocycloalkyl, a substituted fluoroaryl, and an unsubstituted fluoroaryl.

8. The device of claim 1, wherein the F-containing moiety comprises a terminal unit comprising one of: CF₂CF₂H, CF₂CF₃, CH₂CF₂H, and CH₂CF₃.

9. The device of claim 1, wherein the heterocyclic moiety comprises at least one of: a monocyclic structure, a polycyclic structure, and a fused polycyclic structure.

10. The device of claim 1, wherein the heterocyclic moiety is a heteroaryl moiety.

11. The device of claim 1, wherein the heterocyclic moiety is a six-membered heterocyclic moiety.

12. The device of claim 1, wherein the heterocyclic moiety is a triazine moiety.

13. The device of claim 1, wherein the F-containing moiety comprises at least one of: a fluoroalkyl moiety, and a fluoroalkoxy moiety.

14. The device of claim 4, wherein the linker moiety comprises at least one of: an ether, a secondary amine, a tertiary amine, a substituted alkylene, an unsubstituted alkylene, a substituted fluoroalkylene, an unsubstituted fluoroalkylene, a substituted aryl, an unsubstituted aryl, a substituted fluoroaryl, an unsubstituted fluoroaryl, a substituted heteroaryl, and an unsubstituted heteroaryl, group.

15. The device of claim 1, wherein the F-containing moiety comprises at least one of: a C₃-C₁₅ fluoroalkyl, and a C₃-C₁₅ fluoroalkoxy.

16. The device of claim 1, further comprising first and second electrodes, and an active region comprising at least one semiconducting layer, the active region bounded, in a longitudinal aspect of the device by the electrodes and confined, in a lateral aspect of the device, to an emissive region defined by the electrodes.

17. The device of claim 16, wherein the active region is substantially devoid of the compound.

18. The device of claim 1, further comprising:
a patterning coating comprising the compound and disposed on a first layer surface of an underlying layer, in a first portion of the lateral aspect; and
a deposited layer comprising a deposited material and disposed on a second layer surface;
wherein the first portion is substantially devoid of a closed coating of the deposited material.

19. The device of claim 18, wherein the first portion excludes at least a part of the emissive region.

20. The device of claim 18, wherein the second electrode comprises at least a part of the deposited layer as a layer thereof.

21. The device of claim 18, wherein the first portion includes at least a part of the emissive region.

22. The device of claim 18, further comprising an auxiliary electrode comprising the deposited layer as a layer thereof.

23. The device of claim 18, further comprising a conductor electrically coupled with the second electrode.
